(19) United States
(12) Patent Application Publication
(10) Pub. No.: US 2020/0181104 A1
(43) Pub. Date: Jun. 11, 2020

(54) SSAO INHIBITORS AND USES THEREOF

(71) Applicant: METACRINE, INC., San Diego, CA (US)

(72) Inventors: Nicholas D. Smith, San Diego, CA (US); Andrew R. Hudson, San Diego, CA (US); Mi Chen, San Diego, CA (US); Johnny Y. Nagasawa, San Diego, CA (US); Iriny Botrous, San Diego, CA (US)

(21) Appl. No.: 16/791,935

(22) Filed: Feb. 14, 2020

Related U.S. Application Data
(63) Continuation of application No. PCT/US2019/057707, filed on Oct. 23, 2019.
(60) Provisional application No. 62/886,077, filed on Aug. 13, 2019.
(60) Provisional application No. 62/795,386, filed on Jan. 22, 2019.
(60) Provisional application No. 62/750,063, filed on Oct. 24, 2018.

(51) Int. Cl. C07D 305/14; C07C 237/24; C07D 309/14; C07D 295/135; C07D 257/04; C07D 211/62; C07D 205/04; C07D 307/22; C07D 241/04; C07D 263/32; C07D 267/10; C07D 498/08; C07D 493/08; C07D 311/96; C07D 401/04; C07D 239/42; C07D 241/12; C07D 213/64; C07D 491/10; C07C 311/14

(52) U.S. Cl. CPC C07D 305/14; C07C 311/14; C07D 309/14; C07D 295/135; C07D 257/04; C07D 211/62; C07D 205/04; C07D 307/22; C07D 241/04; C07D 263/32; C07D 267/10; C07D 498/08; C07D 493/08; C07D 311/96; C07D 401/04; C07D 239/42; C07D 241/12; C07D 213/64; C07D 491/10; C07C 237/24

(57) ABSTRACT

Described herein are compounds that are semicarbazide-sensitive amine oxidase (SSAO) inhibitors, methods of making such compounds, pharmaceutical compositions and medicaments comprising such compounds, and methods of using such compounds in treating or preventing a liver disease or condition.

### CROSS-REFERENCE

This application is a continuation of International Application No. PCT/US2019/057707, filed Oct. 23, 2019, which claims the benefit of U.S. Provisional Application No. 62/750,063, filed on Oct. 24, 2018, U.S. Provisional Application No. 62/795,386, filed on Jan. 22, 2019, and U.S. Provisional Application No. 62/886,077, filed on Aug. 13, 2019, all of which are herein incorporated by reference in their entirety.

### FIELD OF THE INVENTION

Described herein are compounds that are semicarbazide-sensitive amine oxidase (SSAO), methods of making such compounds, pharmaceutical compositions and medicaments comprising such compounds, and methods of using such compounds in the treatment of conditions, diseases, or disorders associated with SSAO activity.

### BACKGROUND OF THE INVENTION

Semicarbazide-sensitive amine oxidase (SSAO) is a member of the semicarbazide-sensitive amino oxidase family, and is also known as AOC3 (amine oxidase, copper containing 3) or VAP-1 (vascular adhesion protein 1). SSAO is an enzyme that exists both as a membrane-bound and a soluble isoform. It is highly expressed in the lung, aorta, liver and ileum. SSAO has been implicated in the pathogenesis of liver diseases (Weston, C. J. et al., J Neural. Transm. 2011, 118, 1055-1064). SSAO inhibition is a treatment modality for liver diseases or conditions such as fatty liver disease.

### SUMMARY OF THE INVENTION

In one aspect, described herein are SSAO inhibitors and uses thereof. In one aspect, described herein is a compound that has the structure of Formula (I), or a pharmaceutically acceptable salt or solvate thereof:

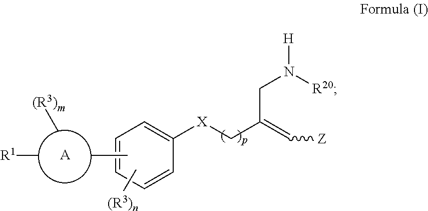

wherein,

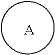

O is a C₃₋₁₀cycloalkyl ring;
X is —O—, —S—, —S(O)₂—, —N(R¹³)—, or —C(R¹³)₂—;
Z is H, F, or Cl;
R¹ is halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁴, —SR⁴, —N(R⁴)(R⁵), —C(O)OR⁴, —OC(O)N(R⁴)(R⁵), —N(R⁶)C(O)N(R⁴)(R⁵), —N(R⁶)C(O)OR⁷, —N(R⁶)S(O)₂R⁷, —C(O)R⁷, —S(O)R⁷, —OC(O)R⁷, —C(O)N(R⁴)(R⁵), —C(O)C(O)N(R⁴)(R⁵), —N(R⁶)C(O)R⁷, —S(O)₂R⁷, —S(O)₂N(R⁴)(R⁵)—, S(═O)(═NH)N(R⁴)(R⁵), —CH₂C(O)N(R⁴)(R⁵), —CH₂N(R⁶)C(O)R⁷, —CH₂S(O)₂R⁷, or —CH₂S(O)₂N(R⁴)(R⁵), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a};
each R² and each R³ are each independently selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)R¹², —S(O)₂R¹², —S(O)₂N(R⁹)(R¹⁰), and —OC(O)R¹², wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14b};
R⁴ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14c};
R⁵ is selected from H, C₁₋₆alkyl, and C₁₋₆haloalkyl; or R⁴ and R⁵, together with the nitrogen to which they are attached, form a C₂₋₉heterocycloalkyl ring optionally substituted with one, two, or three R^{14d};
R⁶ is selected from H, C₁₋₆alkyl, and C₁₋₆haloalkyl;
R⁷ is selected from C₁₋₆alkyl, C₂₋₆alkenyl, C₂₋₆alkynyl, C₃₋₆cycloalkyl, C₂₋₉heterocycloalkyl,
C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14e};
each R⁸ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆ alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14f};
each R⁹ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14g};
each R¹⁰ is independently selected from H and C₁₋₆alkyl; or R⁹ and R¹⁰, together with the nitrogen to which they are attached, form a C₂₋₉heterocycloalkyl ring optionally substituted with one, two, or three R^{14h};
each R¹¹ is independently selected from H, C₁₋₆alkyl, and C₁₋₆haloalkyl;
each R¹² is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R¹⁴ⁱ;
each R¹³ is independently selected from H, C₁₋₆alkyl, and C₁₋₆haloalkyl;
each R^{14a}, R^{14b}, R^{14c}, R^{14d}, R^{14e}, R^{14f}, R^{14g}, R^{14h}, and R¹⁴ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), —OCH₂C(O)OR¹⁶, and —OC(O)R¹⁹, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R⁸)C(O)R¹⁹, —N(R⁸)S(O)₂R¹⁹, —C(O)R¹⁹, —S(O)₂R¹⁹, —S(O)₂N(R¹⁶)(R⁷), and —OC(O)R¹⁹;
each R¹⁵ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁶ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁷ is independently selected from H and C₁₋₆alkyl; or R¹⁶ and R¹⁷, together with the nitrogen to which they are attached, form a C₂₋₉heterocycloalkyl ring;
each R¹⁸ is independently selected from H and C₁₋₆alkyl;
each R¹⁹ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁰ is selected from H and C₁₋₆alkyl;
m is 0, 1, 2, 3, or 4;
n is 0, 1, 2, 3, or 4; and
p is 0 or 1.

Any combination of the groups described above for the various variables is contemplated herein. Throughout the specification, groups and substituents thereof are chosen by one skilled in the field to provide stable moieties and compounds.

In some embodiments is a compound of Formula (I), or a pharmaceutically acceptable salt or solvate thereof, wherein m is 0.

In some embodiments is a compound of Formula (I), or a pharmaceutically acceptable salt or solvate thereof, having the structure of Formula (Ia):

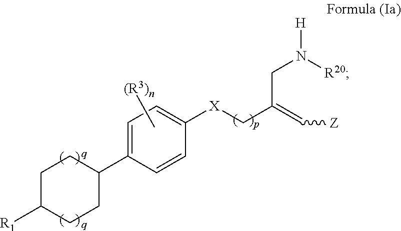

wherein each q is independently 0, 1, or 2.

In some embodiments is a compound of Formula (I), or a pharmaceutically acceptable salt or solvate thereof, having the structure of Formula (Ia′):

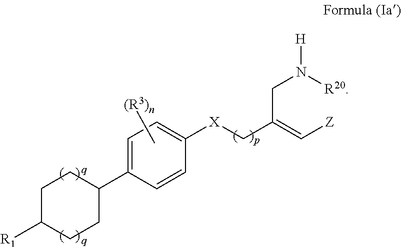

In some embodiments is a compound of Formula (I), or a pharmaceutically acceptable salt or solvate thereof, having the structure of Formula (Iaa):

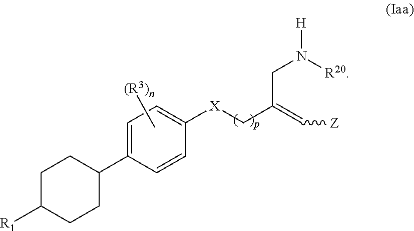

In some embodiments is a compound of Formula (I), or a pharmaceutically acceptable salt or solvate thereof, having the structure of Formula (Iaa′):

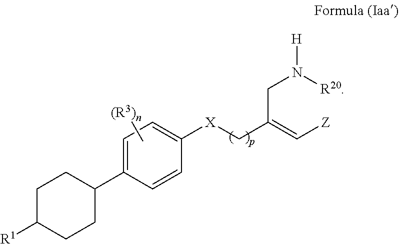

In some embodiments is a compound of Formula (I), or a pharmaceutically acceptable salt or solvate thereof, having the structure of Formula (Ib):

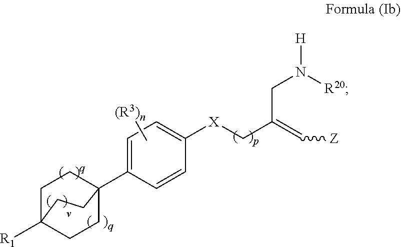

wherein each q is independently 0, 1, or 2; and v is 0, 1, or 2.

In some embodiments is a compound of Formula (I), or a pharmaceutically acceptable salt or solvate thereof, having the structure of Formula (Ib′):

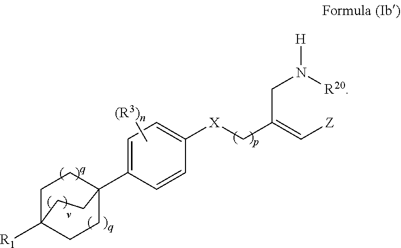

In some embodiments is a compound of Formula (I), or a pharmaceutically acceptable salt or solvate thereof, having the structure of Formula (Ibb):

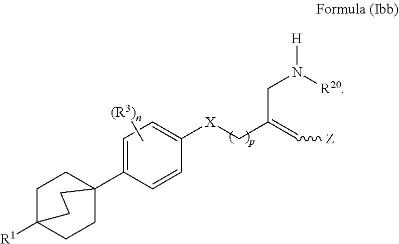

In some embodiments is a compound of Formula (I), or a pharmaceutically acceptable salt or solvate thereof, having the structure of Formula (Ibb′):

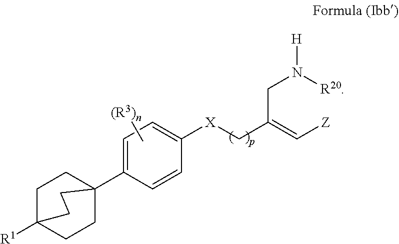

In some embodiments is a compound of Formula (I), or a pharmaceutically acceptable salt or solvate thereof, having the structure of Formula (Ic):

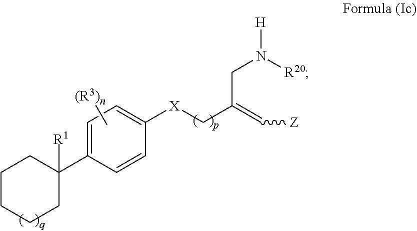

wherein q is 0, 1, or 2.

In some embodiments is a compound of Formula (I), or a pharmaceutically acceptable salt or solvate thereof, having the structure of Formula (Ic′):

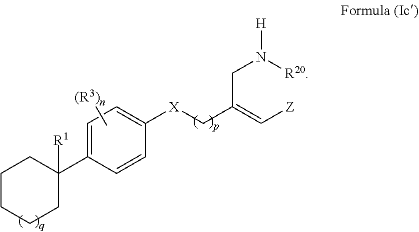

In some embodiments is a compound of Formula (I), (Ia), (Ib), or (Ic), or a pharmaceutically acceptable salt or solvate thereof, wherein R¹ is —OR⁴, —C(O)OR⁴, —OC(O)N(R⁴)(R⁵), —N(R⁶)C(O)R⁷, —N(R⁶)C(O)N(R⁴)(R⁵), —N(R⁶)C(O)OR⁷, —N(R⁶)S(O)₂R⁷, —C(O)R⁷, —C(O)N(R⁴)(R⁵), —C(O)C(O)N(R⁴)(R⁵), —S(O)₂R⁷, —S(O)₂N(R⁴)(R⁵), —S(═O)(═NH)N(R⁴)(R⁵), —CH₂C(O)N(R⁴)(R⁵), —CH₂S(O)₂R⁷, or —CH₂S(O)₂N(R⁴)(R⁵). In some embodiments is a compound of Formula (I), (Ia), (Ib), or (Ic), or a pharmaceutically acceptable salt or solvate thereof, wherein R¹ is —OR⁴, —N(R⁶)C(O)R⁷, —N(R⁶)C(O)N(R⁴)(R⁵), —N(R⁶)S(O)₂R⁷, —C(O)R⁷, —C(O)N(R⁴)(R⁵), or —S(O)₂N(R⁴)(R⁵). In some embodiments is a compound of Formula (I), (Ia), (Ib), or (Ic), or a pharmaceutically acceptable salt or solvate thereof, wherein R¹ is —C(O)N(R⁴)(R⁵). In some embodiments is a compound of Formula (I), (Ia), (Ib), or (Ic), or a pharmaceutically acceptable salt or solvate thereof, wherein R⁴ is selected from H, C₁₋₆alkyl, C₃₋₆cycloalkyl, C₂₋₉heterocycloalkyl, C₆₋₁₀aryl, and C₁₋₉heteroaryl, wherein C₁-6alkyl, C₃₋₆cycloalkyl, C₂₋₉heterocycloalkyl, C₆₋₁₀aryl, and C₁₋₉heteroaryl are optionally substituted with one, two, or three R^{14c}. In some embodiments is a compound of Formula (I), (Ia), (Ib), or (Ic), or a pharmaceutically acceptable salt or solvate thereof, wherein R⁴ is selected from H, C₁₋₆alkyl, C₃₋₆cycloalkyl, and C₂₋₉heterocycloalkyl, wherein C₁₋₆alkyl, C₃₋₆cycloalkyl, and C₂₋₉heterocycloalkyl are optionally substituted with one, two, or three R^{14c}. In some embodiments is a compound of Formula (I), (Ia), (Ib), or (Ic), or a pharmaceutically acceptable salt or solvate thereof, wherein R⁴ is selected from H, C₁₋₆alkyl, and C₂₋₉heterocycloalkyl, wherein C₁₋₆alkyl and C₂₋₉heterocycloalkyl are optionally substituted with one, two, or three R^{14c} In some embodiments is a compound of Formula (I), (Ia), (Ib), or (Ic), or a pharmaceutically acceptable salt or solvate thereof, wherein R⁴ is H. In some embodiments is a compound of Formula (I), (Ia), (Ib), or (Ic), or a pharmaceutically acceptable salt or solvate thereof, wherein R⁴ is unsubstituted C₁₋₆alkyl. In some embodiments is a compound of Formula (I), (Ia), (Ib), or (Ic), or a pharmaceutically acceptable salt or solvate thereof, wherein R⁴ is —CH₃. In some embodiments is a compound of Formula (I), (Ia), (Ib), or (Ic), or a pharmaceutically acceptable salt or solvate thereof, wherein R⁴ is unsubstituted C₂₋₉heterocycloalkyl. In some embodiments is a compound of Formula (I), (Ia), (Ib), or (Ic), or a pharmaceutically acceptable salt or solvate thereof, wherein C₃₋₆cycloalkyl optionally substituted with one or two R^{14c}, In some embodiments is a compound of Formula (I), (Ia), (Ib), or (Ic), or a pharmaceutically acceptable salt or solvate thereof, wherein R⁵ is H. In some embodiments is a compound of Formula (I), (Ia), (Ib), or (Ic), or a pharmaceutically acceptable salt or solvate thereof, wherein R⁵ is unsubstituted C₁₋₆alkyl. In some embodiments is a compound of Formula (I), (Ia), (Ib), or (Ic), or a pharmaceutically acceptable salt or solvate thereof, wherein R⁵ is —CH₃. In some embodiments is a compound of Formula (I), (Ia), (Ib), or (Ic), or a pharmaceutically acceptable salt or solvate thereof, wherein R⁴ and R⁵, together with the nitrogen to which they are attached, form a C₂₋₉heterocycloalkyl ring optionally substituted with one, two, or three R^{14d}. In some embodiments is a compound of Formula (I), (Ia), (Ib), or (Ic), or a pharmaceutically acceptable salt or solvate thereof, wherein R⁴ and R⁵, together with the nitrogen to which they are attached, form a spirocyclic C₂₋₉heterocycloalkyl ring optionally substituted with one, two, or three R^{14d} In some embodiments is a compound of Formula (I), (Ia), (Ib), or (Ic), or a pharmaceutically acceptable salt or solvate thereof, wherein R⁷ is selected from C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14e}. In some embodiments is a compound of Formula (I), (Ia), (Ib), or (Ic), or a pharmaceutically acceptable salt or solvate thereof, wherein R⁷ is selected from C₁₋₆alkyl, C₂₋₉heterocycloalkyl, and C₆₋₁₀aryl, wherein C₁₋₆alkyl, C₂₋₉heterocycloalkyl, and C₆₋₁₀aryl are optionally substituted with one, two, or three R^{14e}. In some embodiments is a compound of Formula (I), (Ia), (Ib), or (Ic), or a pharmaceutically acceptable salt or solvate thereof, wherein R⁷ is C₁₋₆alkyl optionally substituted with one, two, or three R^{14e}. In some embodiments is a compound of Formula (I), (Ia), (Ib), or (Ic), or a pharmaceutically acceptable salt or solvate thereof, wherein R⁷ is unsubstituted C₁₋₆alkyl. In some embodiments is a compound of Formula (I), (Ia), (Ib), or (Ic), or a pharmaceutically acceptable salt or solvate thereof, wherein R⁷ is —CH₃. In some embodiments is a compound of Formula (I), (Ia), (Ib), or (Ic), or a pharmaceutically acceptable salt or solvate thereof, wherein R⁷ is unsubstituted C₂₋₉heterocycloalkyl. In some embodiments is a compound of Formula (I), (Ia), (Ib), or (Ic), or a pharmaceutically acceptable salt or solvate thereof, R⁶ is H. In some embodiments is a compound of Formula (I), (Ia), (Ib), or (Ic), or a pharmaceutically acceptable salt or solvate thereof, wherein R¹ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a}. In some embodiments is a compound of Formula (I), (Ia), (Ib), or (Ic), or a pharmaceutically acceptable salt or solvate thereof, wherein R¹ is C₁₋₆alkyl, C₂₋₉heterocycloalkyl, C₆₋₁₀aryl, or C₁₋₉heteroaryl, wherein C₁₋₆alkyl, C₂₋₉heterocycloalkyl, C₆₋₁₀aryl, and C₁₋₉heteroaryl are optionally substituted with one, two, or three R^{14a}. In some embodiments is a compound of Formula (I), (Ia), (Ib), or (Ic), or a pharmaceutically acceptable salt or solvate thereof, wherein R¹ is C₁₋₆alkyl optionally substituted with one, two, or three R^{14a}. In some embodiments is a compound of Formula (I), (Ia), (Ib), or (Ic), or a pharmaceutically acceptable salt or solvate thereof, wherein R¹ is C₁₋₉heteroaryl optionally substituted with one, two, or three R^{14a}. In some embodiments is a compound of Formula (I), (Ia), (Ib), or (Ic), or a pharmaceutically acceptable salt or solvate thereof, wherein each R³ is independently selected from halogen, —CN, C₁₋₆alkyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)₂R¹², —S(O)₂N(R⁹)(R¹⁰), and —OC(O)R¹², wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14b}. In some embodiments is a compound of Formula (I), (Ia), (Ib), or (Ic), or a pharmaceutically acceptable salt or solvate thereof, wherein each R³ is independently selected from halogen, —CN, C₁₋₆alkyl, C₂₋₉heterocycloalkyl, C₁₋₉heteroaryl, —OR⁸, —N(R⁹)(R¹⁰), —C(O)OR⁹, —C(O)N(R⁹)(R¹⁰), —C(O)R¹², —S(O)₂R¹², S(O)₂N(R⁹)(R¹⁰), wherein C₁₋₆alkyl, C₂₋₉heterocycloalkyl, and C₁₋₉heteroaryl are optionally substituted with one, two, or three R^{14b}. In some embodiments is a compound of Formula (I), (Ia), (Ib), or (Ic), or a pharmaceutically acceptable salt or solvate thereof, wherein each R³ is independently selected from halogen, —CN, C₁₋₆alkyl, —OR⁸, —N(R⁹)(R¹⁰), wherein C₁₋₆alkyl is optionally substituted with one, two, or three R^{14b}. In some embodiments is a compound of Formula (I), (Ia), (Ib), or (Ic), or a pharmaceutically acceptable salt or solvate thereof, wherein n is 1. In some embodiments is a compound of Formula (I), (Ia), (Ib), or (Ic), or a pharmaceutically acceptable salt or solvate thereof, n is 2. In some embodiments is a compound of Formula (I), (Ia), (Ib), or (Ic), or a pharmaceutically acceptable salt or solvate thereof, wherein n is 0. In some embodiments is a compound of Formula (I), (Ia), (Ib), or (Ic), or a pharmaceutically acceptable salt or solvate thereof, wherein X is —O—. In some embodiments is a compound of Formula (I), (Ia), (Ib), or (Ic), or a pharmaceutically acceptable salt or solvate thereof, wherein X is —S(O)₂—. In some embodiments is a compound of Formula (I), (Ia), (Ib), or (Ic), or a pharmaceutically acceptable salt or solvate thereof, wherein X is —CH₂—. In some embodiments is a compound of Formula (I), (Ia), (Ib), or (Ic), or a pharmaceutically acceptable salt or solvate thereof, wherein R²⁰ is H. In some embodiments is a compound of Formula (I), (Ia), (Ib), or (Ic), or a pharmaceutically acceptable salt or solvate thereof, wherein R²⁰ is C₁₋₆alkyl. In some embodiments is a compound of Formula (I), (Ia), (Ib), or (Ic), or a pharmaceutically acceptable salt or solvate thereof, wherein Z is F. In some embodiments is a compound of Formula (I), (Ia), (Ib), or (Ic), or a pharmaceutically acceptable salt or solvate thereof, wherein Z is C₁. In some embodiments is a compound of Formula (I), (Ia), (Ib), or (Ic), or a pharmaceutically acceptable salt or solvate thereof, wherein Z is H. In some embodiments is a compound of Formula (I), (Ia), (Ib), or (Ic), or a pharmaceutically acceptable salt or solvate thereof, p is 0. In some embodiments is a compound of Formula (I), (Ia), (Ib), or (Ic), or a pharmaceutically acceptable salt or solvate thereof, wherein p is 1.

In another aspect, described herein is a pharmaceutical composition comprising a compound of Formula (I), (Ia), (Ib), or (Ic), or a pharmaceutically acceptable salt or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In another aspect, described herein is a method of treating a disease or condition in a mammal that would benefit from SSAO inhibition comprising administering a compound of Formula (I), (Ia), (Ib), or (Ic), or a pharmaceutically acceptable salt or solvate thereof, to the mammal in need thereof. In some embodiments, the disease or condition is a liver condition.

In some embodiments, the compound is administered to the mammal by intravenous administration, subcutaneous administration, oral administration, inhalation, nasal administration, dermal administration, or ophthalmic administration.

In another aspect, described herein is a method of treating or preventing any one of the diseases or conditions described herein comprising administering a therapeutically effective amount of a compound of Formula (I), (Ia), (Ib), or (Ic), or a pharmaceutically acceptable salt or solvate thereof, to a mammal in need thereof.

In another aspect, described herein is a method for the treatment or prevention of a liver condition in a mammal comprising administering a therapeutically effective amount of a compound of Formula (I), (Ia), (Ib), or (Ic), or a pharmaceutically acceptable salt or solvate thereof, to the mammal in need thereof. In other embodiments, the liver condition is amenable to treatment with an SSAO inhibitor. In some embodiments, the method further comprises administering a second therapeutic agent to the mammal in addition to the compound described herein, or a pharmaceutically acceptable salt, or solvate thereof.

In another aspect, described herein is a method of treating or preventing a liver disease or condition in a mammal, comprising administering to the mammal a compound of Formula (I), (Ia), (Ib), or (Ic), or a pharmaceutically acceptable salt or solvate thereof. In some embodiments, the liver disease or condition is nonalcoholic steatohepatitis (NASH), or nonalcoholic fatty liver disease (NAFLD). In some embodiments, the non-alcoholic liver disease or condition is nonalcoholic steatohepatitis (NASH). In some embodiments, the non-alcoholic liver disease or condition is nonalcoholic steatohepatitis (NASH) and is accompanied by liver fibrosis. In some embodiments, the non-alcoholic liver disease or condition is nonalcoholic steatohepatitis (NASH) without liver fibrosis.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by inhalation; and/or (e) administered by nasal administration; or and/or (f) administered by injection to the mammal; and/or (g) administered topically to the mammal; and/or (h) administered by ophthalmic administration; and/or (i) administered rectally to the mammal; and/or (j) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the aforementioned aspects involving the treatment of a disease or condition are further embodiments comprising administering at least one additional agent in addition to the administration of a compound of Formula (I), (Ia), (Ib), or (Ic), or a pharmaceutically acceptable salt or solvate thereof. In various embodiments, each agent is administered in any order, including simultaneously.

In any of the embodiments disclosed herein, the mammal or subject is a human.

In some embodiments, compounds provided herein are administered to a human.

In some embodiments, compounds provided herein are orally administered.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Semicarbazide-sensitive amine oxidase (SSAO) is a member of the semicarbazide-sensitive amino oxidase family, and is also known as AOC3 (amine oxidase, copper containing 3) or VAP-1 (vascular adhesion protein 1). SSAO (AOC3) has two closely related genes in the human genome. AOC1 which corresponds to a diamine oxidase (DAO) found in gut, lung and kidney (Chassande, O. et al., J. Biol. Chem., 1994, 269: 14484-14489) and AOC2, a SSAO with expression in the eye (Imamura, Y. et al., Genomics, 1997, 40: 277-283). AOC4 is a sequence that does not lead to a functional gene product in humans (Schwelberger, H. G. J. Neural Transm., 2007, 1 14: 757-762).

SSAO has at least two physiological functions. In some cases, SSAO functions as an amine oxidase in which primary amines may be oxidized to aldehydes, leading to the release of ammonia and hydrogen peroxide upon regeneration of the cofactor 2,4,5-trihydroxy-phenyl-alanyl-quinone (TPQ). Endogenous substrates include methylamine, dopamine and aminoacetone. Aldehyde products generated under high AOC3 levels can be highly reactive, leading to glycation end products which may be regarded as drivers of diabetes associated inflammatory mechanisms (Mathys, K. C. et al., Biochem. Biophys. Res. Commun., 2002, 297: 863-869). In addition, hydrogen peroxide produced by SSAO can directly lead to direct cellular damage or be sensed by the tissue as a messenger of inflammation and so lead to further propagation of inflammatory processes.

In some cases, SSAO has cell adhesion activity, with SSAO having been shown to be important for leukocyte rolling, adhesion and transmigration in response to inflammatory stimuli (Salmi et al, Antoxidants and Redox Signaling, 2017). Both activities are associated with inflammatory processes.

SSAO was also shown to play a role in extravasation of inflammatory cells from the circulation to sites of inflammation (Salmi M.; Trends Immunol. 2001, 22, 21 1-216). SSAO antibodies have been shown to attenuate inflammatory processes by blocking the adhesion site of the SSAO protein. In addition, inhibitors of the amine oxidase activity of SSAO have been found to interfere with leukocyte rolling, adhesion and extravasation and, in a similar manner to SSAO antibodies, exhibit anti-inflammatory properties.

Recently SSAO has been implicated in the pathogenesis of liver diseases such as fatty liver disease (Weston, C. J. et al., J Neural. Transm. 2011, 118, 1055-1064). In some embodiments, serum SSAO is elevated in patients with fatty liver disease and correlates with histological markers of liver injury. In some embodiments, SSAO has been shown to contribute to liver fibrosis in preclinical models induced by chemical injury and diet induction. SSAO knock-out animals, or SSAO inhibition using an antibody are protective in both of these models (Weston et al; J. Clin. Invest., 2015, 125, 2, 501-520).

### Non-Alcoholic Fatty Liver Disease and Non-Alcoholic Steatohepatitis

Non-alcoholic fatty liver disease (NAFLD) is associated with excessive fat in the liver (steatosis) and in some cases progresses to NASH, which is defined by the histologic hallmarks of inflammation, cell death, and fibrosis. In some instances, primary NASH is associated with insulin resistance, while secondary NASH is caused by medical or surgical conditions, or drugs such as, but not limited to, tamoxifen. In some cases, NASH progresses to advanced fibrosis, hepatocellular carcinoma, or end-stage liver disease requiring liver transplantation.

In some instances, NASH develops as a result of triglyceride (TGs) imbalance. For example, dysfunctional adipocytes secrete pro-inflammatory molecules such as cytokines and chemokines leading to insulin resistance and a failure of lipolysis suppression in the adipocytes. In some instances, this failure of lipolysis suppression leads to a release of free fatty acids (FFAs) into the circulation and uptake within the liver. In some cases, over accumulation of FFAs in the form of triglycerides (TGs) in lipid droplets leads to oxidative stress, mitochondrial dysfunction, and upregulation of pro-inflammatory molecules.

In some embodiments, an SSAO inhibitor disclosed herein is used in the treatment of non-alcoholic steatohepatitis (NASH). In some examples, the SSAO inhibitor reduces NASH the subject by at least 5%, at least 10%, at least 15%, at least 20%, at least 30%, at least 40%, at least 50%, or more. In some cases, NASH is reduced by about 5% to about 50%, by about 5% to about 25%, by about 10% to about 20%, or by about 10% to about 30%. In some instances, the level of NASH is relative to the level of NASH in a subject not treated with the SSAO inhibitor.

In some embodiments, an SSAO inhibitor disclosed herein is used in the treatment of NAFLD. In some examples, the SSAO inhibitor reduces NAFLD in the subject by at least 5%, at least 10%, at least 15%, at least 20%, at least 30%, at least 40%, at least 50%, or more. In some cases, NAFLD is reduced by about 5% to about 50%, by about 5% to about 25%, by about 10% to about 20%, or by about 10% to about 30%. In some instances, the level of NAFLD is relative to the level of NAFLD in a subject not treated with the SSAO inhibitor.

### Ballooning

Hepatocyte ballooning, a feature denoting cellular injury, is a feature of NASH. Ballooning is a feature that denotes progressive NAFL (types 3 and 4). The term applies to enlarged, swollen-appearing hepatocytes; the affected cells are often intermixed in areas of steatosis and, in classic steatohepatitis, in the perivenular regions. Hepatocellular ballooning is most commonly noted in regions of H & E-detectable perisinusoidal fibrosis. Ballooned hepatocytes are most easily noted when they contain MH (either typical or poorly formed). Hepatocyte ballooning is a structural manifestation of microtubular disruption and severe cell injury.

In some embodiments, an SSAO inhibitor disclosed herein reduces liver ballooning in a subject. In some examples, the SSAO inhibitor reduces liver ballooning in the subject by at least 5%, at least 10%, at least 15%, at least 20%, at least 30%, at least 40%, at least 50%, or more. In some instances, liver ballooning is reduced by about 5% to about 50%, by about 5% to about 25%, by about 10% to about 20%, or by about 10% to about 30%. In some instances, the liver ballooning is relative to the level of liver ballooning in a subject not treated with the SSAO inhibitor.

### Compounds

Compounds described herein, including pharmaceutically acceptable salts, prodrugs, active metabolites and pharmaceutically acceptable solvates thereof, are SSAO inhibitors.

In one aspect, described herein are SSAO inhibitors and uses thereof. In one aspect, described herein is a compound that has the structure of Formula (I), or a pharmaceutically acceptable salt or solvate thereof:

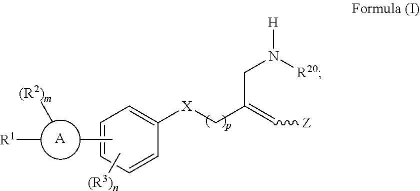

wherein,

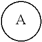

is a C₃₋₁₀cycloalkyl ring;
X is —O—, —S—, —S(O)₂—, —N(R¹³)—, or —C(R¹³)₂—;
Z is H, F, or Cl;
R¹ is halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁴, —SR⁴, —N(R⁴)(R⁵), —C(O)OR⁴, —OC(O)N(R⁴)(R⁵), —N(R⁶)C(O)N(R⁴)(R⁵), —N(R⁶)C(O)OR⁷, —N(R⁶)S(O)₂R⁷, —C(O)R⁷, —S(O)R⁷, —OC(O)R⁷, —C(O)N(R⁴)(R⁵), —C(O)C(O)N(R⁴)(R⁵), —N(R⁶)C(O)R⁷, —S(O)₂R⁷, —S(O)₂N(R⁴)(R⁵)—, S(═O)(═NH)N(R⁴)(R⁵), —CH₂C(O)N(R⁴)(R⁵), —CH₂N(R⁶)C(O)R⁷, —CH₂S(O)₂R⁷, or —CH₂S(O)₂N(R⁴)(R⁵), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a};
each R² and each R³ are each independently selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)R¹², —S(O)₂R¹², —S(O)₂N(R⁹)(R¹⁰), and —OC(O)R¹², wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14b};
R⁴ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14c};
R⁵ is selected from H, C₁₋₆alkyl, and C₁₋₆haloalkyl; or R⁴ and R⁵, together with the nitrogen to which they are attached, form a C₂₋₉heterocycloalkyl ring optionally substituted with one, two, or three R^{14d};
R⁶ is selected from H, C₁₋₆alkyl, and C₁₋₆haloalkyl;
R⁷ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14e};
each R⁸ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14f};
each R⁹ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14g};
each R¹⁰ is independently selected from H and C₁₋₆alkyl; or R⁹ and R¹⁰, together with the nitrogen to which they are attached, form a C₂₋₉heterocycloalkyl ring optionally substituted with one, two, or three R^{14h};
each R¹¹ is independently selected from H, C₁₋₆alkyl, and C₁₋₆haloalkyl;
each R¹² is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R¹⁴ⁱ;
each R¹³ is independently selected from H, C₁₋₆alkyl, and C₁₋₆haloalkyl;
each R^{14a}, R^{14b}, R^{14c}, R^{14d}, R^{14e}, R^{14f}, R^{14g}, R^{14h}, and R¹⁴ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), —OCH₂C(O)OR¹⁶, and —OC(O)R¹⁹, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), and —OC(O)R¹⁹;
each R¹⁵ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁶ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁷ is independently selected from H and C₁₋₆alkyl; or R¹⁶ and R¹⁷, together with the nitrogen to which they are attached, form a C₂₋₉heterocycloalkyl ring;
each R¹⁸ is independently selected from H and C₁₋₆alkyl;
each R¹⁹ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁰ is selected from H and C₁₋₆alkyl;
m is 0, 1, 2, 3, or 4;
n is 0, 1, 2, 3, or 4; and
p is 0 or 1.

For any and all of the embodiments, substituents are selected from among a subset of the listed alternatives. For example, in some embodiments X is —O—, —S—, —S(O)₂—, —N(R¹³)—, or —C(R¹³)₂-. In some embodiments, X is —O—. In some embodiments, X is —S—. In some embodiments, X is —S(O)₂—. In some embodiments, X is —N(R¹³)—. In some embodiments, X is —N(H)—. In some embodiments, X is —C(R¹³)₂-. In some embodiments, X is —CH₂—.

In some embodiments, Z is H, F, or Cl. In some embodiments, Z is F. In some embodiments, Z is Cl. In some embodiments, Z is H.

In some embodiments, p is 1. In some embodiments, p is 0.

In some embodiments,

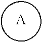

is a cycloheptyl ring. In some embodiments,

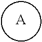

cyclooctyl ring. In some embodiments,

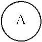

is a cycloheptyl ring. In some embodiments,

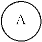

is a cyclohexyl ring. In some embodiments,

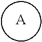

is a cyclopentyl ring. In some embodiments,

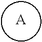

is a cyclobutyl ring. In some embodiments,

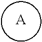

is a cyclopropyl ring.

In some embodiments, R¹ is —OR⁴, —C(O)OR⁴, —OC(O)N(R⁴)(R⁵), —N(R⁶)C(O)R⁷, —N(R⁶)C(O)N(R⁴)(R⁵), —N(R⁶)C(O)OR⁷, —N(R⁶)S(O)₂R⁷, —C(O)R⁷, —C(O)N(R⁴)(R⁵), —C(O)C(O)N(R⁴)(R⁵), —S(O)₂R⁷, —S(O)₂N(R⁴)(R⁵), —S(═O)(═NH)N(R⁴)(R⁵), —CH₂C(O)N(R⁴)(R⁵), —CH₂S(O)₂R⁷, or —CH₂S(O)₂N(R⁴)(R⁵). In some embodiments, R¹ is —OR⁴, —N(R⁶)C(O)R⁷, —N(R⁶)C(O)N(R⁴)(R⁵), —N(R⁶)S(O)₂R⁷, —C(O)R⁷, —C(O)N(R⁴)(R⁵), or —S(O)₂N(R⁴)(R⁵). In some embodiments, R¹ is —OR⁴. In some embodiments, R¹ is —N(R⁶)C(O)R⁷. In some embodiments, R¹ is —N(R⁶)C(O)N(R⁴)(R⁵). In some embodiments, R¹ is —N(R⁶)S(O)₂R⁷. In some embodiments, R¹ is —C(O)R⁷. In some embodiments, R¹ is —C(O)N(R⁴)(R⁵). In some embodiments, R¹ is —S(O)₂N(R⁴)(R⁵). In some embodiments, R⁴ is selected from H, C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14c}. In some embodiments, R⁴ is selected from H, C₁₋₆alkyl, and C₂₋₉heterocycloalkyl, wherein C₁₋₆alkyl and C₂₋₉heterocycloalkyl are optionally substituted with one, two, or three R^{14c}. In some embodiments, R⁴ is H. In some embodiments, R⁴ is unsubstituted C₁₋₆alkyl. In some embodiments, R⁴ is —CH₃. In some embodiments, R⁴ is unsubstituted C₂₋₉heterocycloalkyl. In some embodiments, R⁵ is H. In some embodiments, R⁵ is unsubstituted C₁₋₆alkyl. In some embodiments, R⁵ is —CH₃. In some embodiments, R⁷ is selected from C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14e}. In some embodiments, R⁷ is selected from C₁₋₆alkyl, C₂₋₉heterocycloalkyl, and C₆₋₁₀aryl, wherein C₁₋₆alkyl, C₂₋₉heterocycloalkyl, and C₆₋₁₀aryl are optionally substituted with one, two, or three R^{14e}. In some embodiments, R⁷ is C₁₋₆alkyl optionally substituted with one, two, or three R^{14e}. In some embodiments, R⁷ is unsubstituted C₁₋₆alkyl. In some embodiments, R⁷ is —CH₃. In some embodiments, R⁷ is unsubstituted C₂₋₉heterocycloalkyl. In some embodiments, R⁶ is H. In some embodiments, R⁶ is C₁₋₆alkyl. In some embodiments, R⁶ is C₁₋₆haloalkyl.

In some embodiments, R¹ is —C(O)NH₂. In some embodiments, R¹ is —C(O)N(H)CH₃.

In some embodiments, R¹ is —C(O)N(CH₃)₂. In some embodiments, R¹ is —C(O)N(H)CH₂CH₂CO₂H. In some embodiments, R¹ is —S(O)₂NH₂. In some embodiments, R¹ is —N(H)C(O)CH₃. In some embodiments, R¹ is —N(H)S(O)₂CH₃. In some embodiments, R¹ is —N(H)C(O)NH₂. In some embodiments, R¹ is —OCH₂CO₂H. In some embodiments, R¹ is

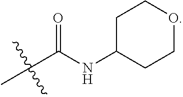 [SMILES: *C(=O)NC1CCOCC1]

In some embodiments, R¹ is

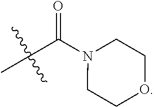 [SMILES: *C(=O)N1CCOCC1]

In some embodiments, R¹ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl, wherein C₁₋₆alkyl, C₂₋₆alkenyl, C₂₋₆alkynyl, C₃. 6cycloalkyl, C₂₋₉heterocycloalkyl, C₆₋₁₀aryl, and C_{1.9}heteroaryl are optionally substituted with one, two, or three R^{14a}. In some embodiments, R¹ is C₁₋₆alkyl, C₂₋₉heterocycloalkyl, C₆₋₁₀aryl, or C₁₋₉heteroaryl, wherein C₁₋₆alkyl, C₂₋₉heterocycloalkyl, C₆₋₁₀aryl, and C₁₋₉heteroaryl are optionally substituted with one, two, or three R^{14a}. In some embodiments, R¹ is C₁₋₆alkyl optionally substituted with one, two, or three R^{14a}. In some embodiments, R¹ is C₁₋₉heteroaryl optionally substituted with one, two, or three R^{14a}.

In some embodiments, R¹ is

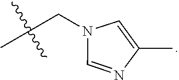 [SMILES: *Cn1cnc(C)c1]

In some embodiments, R¹ is

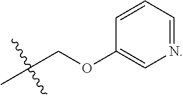 [SMILES: *COc1cccnc1]

In some embodiments, R¹ is —CH₂OCH₂CO₂H. In some embodiments, R¹ is —C(CH₃)₂OCH₂CO₂H. In some embodiments, R¹ is

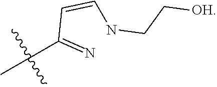 [SMILES: *C(=N)/C=C\NCCO]

In some embodiments, R¹ is

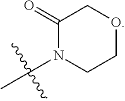 [SMILES: *N1CCOCC1=O]

In some embodiments, m is 0.

In some embodiments, n is 0.

In some embodiments, n is 1 or 2 and each R³ is independently selected from halogen, —CN, C₁₋₆alkyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)₂R¹², —S(O)₂N(R⁹)(R¹⁰), and —OC(O)R¹², wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14b}. In some embodiments, n is 1 or 2 and each R³ is independently selected from halogen, —CN, C₁₋₆alkyl, C₂₋₉heterocycloalkyl, C₁₋₉heteroaryl, —OR⁸, —N(R⁹)(R¹⁰), —C(O)OR⁹, —C(O)N(R⁹)(R¹⁰), —C(O)R¹², —S(O)₂R¹², —S(O)₂N(R⁹)(R¹⁰), wherein C₁₋₆alkyl, C₂₋₉heterocycloalkyl, and C₁₋₉heteroaryl are optionally substituted with one, two, or three R^{14b}. In some embodiments, n is 1 or 2 and each R³ is independently selected from halogen, —CN, C₁₋₆alkyl, —OR⁸, —N(R⁹)(R¹⁰), wherein C₁₋₆alkyl is optionally substituted with one, two, or three R^{14b}.

In some embodiments, R²⁰ is H. In some embodiments, R²⁰ is C₁₋₆alkyl. In some embodiments, R²⁰ is —CH₃.

In some embodiments, the compound has the structure of Formula (Ia), Formula (Ia′), Formula (Iaa) or Formula (Iaa′), or a pharmaceutically acceptable salt or solvate thereof:

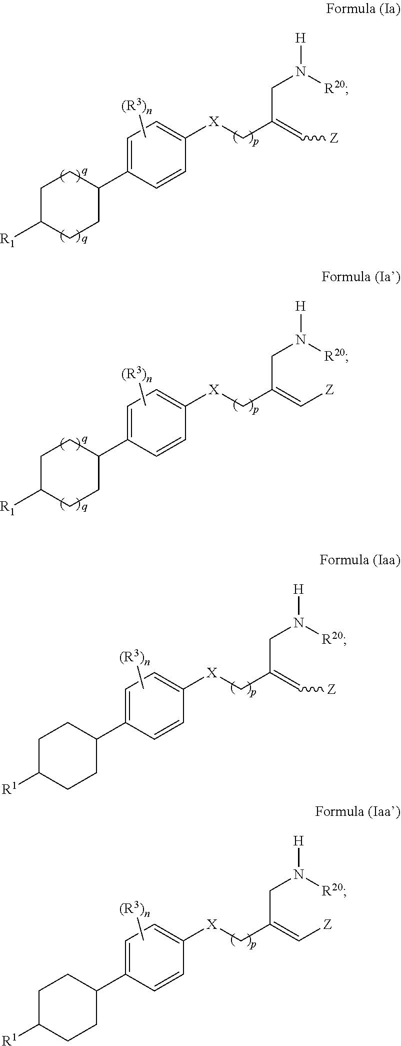

wherein,
   X is —O—, —S—, —S(O)₂—, —N(R¹³)—, or —C(R¹³)₂—;
   Z is H, F, or C₁;
   R¹ is halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁴, —SR⁴, —N(R⁴)(R⁵), —C(O)OR⁴, —OC(O)N(R⁴)(R⁵), —N(R⁶)C(O)N(R⁴)(R⁵), —N(R⁶)C(O)OR⁷, —N(R⁶)S(O)₂R⁷, —C(O)R⁷, —S(O)R⁷, —OC(O)R⁷, —C(O)N(R⁴)(R⁵), —C(O)C(O)N(R⁴)(R⁵), —N(R⁶)C(O)R⁷, —S(O)₂R⁷, —S(O)₂N(R⁴)(R⁵)—, S(═O)(═NH)N(R⁴)(R⁵), —CH₂C(O)N(R⁴)(R⁵), —CH₂N(R⁶)C(O)R⁷, —CH₂S(O)₂R⁷, or —CH₂S(O)₂N(R⁴)(R⁵), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a};
   each R³ is independently selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)R¹², —S(O)₂R¹², —S(O)₂N(R⁹)(R¹⁰), and —OC(O)R¹², wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14b};
   R⁴ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14c};
   R⁵ is selected from H, C₁₋₆alkyl, and C₁₋₆haloalkyl; or R⁴ and R⁵, together with the nitrogen to which they are attached, form a C₂₋₉heterocycloalkyl ring optionally substituted with one, two, or three R^{14d};
   R⁶ is selected from H, C₁₋₆alkyl, and C₁₋₆haloalkyl;
   R⁷ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14e};
   each R⁸ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14f};
   each R⁹ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14g};
   each R¹⁰ is independently selected from H and C₁₋₆alkyl; or R⁹ and R¹⁰, together with the nitrogen to which they are attached, form a C₂₋₉heterocycloalkyl ring optionally substituted with one, two, or three R^{14h};
   each R¹¹ is independently selected from H, C₁₋₆alkyl, and C₁₋₆haloalkyl;
   each R¹² is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R¹⁴ⁱ;
   each R¹³ is independently selected from H, C₁₋₆alkyl, and C₁₋₆haloalkyl;
   each R^{14a}, R^{14b}, R^{14c}, R^{14d}, R^{14e}, R^{14f}, R^{14g}, R^{14h}, and R¹⁴ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), —OCH₂C(O)OR¹⁶, and —OC(O)R¹⁹, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), and —OC(O)R¹⁹;
   each R¹⁵ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
   each R¹⁶ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
   each R¹⁷ is independently selected from H and C₁₋₆alkyl; or R¹⁶ and R¹⁷, together with the nitrogen to which they are attached, form a C₂₋₉heterocycloalkyl ring;
   each R¹⁸ is independently selected from H and C₁₋₆alkyl;
   each R¹⁹ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
   R²⁰ is selected from H and C₁₋₆alkyl;
   n is 0, 1, 2, 3, or 4;
   p is 0 or 1; and
   each q is independently 0, 1, or 2.

For any and all of the embodiments, substituents are selected from among a subset of the listed alternatives. For example, in some embodiments X is —O—, —S—, —S(O)₂—, —N(R¹³)—, or —C(R¹³)₂—. In some embodiments, X is —O—. In some embodiments, X is —S—. In some embodiments, X is —S(O)₂—. In some embodiments, X is —N(R¹³)—. In some embodiments, X is —N(H)—. In some embodiments, X is —C(R¹³)₂—. In some embodiments, X is —CH₂—.

In some embodiments, Z is H, F, or C₁. In some embodiments, Z is F. In some embodiments, Z is Cl. In some embodiments, Z is H.

In some embodiments, p is 1. In some embodiments, p is 0.

In some embodiments, each q is 1. In some embodiments, each q is 0. In some embodiments, each q is 2. In some embodiments, one q is 0 and one q is 1. In some embodiments, one q is 1 and one q is 2.

In some embodiments, R¹ is —OR⁴, —C(O)OR⁴, —OC(O)N(R⁴)(R⁵), —N(R⁶)C(O)R⁷, —N(R⁶)C(O)N(R⁴)(R⁵), —N(R⁶)C(O)OR⁷, —N(R⁶)S(O)₂R⁷, —C(O)R⁷, —C(O)N(R⁴)(R⁵), —C(O)C(O)N(R⁴)(R⁵), —S(O)₂R⁷, —S(O)₂N(R⁴)(R⁵), —S(═O)(═NH)N(R⁴)(R⁵), —CH₂C(O)N(R⁴)(R⁵), —CH₂S(O)₂R⁷, or —CH₂S(O)₂N(R⁴)(R⁵). In some embodiments, R¹ is —OR⁴, —N(R⁶)C(O)R⁷, —N(R⁶)C(O)N(R⁴)(R⁵), —N(R⁶)S(O)₂R⁷, —C(O)R⁷, —C(O)N(R⁴)(R⁵), or —S(O)₂N(R⁴)(R⁵). In some embodiments, R¹ is —OR⁴. In some embodiments, R¹ is —N(R⁶)C(O)R⁷. In some embodiments, R¹ is —N(R⁶)C(O)N(R⁴)(R⁵). In some embodiments, R¹ is —N(R⁶)S(O)₂R⁷. In some embodiments, R¹ is —C(O)R⁷. In some embodiments, R¹ is —C(O)N(R⁴)(R⁵). In some embodiments, R¹ is —S(O)₂N(R⁴)(R⁵). In some embodiments, R⁴ is selected from H, C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14c}. In some embodiments, R⁴ is selected from H, C₁₋₆alkyl, and C₂₋₉heterocycloalkyl, wherein C₁₋₆alkyl and C₂₋₉heterocycloalkyl are optionally substituted with one, two, or three R^{14c}. In some embodiments, R⁴ is H. In some embodiments, R⁴ is unsubstituted C₁₋₆alkyl. In some embodiments, R⁴ is —CH₃. In some embodiments, R⁴ is unsubstituted C₂₋₉heterocycloalkyl. In some embodiments, R⁵ is H. In some embodiments, R⁵ is unsubstituted C₁₋₆alkyl. In some embodiments, R⁵ is —CH₃. In some embodiments, R⁷ is selected from C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14e}. In some embodiments, R⁷ is selected from C₁₋₆alkyl, C₂₋₉heterocycloalkyl, and C₆₋₁₀aryl, wherein C₁₋₆alkyl, C₂₋₉heterocycloalkyl, and C₆₋₁₀aryl are optionally substituted with one, two, or three R^{14e}. In some embodiments, R⁷ is C₁₋₆alkyl optionally substituted with one, two, or three R^{14e}. In some embodiments, R⁷ is unsubstituted C₁₋₆alkyl. In some embodiments, R⁷ is —CH₃. In some embodiments, R⁷ is unsubstituted C₂₋₉heterocycloalkyl. In some embodiments, R⁶ is H. In some embodiments, R⁶ is C₁₋₆alkyl. In some embodiments, R⁶ is C₁₋₆haloalkyl.

In some embodiments, R¹ is —C(O)NH₂. In some embodiments, R¹ is —C(O)N(H)CH₃.

In some embodiments, R¹ is —C(O)N(CH₃)₂. In some embodiments, R¹ is —C(O)N(H)CH₂CH₂CO₂H. In some embodiments, R¹ is —S(O)₂NH₂. In some embodiments, R¹ is —N(H)C(O)CH₃. In some embodiments, R¹ is —N(H)S(O)₂CH₃. In some embodiments, R¹ is —N(H)C(O)NH₂. In some embodiments, R¹ is —OCH₂CO₂H. In some embodiments, R¹ is

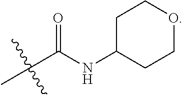 [SMILES: *C(=O)NC1CCOCC1]

In some embodiments, R¹ is

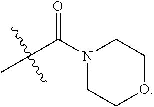 [SMILES: *C(=O)N1CCOCC1]

In some embodiments, R¹ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a}. In some embodiments, R¹ is C₁₋₆alkyl, C₂₋₉heterocycloalkyl, C₆₋₁₀aryl, or C₁₋₉heteroaryl, wherein C₁₋₆alkyl, C₂₋₉heterocycloalkyl, C₆₋₁₀aryl, and C₁₋₉heteroaryl are optionally substituted with one, two, or three R^{14a}. In some embodiments, R¹ is C₁₋₆alkyl optionally substituted with one, two, or three R^{14a}. In some embodiments, R¹ is C₁₋₉heteroaryl optionally substituted with one, two, or three R^{14a}.

In some embodiments, R¹ is

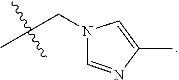 [SMILES: *Cn1cnc(C)c1]

In some embodiments, R¹ is

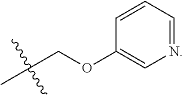 [SMILES: *COc1cccnc1]

In some embodiments, R¹ is —CH₂OCH₂CO₂H. In some embodiments, R¹ is —C(CH₃)₂OCH₂CO₂H. In some embodiments, R¹ is

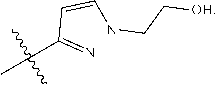 [SMILES: *C(=N)/C=C\NCCO]

In some embodiments, R¹ is

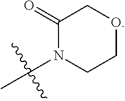 [SMILES: *N1CCOCC1=O]

In some embodiments, n is 0.

In some embodiments, n is 1 or 2 and each R³ is independently selected from halogen, —CN, C₁₋₆alkyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰)—N(R¹¹)C(O)OR¹², —N(R¹¹)C(O)R¹², —N(R¹¹)S(O)₂R¹², —C(O)R¹², —S(O)₂R¹², —S(O)₂N(R⁹)(R¹⁰), and —OC(O)R¹², wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14b}. In some embodiments, n is 1 or 2 and each R³ is independently selected from halogen, —CN, C₁₋₆alkyl, C₂₋₉heterocycloalkyl, C₁₋₉heteroaryl, —OR⁸, —N(R⁹)(R¹⁰), —C(O)OR⁹, —C(O)N(R⁹)(R¹⁰), —C(O)R¹², —S(O)₂R¹², —S(O)₂N(R⁹)(R¹⁰), wherein C₁₋₆alkyl, C₂₋₉heterocycloalkyl, and C₁₋₉heteroaryl are optionally substituted with one, two, or three R^{14b}. In some embodiments, n is 1 or 2 and each R³ is independently selected from halogen, —CN, C₁₋₆alkyl, —OR⁸, —N(R⁹)(R¹⁰), wherein C₁₋₆alkyl is optionally substituted with one, two, or three R^{14b}.

In some embodiments, R²⁰ is H. In some embodiments, R²⁰ is C₁₋₆alkyl. In some embodiments, R²⁰ is —CH₃.

In some embodiments, the compound has the structure of Formula (Ib), Formula (Ib′), Formula (Ibb), or Formula (Ibb′), or a pharmaceutically acceptable salt or solvate thereof:

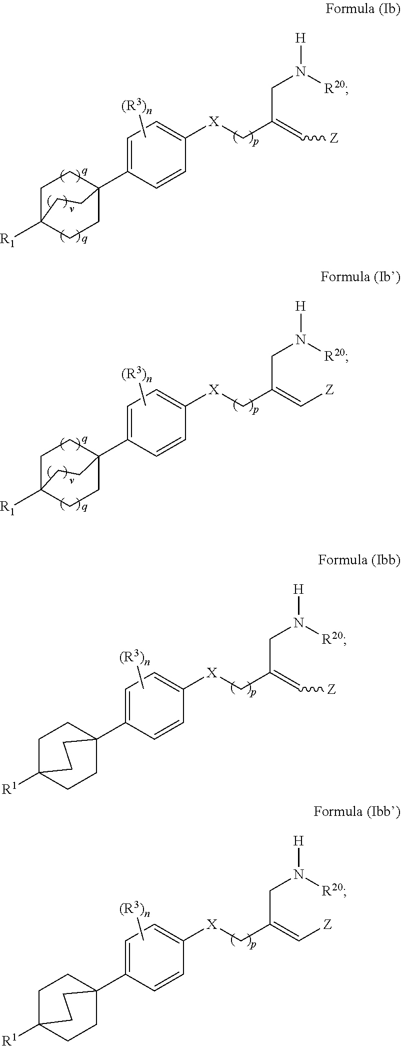

wherein,
X is —O—, —S—, —S(O)₂—, —N(R¹³)—, or —C(R¹³)₂—;
Z is H, F, or C₁;
R¹ is halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁴, —SR⁴, —N(R⁴)(R⁵), —C(O)OR⁴, —OC(O)N(R⁴)(R⁵), —N(R⁶)C(O)N(R⁴)(R⁵), —N(R⁶)C(O)OR⁷, —N(R⁶)S(O)₂R⁷, —C(O)R⁷, —S(O)R⁷, —OC(O)R⁷, —C(O)N(R⁴)(R⁵), —C(O)C(O)N(R⁴)(R⁵), —N(R⁶)C(O)R⁷, —S(O)₂R⁷, —S(O)₂N(R⁴)(R⁵)—, S(═O)(═NH)N(R⁴)(R⁵), —CH₂C(O)N(R⁴)(R⁵), —CH₂N(R⁶)C(O)R⁷, —CH₂S(O)₂R⁷, or —CH₂S(O)₂N(R⁴)(R⁵), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a};
each R³ is independently selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)R¹², —S(O)₂R¹², —S(O)₂N(R⁹)(R¹⁰), and —OC(O)R¹², wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14b};
R⁴ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14c};
R⁵ is selected from H, C₁₋₆alkyl, and C₁₋₆haloalkyl; or R⁴ and R⁵, together with the nitrogen to which they are attached, form a C₂₋₉heterocycloalkyl ring optionally substituted with one, two, or three R^{14d};
R⁶ is selected from H, C₁₋₆alkyl, and C₁₋₆haloalkyl;
R⁷ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14e};
each R⁸ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14f};
each R⁹ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14g};
each R¹⁰ is independently selected from H and C₁₋₆alkyl; or R⁹ and R¹⁰, together with the nitrogen to which they are attached, form a C₂₋₉heterocycloalkyl ring optionally substituted with one, two, or three R^{14h};
each R¹¹ is independently selected from H, C₁₋₆alkyl, and C₁₋₆haloalkyl;
each R¹² is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R¹⁴ⁱ;
each R¹³ is independently selected from H, C₁₋₆alkyl, and C₁₋₆haloalkyl;
each R^{14a}, R^{14b}, R^{14c}, R^{14d}, R^{14e}, R^{14f}, R^{14g}, R^{14h}, and R¹⁴ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), —OCH₂C(O)OR¹⁶, and —OC(O)R¹⁹, wherein C₁₋₆alkyl, C₂₋₆alkenyl, C₂₋₆alkynyl, C₃. 6cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R⁸)C(O)R¹⁹, —N(R⁸)S(O)₂R¹⁹, —C(O)R¹⁹, —S(O)₂R¹⁹, —S(O)₂N(R¹⁶)(R¹⁷), and —OC(O)R¹⁹;
each R¹⁵ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁶ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁷ is independently selected from H and C₁₋₆alkyl; or R¹⁶ and R¹⁷, together with the nitrogen to which they are attached, form a C₂₋₉heterocycloalkyl ring;
each R¹⁸ is independently selected from H and C₁₋₆alkyl;
each R¹⁹ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁰ is selected from H and C₁₋₆alkyl;
n is 0, 1, 2, 3, or 4;
p is 0 or 1;
each q is independently 0, 1, or 2; and
v is 0, 1, or 2.

For any and all of the embodiments, substituents are selected from among a subset of the listed alternatives. For example, in some embodiments X is —O—, —S—, —S(O)₂—, —N(R¹³)—, or —C(R¹³)₂—. In some embodiments, X is —O—. In some embodiments, X is —S—. In some embodiments, X is —S(O)₂—. In some embodiments, X is —N(R¹³)—. In some embodiments, X is —N(H)—. In some embodiments, X is —C(R¹³)₂—. In some embodiments, X is —CH₂—.

In some embodiments, Z is H, F, or C₁. In some embodiments, Z is F. In some embodiments, Z is C₁. In some embodiments, Z is H.

In some embodiments, p is 1. In some embodiments, p is 0.

In some embodiments, each q is 1. In some embodiments, each q is 0. In some embodiments, each q is 2. In some embodiments, one q is 0 and one q is 1. In some embodiments, one q is 1 and one q is 2. In some embodiments, v is 1. In some embodiments, v is 0. In some embodiments, v is 2. In some embodiments, each q is 1 and v is 1. In some embodiments, each q is 1 and v is 2. In some embodiments, each q is 1 and v is 0.

In some embodiments, R¹ is —OR⁴, —C(O)OR⁴, —OC(O)N(R⁴)(R⁵), —N(R⁶)C(O)R⁷, —N(R⁶)C(O)N(R⁴)(R⁵), —N(R⁶)C(O)OR⁷, —N(R⁶)S(O)₂R⁷, —C(O)R⁷, —C(O)N(R⁴)(R⁵), —C(O)C(O)N(R⁴)(R⁵), —S(O)₂R⁷, —S(O)₂N(R⁴)(R⁵), —S(═O)(═NH)N(R⁴)(R⁵), —CH₂C(O)N(R⁴)(R⁵), —CH₂S(O)₂R⁷, or —CH₂S(O)₂N(R⁴)(R⁵). In some embodiments, R¹ is —OR⁴, —N(R⁶)C(O)R⁷, —N(R⁶)C(O)N(R⁴)(R⁵), —N(R⁶)S(O)₂R⁷, —C(O)R⁷, —C(O)N(R⁴)(R⁵), or —S(O)₂N(R⁴)(R⁵). In some embodiments, R¹ is —OR⁴. In some embodiments, R¹ is —N(R⁶)C(O)R⁷. In some embodiments, R¹ is —N(R⁶)C(O)N(R⁴)(R⁵). In some embodiments, R¹ is —N(R⁶)S(O)₂R⁷. In some embodiments, R¹ is —C(O)R⁷. In some embodiments, R¹ is —C(O)N(R⁴)(R⁵). In some embodiments, R¹ is —S(O)₂N(R⁴)(R⁵). In some embodiments, R⁴ is selected from H, C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14c}. In some embodiments, R⁴ is selected from H, C₁₋₆alkyl, and C₂₋₉heterocycloalkyl, wherein C₁₋₆alkyl and C₂₋₉heterocycloalkyl are optionally substituted with one, two, or three R^{14c}. In some embodiments, R⁴ is H. In some embodiments, R⁴ is unsubstituted C₁₋₆alkyl. In some embodiments, R⁴ is —CH₃. In some embodiments, R⁴ is unsubstituted C₂₋₉heterocycloalkyl. In some embodiments, R⁵ is H. In some embodiments, R⁵ is unsubstituted C₁₋₆alkyl. In some embodiments, R⁵ is —CH₃. In some embodiments, R⁷ is selected from C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14e}. In some embodiments, R⁷ is selected from C₁₋₆alkyl, C₂₋₉heterocycloalkyl, and C₆₋₁₀aryl, wherein C₁₋₆alkyl, C₂₋₉heterocycloalkyl, and C₆₋₁₀aryl are optionally substituted with one, two, or three R^{14e}. In some embodiments, R⁷ is C₁₋₆alkyl optionally substituted with one, two, or three R^{14e}. In some embodiments, R⁷ is unsubstituted C₁₋₆alkyl. In some embodiments, R⁷ is —CH₃. In some embodiments, R⁷ is unsubstituted C₂₋₉heterocycloalkyl. In some embodiments, R⁶ is H. In some embodiments, R⁶ is C₁₋₆alkyl. In some embodiments, R⁶ is C₁₋₆haloalkyl.

In some embodiments, R¹ is —C(O)NH₂. In some embodiments, R¹ is —C(O)N(H)CH₃.

In some embodiments, R¹ is —C(O)N(CH₃)₂. In some embodiments, R¹ is —C(O)N(H)CH₂CH₂CO₂H. In some embodiments, R¹ is —S(O)₂NH₂. In some embodiments, R¹ is —N(H)C(O)CH₃. In some embodiments, R¹ is —N(H)S(O)₂CH₃. In some embodiments, R¹ is —N(H)C(O)NH₂. In some embodiments, R¹ is —OCH₂CO₂H. In some embodiments, R¹ is

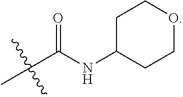 [SMILES: *C(=O)NC1CCOCC1]

In some embodiments, R¹ is

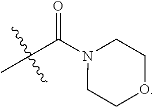 [SMILES: *C(=O)N1CCOCC1]

In some embodiments, R¹ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a}. In some embodiments, R¹ is C₁₋₆alkyl, C₂₋₉heterocycloalkyl, C₆₋₁₀aryl, or C₁₋₉heteroaryl, wherein C₁₋₆alkyl, C₂₋₉heterocycloalkyl, C₆₋₁₀aryl, and C₁₋₉heteroaryl are optionally substituted with one, two, or three R^{14a}. In some embodiments, R¹ is C₁₋₆alkyl optionally substituted with one, two, or three R^{14a}. In some embodiments, R¹ is C₁₋₉heteroaryl optionally substituted with one, two, or three R^{14a}.

In some embodiments, R¹ is

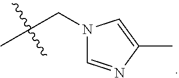 [SMILES: *Cn1cnc(C)c1]

In some embodiments, R¹ is

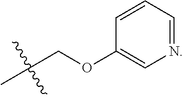 [SMILES: *COc1cccnc1]

In some embodiments, R¹ is —CH₂OCH₂CO₂H. In some embodiments, R¹ is —C(CH₃)₂OCH₂CO₂H. In some embodiments, R¹ is

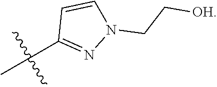 [SMILES: *c1ccn(CCO)n1]

In some embodiments, R¹ is

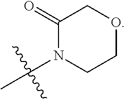 [SMILES: *N1CCOCC1=O]

In some embodiments, n is 0.

In some embodiments, n is 1 or 2 and each R³ is independently selected from halogen, —CN, C₁₋₆alkyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)₂R¹², —S(O)₂N(R⁹)(R¹⁰), and —OC(O)R¹², wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14b}. In some embodiments, n is 1 or 2 and each R³ is independently selected from halogen, —CN, C₁₋₆alkyl, C₂₋₉heterocycloalkyl, C₁₋₉heteroaryl, —OR⁸, —N(R⁹)(R¹⁰), —C(O)OR⁹, —C(O)N(R⁹)(R¹⁰), —C(O)R¹², —S(O)₂R¹², —S(O)₂N(R⁹)(R¹⁰), wherein C₁₋₆alkyl, C₂₋₉heterocycloalkyl, and C₁₋₉heteroaryl are optionally substituted with one, two, or three R^{14b}. In some embodiments, n is 1 or 2 and each R³ is independently selected from halogen, —CN, C₁₋₆alkyl, —OR⁸, —N(R⁹)(R¹⁰), wherein C₁₋₆alkyl is optionally substituted with one, two, or three R^{14b}.

In some embodiments, R²⁰ is H. In some embodiments, R²⁰ is C₁₋₆alkyl. In some embodiments, R²⁰ is —CH₃.

In some embodiments, the compound has the structure of Formula (Ic) or Formula (Ic′), or a pharmaceutically acceptable salt or solvate thereof:

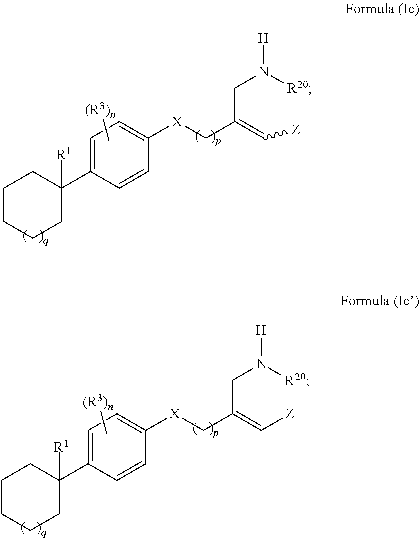

wherein:
X is —O—, —S—, —S(O)₂—, —N(R¹³)—, or —C(R¹³)₂—;
Z is H, F, or Cl;
R¹ is halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁴, —SR⁴, —N(R⁴)(R⁵), —C(O)OR⁴, —OC(O)N(R⁴)(R⁵), —N(R⁶)C(O)N(R⁴)(R⁵), —N(R⁶)C(O)OR⁷, —N(R⁶)S(O)₂R⁷, —C(O)R⁷, —S(O)R⁷, —OC(O)R⁷, —C(O)N(R⁴)(R⁵), —C(O)C(O)N(R⁴)(R⁵), —N(R⁶)C(O)R⁷, —S(O)₂R⁷, —S(O)₂N(R⁴)(R⁵)—, S(═O)(═NH)N(R⁴)(R⁵), —CH₂C(O)N(R⁴)(R⁵), —CH₂N(R⁶)C(O)R⁷, —CH₂S(O)₂R⁷, or —CH₂S(O)₂N(R⁴)(R⁵), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a};
each R³ is independently selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S(O)R¹², —S(O)₂R¹², —S(O)₂N(R⁹)(R¹⁰), and —OC(O)R¹², wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14b};
R⁴ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14c};
R⁵ is selected from H, C₁₋₆alkyl, and C₁₋₆haloalkyl; or R⁴ and R⁵, together with the nitrogen to which they are attached, form a C₂₋₉heterocycloalkyl ring optionally substituted with one, two, or three R^{14d};
R⁶ is selected from H, C₁₋₆alkyl, and C₁₋₆haloalkyl;
R⁷ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14e};
each R⁸ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14f};
each R⁹ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14g};
each R¹⁰ is independently selected from H and C₁₋₆alkyl; or R⁹ and R¹⁰, together with the nitrogen to which they are attached, form a C₂₋₉heterocycloalkyl ring optionally substituted with one, two, or three R^{14h};
each R¹¹ is independently selected from H, C₁₋₆alkyl, and C₁₋₆haloalkyl; each R¹² is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R¹⁴ⁱ;
each R¹³ is independently selected from H, C₁₋₆alkyl, and C₁₋₆haloalkyl;
each R^{14a}, R^{14b}, R^{14c}, R^{14d}, R^{14e}, R^{14f}, R^{14g}, R^{14h}, and R¹⁴ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), —OCH₂C(O)OR¹⁶, and —OC(O)R¹⁹, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), and —OC(O)R¹⁹;
each R¹⁵ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁶ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁷ is independently selected from H and C₁₋₆alkyl; or R¹⁶ and R¹⁷, together with the nitrogen to which they are attached, form a C₂₋₉heterocycloalkyl ring;
each R¹⁸ is independently selected from H and C₁₋₆alkyl;
each R¹⁹ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁰ is selected from H and C₁₋₆alkyl;
n is 0, 1, 2, 3, or 4;
p is 0 or 1; and
q is 0, 1, or 2.

For any and all of the embodiments, substituents are selected from among a subset of the listed alternatives. For example, in some embodiments X is —O—, —S—, —S(O)₂—, —N(R¹³)—, or —C(R¹³)₂—. In some embodiments, X is —O—. In some embodiments, X is —S—. In some embodiments, X is —S(O)₂—. In some embodiments, X is —N(R¹³)—. In some embodiments, X is —N(H)—. In some embodiments, X is —C(R¹³)₂—. In some embodiments, X is —CH₂—.

In some embodiments, Z is H, F, or C₁. In some embodiments, Z is F. In some embodiments, Z is Cl. In some embodiments, Z is H.

In some embodiments, p is 1. In some embodiments, p is 0.

In some embodiments, q is 2. In some embodiments, q is 1. In some embodiments, q is 0.

In some embodiments, R¹ is —OR⁴, —C(O)OR⁴, —OC(O)N(R⁴)(R⁵), —N(R⁶)C(O)R⁷, —N(R⁶)C(O)N(R⁴)(R⁵), —N(R⁶)C(O)OR⁷, —N(R⁶)S(O)₂R⁷, —C(O)R⁷, —C(O)N(R⁴)(R⁵), —C(O)C(O)N(R⁴)(R⁵), —S(O)₂R⁷, —S(O)₂N(R⁴)(R⁵), —S(═O)(═NH)N(R⁴)(R⁵), —CH₂C(O)N(R⁴)(R⁵), —CH₂S(O)₂R⁷, or —CH₂S(O)₂N(R⁴)(R⁵). In some embodiments, R¹ is —OR⁴, —N(R⁶)C(O)R⁷, —N(R⁶)C(O)N(R⁴)(R⁵), —N(R⁶)S(O)₂R⁷, —C(O)R⁷, —C(O)N(R⁴)(R⁵), or —S(O)₂N(R⁴)(R⁵). In some embodiments, R¹ is —OR⁴. In some embodiments, R¹ is —N(R⁶)C(O)R⁷. In some embodiments, R¹ is —N(R⁶)C(O)N(R⁴)(R⁵). In some embodiments, R¹ is —N(R⁶)S(O)₂R⁷. In some embodiments, R¹ is —C(O)R⁷. In some embodiments, R¹ is —C(O)N(R⁴)(R⁵). In some embodiments, R¹ is —S(O)₂N(R⁴)(R⁵). In some embodiments, R⁴ is selected from H, C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14c}. In some embodiments, R⁴ is selected from H, C₁₋₆alkyl, and C₂₋₉heterocycloalkyl, wherein C₁₋₆alkyl and C₂₋₉heterocycloalkyl are optionally substituted with one, two, or three R^{14c}. In some embodiments, R⁴ is H. In some embodiments, R⁴ is unsubstituted C₁₋₆alkyl. In some embodiments, R⁴ is —CH₃. In some embodiments, R⁴ is unsubstituted C₂₋₉heterocycloalkyl. In some embodiments, R⁵ is H. In some embodiments, R⁵ is unsubstituted C₁₋₆alkyl. In some embodiments, R⁵ is —CH₃. In some embodiments, R⁷ is selected from C₁₋₆alkyl, C₃₋₆cycloalkyl, C₂₋₉heterocycloalkyl, C₆₋₁₀aryl, and C₁₋₉heteroaryl, wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14e}. In some embodiments, R⁷ is selected from C₁₋₆alkyl, C₂₋₉heterocycloalkyl, and C₆₋₁₀aryl, wherein C₁₋₆alkyl, C₂₋₉heterocycloalkyl, and C₆₋₁₀aryl are optionally substituted with one, two, or three R^{14e}. In some embodiments, R⁷ is C₁₋₆alkyl optionally substituted with one, two, or three R^{14e}. In some embodiments, R⁷ is unsubstituted C₁₋₆alkyl. In some embodiments, R⁷ is —CH₃. In some embodiments, R⁷ is unsubstituted C₂₋₉heterocycloalkyl. In some embodiments, R⁶ is H. In some embodiments, R⁶ is C₁₋₆alkyl. In some embodiments, R⁶ is C₁₋₆haloalkyl.

In some embodiments, R¹ is —C(O)NH₂. In some embodiments, R¹ is —C(O)N(H)CH₃.

In some embodiments, R¹ is —C(O)N(CH₃)₂. In some embodiments, R¹ is —C(O)N(H)CH₂CH₂CO₂H. In some embodiments, R¹ is —S(O)₂NH₂. In some embodiments, R¹ is —N(H)C(O)CH₃. In some embodiments, R¹ is —N(H)S(O)₂CH₃. In some embodiments, R¹ is —N(H)C(O)NH₂. In some embodiments, R¹ is —OCH₂CO₂H. In some embodiments, R¹ is

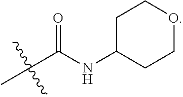 [SMILES: *C(=O)NC1CCOCC1]

In some embodiments, R¹ is

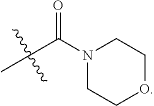 [SMILES: *C(=O)N1CCOCC1]

In some embodiments, R¹ is C₁₋₆alkyl, C₂₋₆alkenyl, C₂₋₆alkynyl, C₃₋₆cycloalkyl, C₂₋₉heterocycloalkyl, C₆₋₁₀aryl, or C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a}. In some embodiments, R¹ is C₁₋₆alkyl, C₂₋₉heterocycloalkyl, C₆₋₁₀aryl, or C₁₋₉heteroaryl, wherein C₁₋₆alkyl, C₂₋₉heterocycloalkyl, C₆₋₁₀aryl, and C₁₋₉heteroaryl are optionally substituted with one, two, or three R^{14a}. In some embodiments, R¹ is C₁₋₆alkyl optionally substituted with one, two, or three R^{14a}. In some embodiments, R¹ is C₁₋₉heteroaryl optionally substituted with one, two, or three R^{14a}.

In some embodiments, R¹ is

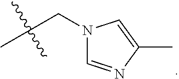 [SMILES: *Cn1cnc(C)c1]

In some embodiments, R¹ is

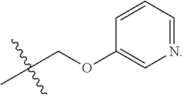 [SMILES: *COc1cccnc1]

In some embodiments, R¹ is —CH₂OCH₂CO₂H. In some embodiments, R¹ is —C(CH₃)₂OCH₂CO₂H. In some embodiments, R¹ is

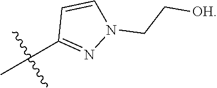 [SMILES: *c1ccn(CCO)n1]

In some embodiments, R¹ is

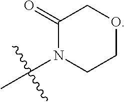 [SMILES: *N1CCOCC1=O]

In some embodiments, n is 0.

In some embodiments, n is 1 or 2 and each R³ is independently selected from halogen, —CN, C₁₋₆alkyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R¹¹)S(O)₂R¹², —C(O)R¹², —S( )₂R¹², —S(O)₂N(R⁹)(R¹⁰), and —OC(O)R¹², wherein C₁₋₆alkyl, C₃₋₆cycloalkyl, C₂₋₉heterocycloalkyl, C₆₋₁₀aryl, and C₁₋₉heteroaryl are optionally substituted with one, two, or three R^{14b}. In some embodiments, n is 1 or 2 and each R³ is independently selected from halogen, —CN, C₁₋₆alkyl, C₂₋₉heterocycloalkyl, C₁₋₉heteroaryl, —OR⁸, —N(R⁹)(R¹⁰), —C(O)OR⁹, —C(O)N(R⁹)(R¹⁰), —C(O)R¹², —S(O)₂R¹², —S(O)₂N(R⁹)(R¹⁰), wherein C₁₋₆alkyl, C₂₋₉heterocycloalkyl, and C₁₋₉heteroaryl are optionally substituted with one, two, or three R^{14b}. In some embodiments, n is 1 or 2 and each R³ is independently selected from halogen, —CN, C₁₋₆alkyl, —OR⁸, —N(R⁹)(R¹⁰), wherein C₁₋₆alkyl is optionally substituted with one, two, or three R^{14b}.

In some embodiments, R²⁰ is H. In some embodiments, R²⁰ is C₁₋₆alkyl. In some embodiments, R²⁰ is —CH₃.

Any combination of the groups described above for the various variables is contemplated herein. Throughout the specification, groups and substituents thereof are chosen by one skilled in the field to provide stable moieties and compounds.

In some embodiments, compounds described herein include, but are not limited to, those described in Table 1.

TABLE 1

| Compound | Structure | Chemical Name |
|---|---|---|
| 1 | 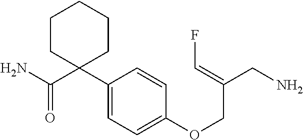 [SMILES: NC/C(=C\F)COc1ccc(C2(C(N)=O)CCCCC2)cc1] | (E)-1-(4-((2-(aminomethyl)-3- fluoroallyl)oxy)phenyl)cyclohexane- carboxamide |
| 1.01 | 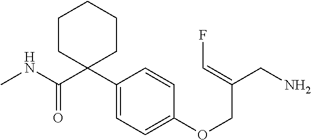 [SMILES: CNC(=O)C1(c2ccc(OC/C(=C/F)CN)cc2)CCCCC1] | (E)-1-(4-((2-(aminomethyl)-3- fluoroallyl)oxy)phenyl)-N- methylcyclohexanecarboxamide |
| 2 | 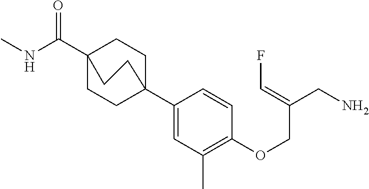 [SMILES: CNC(=O)C12CCC(c3ccc(OC/C(=C/F)CN)c(C)c3)(CC1)CC2] | (E)-4-(4-((2-(aminomethyl)-3- fluoroallyl)oxy)phenyl)-N- methylbicyclo[2.2.2]octane-1- carboxamide |
| 2.01 | 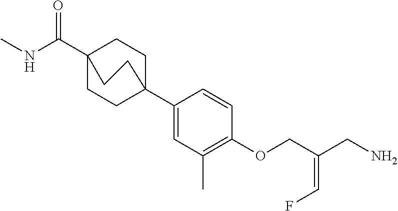 [SMILES: CNC(=O)C12CCC(c3ccc(OC/C(=C\F)CN)c(C)c3)(CC1)CC2] | (Z)-4-(4-((2-(aminomethyl)-3- fluoroallyl)oxy)-3-methylphenyl)-N- methylbicyclo[2.2.2]octane-1- carboxamide |
| 2.02 | 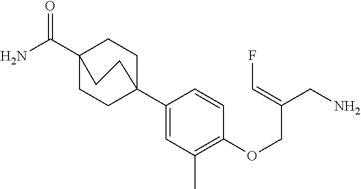 [SMILES: Cc1cc(C23CCC(C(N)=O)(CC2)CC3)ccc1OC/C(=C/F)CN] | (E)-4-(4-((2-(aminomethyl)-3- fluoroallyl)oxy)-3- methylphenyl)bicyclo[2.2.2]octane-1- carboxamide |
| 2.03 | 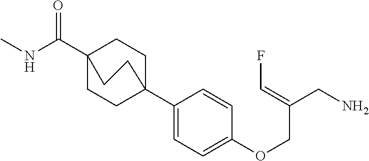 [SMILES: CNC(=O)C12CCC(c3ccc(OC/C(=C/F)CN)cc3)(CC1)CC2] | (E)-4-(4-((2-(aminomethyl)-3- fluoroallyl)oxy)phenyl)-N- methylbicyclo[2.2.2]octane-1- carboxamide |
| 2.04 | 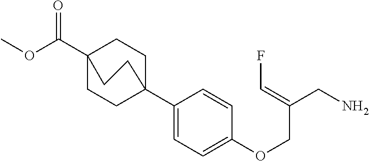 [SMILES: COC(=O)C12CCC(c3ccc(OC/C(=C/F)CN)cc3)(CC1)CC2] | (E)-methyl 4-(4-((2-(aminomethyl)-3- fluoroallyl)oxy)phenyl)bicyclo[2.2.2] octane-1-carboxylate |
| 2.05 | 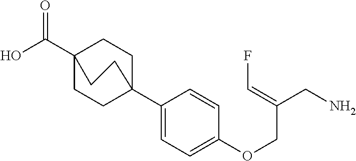 [SMILES: NC/C(=C\F)COc1ccc(C23CCC(C(=O)O)(CC2)CC3)cc1] | (E)-4-(4-((2-(aminomethyl)-3- fluoroallyl)oxy)phenyl)bicyclo[2.2.2] octane-1-carboxylic acid |
| 2.06 | 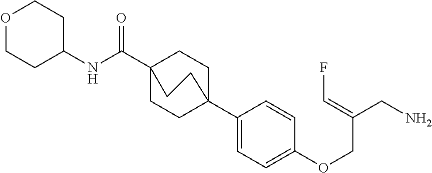 [SMILES: NC/C(=C\F)COc1ccc(C23CCC(C(=O)NC4CCOCC4)(CC2)CC3)cc1] | (E)-4-(4-((2-(aminomethyl)-3- fluoroallyl)oxy)phenyl)-N-(tetrahydro- 2H-pyran-4-yl)bicyclo[2.2.2]octane-1- carboxamide |
| 2.06b | 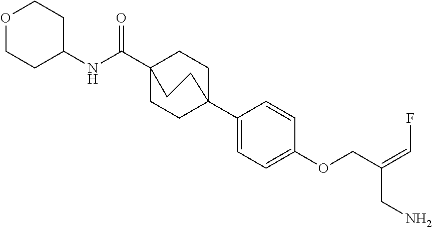 [SMILES: NC/C(=C/F)COc1ccc(C23CCC(C(=O)NC4CCOCC4)(CC2)CC3)cc1] | (Z)-4-(4-((2-(aminomethyl)-3- fluoroallyl)oxy)phenyl)-N-(tetrahydro- 2H-pyran-4-yl)bicyclo[2.2.2]octane-1- carboxamide |
| 2.07 | 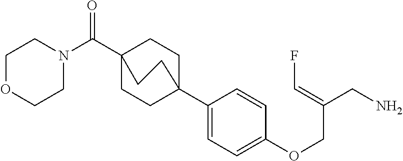 [SMILES: NC/C(=C\F)COc1ccc(C23CCC(C(=O)N4CCOCC4)(CC2)CC3)cc1] | (E)-(4-(4-((2-(aminomethyl)-3- fluoroallyl)oxy)phenyl)bicyclo[2.2.2] octan-1-yl)(morpholino)methanone |
| 2.08 | 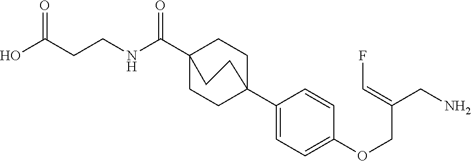 [SMILES: NC/C(=C\F)COc1ccc(C23CCC(C(=O)NCCC(=O)O)(CC2)CC3)cc1] | (E)-3-(4-(4-((2-(aminomethyl)-3- fluoroallyl)oxy)phenyl)bicyclo[2.2.2] octane-1-carboxamido)propanoic acid |
| 2.09 | 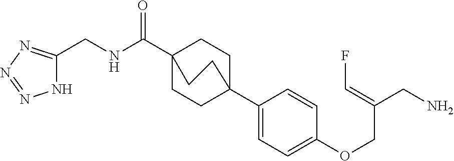 [SMILES: NC/C(=C\F)COc1ccc(C23CCC(C(=O)NCc4nnn[nH]4)(CC2)CC3)cc1] | (E)-N-((1H-tetrazol-5-yl)methyl)-4-(4- ((2-(aminomethyl)-3- fluoroallyl)oxy)phenyl)bicyclo[2.2.2] octane-1-carboxamide |
| 2.10 | 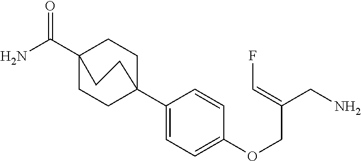 [SMILES: NC/C(=C\F)COc1ccc(C23CCC(C(N)=O)(CC2)CC3)cc1] | (E)-4-(4-((2-(aminomethyl)-3- fluoroallyl)oxy)phenyl)bicyclo[2.2.2] octane-1-carboxamide |
| 2.11 | 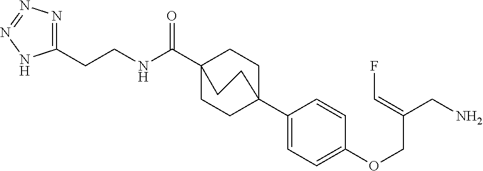 [SMILES: NC/C(=C\F)COc1ccc(C23CCC(C(=O)NCCc4nnn[nH]4)(CC2)CC3)cc1] | (E)-N-(2-(1H-tetrazol-5-yl)ethyl)-4-(4- ((2-(aminomethyl)-3- fluoroallyl)oxy)phenyl)bicyclo[2.2.2] octane-1-carboxamide |
| 2.12 | 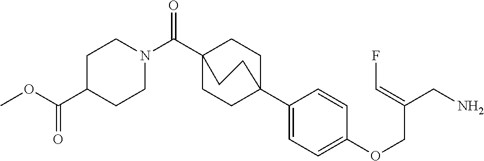 [SMILES: COC(=O)C1CCN(C(=O)C23CCC(c4ccc(OC/C(=C/F)CN)cc4)(CC2)CC3)CC1] | (E)-methyl 1-(4-(4-((2-(aminomethyl)- 3- fluoroallyl)oxy)phenyl)bicyclo[2.2.2] octane-1-carbonyl)piperidine-4- carboxylate |
| 2.13 | 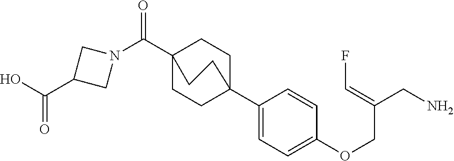 [SMILES: NC/C(=C\F)COc1ccc(C23CCC(C(=O)N4CC(C(=O)O)C4)(CC2)CC3)cc1] | (E)-1-(4-(4-((2-(aminomethyl)-3- fluoroallyl)oxy)phenyl)bicyclo[2.2.2] octane-1-carbonyl)azetidine-3- carboxylic acid |
| 2.14 | 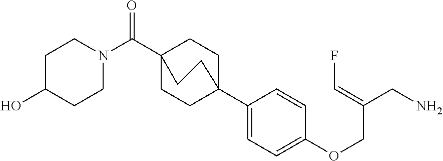 [SMILES: NC/C(=C\F)COc1ccc(C23CCC(C(=O)N4CCC(O)CC4)(CC2)CC3)cc1] | (E)-(4-(4-((2-(aminomethyl)-3- fluoroallyl)oxy)phenyl)bicyclo[2.2.2] octan-1-yl)(4-hydroxypiperidin-1- yl)methanone |
| 2.15 | 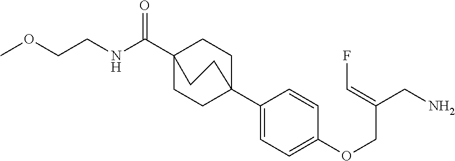 [SMILES: COCCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- methoxyethyl)bicyclo[2.2.2]octane-1- carboxamide |
| 2.16 | 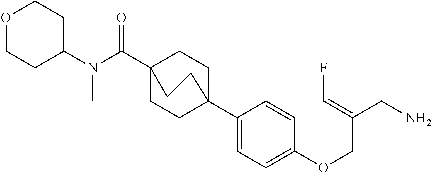 [SMILES: CN(C(=O)C12CCC(c3ccc(OC/C(=C/F)CN)cc3)(CC1)CC2)C1CCOCC1] | (E)-4-(4-((2-(aminomethyl)-3- fluoroallyl)oxy)phenyl)-N-methyl-N- (tetrahydro-2H-pyran-4- yl)bicyclo[2.2.2]octane-1-carboxamide |
| 2.17 | 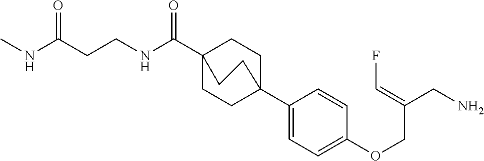 [SMILES: CNC(=O)CCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- (methylamino)-3- oxopropyl)bicyclo[2.2.2]octane-1- carboxamide |
| 2.18 | 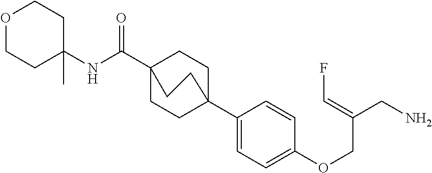 [SMILES: CC1(NC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-(4- methyltetrahydro-2H-pyran-4- yl)bicyclo[2.2.2]octane-1-carboxamide |
| 2.19 | 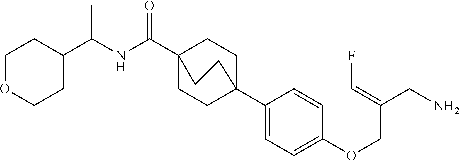 [SMILES: CC(NC(=O)C12CCC(c3ccc(OC/C(=C/F)CN)cc3)(CC1)CC2)C1CCOCC1] | (E)-4-(4-((2-(aminomethyl)-3- fluoroallyl)oxy)phenyl)-N-(1- (tetrahydro-2H-pyran-4- yl)ethyl)bicyclo[2.2.2]octane-1- carboxamide |
| 2.20 | 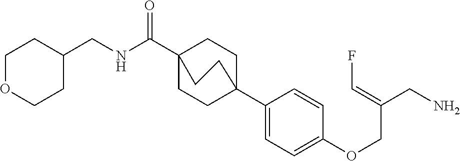 [SMILES: NC/C(=C\F)COc1ccc(C23CCC(C(=O)NCC4CCOCC4)(CC2)CC3)cc1] | (E)-4-(4-((2-(aminomethyl)-3- fluoroallyl)oxy)phenyl)-N- ((tetrahydro-2H-pyran-4- yl)methyl)bicyclo[2.2.2]octane-1- carboxamide |
| 2.21 | 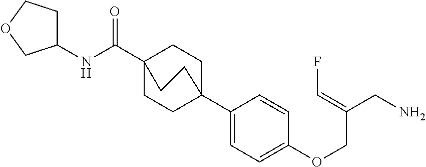 [SMILES: NC/C(=C\F)COc1ccc(C23CCC(C(=O)NC4CCOC4)(CC2)CC3)cc1] | (E)-4-(4-((2-(aminomethyl)-3- fluoroallyl)oxy)phenyl)-N- (tetrahydrofuran-3- yl)bicyclo[2.2.2]octane-1-carboxamide |
| 2.22 | 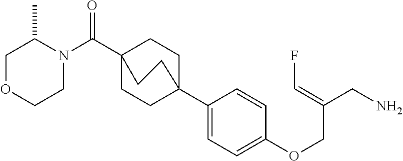 [SMILES: C[C@H]1COCCN1C(=O)C12CCC(c3ccc(OC/C(=C/F)CN)cc3)(CC1)CC2] | (S,E)-(4-(4-((2-(aminomethyl)-3- fluoroallyl)oxy)phenyl)bicyclo[2.2.2] octan-1-yl)(3- methylmorpholino)methanone |
| 2.23 | 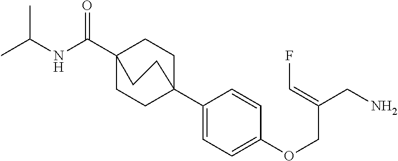 [SMILES: CC(C)NC(=O)C12CCC(c3ccc(OC/C(=C/F)CN)cc3)(CC1)CC2] | (E)-4-(4-((2-(aminomethyl)-3- fluoroallyl)oxy)phenyl)-N- isopropylbicyclo[2.2.2]octane-1- carboxamide |
| 2.24 | 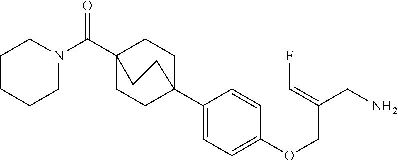 [SMILES: NC/C(=C\F)COc1ccc(C23CCC(C(=O)N4CCCCC4)(CC2)CC3)cc1] | (E)-(4-(4-((2-(aminomethyl)-3- fluoroallyl)oxy)phenyl)bicyclo[2.2.2] octan-1-yl)(piperidin-1-yl)methanone |
| 2.25 | 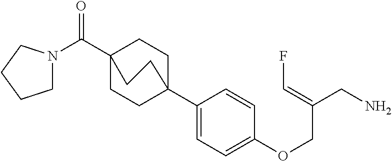 [SMILES: NC/C(=C\F)COc1ccc(C23CCC(C(=O)N4CCCC4)(CC2)CC3)cc1] | (E)-(4-(4-((2-(aminomethyl)-3- fluoroallyl)oxy)phenyl)bicyclo[2.2.2] octan-1-yl)(pyrrolidin-1-yl)methanone |
| 2.26 | 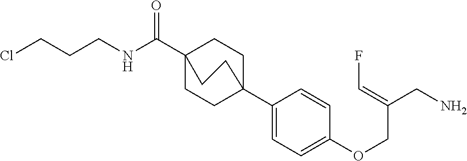 [SMILES: NC/C(=C\F)COc1ccc(C23CCC(C(=O)NCCCCl)(CC2)CC3)cc1] | (E)-4-(4-((2-(aminomethyl)-3- fluoroallyl)oxy)phenyl)-N-(3- chloropropyl)bicyclo[2.2.2]octane-1- carboxamide |
| 2.27 | 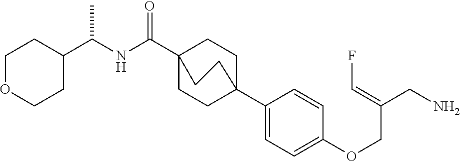 [SMILES: C[C@H](NC(=O)C12CCC(c3ccc(OC/C(=C/F)CN)cc3)(CC1)CC2)C1CCOCC1] | (S,E)-4-(4-((2-(aminomethyl)-3- fluoroallyl)oxy)phenyl)-N-(1- (tetrahydro-2H-pyran-4- yl)ethyl)bicyclo[2.2.2]octane-1- carboxamide |
| 2.28 | 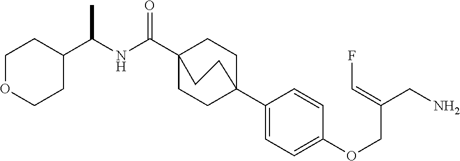 [SMILES: C[C@@H](NC(=O)C12CCC(c3ccc(OC/C(=C/F)CN)cc3)(CC1)CC2)C1CCOCC1] | (R,E)-4-(4-((2-(aminomethyl)-3- fluoroallyl)oxy)phenyl)-N-(1- (tetrahydro-2H-pyran-4- yl)ethyl)bicyclo[2.2.2]octane-1- carboxamide |
| 2.29 | 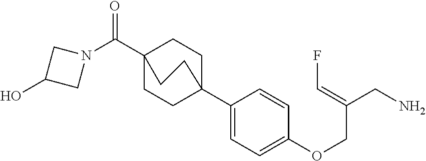 [SMILES: NC/C(=C\F)COc1ccc(C23CCC(C(=O)N4CC(O)C4)(CC2)CC3)cc1] | (E)-(4-(4-((2-(aminomethyl)-3- fluoroallyl)oxy)phenyl)bicyclo[2.2.2] octan-1-yl)(3-hydroxyazetidin-1- yl)methanone |
| 2.30 | 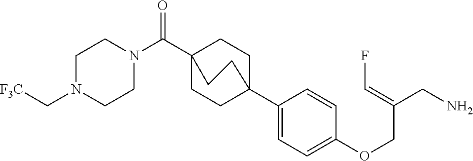 [SMILES: NC/C(=C\F)COc1ccc(C23CCC(C(=O)N4CCN(CC(F)(F)F)CC4)(CC2)CC3)cc1] | (E)-(4-(4-((2-(aminomethyl)-3- fluoroallyl)oxy)phenyl)bicyclo[2.2.2] octan-1-yl)(4-(2,2,2- trifluoroethyl)piperazin-1- yl)methanone |
| 2.31 | 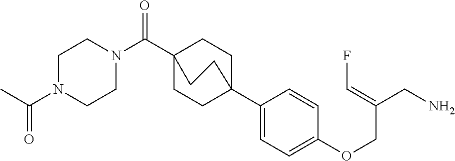 [SMILES: CC(=O)N1CCN(C(=O)C23CCC(c4ccc(OC/C(=C/F)CN)cc4)(CC2)CC3)CC1] | (E)-1-(4-(4-(4-((2-(aminomethyl)-3- fluoroallyl)oxy)phenyl)bicyclo[2.2.2] octane-1-carbonyl)piperazin-1- yl)ethanone |
| 2.32 | 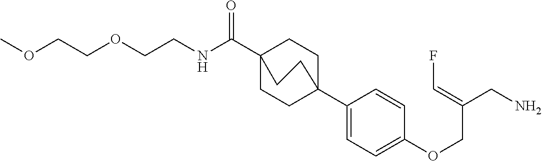 [SMILES: COCCOCCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2-(2- methoxyethoxy)ethyl)bicyclo[2.2.2] octane-1-carboxamide |
| 2.33 | 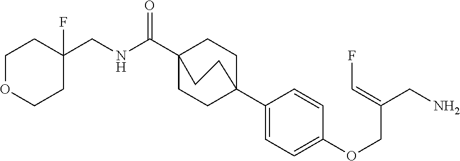 [SMILES: NC/C(=C\F)COc1ccc(C23CCC(C(=O)NCC4(F)CCOCC4)(CC2)CC3)cc1] | (E)-4-(4-((2-(aminomethyl)-3- fluoroallyl)oxy)phenyl)-N-((4- fluorotetrahydro-2H-pyran-4- yl)methyl)bicyclo[2.2.2]octane-1- carboxamide |
| 2.34 | 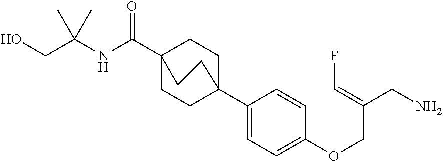 [SMILES: CC(C)(CO)NC(=O)C12CCC(c3ccc(OC/C(=C/F)CN)cc3)(CC1)CC2] | (E)-4-(4-((2-(aminomethyl)-3- fluoroallyl)oxy)phenyl)-N-(1-hydroxy- 2-methylpropan-2- yl)bicyclo[2.2.2]octane-1-carboxamide |
| 2.35 | 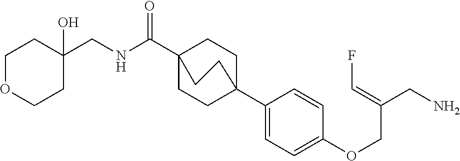 [SMILES: NC/C(=C\F)COc1ccc(C23CCC(C(=O)NCC4(O)CCOCC4)(CC2)CC3)cc1] | (E)-4-(4-((2-(aminomethyl)-3- fluoroallyl)oxy)phenyl)-N-((4- hydroxytetrahydro-2H-pyran-4- yl)methyl)bicyclo[2.2.2]octane-1- carboxamide |
| 2.36 | 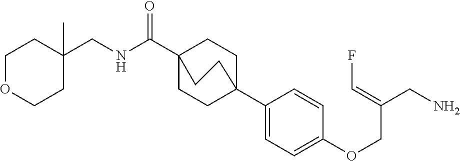 [SMILES: CC1(CNC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-((4- methyltetrahydro-2H-pyran-4- yl)methyl)bicyclo[2.2.2]octane-1- carboxamide |
| 2.37 | 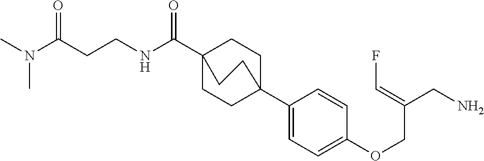 [SMILES: CN(C)C(=O)CCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- (dimethylamino)-3- oxopropyl)bicyclo[2.2.2]octane-1- carboxamide |
| 2.38 | 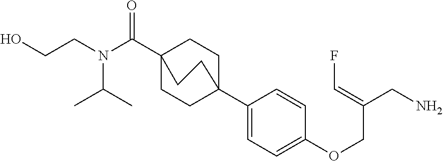 [SMILES: CC(C)N(CCO)C(=O)C12CCC(c3ccc(OC/C(=C/F)CN)cc3)(CC1)CC2] | (E)-4-(4-((2-(aminomethyl)-3- fluoroallyl)oxy)phenyl)-N-(2- hydroxyethyl)-N- isopropylbicyclo[2.2.2]octane-1- carboxamide |
| 2.39 | 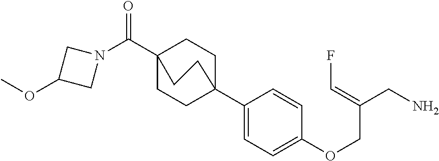 [SMILES: COC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo[2.2.2] octan-1-yl)(3-methoxyazetidin-1- yl)methanone |
| 2.40 | 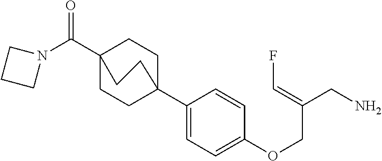 [SMILES: NC/C(=C\F)COc1ccc(C23CCC(C(=O)N4CCC4)(CC2)CC3)cc1] | (E)-(4-(4-((2-(aminomethyl)-3- fluoroallyl)oxy)phenyl)bicyclo[2.2.2] octan-1-yl)(azetidin-1-yl)methanone |
| 2.41 | 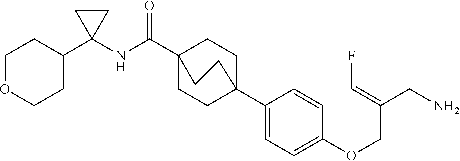 [SMILES: NC/C(=C\F)COc1ccc(C23CCC(C(=O)NC4(C5CCOCC5)CC4)(CC2)CC3)cc1] | (E)-4-(4-((2-(aminomethyl)-3- fluoroallyl)oxy)phenyl)-N-(1- (tetrahydro-2H-pyran-4- yl)cyclopropyl)bicyclo[2.2.2]octane-1- carboxamide |
| 2.42 | 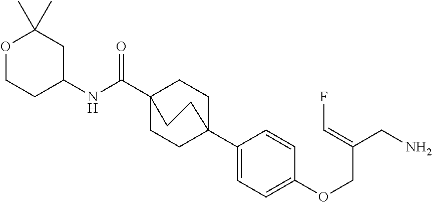 [SMILES: CC1(C)CC(NC(=O)C23CCC(c4ccc(OC/C(=C/F)CN)cc4)(CC2)CC3)CCO1] | (E)-4-(4-((2-(aminomethyl)-3- fluoroallyl)oxy)phenyl)-N-(2,2- dimethyltetrahydro-2H-pyran-4- yl)bicyclo[2.2.2]octane-1-carboxamide |
| 2.43 | 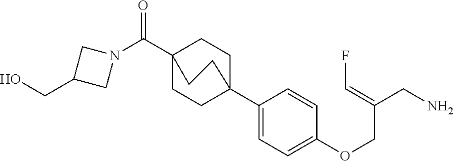 [SMILES: NC/C(=C\F)COc1ccc(C23CCC(C(=O)N4CC(CO)C4)(CC2)CC3)cc1] | (E)-(4-(4-((2-(aminomethyl)-3- fluoroallyl)oxy)phenyl)bicyclo[2.2.2] otan-1-yl)(3-(hydroxymethyl)azetidin- 1-yl)methanone |
| 2.44 | 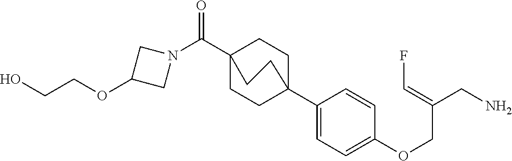 [SMILES: NC/C(=C\F)COc1ccc(C23CCC(C(=O)N4CC(OCCO)C4)(CC2)CC3)cc1] | (E)-(4-(4-((2-(aminomethyl)-3- fluoroallyl)oxy)phenyl)bicyclo[2.2.2] octan-1-yl)(3-(2- hydroxyethoxy)azetidin-1- yl)methanone |
| 2.45 | 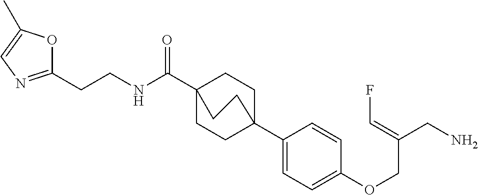 [SMILES: Cc1cnc(CCNC(=O)C23CCC(c4ccc(OC/C(=C/F)CN)cc4)(CC2)CC3)o1] | (E)-4-(4-((2-(aminomethyl)-3- fluoroallyl)oxy)phenyl)-N-(2-(5- methyloxazol-2- yl)ethyl)bicyclo[2.2.2]octane-1- carboxamide |
| 2.46 | 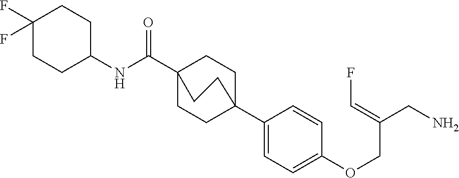 [SMILES: NC/C(=C\F)COc1ccc(C23CCC(C(=O)NC4CCC(F)(F)CC4)(CC2)CC3)cc1] | (E)-4-(4-((2-(aminomethyl)-3- fluoroallyl)oxy)phenyl)-N-(4,4- difluorocyclohexyl)bicyclo[2.2.2] octane-1-carboxamide |
| 2.47 | 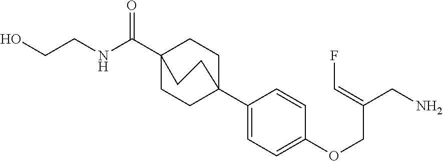 [SMILES: NC/C(=C\F)COc1ccc(C23CCC(C(=O)NCCO)(CC2)CC3)cc1] | (E)-4-(4-((2-(aminomethyl)-3- fluoroallyl)oxy)phenyl)-N-(2- hydroxyethyl)bicyclo[2.2.2]octane-1- carboxamide |
| 2.48 | 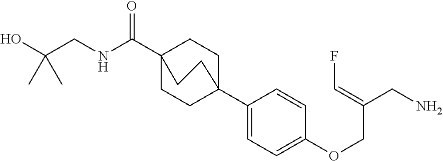 [SMILES: CC(C)(O)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2-hydroxy- 2-methylpropyl)bicyclo[2.2.2]octane- 1-carboxamide |
| 2.49 | 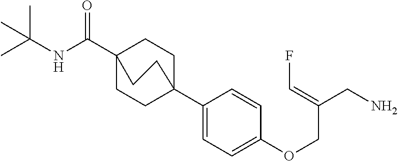 [SMILES: CC(C)(C)NC(=O)C12CCC(c3ccc(OC/C(=C/F)CN)cc3)(CC1)CC2] | (E)-4-(4-((2-(aminomethyl)-3- fluoroallyl)oxy)phenyl)-N-(tert- butyl)bicyclo[2.2.2]octane-1- carboxamide |
| 2.50 | 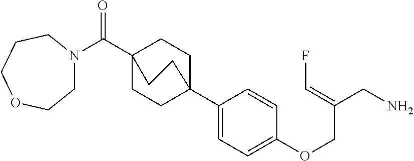 [SMILES: NC/C(=C\F)COc1ccc(C23CCC(C(=O)N4CCCOCC4)(CC2)CC3)cc1] | (E)-(4-(4-((2-(aminomethyl)-3- fluoroallyl)oxy)phenyl)bicyclo[2.2.2] octan-1-yl)(1,4-oxazepan-4- yl)methanone |
| 2.51 | 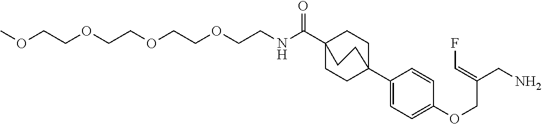 [SMILES: COCCOCCOCCOCCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2,5,8,11- tetraoxatridecan-13- yl)bicyclo[2.2.2]octane-1-carboxamide |
| 2.52 | 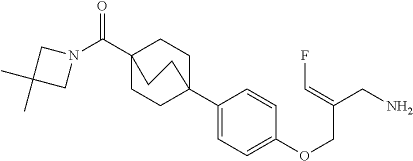 [SMILES: CC1(C)CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo[2.2.2] octan-1-yl)(3,3-dimethylazetidin-1- yl)methanone |
| 2.53 | 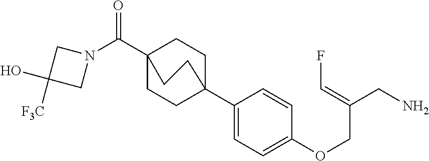 [SMILES: NC/C(=C\F)COc1ccc(C23CCC(C(=O)N4CC(O)(C(F)(F)F)C4)(CC2)CC3)cc1] | (E)-(4-(4-((2-(aminomethyl)-3- fluoroallyl)oxy)phenyl)bicyclo[2.2.2] octan-1-yl)(3-hydroxy-3- (trifluoromethyl)azetidin-1- yl)methanone |
| 2.54 | 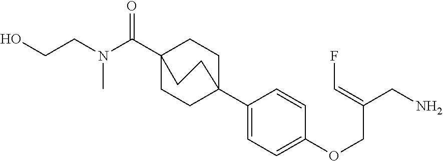 [SMILES: CN(CCO)C(=O)C12CCC(c3ccc(OC/C(=C/F)CN)cc3)(CC1)CC2] | (E)-4-(4-((2-(aminomethyl)-3- fluoroallyl)oxy)phenyl)-N-(2- hydroxyethyl)-N- methylbicyclo[2.2.2]octane-1- carboxamide |
| 2.55 | 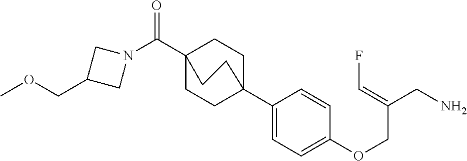 [SMILES: COCC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo[2.2.2] octan-1-yl)(3-(methoxymethyl)azetidin- 1-yl)methanone |
| 2.56 | 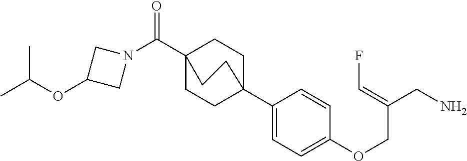 [SMILES: CC(C)OC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo[2.2.2] octan-1-yl)(3-isopropoxyazetidin-1- yl)methanone |
| 2.57 | 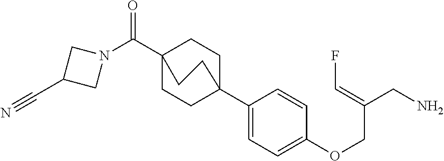 [SMILES: N#CC1CN(C(=O)C23CCC(c4ccc(OC/C(=C/F)CN)cc4)(CC2)CC3)C1] | (E)-1-(4-(4-((2-(aminomethyl)-3- fluoroallyl)oxy)phenyl)bicyclo[2.2.2] octane-1-carbonyl)azetidine-3- carbonitrile |
| 2.58 | 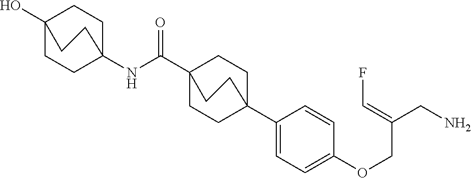 [SMILES: NC/C(=C\F)COc1ccc(C23CCC(C(=O)NC45CCC(O)(CC4)CC5)(CC2)CC3)cc1] | (E)-4-(4-((2-(aminomethyl)-3- fluoroallyl)oxy)phenyl)-N-(4- hydroxybicyclo[2.2.2]octan-1- yl)bicyclo[2.2.2]octane-1-carboxamide |
| 2.59 | 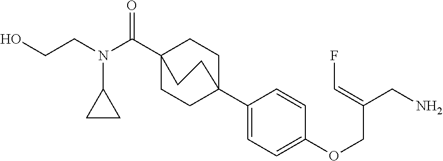 [SMILES: NC/C(=C\F)COc1ccc(C23CCC(C(=O)N(CCO)C4CC4)(CC2)CC3)cc1] | (E)-4-(4-((2-(aminomethyl)-3- fluoroallyl)oxy)phenyl)-N- cyclopropyl-N-(2- hydroxyethyl)bicyclo[2.2.2]octane-1- carboxamide |
| 2.60 | 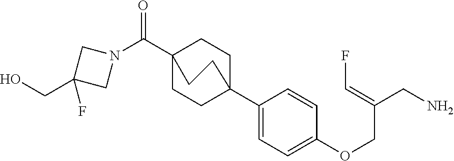 [SMILES: NC/C(=C\F)COc1ccc(C23CCC(C(=O)N4CC(F)(CO)C4)(CC2)CC3)cc1] | (E)-(4-(4-((2-(aminomethyl)-3- fluoroallyl)oxy)phenyl)bicyclo[2.2.2] octan-1-yl)(3-fluoro-3- (hydroxymethyl)azetidin-1- yl)methanone |
| 2.61 | 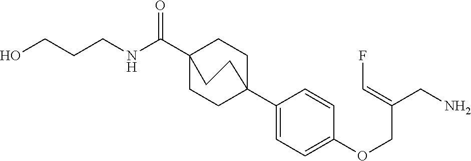 [SMILES: NC/C(=C\F)COc1ccc(C23CCC(C(=O)NCCCO)(CC2)CC3)cc1] | (E)-4-(4-((2-(aminomethyl)-3- fluoroallyl)oxy)phenyl)-N-(3- hydroxypropyl)bicyclo[2.2.2]octane-1- carboxamide |
| 2.62 | 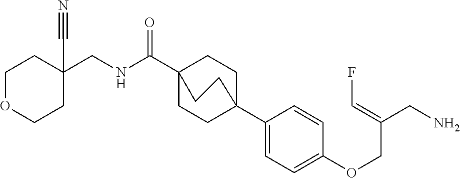 [SMILES: N#CC1(CNC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-((4- cyanotetrahydro-2H-pyran-4- yl)methyl)bicyclo[2.2.2]octane-1- carboxamide |
| 2.63 | 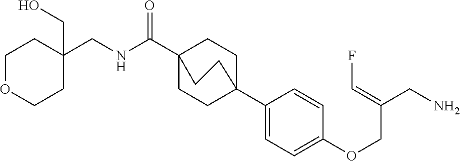 [SMILES: NC/C(=C\F)COc1ccc(C23CCC(C(=O)NCC4(CO)CCOCC4)(CC2)CC3)cc1] | (E)-4-(4-((2-(aminomethyl)-3- fluoroallyl)oxy)phenyl)-N-((4- (hydroxymethyl)tetrahydro-2H-pyran- 4-yl)methyl)bicyclo[2.2.2]octane-1- carboxamide |
| 2.64 | 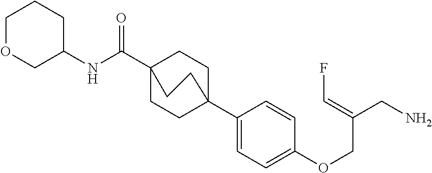 [SMILES: NC/C(=C\F)COc1ccc(C23CCC(C(=O)NC4CCCOC4)(CC2)CC3)cc1] | (E)-4-(4-((2-(aminomethyl)-3- fluoroallyl)oxy)phenyl)-N-(tetrahydro- 2H-pyran-3-yl)bicyclo[2.2.2]octane-1- carboxamide |
| 2.65 | 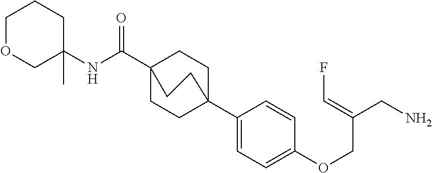 [SMILES: CC1(NC(=O)C23CCC(c4ccc(OC/C(=C/F)CN)cc4)(CC2)CC3)CCCOC1] | (E)-4-(4-((2-(aminomethyl)-3- fluoroallyl)oxy)phenyl)-N-(3- methyltetrahydro-2H-pyran-3- yl)bicyclo[2.2.2]octane-1-carboxamide |
| 2.66 | 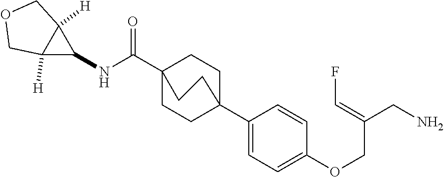 | 4-(4-(((E)-2-(aminomethyl)-3- fluoroallyl)oxy)phenyl)-N- ((1R,5S,6s)-3-oxabicyclo[3.1.0]hexan- 6-yl)bicyclo[2.2.2]octane-1- carboxamide |
| 2.67 | 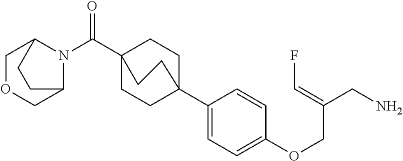 [SMILES: NC/C(=C\F)COc1ccc(C23CCC(C(=O)N4C5CCC4COC5)(CC2)CC3)cc1] | (E)-(4-(4-((2-(aminomethyl)-3- fluoroallyl)oxy)phenyl)bicyclo[2.2.2] octan-1-yl)(3-oxa-8- azabicyclo[3.2.1]octan-8- yl)methanone |
| 2.68 | 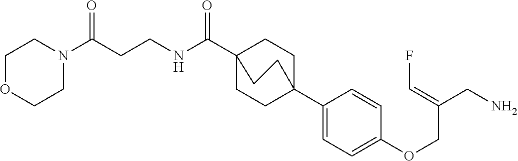 [SMILES: NC/C(=C\F)COc1ccc(C23CCC(C(=O)NCCC(=O)N4CCOCC4)(CC2)CC3)cc1] | (E)-4-(4-((2-(aminomethyl)-3- fluoroallyl)oxy)phenyl)-N-(3- morpholino-3- oxopropyl)bicyclo[2.2.2]octane-1- carboxamide |
| 2.69 | 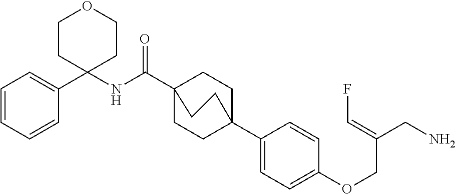 [SMILES: NC/C(=C\F)COc1ccc(C23CCC(C(=O)NC4(c5ccccc5)CCOCC4)(CC2)CC3)cc1] | (E)-4-(4-((2-(aminomethyl)-3- fluoroallyl)oxy)phenyl)-N-(4- phenyltetrahydro-2H-pyran-4- yl)bicyclo[2.2.2]octane-1-carboxamide |
| 2.70 | 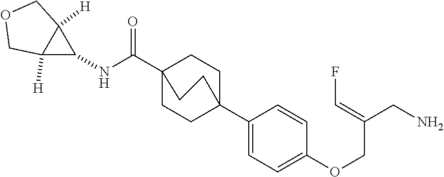 | 4-(4-(((E)-2-(aminomethyl)-3- fluoroallyl)oxy)phenyl)-N-((1R,5S,6r)- 3-oxabicyclo[3.1.0]hexan-6- yl)bicyclo[2.2.2]octane-1-carboxamide |
| 2.71 | 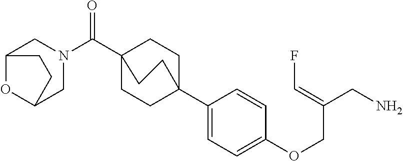 [SMILES: NC/C(=C\F)COc1ccc(C23CCC(C(=O)N4CC5CCC(C4)O5)(CC2)CC3)cc1] | (E)-(4-(4-((2-(aminomethyl)-3- fluoroallyl)oxy)phenyl)bicyclo[2.2.2] octan-1-yl)(8-oxa-3- azabicyclo[3.2.1]octan-3- yl)methanone |
| 2.72 | 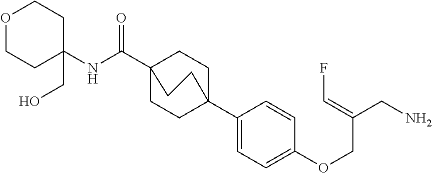 [SMILES: NC/C(=C\F)COc1ccc(C23CCC(C(=O)NC4(CO)CCOCC4)(CC2)CC3)cc1] | (E)-4-(4-((2-(aminomethyl)-3- fluoroallyl)oxy)phenyl)-N-(4- (hydroxymethyl)tetrahydro-2H-pyran- 4-yl)bicyclo[2.2.2]octane-1- carboxamide |
| 2.73 | 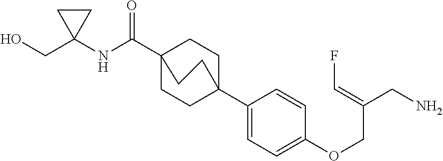 [SMILES: NC/C(=C\F)COc1ccc(C23CCC(C(=O)NC4(CO)CC4)(CC2)CC3)cc1] | (E)-4-(4-((2-(aminomethyl)-3- fluoroallyl)oxy)phenyl)-N-(1- (hydroxymethyl)cyclopropyl)bicyclo [2.2.2]octane-1-carboxamide |
| 2.74 | 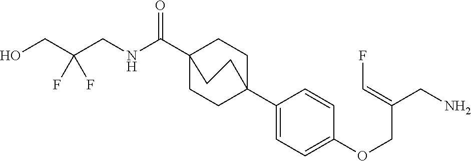 [SMILES: NC/C(=C\F)COc1ccc(C23CCC(C(=O)NCC(F)(F)CO)(CC2)CC3)cc1] | (E)-4-(4-((2-(aminomethyl)-3- fluoroallyl)oxy)phenyl)-N-(2,2- difluoro-3- hydroxypropyl)bicyclo[2.2.2]octane-1- carboxamide |
| 2.75 | 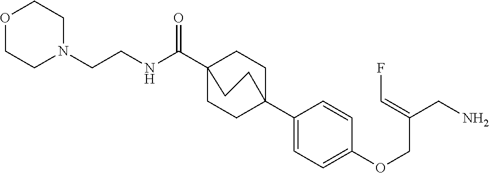 [SMILES: NC/C(=C\F)COc1ccc(C23CCC(C(=O)NCCN4CCOCC4)(CC2)CC3)cc1] | (E)-4-(4-((2-(aminomethyl)-3- fluoroallyl)oxy)phenyl)-N-(2- morpholinoethyl)bicyclo[2.2.2]octane- 1-carboxamide |
| 2.76 | 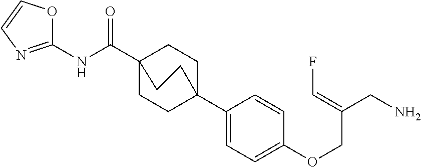 [SMILES: NC/C(=C\F)COc1ccc(C23CCC(C(=O)Nc4ncco4)(CC2)CC3)cc1] | (E)-4-(4-((2-(aminomethyl)-3- fluoroallyl)oxy)phenyl)-N-(oxazol-2- yl)bicyclo[2.2.2]octane-1-carboxamide |
| 2.77 | 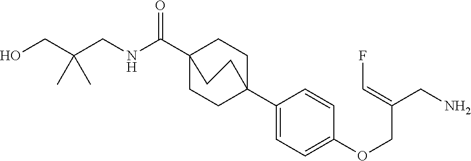 [SMILES: CC(C)(CO)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3-hydroxy- 2,2- dimethylpropyl)bicyclo[2.2.2]octane- 1-carboxamide |
| 2.78 | 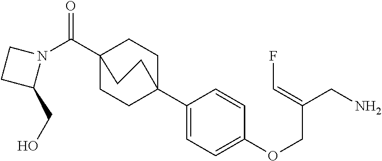 [SMILES: NC/C(=C\F)COc1ccc(C23CCC(C(=O)N4CC[C@@H]4CO)(CC2)CC3)cc1] | (R,E)-(4-(4-((2-(aminomethyl)-3- fluoroallyl)oxy)phenyl)bicyclo[2.2.2] octan-1-yl)(2-(hydroxymethyl)azetidin- 1-yl)methanone |
| 2.79 | 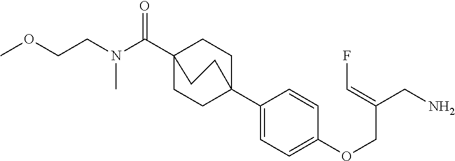 [SMILES: COCCN(C)C(=O)C12CCC(c3ccc(OC/C(=C/F)CN)cc3)(CC1)CC2] | (E)-4-(4-((2-(aminomethyl)-3- fluoroallyl)oxy)phenyl)-N-(2- methoxyethyl)-N- methylbicyclo[2.2.2]octane-1- carboxamide |
| 2.80 | 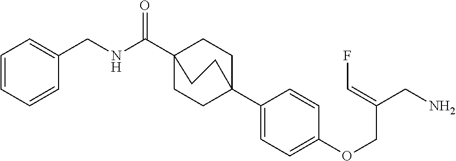 [SMILES: NC/C(=C\F)COc1ccc(C23CCC(C(=O)NCc4ccccc4)(CC2)CC3)cc1] | (E)-4-(4-((2-(aminomethyl)-3- fluoroallyl)oxy)phenyl)-N- benzylbicyclo[2.2.2]octane-1- carboxamide |
| 2.81 | 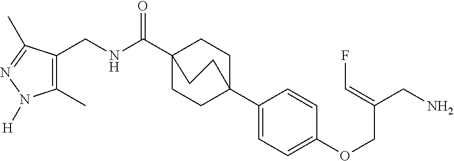 [SMILES: Cc1n[nH]c(C)c1CNC(=O)C12CCC(c3ccc(OC/C(=C/F)CN)cc3)(CC1)CC2] | (E)-4-(4-((2-(aminomethyl)-3- fluoroallyl)oxy)phenyl)-N-((3,5- dimethyl-1H-pyrazol-4- yl)methyl)bicyclo[2.2.2]octane-1- carboxamide |
| 2.82 | 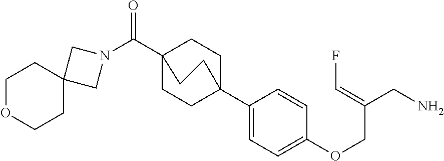 [SMILES: NC/C(=C\F)COc1ccc(C23CCC(C(=O)N4CC5(CCOCC5)C4)(CC2)CC3)cc1] | (E)-(4-(4-((2-(aminomethyl)-3- fluoroallyl)oxy)phenyl)bicyclo[2.2.2] octan-1-yl)(7-oxa-2- azaspiro[3.5]nonan-2-yl)methanone |
| 2.83 | 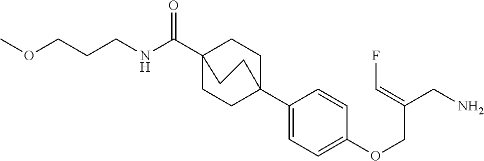 [SMILES: COCCCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- methoxypropyl)bicyclo[2.2.2]octane- 1-carboxamide |
| 2.84 | 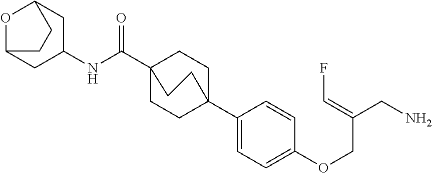 [SMILES: NC/C(=C\F)COc1ccc(C23CCC(C(=O)NC4CC5CCC(C4)O5)(CC2)CC3)cc1] | (E)-4-(4-((2-(aminomethyl)-3- fluoroallyl)oxy)phenyl)-N-(8- oxabicyclo[3.2.1]octan-3- yl)bicyclo[2.2.2]octane-1-carboxamide |
| 2.85 | 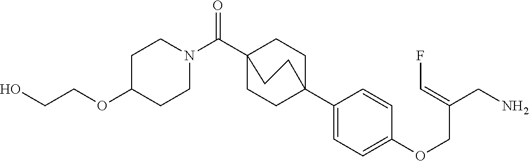 [SMILES: NC/C(=C\F)COc1ccc(C23CCC(C(=O)N4CCC(OCCO)CC4)(CC2)CC3)cc1] | (E)-(4-(4-((2-(aminomethyl)-3- fluoroallyl)oxy)phenyl)bicyclo[2.2.2] octan-1-yl)(4-(2- hydroxyethoxy)piperidin-1- yl)methanone |
| 2.86 | 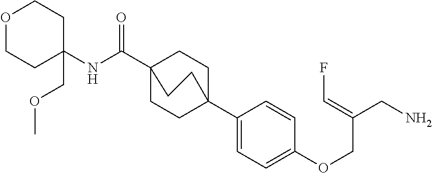 [SMILES: COCC1(NC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-(4- (methoxymethyl)tetrahydro-2H-pyran- 4-yl)bicyclo[2.2.2]octane-1- carboxamide |
| 2.87 | 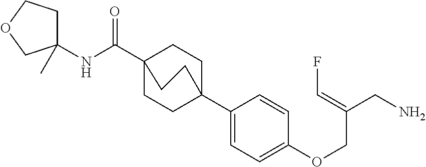 [SMILES: CC1(NC(=O)C23CCC(c4ccc(OC/C(=C/F)CN)cc4)(CC2)CC3)CCOC1] | (E)-4-(4-((2-(aminomethyl)-3- fluoroallyl)oxy)phenyl)-N-(3- methyltetrahydrofuran-3- yl)bicyclo[2.2.2]octane-1-carboxamide |
| 2.88 | 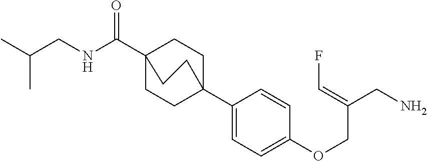 [SMILES: CC(C)CNC(=O)C12CCC(c3ccc(OC/C(=C/F)CN)cc3)(CC1)CC2] | (E)-4-(4-((2-(aminomethyl)-3- fluoroallyl)oxy)phenyl)-N- isobutylbicyclo[2.2.2]octane-1- carboxamide |
| 2.89 | 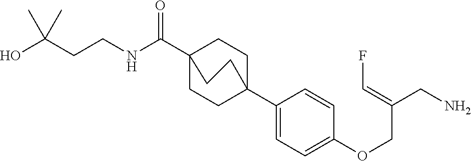 [SMILES: CC(C)(O)CCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3-hydroxy- 3-methylbutyl)bicyclo[2.2.2]octane-1- carboxamide |
| 2.90 | 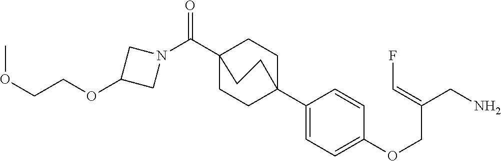 [SMILES: COCCOC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo[2.2.2] octan-1-yl)(3-(2- methoxyethoxy)azetidin-1- yl)methanone |
| 2.91 | 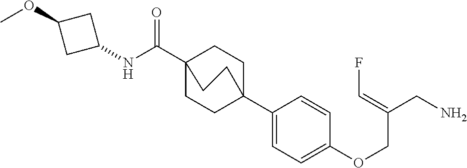 | 4-(4-(((E)-2-(aminomethyl)-3- fluoroallyl)oxy)phenyl)-N-((1r,3r)-3- methoxycyclobutyl)bicyclo[2.2.2] octane-1-carboxamide |
| 2.92 | 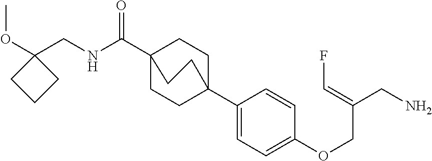 [SMILES: COC1(CNC(=O)C23CCC(c4ccc(OC/C(=C/F)CN)cc4)(CC2)CC3)CCC1] | (E)-4-(4-((2-(aminomethyl)-3- fluoroallyl)oxy)phenyl)-N-((1- methoxycyclobutyl)methyl)bicyclo [2.2.2]octane-1-carboxamide |
| 2.93 | 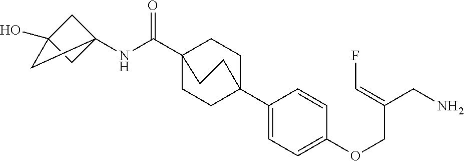 [SMILES: NC/C(=C\F)COc1ccc(C23CCC(C(=O)NC45CC(O)(C4)C5)(CC2)CC3)cc1] | (E)-4-(4-((2-(aminomethyl)-3- fluoroallyl)oxy)phenyl)-N-(3- hydroxybicyclo[1.1.1]pentan-1- yl)bicyclo[2.2.2]octane-1-carboxamide |
| 2.94 | 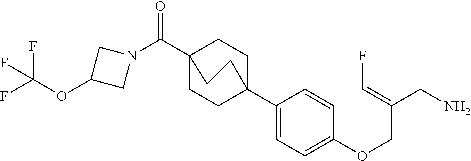 [SMILES: NC/C(=C\F)COc1ccc(C23CCC(C(=O)N4CC(OC(F)(F)F)C4)(CC2)CC3)cc1] | (E)-(4-(4-((2-(aminomethyl)-3- fluoroallyl)oxy)phenyl)bicyclo[2.2.2] octan-1-yl)(3- (trifluoromethoxy)azetidin-1- yl)methanone |
| 2.95 | 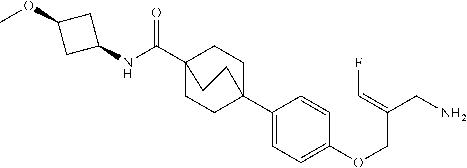 [SMILES: CO[C@H]1C[C@@H](NC(=O)C23CCC(c4ccc(OC/C(=C/F)CN)cc4)(CC2)CC3)C1] | 4-(4-(((E)-2-(aminomethyl)-3- fluoroallyl)oxy)phenyl)-N-((1s,3s)-3- methoxycyclobutyl)bicyclo[2.2.2] octane-1-carboxamide |
| 2.96 | 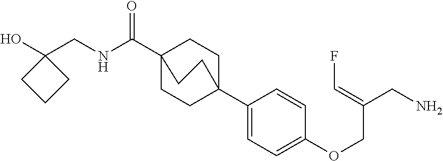 [SMILES: NC/C(=C\F)COc1ccc(C23CCC(C(=O)NCC4(O)CCC4)(CC2)CC3)cc1] | (E)-4-(4-((2-(aminomethyl)-3- fluoroallyl)oxy)phenyl)-N-((1- hydroxycyclobutyl)methyl)bicyclo [2.2.2]octane-1-carboxamide |
| 2.97 | 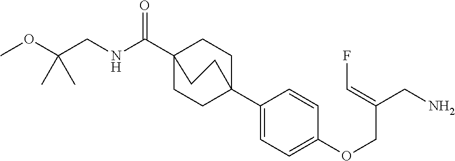 [SMILES: COC(C)(C)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- methoxy-2- methylpropyl)bicyclo[2.2.2]octane-1- carboxamide |
| 2.98 | 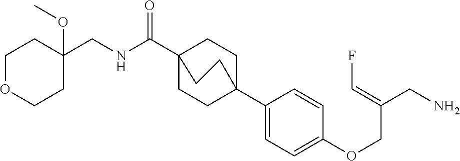 [SMILES: COC1(CNC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-((4- methoxytetrahydro-2H-pyran-4- yl)methyl)bicyclo[2.2.2]octane-1- carboxamide |
| 2.99 | 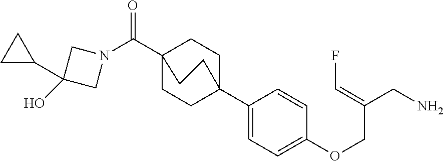 [SMILES: NC/C(=C\F)COc1ccc(C23CCC(C(=O)N4CC(O)(C5CC5)C4)(CC2)CC3)cc1] | (E)-(4-(4-((2-(aminomethyl)-3- fluoroallyl)oxy)phenyl)bicyclo[2.2.2] octan-1-yl)(3-cyclopropyl-3- hydroxyazetidin-1-yl)methanone |
| 2.100 | 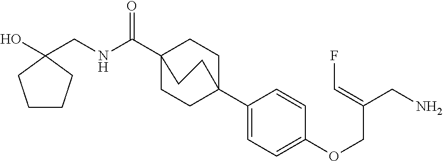 [SMILES: NC/C(=C\F)COc1ccc(C23CCC(C(=O)NCC4(O)CCCC4)(CC2)CC3)cc1] | (E)-4-(4-((2-(aminomethyl)-3- fluoroallyl)oxy)phenyl)-N-((1- hydroxycyclopentyl)methyl)bicyclo [2.2.2]octane-1-carboxamide |
| 2.101 | 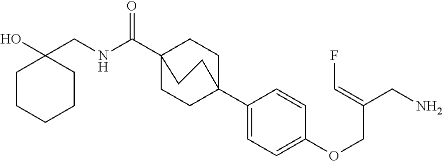 [SMILES: NC/C(=C\F)COc1ccc(C23CCC(C(=O)NCC4(O)CCCCC4)(CC2)CC3)cc1] | (E)-4-(4-((2-(aminomethyl)-3- fluoroallyl)oxy)phenyl)-N-((1- hydroxycyclohexyl)methyl)bicyclo [2.2.2]octane-1-carboxamide |
| 2.102 | 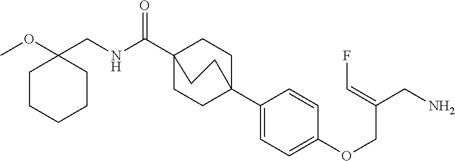 [SMILES: COC1(CNC(=O)C23CCC(c4ccc(OC/C(=C/F)CN)cc4)(CC2)CC3)CCCCC1] | (E)-4-(4-((2-(aminomethyl)-3- fluoroallyl)oxy)phenyl)-N-((1- methoxycyclohexyl)methyl)bicyclo [2.2.2]octane-1-carboxamide |
| 2.103 | 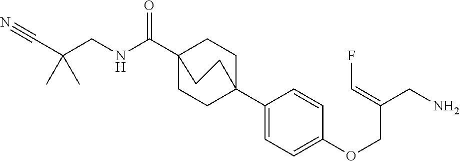 [SMILES: CC(C)(C#N)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2-cyano-2- methylpropyl)bicyclo[2.2.2]octane-1- carboxamide |
| 2.104 | 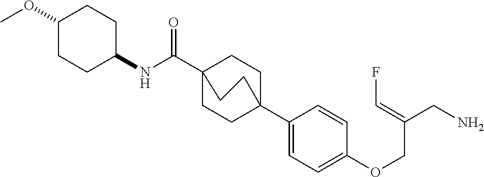 | 4-(4-(((E)-2-(aminomethyl)-3- fluoroallyl)oxy)phenyl)-N-((1r,4r)-4- methoxycyclohexyl)bicyclo[2.2.2] octane-1-carboxamide |
| 2.105 | 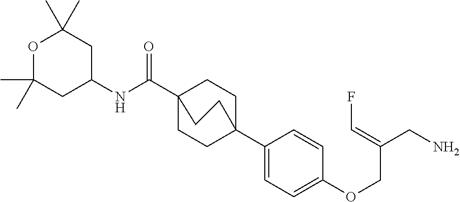 [SMILES: CC1(C)CC(NC(=O)C23CCC(c4ccc(OC/C(=C/F)CN)cc4)(CC2)CC3)CC(C)(C)O1] | (E)-4-(4-((2-(aminomethyl)-3- fluoroallyl)oxy)phenyl)-N-(2,2,6,6- tetramethyltetrahydro-2H-pyran-4- yl)bicyclo[2.2.2]octane-1-carboxamide |
| 2.106 | 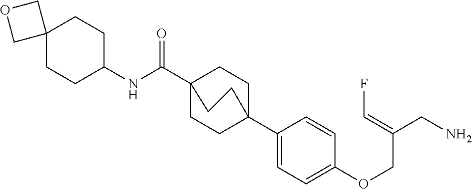 [SMILES: NC/C(=C\F)COc1ccc(C23CCC(C(=O)NC4CCC5(CC4)COC5)(CC2)CC3)cc1] | (E)-4-(4-((2-(aminomethyl)-3- fluoroallyl)oxy)phenyl)-N-(2- oxaspiro[3.5]nonan-7- yl)bicyclo[2.2.2]octane-1-carboxamide |
| 2.107 | 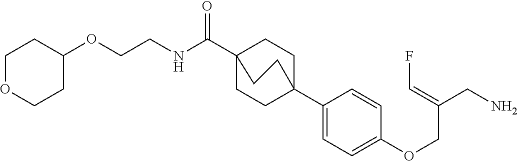 [SMILES: NC/C(=C\F)COc1ccc(C23CCC(C(=O)NCCOC4CCOCC4)(CC2)CC3)cc1] | (E)-4-(4-((2-(aminomethyl)-3- fluoroallyl)oxy)phenyl)-N-(2- ((tetrahydro-2H-pyran-4- yl)oxy)ethyl)bicyclo[2.2.2]octane-1- carboxamide |
| 2.108 | 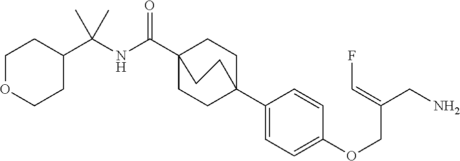 [SMILES: CC(C)(NC(=O)C12CCC(c3ccc(OC/C(=C/F)CN)cc3)(CC1)CC2)C1CCOCC1] | (E)-4-(4-((2-(aminomethyl)-3- fluoroallyl)oxy)phenyl)-N-(2- (tetrahydro-2H-pyran-4-yl)propan-2- yl)bicyclo[2.2.2]octane-1-carboxamide |
| 2.109 | 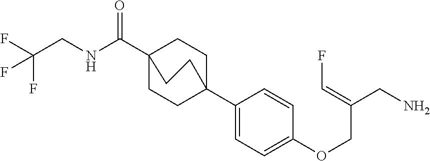 [SMILES: NC/C(=C\F)COc1ccc(C23CCC(C(=O)NCC(F)(F)F)(CC2)CC3)cc1] | (E)-4-(4-((2-(aminomethyl)-3- fluoroallyl)oxy)phenyl)-N-(2,2,2- trifluoroethyl)bicyclo[2.2.2]octane-1- carboxamide |
| 2.110 | 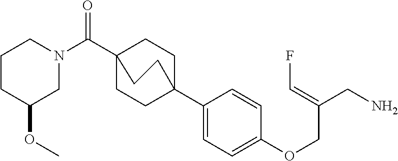 [SMILES: CO[C@H]1CCCN(C(=O)C23CCC(c4ccc(OC/C(=C/F)CN)cc4)(CC2)CC3)C1] | (S,E)-(4-(4-((2-(aminomethyl)-3- fluoroallyl)oxy)phenyl)bicyclo[2.2.2] octan-1-yl)(3-methoxypiperidin-1- yl)methanone |
| 2.111 | 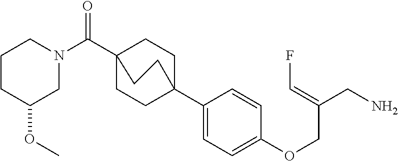 [SMILES: CO[C@@H]1CCCN(C(=O)C23CCC(c4ccc(OC/C(=C/F)CN)cc4)(CC2)CC3)C1] | (R,E)-(4-(4-((2-(aminomethyl)-3- fluoroallyl)oxy)phenyl)bicyclo[2.2.2] octan-1-yl)(3-methoxypiperidin-1- yl)methanone |
| 2.112 | 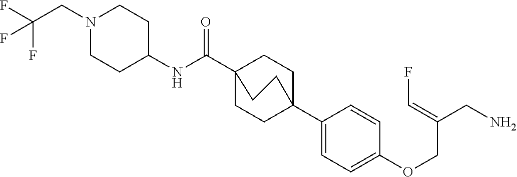 [SMILES: NC/C(=C\F)COc1ccc(C23CCC(C(=O)NC4CCN(CC(F)(F)F)CC4)(CC2)CC3)cc1] | (E)-4-(4-((2-(aminomethyl)-3- fluoroallyl)oxy)phenyl)-N-(1-(2,2,2- trifluoroethyl)piperidin-4- yl)bicyclo[2.2.2]octane-1-carboxamide |
| 2.113 | 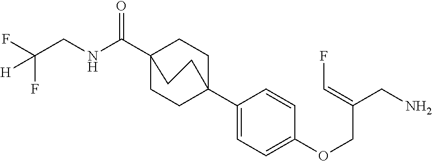 [SMILES: NC/C(=C\F)COc1ccc(C23CCC(C(=O)NCC(F)F)(CC2)CC3)cc1] | (E)-4-(4-((2-(aminomethyl)-3- fluoroallyl)oxy)phenyl)-N-(2,2- difluoroethyl)bicyclo[2.2.2]octane-1- carboxamide |
| 2.114 | 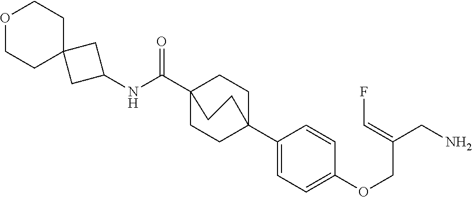 [SMILES: NC/C(=C\F)COc1ccc(C23CCC(C(=O)NC4CC5(CCOCC5)C4)(CC2)CC3)cc1] | (E)-4-(4-((2-(aminomethyl)-3- fluoroallyl)oxy)phenyl)-N-(7- oxaspiro[3.5]nonan-2- yl)bicyclo[2.2.2]octane-1-carboxamide |
| 2.115 | 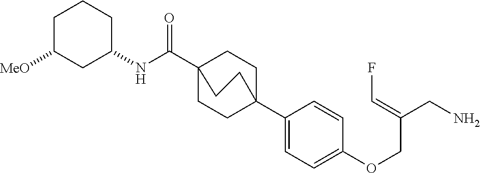 | 4-(4-(((E)-2-(aminomethyl)-3- fluoroallyl)oxy)phenyl)-N-((1S,3R)-3- methoxycyclohexyl)bicyclo[2.2.2] octane-1-carboxamide |
| 2.116 | 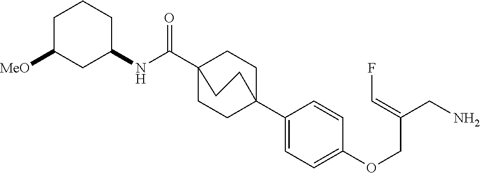 | 4-(4-(((E)-2-(aminomethyl)-3- fluoroallyl)oxy)phenyl)-N-((1R,3S)-3- methoxycyclohexyl)bicyclo[2.2.2] octane-1-carboxamide |
| 2.117 | 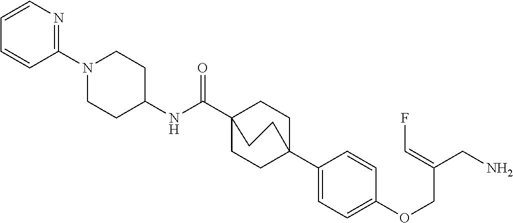 [SMILES: NC/C(=C\F)COc1ccc(C23CCC(C(=O)NC4CCN(c5ccccn5)CC4)(CC2)CC3)cc1] | (E)-4-(4-((2-(aminomethyl)-3- fluoroallyl)oxy)phenyl)-N-(1-(pyridin- 2-yl)piperidin-4- yl)bicyclo[2.2.2]octane-1-carbxamide |
| 2.118 | 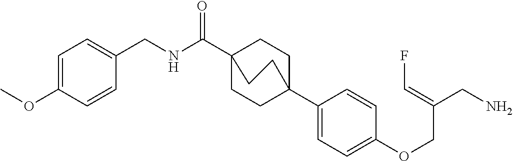 [SMILES: COc1ccc(CNC(=O)C23CCC(c4ccc(OC/C(=C/F)CN)cc4)(CC2)CC3)cc1] | (E)-4-(4-((2-(aminomethyl)-3- fluoroallyl)oxy)phenyl)-N-(4- methoxybenzyl)bicyclo[2.2.2]octane- 1-carboxamide |
| 2.119 | 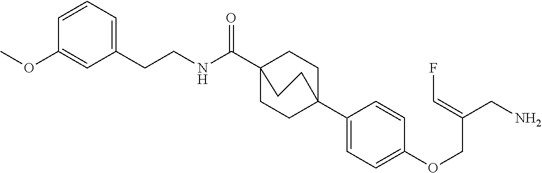 [SMILES: COc1cccc(CCNC(=O)C23CCC(c4ccc(OC/C(=C/F)CN)cc4)(CC2)CC3)c1] | (E)-4-(4-((2-(aminomethyl)-3- fluoroallyl)oxy)phenyl)-N-(3- methoxyphenethyl)bicyclo[2.2.2] octane-1-carboxamide |
| 2.120 | 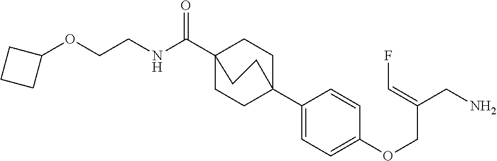 [SMILES: NC/C(=C\F)COc1ccc(C23CCC(C(=O)NCCOC4CCC4)(CC2)CC3)cc1] | (E)-4-(4-((2-(aminomethyl)-3- fluoroallyl)oxy)phenyl)-N-(2- cyclobutoxyethyl)bicyclo[2.2.2] octane-1-carboxamide |
| 2.121 | 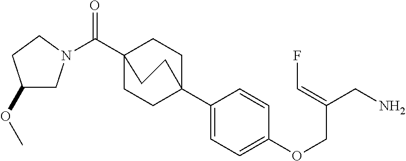 [SMILES: CO[C@H]1CCN(C(=O)C23CCC(c4ccc(OC/C(=C/F)CN)cc4)(CC2)CC3)C1] | (S,E)-(4-(4-((2-(aminomethyl)-3- fluoroallyl)oxy)phenyl)bicyclo[2.2.2] octan-1-yl)(3-mthoxypyrrolidin-1- yl)methanone |
| 2.122 | 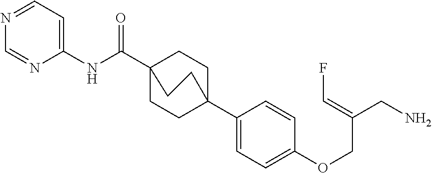 [SMILES: NC/C(=C\F)COc1ccc(C23CCC(C(=O)Nc4ccncn4)(CC2)CC3)cc1] | (E)-4-(4-((2-(aminomethyl)-3- fluoroallyl)oxy)phenyl)-N-(pyrimidin- 4-yl)bicyclo[2.2.2]octane-1- carboxamide |
| 2.123 | 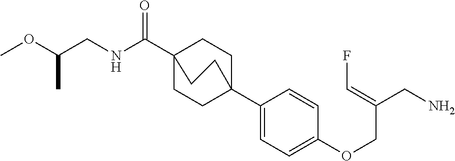 [SMILES: CO[C@H](C)CNC(=O)C12CCC(c3ccc(OC/C(=C/F)CN)cc3)(CC1)CC2] | (R,E)-4-(4-((2-(aminomethyl)-3- fluoroallyl)oxy)phenyl)-N-(2- methoxypropyl)bicyclo[2.2.2]octane- 1-carboxamide |
| 2.124 | 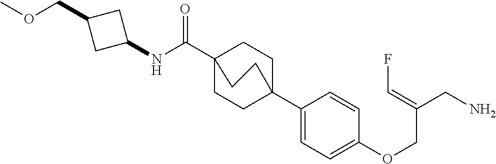 | 4-(4-(((E)-2-(aminomethyl)-3- fluoroallyl)oxy)phenyl)-N-((1s,3s)-3- (methoxymethyl)cyclobutyl)bicyclo [2.2.2]octane-1-carboxamide |
| 2.125 | 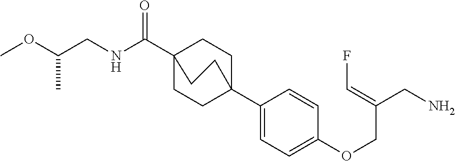 [SMILES: CO[C@@H](C)CNC(=O)C12CCC(c3ccc(OC/C(=C/F)CN)cc3)(CC1)CC2] | (S,E)-4-(4-((2-(aminomethyl)-3- fluoroallyl)oxy)phenyl)-N-(2- methoxypropyl)bicyclo[2.2.2]octane- 1-carboxamide |
| 2.126 | 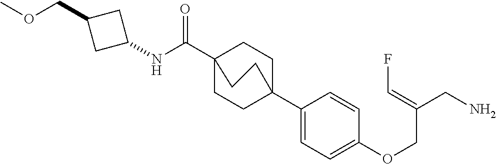 | 4-(4-(((E)-2-(aminomethyl)-3- fluoroallyl)oxy)phenyl)-N-((1r,3r)-3- (methoxymethyl)cyclobutyl)bicyclo [2.2.2]octane-1-carboxamide |
| 2.127 | 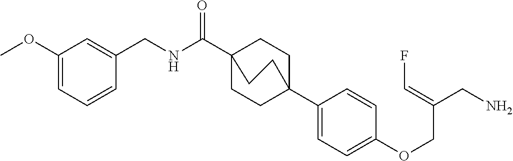 [SMILES: COc1cccc(CNC(=O)C23CCC(c4ccc(OC/C(=C/F)CN)cc4)(CC2)CC3)c1] | (E)-4-(4-((2-(aminomethyl)-3- fluoroallyl)oxy)phenyl)-N-(3- methoxybenzyl)bicyclo[2.2.2]octane- 1-carboxamide |
| 2.128 | 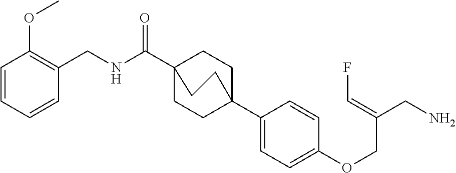 [SMILES: COc1ccccc1CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- methoxybenzyl)bicyclo[2.2.2]octane- 1-carboxamide |
| 2.129 | 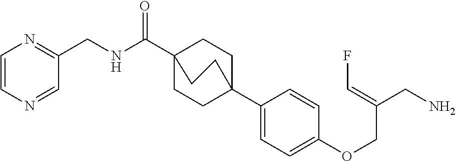 [SMILES: NC/C(=C\F)COc1ccc(C23CCC(C(=O)NCc4cnccn4)(CC2)CC3)cc1] | (E)-4-(4-((2-(aminomethyl)-3- fluoroallyl)oxy)phenyl)-N-(pyrazin-2- ylmethyl)bicyclo[2.2.2]octane-1- carboxamide |
| 2.130 | 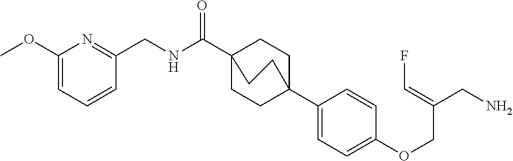 [SMILES: COc1cccc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((6- methoxypyridin-2- yl)methyl)bicyclo[2.2.2]octane-1- carboxamide |
| 2.131 | 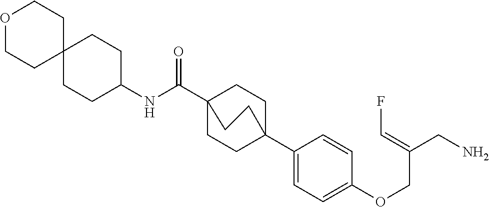 [SMILES: NC/C(=C\F)COc1ccc(C23CCC(C(=O)NC4CCC5(CCOCC5)CC4)(CC2)CC3)cc1] | (E)-4-(4-((2-(aminomethyl)-3- fluoroallyl)oxy)phenyl)-N-(3- oxaspiro[5.5]undecan-9- yl)bicyclo[2.2.2]octane-1-carboxamide |
| 2.132 | 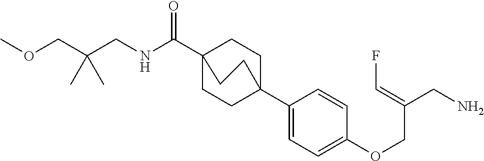 [SMILES: COCC(C)(C)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- methoxy-2,2- dimethylpropyl)bicyclo[2.2.2]octane- 1-carboxamide |
| 2.133 | 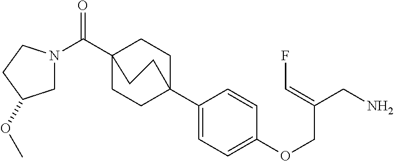 [SMILES: CO[C@@H]1CCN(C(=O)C23CCC(c4ccc(OC/C(=C/F)CN)cc4)(CC2)CC3)C1] | (R,E)-(4-(4-((2-(aminomethyl)-3- fluoroallyl)oxy)phenyl)bicyclo[2.2.2] octan-1-yl)(3-methoxypyrrolidin-1- yl)methanone |
| 2.134 | 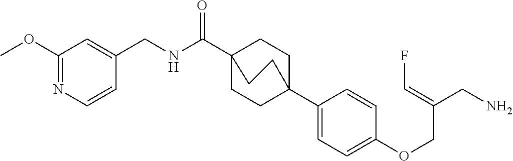 [SMILES: COc1cc(CNC(=O)C23CCC(c4ccc(OC/C(=C/F)CN)cc4)(CC2)CC3)ccn1] | (E)-4-(4-((2-(aminomethyl)-3- fluoroallyl)oxy)phenyl)-N-((2- methoxypyridin-4- yl)methyl)bicyclo[2.2.2]octane-1- carboxamide |
| 2.135 | 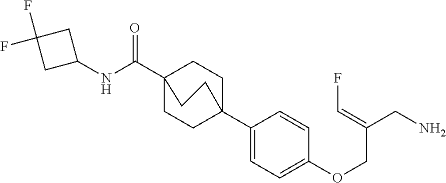 [SMILES: NC/C(=C\F)COc1ccc(C23CCC(C(=O)NC4CC(F)(F)C4)(CC2)CC3)cc1] | (E)-4-(4-((2-(aminomethyl)-3- fluoroallyl)oxy)phenyl)-N-(3,3- difluorocyclobutyl)bicyclo[2.2.2] octane-1-carboxamide |
| 2.136 | 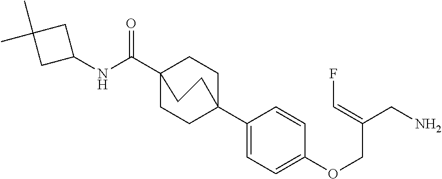 [SMILES: CC1(C)CC(NC(=O)C23CCC(c4ccc(OC/C(=C/F)CN)cc4)(CC2)CC3)C1] | (E)-4-(4-((2-(aminomethyl)-3- fluoroallyl)oxy)phenyl)-N-(3,3- dimethylcyclobutyl)bicyclo[2.2.2] octane-1-carboxamide |
| 2.137 | 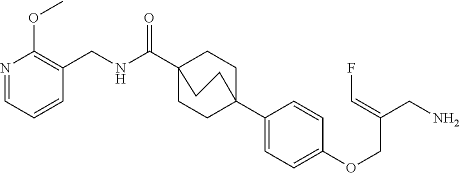 [SMILES: COc1ncccc1CNC(=O)C12CCC(c3ccc(OC/C(=C/F)CN)cc3)(CC1)CC2] | (E)-4-(4-((2-(aminomethyl)-3- fluoroallyl)oxy)phenyl)-N-((2- methoxypyridin-3- yl)methyl)bicyclo[2.2.2]octane-1- carboxamide |
| 2.138 | 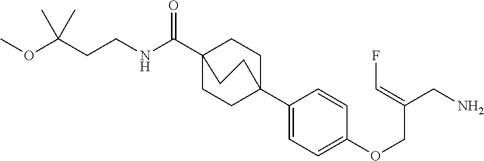 [SMILES: COC(C)(C)CCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- methoxy-3- methylbutyl)bicyclo[2.2.2]octane-1- carboxamide |
| 2.139 | 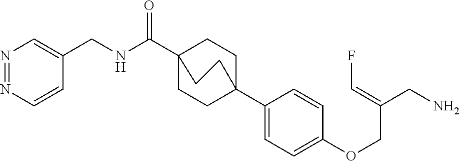 [SMILES: NC/C(=C\F)COc1ccc(C23CCC(C(=O)NCc4ccnnc4)(CC2)CC3)cc1] | (E)-4-(4-((2-(aminomethyl)-3- fluoroallyl)oxy)phenyl)-N-(pyridazin- 4-ylmethyl)bicyclo[2.2.2]octane-1- carboxamide |
| 2.140 | 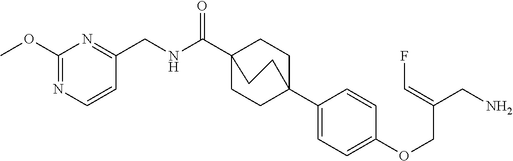 [SMILES: COc1nccc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((2- methoxypyrimidin-4- yl)methyl)bicyclo[2.2.2]octane-1- carboxamide |
| 2.141 | 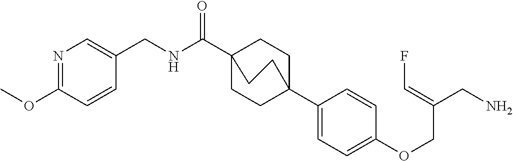 [SMILES: COc1ccc(CNC(=O)C23CCC(c4ccc(OC/C(=C/F)CN)cc4)(CC2)CC3)cn1] | (E)-4-(4-((2-(aminomethyl)-3- fluoroallyl)oxy)phenyl)-N-((6- methoxypyridin-3- yl)methyl)bicyclo[2.2.2]octane-1- carboxamide |
| 2.142 | 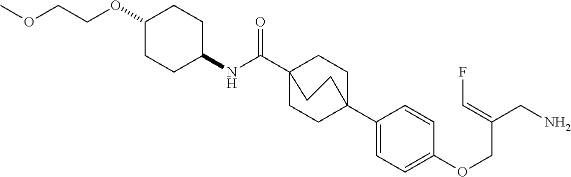 | 4-(4-(((E)-2-(aminomethyl)-3- fluoroallyl)oxy)phenyl)-N-((1r,4r)-4- (2- methoxyethoxy)cyclohexyl)bicyclo [2.2.2]octane-1-carboxamide |
| 2.143 | 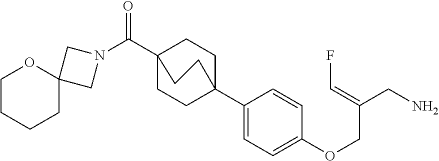 [SMILES: NC/C(=C\F)COc1ccc(C23CCC(C(=O)N4CC5(CCCCO5)C4)(CC2)CC3)cc1] | (E)-(4-(4-((2-(aminomethyl)-3- fluoroallyl)oxy)phenyl)bicyclo[2.2.2] octan-1-yl)(5-oxa-2- azaspiro[3.5]nonan-2-yl)methanone |
| 2.144 | 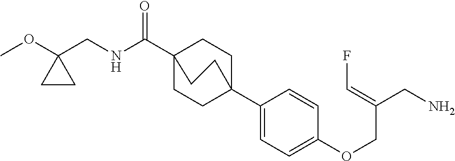 [SMILES: COC1(CNC(=O)C23CCC(c4ccc(OC/C(=C/F)CN)cc4)(CC2)CC3)CC1] | (E)-4-(4-((2-(aminomethyl)-3- fluoroallyl)oxy)phenyl)-N-((1- methoxycyclopropyl)methyl)bicyclo [2.2.2]octane-1-carboxamide |
| 2.145 | 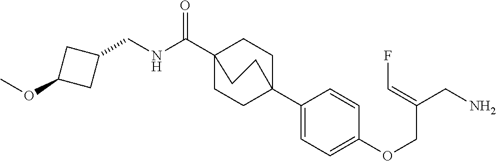 [SMILES: CO[C@H]1C[C@H](CNC(=O)C23CCC(c4ccc(OC/C(=C/F)CN)cc4)(CC2)CC3)C1] | 4-(4-(((E)-2-(aminomethyl)-3- fluoroallyl)oxy)phenyl)-N-(((1r,3r)-3- methoxycyclobutyl)methyl)bicyclo [2.2.2]octane-1-carboxamide |
| 2.146 | 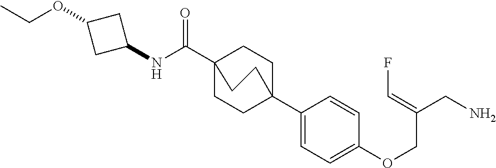 | 4-(4-(((E)-2-(aminomethyl)-3- fluoroallyl)oxy)phenyl)-N-((1r,3r)-3- ethoxycyclobutyl)bicyclo[2.2.2]octane- 1-carboxamide |
| 2.147 | 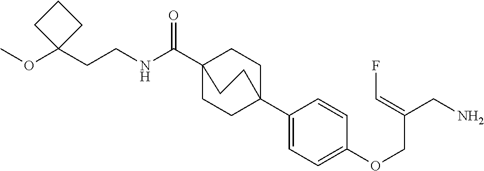 [SMILES: COC1(CCNC(=O)C23CCC(c4ccc(OC/C(=C/F)CN)cc4)(CC2)CC3)CCC1] | (E)-4-(4-((2-(aminomethyl)-3- fluoroallyl)oxy)phenyl)-N-(2-(1- methoxycyclobutyl)ethyl)bicyclo[2.2.2] octane-1-carboxamide |
| 2.148 | 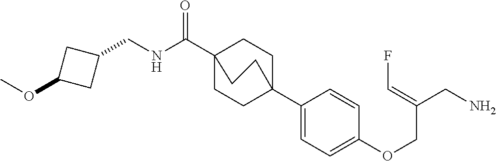 [SMILES: CO[C@H]1C[C@H](CNC(=O)C23CCC(c4ccc(OC/C(=C/F)CN)cc4)(CC2)CC3)C1] | 4-(4-(((E)-2-(aminomethyl)-3- fluoroallyl)oxy)phenyl)-N-(((1s,3s)-3- methoxycyclobutyl)methyl)bicyclo [2.2.2]octane-1-carboxamide |
| 2.149 | 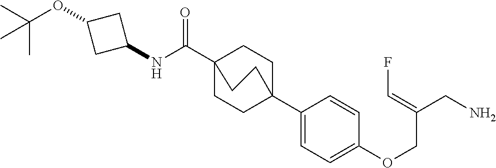 | 4-(4-(((E)-2-(aminomethyl)-3- fluoroallyl)oxy)phenyl)-N-((1s,3s)-3- (tert- butoxy)cyclobutyl)bicyclo[2.2.2] octane-1-carboxamide |
| 2.150 | 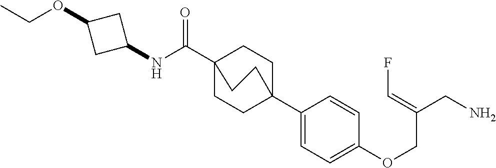 | 4-(4-(((E)-2-(aminomethyl)-3- fluoroallyl)oxy)phenyl)-N-((1s,3s)-3- ethoxycyclobutyl)bicyclo[2.2.2]octane- 1-carboxamide |
| 2.151 | 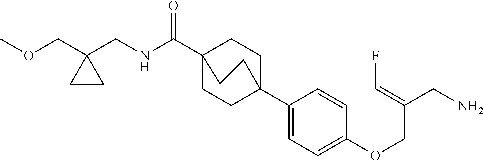 [SMILES: COCC1(CNC(=O)C23CCC(c4ccc(OC/C(=C/F)CN)cc4)(CC2)CC3)CC1] | (E)-4-(4-((2-(aminomethyl)-3- fluoroallyl)oxy)phenyl)-N-((1- (methoxymethyl)cyclopropyl)methyl) bicyclo[2.2.2]octane-1-carboxamide |
| 2.152 | 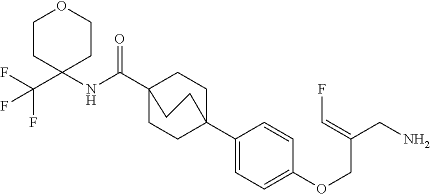 [SMILES: NC/C(=C\F)COc1ccc(C23CCC(C(=O)NC4(C(F)(F)F)CCOCC4)(CC2)CC3)cc1] | (E)-4-(4-((2-(aminomethyl)-3- fluoroallyl)oxy)phenyl)-N-(4- (trifluoromethyl)tetrahydro-2H-pyran- 4-yl)bicyclo[2.2.2]octane-1- carboxamide |
| 2.153 | 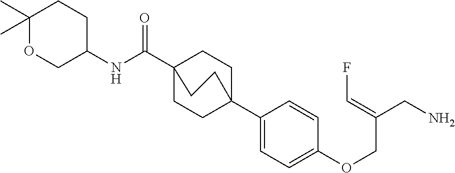 [SMILES: CC1(C)CCC(NC(=O)C23CCC(c4ccc(OC/C(=C/F)CN)cc4)(CC2)CC3)CO1] | (E)-4-(4-((2-(aminomethyl)-3- fluoroallyl)oxy)phenyl)-N-(6,6- dimethyltetrahydro-2H-pyran-3- yl)bicyclo[2.2.2]octane-1-carboxamide |
| 2.154 | 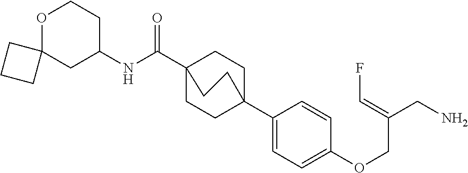 [SMILES: NC/C(=C\F)COc1ccc(C23CCC(C(=O)NC4CCOC5(CCC5)C4)(CC2)CC3)cc1] | (E)-4-(4-((2-(aminomethyl)-3- fluoroallyl)oxy)phenyl)-N-(5- oxaspiro[3.5]nonan-8- yl)bicyclo[2.2.2]octane-1-carboxamide |
| 2.155 | 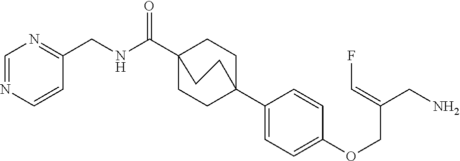 [SMILES: NC/C(=C\F)COc1ccc(C23CCC(C(=O)NCc4ccncn4)(CC2)CC3)cc1] | (E)-4-(4-((2-(aminomethyl)-3- fluoroallyl)oxy)phenyl)-N-(pyrimidin- 4-ylmethyl)bicyclo[2.2.2]octane-1- carboxamide |
| 2.156 | 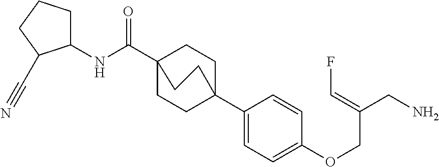 [SMILES: N#CC1CCCC1NC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- cyanocyclopentyl)bicyclo[2.2.2]octane- 1-carboxamide |
| 2.157 | 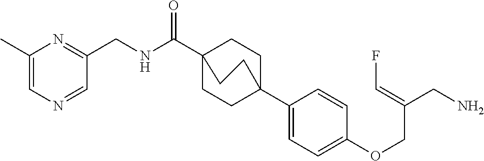 [SMILES: Cc1cncc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((6- methylpyrazin-2- yl)methyl)bicyclo[2.2.2]ctane-1- carboxamide |
| 2.158 | 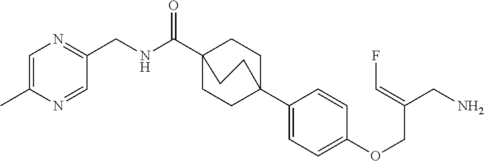 [SMILES: Cc1cnc(CNC(=O)C23CCC(c4ccc(OC/C(=C/F)CN)cc4)(CC2)CC3)cn1] | (E)-4-(4-((2-(aminomethyl)-3- fluoroallyl)oxy)phenyl)-N-((5- methylpyrazin-2- yl)methyl)bicyclo[2.2.2]octane-1- carboxamide |
| 2.159 | 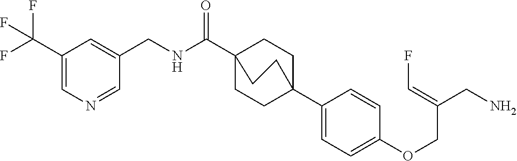 [SMILES: NC/C(=C\F)COc1ccc(C23CCC(C(=O)NCc4cncc(C(F)(F)F)c4)(CC2)CC3)cc1] | (E)-4-(4-((2-(aminomethyl)-3- fluoroallyl)oxy)phenyl)-N-((5- (trifluoromethyl)pyridin-3- yl)methyl)bicyclo[2.2.2]octane-1- carboxamide |
| 2.160 | 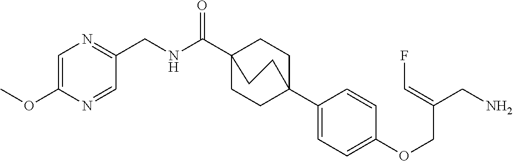 [SMILES: COc1cnc(CNC(=O)C23CCC(c4ccc(OC/C(=C/F)CN)cc4)(CC2)CC3)cn1] | (E)-4-(4-((2-(aminomethyl)-3- fluoroallyl)oxy)phenyl)-N-((5- methoxypyrazin-2- yl)methyl)bicyclo[2.2.2]octane-1- carboxamide |
| 2.161 | 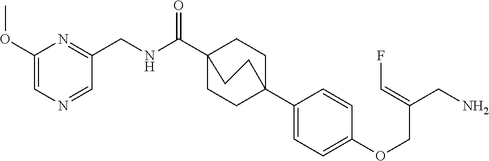 [SMILES: COc1cncc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((6- methoxypyrazin-2- yl)methyl)bicyclo[2.2.2]octane-1- carboxamide |
| 3 | 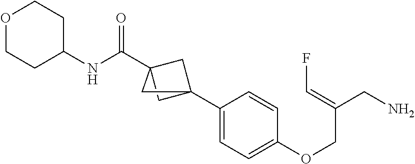 [SMILES: NC/C(=C\F)COc1ccc(C23CC(C(=O)NC4CCOCC4)(C2)C3)cc1] | (E)-3-(4-((2-(aminomethyl)-3- fluoroallyl)oxy)phenyl)-N-(tetrahydro- 2H-pyran-4-yl)bicyclo[1.1.1]pentane- 1-carboxamide |
| 3.01 | 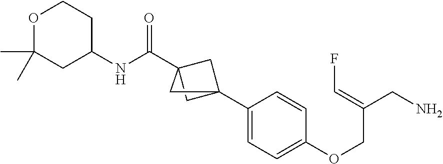 [SMILES: CC1(C)CC(NC(=O)C23CC(c4ccc(OC/C(=C/F)CN)cc4)(C2)C3)CCO1] | (E)-3-(4-((2-(aminomethyl)-3- fluoroallyl)oxy)phenyl)-N-(2,2- dimethyltetrahydro-2H-pyran-4- yl)bicyclo[1.1.1]pentane-1- carboxamide |
| 3.02 | 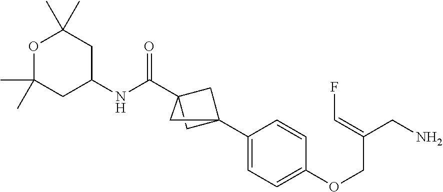 [SMILES: CC1(C)CC(NC(=O)C23CC(c4ccc(OC/C(=C/F)CN)cc4)(C2)C3)CC(C)(C)O1] | (E)-3-(4-((2-(aminomethyl)-3- fluoroallyl)oxy)phenyl)-N-(2,2,6,6- tetramethyltetrahydro-2H-pyran-4- yl)bicyclo[1.1.1]pentane-1- carboxamide |
| 3.03 | 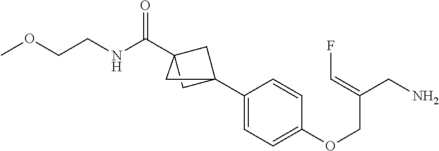 [SMILES: COCCNC(=O)C12CC(c3ccc(OC/C(=C/F)CN)cc3)(C1)C2] | (E)-3-(4-((2-(aminomethyl)-3- fluoroallyl)oxy)phenyl)-N-(2- methoxyethyl)bicyclo[1.1.1]pentane-1- carboxamide |
| 3.04 | 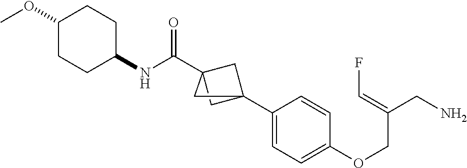 | 3-(4-(((E)-2-(aminomethyl)-3- fluoroallyl)oxy)phenyl)-N-((1r,4r)-4- methoxycyclohexyl)bicyclo[1.1.1] pentane-1-carboxamide |
| 3.05 | 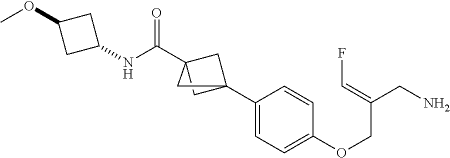 | 3-(4-(((E)-2-(aminomethyl)-3- fluoroallyl)oxy)phenyl)-N-((1r,3r)-3- methoxycyclobutyl)bicyclo[1.1.1] pentane-1-carboxamide |
| 4 | 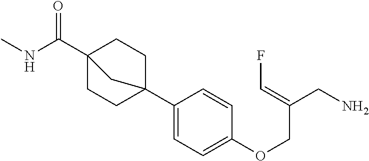 [SMILES: CNC(=O)C12CCC(c3ccc(OC/C(=C/F)CN)cc3)(CC1)C2] | (E)-4-(4-((2-(aminomethyl)-3- fluoroallyl)oxy)phenyl)-N- methylbicyclo[2.2.1]heptane-1- carboxamide |
| 4.01 | 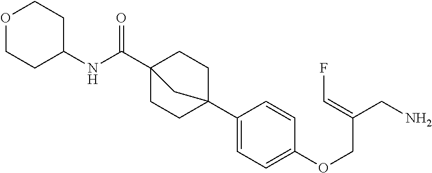 [SMILES: NC/C(=C\F)COc1ccc(C23CCC(C(=O)NC4CCOCC4)(CC2)C3)cc1] | (E)-4-(4-((2-(aminomethyl)-3- fluoroallyl)oxy)phenyl)-N-(tetrahydro- 2H-pyran-4-yl)bicyclo[2.2.1]heptane- 1-carboxamide |
| 5 | 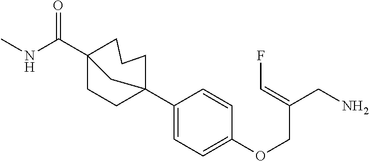 [SMILES: CNC(=O)C12CCCC(c3ccc(OC/C(=C/F)CN)cc3)(CC1)C2] | (E)-5-(4-((2-(aminomethyl)-3- fluoroallyl)oxy)phenyl)-N- methylbicyclo[3.2.1]octane-1- carboxamide |
| 5.01 | 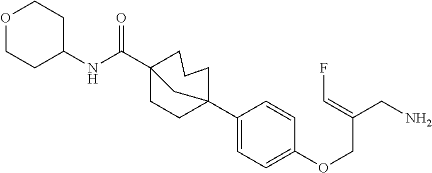 [SMILES: NC/C(=C\F)COc1ccc(C23CCCC(C(=O)NC4CCOCC4)(CC2)C3)cc1] | (E)-5-(4-((2-(aminomethyl)-3- fluoroallyl)oxy)phenyl)-N-(tetrahydro- 2H-pyran-4-yl)bicyclo[3.2.1]octane-1- carboxamide |
| 6 | 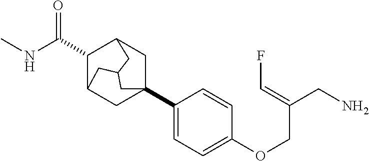 [SMILES: CNC(=O)[C@H]1C2CC3CC1C[C@@](c1ccc(OC/C(=C/F)CN)cc1)(C3)C2] | (E)-5-(4-((2-(aminomethyl)-3- fluoroallyl)oxy)phenyl)-N- methyladamantane-2-carboxamide |
| 6.01 | 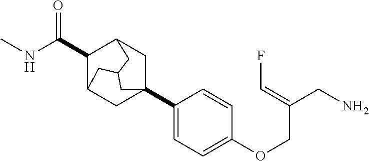 [SMILES: CNC(=O)[C@H]1C2CC3CC1C[C@](c1ccc(OC/C(=C/F)CN)cc1)(C3)C2] | (E)-5-(4-((2-(aminomethyl)-3- fluoroallyl)oxy)phenyl)-N- methyladamantane-2-carboxamide |
| 6.02 | 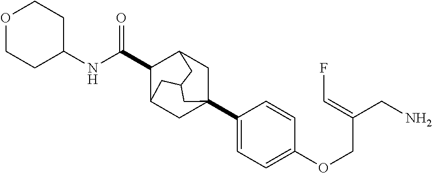 [SMILES: NC/C(=C\F)COc1ccc([C@]23CC4CC(C2)[C@@H](C(=O)NC2CCOCC2)C(C4)C3)cc1] | (E)-5-(4-((2-(aminomethyl)-3- fluoroallyl)oxy)phenyl)-N-(tetrahydro- 2H-pyran-4-yl)adamantane-2- carboxamide |
| 6.03 | 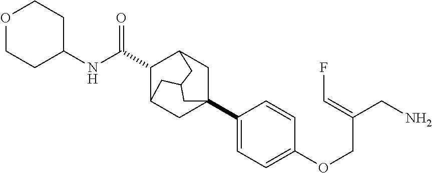 [SMILES: NC/C(=C\F)COc1ccc([C@]23CC4CC(C2)[C@H](C(=O)NC2CCOCC2)C(C4)C3)cc1] | (E)-5-(4-((2-(aminomethyl)-3- fluoroallyl)oxy)phenyl)-N-(tetrahydro- 2H-pyran-4-yl)adamantane-2- carboxamide |
| 6.04 | 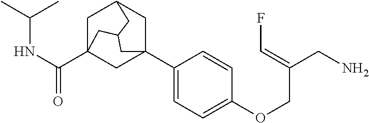 [SMILES: CC(C)NC(=O)C12CC3CC(C1)CC(c1ccc(OC/C(=C/F)CN)cc1)(C3)C2] | (E)-3-(4-((2-(aminomethyl)-3- fluoroallyl)oxy)phenyl)-N- isopropyladamantane-1-carboxamide |
| 7 | 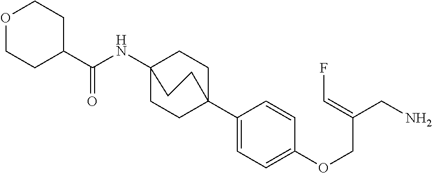 [SMILES: NC/C(=C\F)COc1ccc(C23CCC(NC(=O)C4CCOCC4)(CC2)CC3)cc1] | (E)-N-(4-(4-((2-(aminomethyl)-3- fluoroallyl)oxy)phenyl)bicyclo[2.2.2] octan-1-yl)tetrahydro-2H-pyran-4- carboxamide |
| 7.01 | 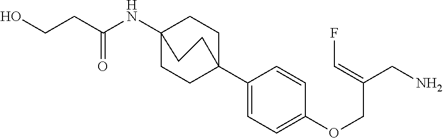 [SMILES: NC/C(=C\F)COc1ccc(C23CCC(NC(=O)CCO)(CC2)CC3)cc1] | (E)-N-(4-(4-((2-(aminomethyl)-3- fluoroallyl)oxy)phenyl)bicyclo[2.2.2] octan-1-yl)-3-hydroxypropanamide |
| 7.02 | 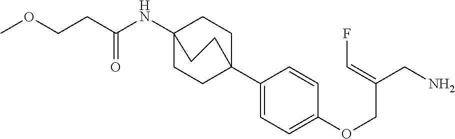 [SMILES: COCCC(=O)NC12CCC(c3ccc(OC/C(=C/F)CN)cc3)(CC1)CC2] | (E)-N-(4-(4-((2-(aminomethyl)-3- fluoroallyl)oxy)phenyl)bicyclo[2.2.2] octan-1-yl)-3-methoxypropanamide |
| 7.03 | 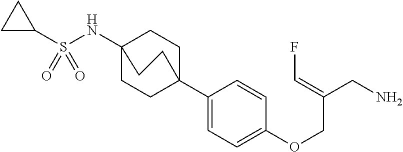 [SMILES: NC/C(=C\F)COc1ccc(C23CCC(NS(=O)(=O)C4CC4)(CC2)CC3)cc1] | (E)-N-(4-(4-((2-(aminomethyl)-3- fluoroallyl)oxy)phenyl)bicyclo[2.2.2] octan-1-yl)cyclopropanesulfonamide |
| 8 | 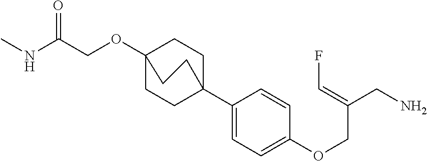 [SMILES: CNC(=O)COC12CCC(c3ccc(OC/C(=C/F)CN)cc3)(CC1)CC2] | (E)-2-((4-(4-((2-(aminomethyl)-3- fluoroallyl)oxy)phenyl)bicyclo[2.2.2] octan-1-yl)oxy)-N-methylacetamide |
| 8.01 | 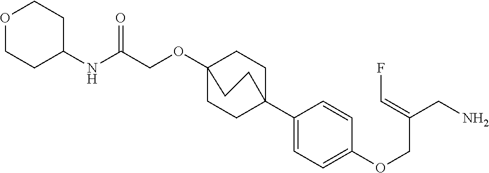 [SMILES: NC/C(=C\F)COc1ccc(C23CCC(OCC(=O)NC4CCOCC4)(CC2)CC3)cc1] | (E)-2-((4-(4-((2-(aminomethyl)-3- fluoroallyl)oxy)phenyl)bicyclo[2.2.2] octan-1-yl)oxy)-N-(tetrahydro-2H- pyran-4-yl)acetamide |
| 8.02 | 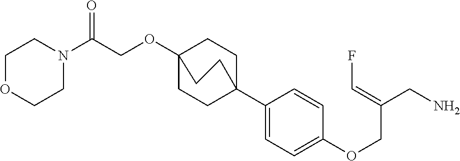 [SMILES: NC/C(=C\F)COc1ccc(C23CCC(OCC(=O)N4CCOCC4)(CC2)CC3)cc1] | (E)-2-((4-(4-((2-(aminomethyl)-3- fluoroallyl)oxy)phenyl)bicyclo[2.2.2] octan-1-yl)oxy)-1-morpholinoethanone |
| 8.03 | 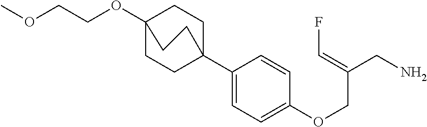 [SMILES: COCCOC12CCC(c3ccc(OC/C(=C/F)CN)cc3)(CC1)CC2] | (E)-3-fluoro-2-((4-(4-(2- methoxyethoxy)bicyclo[2.2.2]octan-1- yl)phenoxy)methyl)prop-2-en-1-amine |
| 9 | 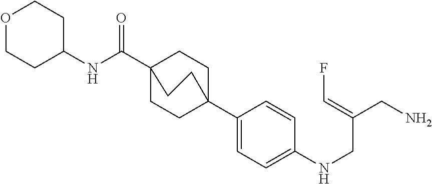 [SMILES: NC/C(=C\F)CNc1ccc(C23CCC(C(=O)NC4CCOCC4)(CC2)CC3)cc1] | (E)-4-(4-((2-(aminomethyl)-3- fluoroallyl)amino)phenyl)-N- (tetrahydro-2H-pyran-4- yl)bicyclo[2.2.2]octane-1-carboxamide |
| 10 | 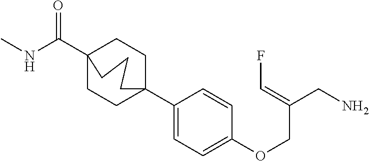 [SMILES: CNC(=O)C12CCCC(c3ccc(OC/C(=C/F)CN)cc3)(CC1)CC2] | (E)-5-(4-((2-(aminomethyl)-3- fluoroallyl)oxy)phenyl)-N- methylbicyclo[3.2.2]nonane-1- carboxamide |
| 10.01 | 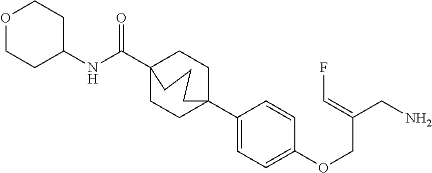 [SMILES: NC/C(=C\F)COc1ccc(C23CCCC(C(=O)NC4CCOCC4)(CC2)CC3)cc1] | (E)-5-(4-((2-(aminomethyl)-3- fluoroallyl)oxy)phenyl)-N-(tetrahydro- 2H-pyran-4-yl)bicyclo[3.2.2]nonane- 1-carboxamide |
| 11 | 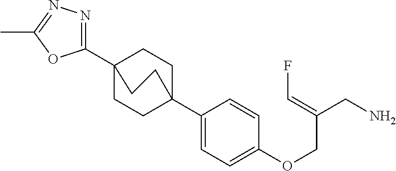 [SMILES: Cc1nnc(C23CCC(c4ccc(OC/C(=C/F)CN)cc4)(CC2)CC3)o1] | (E)-3-fluoro-2-((4-(5-(5-methyl-1,3,4- oxadiazol-2-yl)bicyclo[3.2.2]nonan-1- yl)phenoxy)methyl)prop-2-en-1-amine |
| 11.01 | 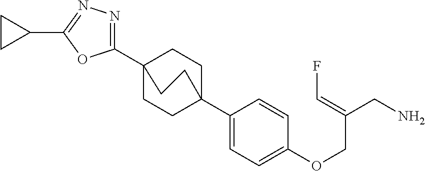 [SMILES: NC/C(=C\F)COc1ccc(C23CCC(c4nnc(C5CC5)o4)(CC2)CC3)cc1] | (E)-2-((4-(4-(5-cyclopropyl-1,3,4- oxadiazol-2-yl)bicyclo[2.2.2]octan-1- yl)phenoxy)methyl)-3-fluoroprop-2- en-1-amine |
| 11.02 | 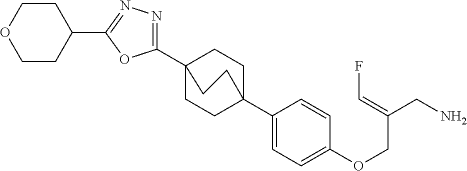 [SMILES: NC/C(=C\F)COc1ccc(C23CCC(c4nnc(C5CCOCC5)o4)(CC2)CC3)cc1] | (E)-3-fluoro-2-((4-(4-(5-(tetrahydro- 2H-pyran-4-yl)-1,3,4-oxadiazol-2- yl)bicyclo[2.2.2]octan-1- yl)phenoxy)methyl)prop-2-en-1-amine |

In some embodiments, provided herein is a pharmaceutically acceptable salt or solvate of a compound that is described in Table 1.

In some embodiments, compounds described herein include, but are not limited to, those described in Table 2.

TABLE 2

| Structure | Chemical Name |
|---|---|
| 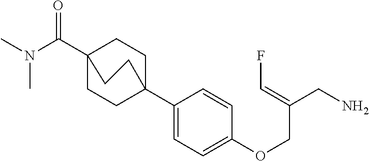 [SMILES: CN(C)C(=O)C12CCC(c3ccc(OC/C(=C/F)CN)cc3)(CC1)CC2] | (E)-4-(4-((2-(aminomethyl)-3- fluoroallyl)oxy)phenyl)-N,N- dimethylbicyclo[2.2.2]octane-1-carboxamide |
| 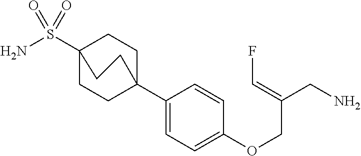 [SMILES: NC/C(=C\F)COc1ccc(C23CCC(S(N)(=O)=O)(CC2)CC3)cc1] | (E)-4-(4-((2-(aminomethyl)-3- fluoroallyl)oxy)phenyl)bicyclo[2.2.2]octane-1- sulfonamide |
| 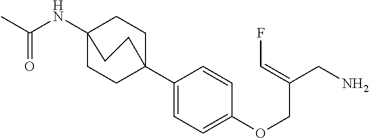 [SMILES: CC(=O)NC12CCC(c3ccc(OC/C(=C/F)CN)cc3)(CC1)CC2] | (E)-N-(4-(4-((2-(aminomethyl)-3- fluoroallyl)oxy)phenyl)bicyclo[2.2.2]octan-1- yl)acetamide |
| 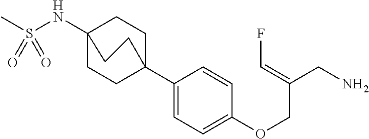 [SMILES: CS(=O)(=O)NC12CCC(c3ccc(OC/C(=C/F)CN)cc3)(CC1)CC2] | (E)-N-(4-(4-((2-(aminomethyl)-3- fluoroallyl)oxy)phenyl)bicyclo[2.2.2]octan-1- yl)methanesulfonamide |
| 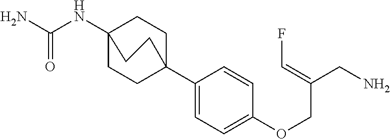 [SMILES: NC/C(=C\F)COc1ccc(C23CCC(NC(N)=O)(CC2)CC3)cc1] | (E)-1-(4-(4-((2-(aminomethyl)-3- fluoroallyl)oxy)phenyl)bicyclo[2.2.2]octan-1- yl)urea |
| 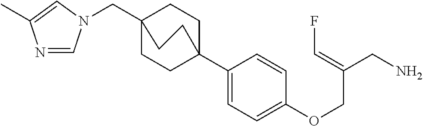 [SMILES: Cc1cn(CC23CCC(c4ccc(OC/C(=C/F)CN)cc4)(CC2)CC3)cn1] | (E)-3-fluoro-2-((4-(4-((4-methyl-1H-imidazol-1- yl)methyl)bicyclo[2.2.2]octan-1- yl)phenoxy)methyl)prop-2-en-1-amine |
| 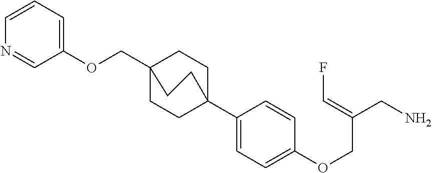 [SMILES: NC/C(=C\F)COc1ccc(C23CCC(COc4cccnc4)(CC2)CC3)cc1] | (E)-3-fluoro-2-((4-(4-((pyridin-3- yloxy)methyl)bicyclo[2.2.2]octan-1- yl)phenoxy)methyl)prop-2-en-1-amine |
| 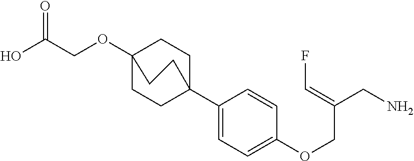 [SMILES: NC/C(=C\F)COc1ccc(C23CCC(OCC(=O)O)(CC2)CC3)cc1] | (E)-2-((4-(4-((2-(aminomethyl)-3- fluoroallyl)oxy)phenyl)bicyclo[2.2.2]octan-1- yl)oxy)acetic acid |
| 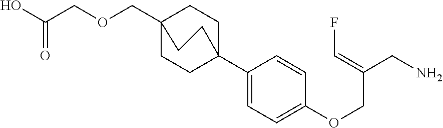 [SMILES: NC/C(=C\F)COc1ccc(C23CCC(COCC(=O)O)(CC2)CC3)cc1] | (E)-2-((4-(4-((2-(aminomethyl)-3- fluoroallyl)oxy)phenyl)bicyclo[2.2.2]octan-1- yl)methoxy)acetic acid |
| 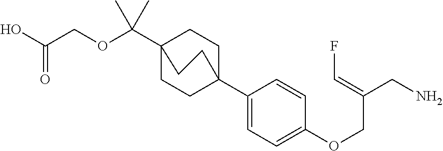 [SMILES: CC(C)(OCC(=O)O)C12CCC(c3ccc(OC/C(=C/F)CN)cc3)(CC1)CC2] | (E)-2-((2-(4-(4-((2-(aminomethyl)-3- fluoroallyl)oxy)phenyl)bicyclo[2.2.2]octan-1- yl)propan-2-yl)oxy)acetic acid |
| 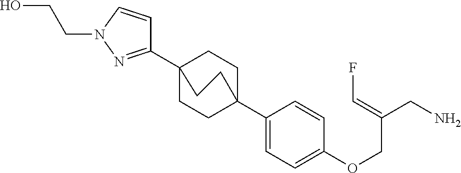 [SMILES: NC/C(=C\F)COc1ccc(C23CCC(c4ccn(CCO)n4)(CC2)CC3)cc1] | (E)-2-(3-(4-(4-((2-(aminomethyl)-3- fluoroallyl)oxy)phenyl)bicyclo[2.2.2]octan-1-yl)- 1H-pyrazol-1-yl)ethanol |
| 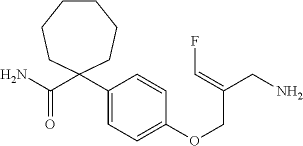 [SMILES: NC/C(=C\F)COc1ccc(C2(C(N)=O)CCCCCC2)cc1] | (E)-1-(4-((2-(aminomethyl)-3- fluoroallyl)oxy)phenyl)cycloheptanecarboxamide |
| 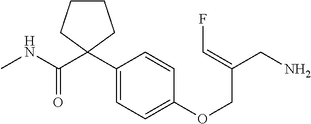 [SMILES: CNC(=O)C1(c2ccc(OC/C(=C/F)CN)cc2)CCCC1] | (E)-1-(4-((2-(aminomethyl)-3- fluoroallyl)oxy)phenyl)-N- methylcyclopentanecarboxamide |
| 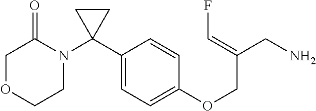 [SMILES: NC/C(=C\F)COc1ccc(C2(N3CCOCC3=O)CC2)cc1] | (E)-4-(1-(4-((2-(aminomethyl)-3- fluoroallyl)oxy)phenyl)cyclopropyl)morpholin-3- one |
| 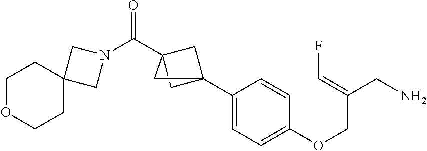 [SMILES: NC/C(=C\F)COc1ccc(C23CC(C(=O)N4CC5(CCOCC5)C4)(C2)C3)cc1] | (E)-(3-(4-((2-(aminomethyl)-3- fluoroallyl)oxy)phenyl)bicyclo[1.1.1]pentan-1- yl)(7-oxa-2-azaspiro[3.5]nonan-2-yl)methanone |
| 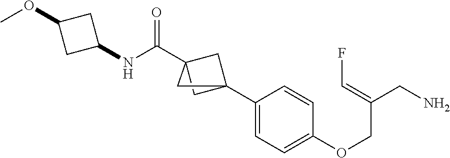 | 3-(4-(((E)-2-(aminomethyl)-3- fluoroallyl)oxy)phenyl)-N-((1s,3s)-3- methoxycyclobutyl)bicyclo[1.1.1]pentane-1- carboxamide |
| 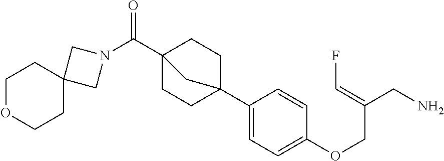 [SMILES: NC/C(=C\F)COc1ccc(C23CCC(C(=O)N4CC5(CCOCC5)C4)(CC2)C3)cc1] | (E)-(4-(4-((2-(aminomethyl)-3- fluoroallyl)oxy)phenyl)bicyclo[2.2.1]heptan-1- yl)(7-oxa-2-azaspiro[3.5]nonan-2-yl)methanone |
| 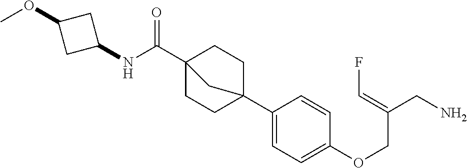 | 4-(4-(((E)-2-(aminomethyl)-3- fluoroallyl)oxy)phenyl)-N-((1s,3s)-3- methoxycyclobutyl)bicyclo[2.2.1]heptane-1- carboxamide |
| 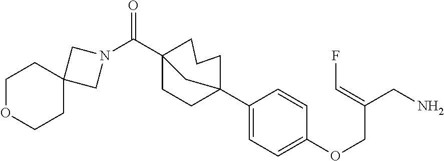 [SMILES: NC/C(=C\F)COc1ccc(C23CCCC(C(=O)N4CC5(CCOCC5)C4)(CC2)C3)cc1] | (E)-(5-(4-((2-(aminomethyl)-3- fluoroallyl)oxy)phenyl)bicyclo[3.2.1]octan-1- yl)(7-oxa-2-azaspiro[3.5]nonan-2-yl)methanone |
| 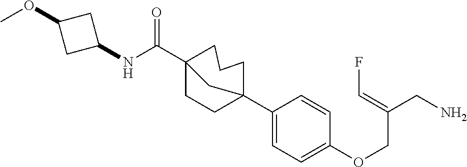 | 5-(4-(((E)-2-(aminomethyl)-3- fluoroallyl)oxy)phenyl)-N-((1s,3s)-3- methoxycyclobutyl)bicyclo[3.2.1]octane-1- carboxamide |
| 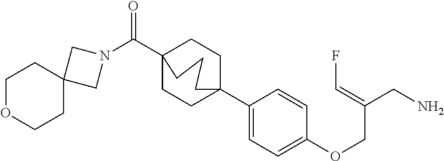 [SMILES: NC/C(=C\F)COc1ccc(C23CCCC(C(=O)N4CC5(CCOCC5)C4)(CC2)CC3)cc1] | (E)-(5-(4-((2-(aminomethyl)-3- fluoroallyl)oxy)phenyl)bicyclo[3.2.2]nonan-1- yl)(7-oxa-2-azaspiro[3.5]nonan-2-yl)methanone |
| 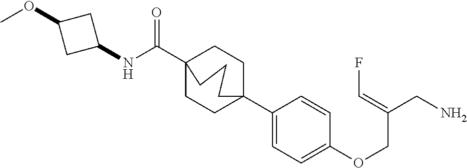 | 5-(4-(((E)-2-(aminomethyl)-3- fluoroallyl)oxy)phenyl)-N-((1s,3s)-3- methoxycyclobutyl)bicyclo[3.2.2]nonane-1- carboxamide |
| 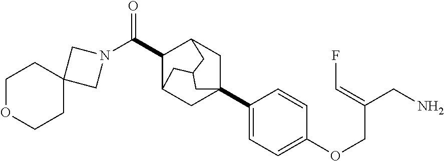 [SMILES: NC/C(=C\F)COc1ccc([C@]23CC4CC(C2)[C@@H](C(=O)N2CC5(CCOCC5)C2)C(C4)C3)cc1] | ((2s,5r)-5-(4-(((E)-2-(aminomethyl)-3- fluoroallyl)oxy)phenyl)adamantan-2-yl)(7-oxa-2- azaspiro[3.5]nonan-2-yl)methanone |
| 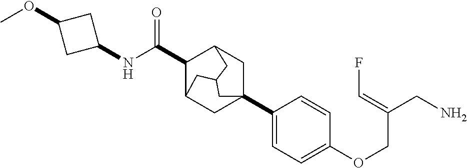 | (2s,5R)-5-(4-(((E)-2-(aminomethyl)-3- fluoroallyl)oxy)phenyl)-N-((1s,3S)-3- methoxycyclobutyl)adamantane-2-carboxamide |
| 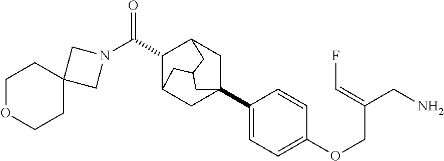 [SMILES: NC/C(=C\F)COc1ccc([C@]23CC4CC(C2)[C@H](C(=O)N2CC5(CCOCC5)C2)C(C4)C3)cc1] | ((2r,5s)-5-(4-(((E)-2-(aminomethyl)-3- fluoroallyl)oxy)phenyl)adamantan-2-yl)(7-oxa-2- azaspiro[3.5]nonan-2-yl)methanone |
| 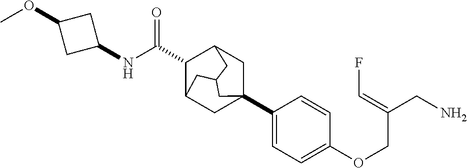 | (2r,5S)-5-(4-(((E)-2-(aminomethyl)-3- fluoroallyl)oxy)phenyl)-N-((1s,3S)-3- methoxycyclobutyl)adamantane-2-carboxamide |
| 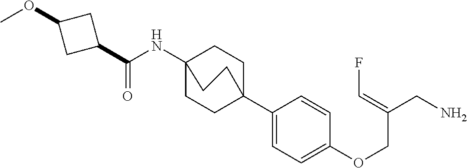 | (1s,3s)-N-(4-(4-(((E)-2-(aminomethyl)-3- fluoroallyl)oxy)phenyl)bicyclo[2.2.2]octan-1-yl)- 3-methoxycyclobutanecarboxamide |

In some embodiments, provided herein is a pharmaceutically acceptable salt or solvate of a compound that is described in Table 2.

In one aspect, compounds described herein are in the form of pharmaceutically acceptable salts. As well, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound described herein with an acid to provide a “pharmaceutically acceptable acid addition salt.” In some embodiments, the compound described herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; monomethyl fumarate, naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound described herein is prepared as a chloride salt, sulfate salt, bromide salt, mesylate salt, maleate salt, citrate salt or phosphate salt.

In some embodiments, pharmaceutically acceptable salts are obtained by reacting a compound described herein with a base to provide a “pharmaceutically acceptable base addition salt.”

In some embodiments, the compound described herein is acidic and is reacted with a base. In such situations, an acidic proton of the compound described herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methyl amine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of isolating or purifying the compound with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

The methods and formulations described herein include the use of N-oxides (if appropriate), crystalline forms (also known as polymorphs), or pharmaceutically acceptable salts of compounds described herein, as well as active metabolites of these compounds having the same type of activity.

In some embodiments, sites on the organic radicals (e.g. alkyl groups, aromatic rings) of compounds described herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, fluorine and chlorine, such as, for example, ²H, ³H, ¹³C, ¹⁴C, ¹⁵N, ¹⁸O, ¹⁷O, ³⁵S, ⁸F, ³⁶Cl. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or reduced dosage requirements. In some embodiments, one or more hydrogen atoms of the compounds described herein is replaced with deuterium.

In some embodiments, the compounds described herein possess one or more stereocenters and each stereocenter exists independently in either the R or S configuration. The compounds presented herein include all diastereomeric, enantiomeric, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns. In certain embodiments, compounds described herein are prepared as their individual stereoisomers by reacting a racemic mixture of the compound with an optically active resolving agent to form a pair of diastereoisomeric compounds/salts, separating the diastereomers and recovering the optically pure enantiomers. In some embodiments, resolution of enantiomers is carried out using covalent diastereomeric derivatives of the compounds described herein. In another embodiment, diastereomers are separated by separation/resolution techniques based upon differences in solubility. In other embodiments, separation of steroisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley and Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. The prodrug may be a substrate for a transporter. Further or alternatively, the prodrug also has improved solubility in pharmaceutical compositions over the parent drug. In some embodiments, the design of a prodrug increases the effective water solubility. An example, without limitation, of a prodrug is a compound described herein, which is administered as an ester (the “prodrug”) but then is metabolically hydrolyzed to provide the active entity. A further example of a prodrug is a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the compound. In certain embodiments, a prodrug is enzymatically metabolized by one or more steps or processes to the biologically, pharmaceutically or therapeutically active form of the compound.

Prodrugs of the compounds described herein include, but are not limited to, esters, ethers, carbonates, thiocarbonates, N-acyl derivatives, N-acyloxyalkyl derivatives, quaternary derivatives of tertiary amines, N-Mannich bases, Schiff bases, amino acid conjugates, phosphate esters, and sulfonate esters. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. In some embodiments, a hydroxyl group in the compounds disclosed herein is used to form a prodrug, wherein the hydroxyl group is incorporated into an acyloxyalkyl ester, alkoxycarbonyloxyalkyl ester, alkyl ester, aryl ester, phosphate ester, sugar ester, ether, and the like. In some embodiments, a hydroxyl group in the compounds disclosed herein is a prodrug wherein the hydroxyl is then metabolized in vivo to provide a carboxylic acid group. In some embodiments, a carboxyl group is used to provide an ester or amide (i.e. the prodrug), which is then metabolized in vivo to provide a carboxylic acid group. In some embodiments, compounds described herein are prepared as alkyl ester prodrugs.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound.

Prodrug forms of the herein described compounds, wherein the prodrug is metabolized in vivo to produce a compound described herein as set forth herein are included within the scope of the claims. In some cases, some of the herein-described compounds is a prodrug for another derivative or active compound. In some embodiments, a prodrug of the compound disclosed herein permits targeted delivery of the compound to a particular region of the gastrointestinal tract. Formation of a pharmacologically active metabolite by the colonic metabolism of drugs is a commonly used “prodrug” approach for the colon-specific drug delivery systems.

In some embodiments, a prodrug is formed by the formation of a covalent linkage between drug and a carrier in such a manner that upon oral administration the moiety remains intact in the stomach and small intestine. This approach involves the formation of prodrug, which is a pharmacologically inactive derivative of a parent drug molecule that requires spontaneous or enzymatic transformation in the biological environment to release the active drug. Formation of prodrugs has improved delivery properties over the parent drug molecule. The problem of stability of certain drugs from the adverse environment of the upper gastrointestinal tract can be eliminated by prodrug formation, which is converted into parent drug molecule once it reaches into the colon. Site specific drug delivery through site specific prodrug activation may be accomplished by the utilization of some specific property at the target site, such as altered pH or high activity of certain enzymes relative to the non-target tissues for the prodrug-drug conversion.

In some embodiments, covalent linkage of the drug with a carrier forms a conjugate conjugate. Such conjugates include, but are not limited to, azo bond conjugates, glycoside conjugates, glucuronide conjugates, cyclodextrin conjugates, dextran conjugates or amino-acid conjugates.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

In some embodiments, the compounds described herein are rapidly metabolized following absorption from the gastro-intestinal tract to metabolites that have greatly reduced SSAO inhibitor activity.

In additional or further embodiments, the compounds are rapidly metabolized in plasma.

In additional or further embodiments, the compounds are rapidly metabolized by the intestines.

In additional or further embodiments, the compounds are rapidly metabolized by the liver.

### Synthesis of Compounds

Compounds described herein are synthesized using standard synthetic techniques or using methods known in the art in combination with methods described herein.

Unless otherwise indicated, conventional methods of mass spectroscopy, NMR, HPLC, protein chemistry, biochemistry, recombinant DNA techniques and pharmacology are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions. The starting materials are available from commercial sources or are readily prepared.

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

The compounds described herein are prepared by the general synthetic routes described below in Schemes 1 to 7.

In some embodiments, intermediates used in the preparation of compounds described herein are prepared as outlined in Scheme 1.

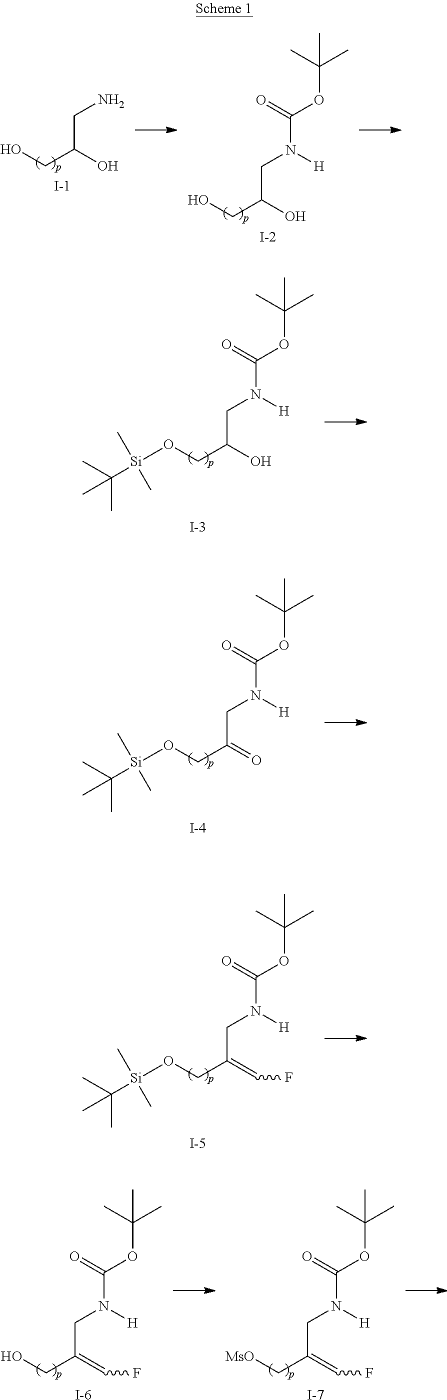

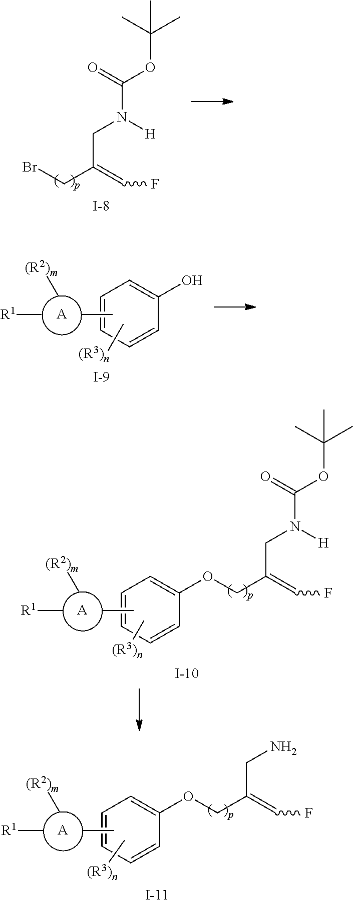

In Scheme 1, substituents R¹, R², R³, m, n and p are as described herein.

In some embodiments, intermediate I-1 is reacted to incorporate a suitable amine protecting group. In some embodiments, a suitable protecting group is a Boc group to provide intermediate I-2. In some embodiments suitable conditions include the use of Boc₂O with an appropriate base and solvent or solvent mixture at an appropriate time and at an appropriate temperature. In some embodiments the base is an organic base such as triethylamine. In some embodiments the appropriate solvent is an alcohol solvent such as methanol. In some embodiments, the appropriate time and appropriate temperature is about 2 to about 18 hours (overnight) hours at about room temperature.

In some embodiments, I-2 is subjected to suitable conditions to incorporate a primary alcohol protecting group. In some embodiments, a suitable protecting group is a silyl protecting group such as t-butyldimethylsilyl (TBS) to provide intermediate I-3. In some embodiments, conditions include the use an appropriate reagent such as TBS-Cl using an appropriate base and solvent or solvent mixture at an appropriate time and at an appropriate temperature. In some embodiments, the appropriate base is imidazole. In some embodiments, the appropriate solvent is a chlorinated solvent such as dichloromethane. In some embodiments, the suitable temperature is about 0° C. to room temperature and the appropriate amount of time is about 18 hours (overnight).

In some embodiments, I-3 is reacted under suitable oxidization conditions to provide intermediate I-4. In some embodiments, the alcohol is oxidized under Swern oxidation conditions using an appropriate reagent such as oxalyl chloride with an appropriate base in an appropriate solvent or solvent mixture at an appropriate temperature and an appropriate amount of time. In some embodiments, the appropriate base is an organic base such as triethylamine. In some embodiments, the appropriate the solvent is a chlorinated solvent such as dichloromethane. In some embodiments, the suitable temperature is about −78° C. to room temperature and the appropriate amount of time is about 2 to 18 hours (overnight).

In some embodiments, I-4 is reacted under suitable one carbon-homologation conditions to provide I-5. In some embodiments, suitable one carbon-homologation conditions include the use of phosphonium reagents. In some embodiments, suitable one-carbon-homologation conditions includes pre-treating either (fluoromethyl)triphenylphosphonium bromide or tetrafluoroborate salt with an appropriate base, with an appropriate solvent for an appropriate amount of time at an appropriate temperature before the addition of I-4. In some embodiments, the appropriate base is NaHMDS. In some embodiments, the appropriate solvent is an ethereal solvent such as THF. In some embodiments, the appropriate temperature and amount of time before addition to I-4 is about 30 mins at about −20° C. In some embodiments, after I-4 is added the reaction is continued for an additional about 2 to 18 hours (overnight) at about room temperature. In some embodiments, I-5 was isolated as mixture of regioisomers. In some embodiments, the regioisomers of I-5 were separated by flash chromatography under appropriate conditions. In some embodiments, appropriate conditions for separation of the regioisomers was flash chromatography using silica gel, eluting with an appropriate solvent system such as a mixture of hexanes and ethyl acetate.

In some embodiments, I-5 is reacted under suitable conditions to remove the alcohol protecting group to provide intermediate I-6. In some embodiments, appropriate conditions include using tetrabutylammonium fluoride in a suitable solvent at an appropriate temperature and amount of time. In some embodiments, the appropriate solvent is an ethereal solvent such as THF. In some embodiments, the appropriate temperature and time is about 1 to 18 hour (overnight) at about room temperature. In some embodiments, further purification via flash chromatography using an appropriate solvent system provide the pure E- or Z-isomers of I-6. In some embodiments, appropriate conditions for separation of the regioisomers was flash chromatography using silica gel, eluting with an appropriate solvent system such as a mixture of hexanes and ethyl acetate.

In some embodiments, I-6 is reacted under suitable conditions to provide intermediate I-7. In some embodiments, appropriate conditions include using methanesulfonyl chloride using an appropriate base and solvent or solvent mixture at an appropriate time and at an appropriate temperature. In some embodiments, the appropriate base is an organic base such as triethylamine. In some embodiments, the appropriate solvent is acetone. In some embodiments, the appropriate temperature and time is about 0° C. and about 1 h. In some embodiments, the reaction mixture is filtered and the filtrate is used directly as a solution of intermediate I-7 in acetone.

In some embodiments, I-7 is reacted under suitable conditions to provide intermediate bromide I-8. In some embodiments, appropriate conditions include using lithium bromide in an appropriate solvent or solvent mixture at an appropriate time and at an appropriate temperature. In some embodiments, the appropriate solvent is acetone. In some embodiments, the appropriate temperature and time is about room temperature and about 1 h.

In some embodiments, I-8 is reacted with intermediate I-9 under suitable conditions to provide intermediate I-10. In some embodiments, I-9 is 1-(4-hydroxyphenyl)cyclohexanecarboxamide, 1-(4-hydroxyphenyl)-N-methylcyclohexanecarboxamide, 4-(4-hydroxy-3-methylphenyl)-N-methylbicyclo[2.2.2]octane-1-carboxamide, 4-(4-hydroxy-3-methylphenyl)bicyclo[2.2.2]octane-1-carboxamide or 4-(4-hydroxyphenyl)-N-methylbicyclo[2.2.2]octane-1-carboxamide. In some embodiments, appropriate conditions include using an appropriate base with an appropriate solvent or solvent mixture at an appropriate time and at an appropriate temperature. In some embodiments, the appropriate base in an inorganic base such as cesium carbonate or potassium carbonate. In some embodiments, the appropriate solvent is THF, DMF or DMA. In some embodiments, the reaction temperature is about room temperature to about 90° C. and the reaction time is about 18 hours (overnight).

In some embodiments, I-10 is reacted with an appropriate acid with an appropriate solvent or solvent mixture at an appropriate time and at an appropriate temperature to provide compound I-11. In some embodiments, the appropriate acid is HCl or TFA. In some embodiments, the appropriate solvent is Et₂O, dioxane, MeOH, EtOH, EtOAc or DCM. In some embodiments the reaction is conducted in TFA only. In some embodiments, the reaction temperature is about room temperature to about 60° C. and the reaction time is about 2 to about 18 hours (overnight). In some embodiments, I-10 is treated with HCl after reaction with TFA to isolate the hydrochloride salt of compound I-11.

In some embodiments, intermediate I-9 used in the preparation of compounds I-10 described herein are prepared as outlined in Scheme 2.

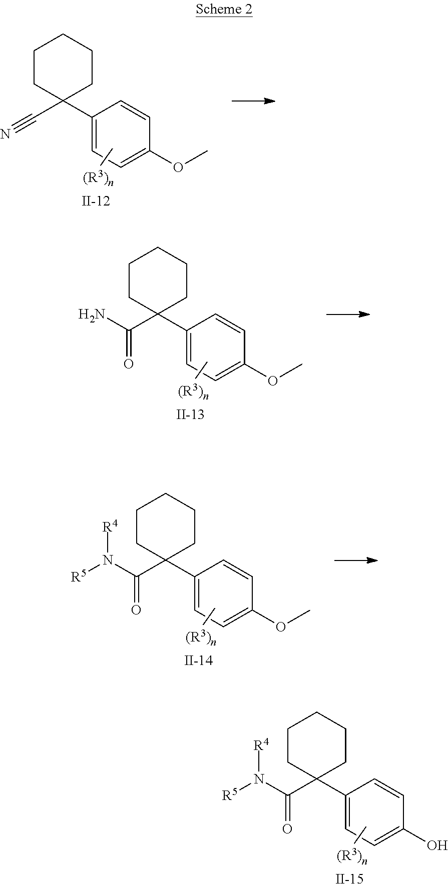

In Scheme 2, substituents R³, R⁴, R⁵, and n are as described herein. In some embodiments, n is 0. In some embodiments, R⁴ and R⁵ are H. In some embodiments, R⁴ is methyl and R⁵ is H.

In some embodiments intermediate II-13 is prepared from nitrile II-12 under appropriate hydrolysis conditions. In some embodiments suitable hydrolysis conditions include but are not limited to the use of a suitable reagent in an appropriate solvent for an appropriate time and at an appropriate temperature. In some embodiments, the appropriate reagent is potassium hydroxide. In some embodiments, the appropriate solvent is 2-methylbutan-2-ol. In some embodiments, the appropriate temperature is about 120° C. In some embodiments, the appropriate time is about 8 hours.

In some embodiments intermediate II-14 is prepared from intermediate II-13 under suitable alkylation conditions. Suitable alkylation conditions include but are not limited to use of a suitable alkylating reagent and a suitable base in an appropriate solvent for an appropriate time and at an appropriate temperature. In some embodiments, the appropriate alkylating reagent is iodomethane. In some embodiments, the appropriate base is sodium hydride. In some embodiments, the appropriate solvent is THF. In some embodiments, the appropriate temperature is about 0° C. In some embodiments, the appropriate time is about 18 hours (overnight).

In some embodiments, intermediate II-14 is reacted under suitable conditions to provide phenol II-15. In some embodiments, appropriate conditions include using an appropriate reagent with an appropriate solvent at an appropriate time and at an appropriate temperature. In some embodiments, the appropriate reagent is boron tribromide. In some embodiments, the appropriate solvent is DCM. In some embodiments, the reaction temperature is about −78° C. and the reaction time is about 3 hours.

In some embodiments, intermediates used in the preparation of compounds described herein are prepared as outlined in Scheme 3.

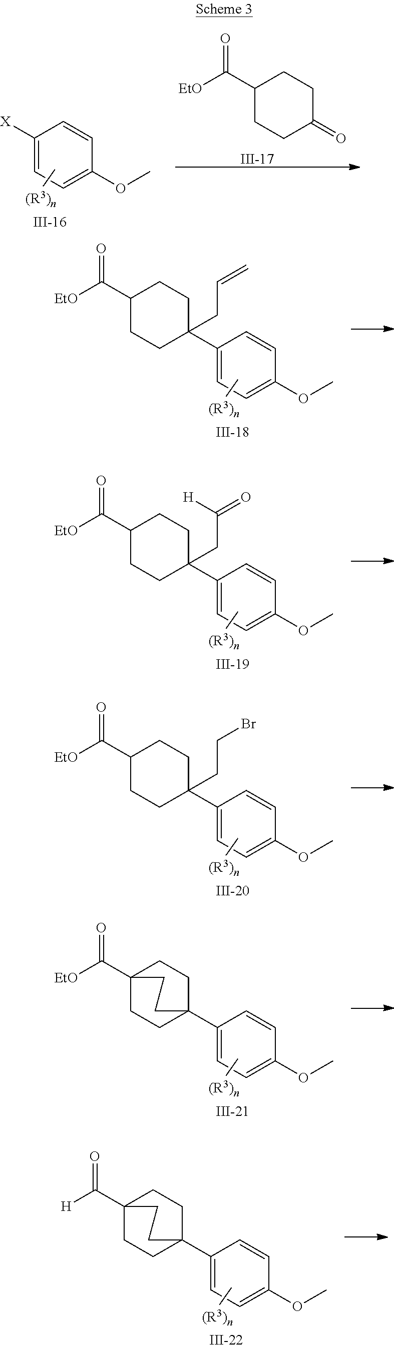

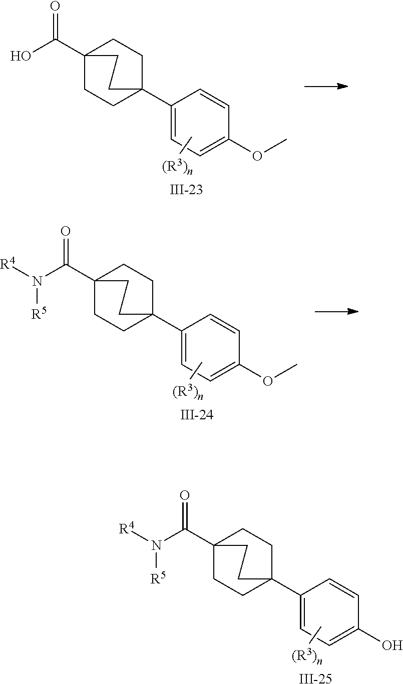

In Scheme 3, substituents X, R³, R⁴, R⁵, and n are described herein. In some embodiments, X is a halide. In some embodiments, the halide is chloride, bromide or iodide. In some embodiments, n is 0. In some embodiments, n is 1 and R³ is methyl. In some embodiments, R⁴ and R⁵ are H. In some embodiments, R⁴ is methyl and R⁵ is H.

In some embodiments, halide III-16 is cooled to a suitable temperature, reacted under suitable metal-halogen exchange conditions with an appropriate solvent for an appropriate time and at an appropriate temperature, and then later reacted with an appropriate ketone III-17 for an appropriate time and at an appropriate temperature to provide a tertiary alcohol. In some embodiments, suitable metal-halogen exchange conditions include an organometallic reagent. In some embodiments, the appropriate solvent is THF. In some embodiments, the organometallic reagent is an alkyllithium. In some embodiments, the alkyllithium is n-butyllithium. In some embodiments, III-16 is cooled to about −78° C. before addition of the organometallic reagent. In some embodiments, III-16 is reacted for about one hour at about −78° C. before addition of ketone III-17. In some embodiments, III-16 is reacted for about 2 hours after the addition of ketone III-17. In some embodiments, the appropriate temperature for reacting III-16 and ketone III-17 is about −78° C. In some embodiments, the tertiary alcohol is reacted under appropriate allylation conditions which include use of an allylating reagent and a Lewis acid, in an appropriate solvent for an appropriate time and at an appropriate temperature to form III-18. In some embodiments, the appropriate allylating reagent is allyltrimethylsilane. In some embodiments, the appropriate Lewis acid is BF₃—OEt₂. In some embodiments, the appropriate solvent is DCM. In some embodiments, the appropriate temperature for the appropriate time is about −78° C. for about 1 hour. In some embodiments, the reaction is further warmed to about room temperature for overnight. In some embodiments, the appropriate temperature for the appropriate time is about 0° C. for overnight.

In some embodiments, III-18 is reacted under suitable oxidative cleavage conditions for an appropriate time period, in an appropriate solvent, and at an appropriate temperature to produce III-19. In some embodiments, oxidative cleavage conditions include the use of an osmium reagent and N-methylmorpholine N-oxide to form an intermediate diol. In some embodiments, the osmium reagent is OsO₄ or K₂OsO₄-2H₂O. In some embodiments, the appropriate solvent is an ACN/water mixture. In some embodiments, the appropriate temperature for the appropriate time is from about 0° C. to about room temperature for overnight. In some embodiments, the diol is cleaved to form II-19 under the appropriate oxidative cleavage conditions for an appropriate time period, in an appropriate solvent, and at an appropriate temperature. In some embodiments, appropriate oxidative cleavage conditions include the use of NaIO₄. In some embodiments, the appropriate solvent is a THF/water mixture. In some embodiments, the appropriate temperature for the appropriate time is from about 0° C. to about room temperature for overnight.

In some embodiments, III-19 is reduced to a primary alcohol under suitable reducing conditions, and then halogenated under suitable halogenation conditions to produce III-20. In some embodiments, suitable reducing conditions include the use of a borohydride reagent. In some embodiments, the reducing conditions include the use of NaBH₄ in an appropriate solvent, at an appropriate temperature for an appropriate amount of time. In some embodiments, the appropriate solvent is THF. In some embodiments, the appropriate temperature for the appropriate time is about 0° C. for about 1 hour. In some embodiments, the reaction is warmed to about room temperature for overnight. In some embodiments, the primary alcohol is reacted under suitable halogenation conditions to produce an alkyl halide. In some embodiments, suitable halogenation conditions are bromination conditions that include use of CBr₄ in an appropriate solvent at an appropriate initial temperature followed by PPh₃ in the appropriate solvent, at an appropriate temperature for an appropriate time. In some embodiments, the appropriate solvent is a halogenated solvent, such as DCM. In some embodiments, the appropriate initial temperature is about 0° C. In some embodiments, the appropriate temperature and time after addition of PPh₃ is about 0° C. for about 1 hour. In some embodiments, an appropriate solvent for addition of PPh₃ is THF. In some embodiments, the reaction is further warmed to about room temperature for overnight.

In some embodiments, III-20 is subjected to intramolecular alkylation conditions to form III-21. In some embodiments, intramolecular alkylation conditions include a suitable base. In some embodiments, the suitable base is lithium diisopropylamide in an appropriate solvent, at an appropriate temperature for an appropriate amount of time. In some embodiments, the appropriate solvent is a HMPA and THF mixture. In some embodiments, the appropriate temperature for the appropriate amount of time is about −78° C. for about 3 hours or from about −78° C. to room temperature for overnight.

In some embodiments, ester III-21 is reduced to an alcohol by suitable reduction conditions followed by oxidation to aldehyde III-22 by suitable oxidation conditions. In some embodiments, suitable reduction conditions include the use of DIBALH in an appropriate solvent at an appropriate temperature for an appropriate time. In some embodiments, the appropriate solvent is DCM. In some embodiments, the appropriate temperature for the appropriate time is about −78° C. for about 1 hour. In some embodiments, the reaction is further warmed to about room temperature for about two hours to produce an alcohol. In some embodiments, suitable oxidation conditions are chromium-based oxidations. In some embodiments, suitable oxidation conditions include the use of PCC in an appropriate solvent at an appropriate temperature for an appropriate time. In some embodiments, silica gel is added. In some embodiments, the appropriate solvent is DCM. In some embodiments, the appropriate temperature is about room temperature for about 2 hours. Alternatively, in some embodiments, the oxidation conditions include the use of oxalyl chloride and DMSO with an amine base in an appropriate solvent at an appropriate temperature for an appropriate time. In some embodiments, the appropriate amine base is TEA. In some embodiments, the appropriate solvent is DCM. In some embodiments, the appropriate temperature for the appropriate amount of time is about −78° C. for about 1 hour.

In some embodiments, carboxylic acid III-23 is prepared from aldehyde III-22 under appropriate oxidation conditions. In some embodiments suitable oxidation conditions include but are not limited to use of Jones reagent in an appropriate solvent for an appropriate time and at an appropriate temperature. In some embodiments, the appropriate solvent is acetone. In some embodiments, the appropriate time is about 2 hours. In some embodiments, the suitable temperature is about room temperature.

In some embodiments, intermediate III-23 is reacted under suitable amide coupling conditions to provide amide III-24. In some embodiments, appropriate amide coupling conditions include using an appropriate coupling reagent and amine with an appropriate base and solvent at an appropriate time and at an appropriate temperature. In some embodiments, the appropriate coupling reagent is HATU. In some embodiments, the appropriate amine is methylamine. In some embodiments, the appropriate base is diisopropylethylamine. In some embodiments, the appropriate solvent is DMF. In some embodiments, the reaction temperature is about room temperature and the reaction time is about 1 hour.

In some embodiments, intermediate III-24 is reacted under suitable conditions to provide phenol III-25. In some embodiments, appropriate conditions include using an appropriate reagent with an appropriate solvent at an appropriate time and at an appropriate temperature. In some embodiments, the appropriate reagent is boron tribromide. In some embodiments, the appropriate solvent is DCM. In some embodiments, the reaction temperature is about −78° C. and the reaction time is about 3 hours.

In some embodiments, compounds described herein are prepared as outlined in Scheme 4.

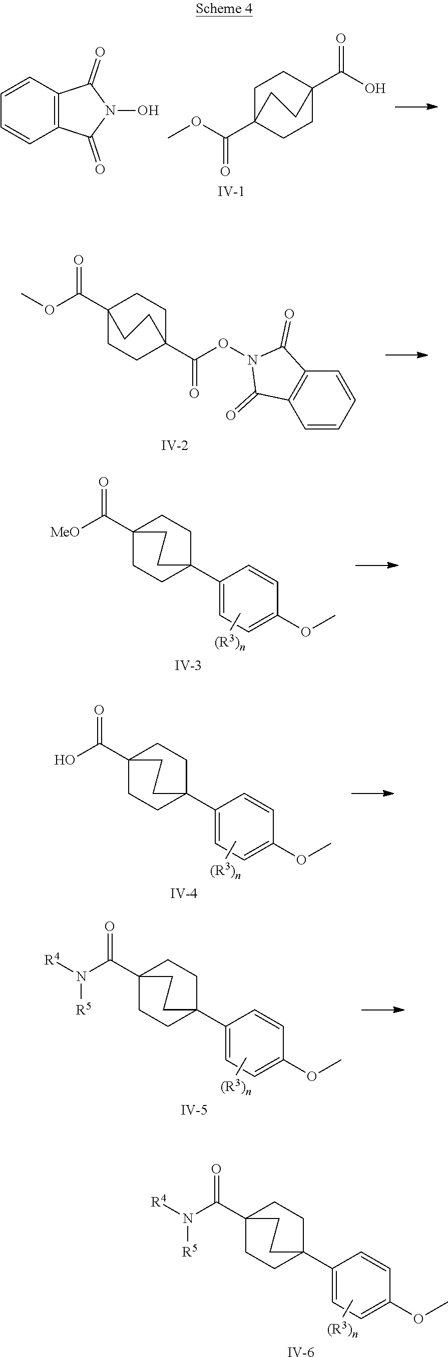

In Scheme 4, substituents R³, R⁴, R⁵, and n are described herein. In some embodiments, n is 0. In some embodiments, R⁴ is methyl and R⁵ is H.

In some embodiments, 1,4-endoethylenecyclohexyl carboxylic acid IV-1 is reacted with N-hydroxyphthalimide under suitable coupling reaction conditions to provide IV-2. In some embodiments, suitable coupling reaction conditions include an appropriate coupling agent, an appropriate base, and an appropriate solvent for an appropriate time and at an appropriate temperature. In some embodiments, the coupling agent is N,N-diisopropylcarbodiimide. In some embodiments, the base is DMAP. In some embodiments, the solvent is DCM or DCE. In some embodiments, the time and the temperature are overnight and room temperature.

In some embodiments, IV-2 is reacted under suitable aryl-alkyl cross-coupling reaction conditions to provide aryl-alkyl IV-3. In some embodiments, the suitable aryl-alkyl cross-coupling reaction conditions include nickel. In some embodiments, suitable aryl-alkyl cross-coupling reaction conditions include an appropriate source of Ni, an appropriate arylzinc reagent, an appropriate auxiliary ligand, and a solvent, for an appropriate time at an appropriate temperature. In some embodiments, the source of Ni is nickel(II) acetylacetonate. In some embodiments, the source of Ni is a Ni(II) halide or a solvate thereof. In some embodiments, the Ni(II) halide is a Ni(II) chloride or Ni(II) bromide. In some embodiments, the arylzinc reagent is a substituted phenylzinc reagent. In some embodiments, the substituted phenylzinc reagent is a methoxyphenylzinc reagent. In some embodiments, the methoxyphenylzinc reagent is bis(4-methoxyphenyl)zinc. In some embodiments, the auxiliary ligand is an alkyl-substituted 2,2′-bispyridine. In some embodiments, the alkyl-substituted 2,2′-bispyridine is 6,6′-dimethyl-2,2′-bispyridine or 4,4′-di-tert-butyl-2,2′-bispyridine. In some embodiments, the alkyl-substituted 2,2′-bispyridine is 6,6′-dimethyl-2,2′-bispyridine. In some embodiments, the solvent is acetonitrile, DMF, THF or combinations thereof. In some embodiments, the solvent is acetonitrile. In some embodiments, the time and the temperature are overnight and 80° C.

In some embodiments, carboxylic acid IV-4 is prepared from ester IV-3 under appropriate hydrolysis conditions. In some embodiments suitable hydrolysis conditions include but are not limited to use of a suitable reagent in an appropriate solvent or solvent mixture for an appropriate time and at an appropriate temperature. In some embodiments, the appropriate reagent is lithium hydroxide. In some embodiments, the appropriate solvent mixture is THF and H₂O. In some embodiments, the appropriate temperature is about 60° C. In some embodiments, the appropriate time is about 18 hours (overnight).

In some embodiments, intermediate IV-4 is reacted under suitable amide coupling conditions to provide amide IV-5. In some embodiments, appropriate amide coupling conditions include using an appropriate coupling reagent and amine with an appropriate base and solvent at an appropriate time and at an appropriate temperature. In some embodiments, the appropriate coupling reagent is HATU. In some embodiments, the appropriate amine is methylamine. In some embodiments, the appropriate base is diisopropylethylamine. In some embodiments, the appropriate solvent is DMF. In some embodiments, the reaction temperature is about room temperature and the reaction time is about 1 hour.

In some embodiments, intermediate IV-5 is reacted under suitable conditions to provide phenol IV-6. In some embodiments, appropriate conditions include using an appropriate reagent with an appropriate solvent at an appropriate time and at an appropriate temperature. In some embodiments, the appropriate reagent is boron tribromide. In some embodiments, the appropriate solvent is DCM. In some embodiments, the reaction temperature is about −78° C. to room temperature and the reaction time is about 1 hour.

In some embodiments, compounds are prepared as described in the Examples.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e. a C₁-C₁₀alkyl. Whenever it appears herein, a numerical range such as “1 to 10” refers to each integer in the given range; e.g., “1 to 10 carbon atoms” means that the alkyl group consist of 1 carbon atom, 2 carbon atoms, 3 carbon atoms, 4 carbon atoms, 5 carbon atoms, 6 carbon atoms, etc., up to and including 10 carbon atoms, although the present definition also covers the occurrence of the term “alkyl” where no numerical range is designated. In some embodiments, an alkyl is a C₁-C₆alkyl. In one aspect, the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl.

An “alkylene” group refers refers to a divalent alkyl radical. Any of the above mentioned monovalent alkyl groups may be an alkylene by abstraction of a second hydrogen atom from the alkyl. In some embodiments, an alkylene is a C₁-C₆alkylene. In other embodiments, an alkylene is a C₁-C₄alkylene. In certain embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises two carbon atoms (e.g., C₂ alkylene). In other embodiments, an alkylene comprises two to four carbon atoms (e.g., C₂-C₄ alkylene). Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Deuteroalkyl” refers to an alkyl group where 1 or more hydrogen atoms of an alkyl are replaced with deuterium.

The term “alkenyl” refers to a type of alkyl group in which at least one carbon-carbon double bond is present. In one embodiment, an alkenyl group has the formula —C(R)═CR₂, wherein R refers to the remaining portions of the alkenyl group, which may be the same or different. In some embodiments, R is H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

An “alkoxy” group refers to a (alkyl)O— group, where alkyl is as defined herein.

The term “alkylamine” refers to the —N(alkyl)ₓH_{y} group, where x is 0 and y is 2, or where x is 1 and y is 1, or where x is 2 and y is 0.

The term “aromatic” refers to a planar ring having a delocalized it-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon or nitrogen atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycle includes cycloalkyl and aryl.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a C₆-C₁₀aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e. skeletal atoms) is a carbon atom. In some embodiments, cycloalkyls are spirocyclic or bridged compounds. In some embodiments, cycloalkyls are optionally fused with an aromatic ring, and the point of attachment is at a carbon that is not an aromatic ring carbon atom. Cycloalkyl groups include groups having from 3 to 10 ring atoms. In some embodiments, cycloalkyl groups are selected from among cyclopropyl, cyclobutyl, cyclopentyl, cyclopentenyl, cyclohexyl, cyclohexenyl, cycloheptyl, cyclooctyl, spiro[2.2]pentyl, norbornyl and bicyclo[1.1.1]pentyl. In some embodiments, a cycloalkyl is a C₃-C₆cycloalkyl. In some embodiments, a cycloalkyl is a monocyclic cycloalkyl. Monocyclic cycloalkyls include, but are not limited to, cyclopropyl, cyclobutyl, cyclopentyl, cyclohexyl, cycloheptyl, and cyclooctyl. Polycyclic cycloalkyls include, for example, adamantyl, norbornyl (i.e., bicyclo[2.2.2]octyl and bicyclo[2.2.1]heptanyl), norbornenyl, decalinyl, 7,7-dimethyl-bicyclo[2.2.1]heptanyl, and the like.

The term “halo” or, alternatively, “halogen” or “halide” means fluoro, chloro, bromo or iodo. In some embodiments, halo is fluoro, chloro, or bromo.

The term “haloalkyl” refers to an alkyl in which one or more hydrogen atoms are replaced by a halogen atom. In one aspect, a fluoroalkyl is a C₁-C₆fluoroalkyl.

The term “fluoroalkyl” refers to an alkyl in which one or more hydrogen atoms are replaced by a fluorine atom. In one aspect, a fluoroalkyl is a C₁-C₆fluoroalkyl. In some embodiments, a fluoroalkyl is selected from trifluoromethyl, difluoromethyl, fluoromethyl, 2,2,2-trifluoroethyl, 1-fluoromethyl-2-fluoroethyl, and the like.

The term “heteroalkyl” refers to an alkyl group in which one or more skeletal atoms of the alkyl are selected from an atom other than carbon, e.g., oxygen, nitrogen (e.g. —NH—, —N(alkyl)-, sulfur, or combinations thereof. A heteroalkyl is attached to the rest of the molecule at a carbon atom of the heteroalkyl. In one aspect, a heteroalkyl is a C₁-C₆heteroalkyl.

The term “heteroalkylene” refers refers to a divalent heteroalkyl radical.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. In some embodiments, heterocycles are monocyclic, bicyclic, polycyclic, spirocyclic or bridged compounds. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 2-azabicyclo[2.2.2]octanyl, 3-azabicyclo[3.2.1]octanyl, 5-azabicyclo[2.1.1]hexanyl, 6-azabicyclo[3.1.1]heptanyl, 7-azabicyclo[2.2.1]heptanyl, 8-azabicyclo[3.2.1]octanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” or “heteroalicyclic” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. The term heteroalicyclic also includes all ring forms of the carbohydrates, including but not limited to the monosaccharides, the disaccharides and the oligosaccharides. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “oxo” refers to the ═O radical.

The term “bond” or “single bond” refers to a chemical bond between two atoms, or two moieties when the atoms joined by the bond are considered to be part of larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from D, halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, alkenyl, alkynyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from D, halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy, —SC₁-C₄alkyl, —S(═O)C₁-C₄alkyl, and —S(═O)₂C₁-C₄alkyl. In some embodiments, optional substituents are independently selected from D, halogen, —CN, —NH₂, —OH, —NH(CH₃), —N(CH₃)₂, —CH₃, —CH₂CH₃, —CF₃, —OCH₃, and —OCF₃. In some embodiments, substituted groups are substituted with one or two of the preceding groups. In some embodiments, an optional substituent on an aliphatic carbon atom (acyclic or cyclic) includes oxo (═O).

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, compounds described herein can be administered locally to the area in need of treatment, by for example, local infusion during surgery, topical application such as creams or ointments, injection, catheter, or implant. The administration can also be by direct injection at the site of a diseased tissue or organ.

In some embodiments, pharmaceutical compositions suitable for oral administration are presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

Pharmaceutical compositions may also be formulated in rectal compositions such as suppositories or retention enemas, e.g., containing conventional suppository bases such as cocoa butter, polyethylene glycol, or other glycerides.

Pharmaceutical compositions may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Pharmaceutical compositions suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation.

Pharmaceutical compositions for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, pharmaceutical preparations may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

In some embodiments, a compound disclosed herein is formulated in such a manner that delivery of the compound to a particular region of the gastrointestinal tract is achieved. For example, a compound disclosed herein is formulated for oral delivery with bioadhesive polymers, pH-sensitive coatings, time dependent, biodegradable polymers, microflora activated systems, and the like, in order to effect delivering of the compound to a particular region of the gastrointestinal tract.

In some embodiments, a compound disclosed herein is formulated to provide a controlled release of the compound. Controlled release refers to the release of the compound described herein from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

Approaches to deliver the intact therapeutic compound to the particular regions of the gastrointestinal tract (e.g. such as the colon), include:

(i) Coating with polymers: The intact molecule can be delivered to the colon without absorbing at the upper part of the intestine by coating of the drug molecule with the suitable polymers, which degrade only in the colon.

(ii) Coating with pH-sensitive polymers: The majority of enteric and colon targeted delivery systems are based on the coating of tablets or pellets, which are filled into conventional hard gelatin capsules. Most commonly used pH-dependent coating polymers are methacrylic acid copolymers, commonly known as Eudragit® S, more specifically Eudragit® L and Eudragit® S. Eudragit® L100 and S 100 are copolymers of methacrylic acid and methyl methacrylate.

(iii) Coating with biodegradable polymers;

(iv) Embedding in matrices;

(v) Embedding in biodegradable matrices and hydrogels;

(vi) Embedding in pH-sensitive matrices;

(vii) Timed release systems;

(viii) Redox-sensitive polymers;

(ix) Bioadhesive systems;

(x) Coating with microparticles;

(xi) Osmotic controlled drug delivery;

Another approach towards colon-targeted drug delivery or controlled-release systems includes embedding the drug in polymer matrices to trap it and release it in the colon. These matrices can be pH-sensitive or biodegradable. Matrix-Based Systems, such as multi-matrix (MMX)-based delayed-release tablets, ensure the drug release in the colon.

Additional pharmaceutical approaches to targeted delivery of therapeutics to particular regions of the gastrointestinal tract are known. Chourasia M K, Jain S K, Pharmaceutical approaches to colon targeted drug delivery systems, J Pharm Pharm Sci. 2003 January-April; 6(1):33-66. Patel M, Shah T, Amin A. Therapeutic opportunities in colon-specific drug-delivery systems Crit Rev Ther Drug Carrier Syst. 2007; 24(2): 147-202. Kumar P, Mishra B. Colon targeted drug delivery systems—an overview. Curr Drug Deliv. 2008 July; 5(3):186-98. Van den Mooter G. Colon drug delivery. Expert Opin Drug Deliv. 2006 January; 3(1): III-25. Seth Amidon, Jack E. Brown, and Vivek S. Dave, Colon-Targeted Oral Drug Delivery Systems: Design Trends and Approaches, AAPS PharmSciTech. 2015 August; 16(4): 731-741.

It should be understood that in addition to the ingredients particularly mentioned above, the compounds and compositions described herein may include other agents conventional in the art having regard to the type of formulation in question, for example those suitable for oral administration may include flavoring agents.

### Methods of Dosing and Treatment Regimens

In one embodiment, the compounds described herein, or a pharmaceutically acceptable salt thereof, are used in the preparation of medicaments for the treatment of diseases or conditions in a mammal that would benefit from administration of an SSAO inhibitor. Methods for treating any of the diseases or conditions described herein in a mammal in need of such treatment, involves administration of pharmaceutical compositions that include at least one compound described herein or a pharmaceutically acceptable salt, active metabolite, prodrug, or pharmaceutically acceptable solvate thereof, in therapeutically effective amounts to said mammal.

In some embodiments disclosed herein, are methods of administering an SSAO inhibitor in combination with an additional therapeutic agent.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments, wherein the patient's condition does not improve, upon the doctor's discretion, the compounds are administered chronically, that is, for an extended period of time, including throughout the duration of the patient's life in order to ameliorate or otherwise control or limit the symptoms of the patient's disease or condition.

In certain embodiments, wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of the compound, including further embodiments in which (i) the compound is administered once a day; or (ii) the compound is administered to the mammal multiple times over the span of one day.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 1 year.

In certain instances, it is appropriate to administer at least one compound described herein, or a pharmaceutically acceptable salt thereof, in combination with one or more other therapeutic agents.

In one embodiment, the therapeutic effectiveness of one of the compounds described herein is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering one of the compounds described herein with another agent (which also includes a therapeutic regimen) that also has therapeutic benefit.

In one specific embodiment, a compound described herein, or a pharmaceutically acceptable salt thereof, is co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient may be additive of the two therapeutic agents or the patient may experience a synergistic benefit.

In certain embodiments, different therapeutically-effective dosages of the compounds disclosed herein will be utilized in formulating pharmaceutical composition and/or in treatment regimens when the compounds disclosed herein are administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. It also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, the compound provided herein is administered either simultaneously with the one or more other therapeutic agents, or sequentially.

In combination therapies, the multiple therapeutic agents (one of which is one of the compounds described herein) are administered in any order or even simultaneously. If administration is simultaneous, the multiple therapeutic agents are, by way of example only, provided in a single, unified form, or in multiple forms (e.g., as a single pill or as two separate pills).

The compounds described herein, or a pharmaceutically acceptable salt thereof, as well as combination therapies, are administered before, during or after the occurrence of a disease or condition, and the timing of administering the composition containing a compound varies. Thus, in one embodiment, the compounds described herein are used as a prophylactic and are administered continuously to subjects with a propensity to develop conditions or diseases in order to prevent the occurrence of the disease or condition. In another embodiment, the compounds and compositions are administered to a subject during or as soon as possible after the onset of the symptoms. In specific embodiments, a compound described herein is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a compound described herein or a formulation containing the compound is administered for at least 2 weeks, about 1 month to about 5 years.

In some embodiments, an SSAO inhibitor is administered in combination with an additional therapeutic agent for the treatment of a liver disease or condition. In some embodiments, the additional therapeutic agent is selected from an FXR agonist, an ACC inhibitor, and an ASK-1 inhibitor, or a combination thereof.

### EXAMPLES

The following examples are provided for illustrative purposes only and not to limit the scope of the claims provided herein.

As used above, and throughout the description of the invention, the following abbreviations, unless otherwise indicated, shall be understood to have the following meanings:
ACN or MeCN acetonitrile
AcOH acetic acid
Ac acetyl
BINAP 2,2′-bis(diphenylphosphino)-1,1′-binaphthalene
Bn benzyl
BOC or Boc tert-butyl carbamate
t-Bu tert-butyl
Cy cyclohexyl
DBA or dba dibenzylideneacetone
DCE dichloroethane (ClCH₂CH₂Cl)
DCM dichloromethane (CH₂Cl₂)
DIPEA or DIEA diisopropylethylamine
DMAP 4-(N,N-dimethylamino)pyridine
DME 1,2-dimethoxyethane
DMF N,N-dimethylformamide
DMA N,N-dimethylacetamide
DMSO dimethylsulfoxide
Dppf or dppf 1,1′-bis(diphenylphosphino)ferrocene
EEDQ 2-Ethoxy-1-ethoxycarbonyl-1,2-dihydroquinoline
eq equivalent(s)
Et ethyl
Et₂O diethyl ether
EtOH ethanol
EtOAc ethyl acetate
HATU 1-[bis(dimethylamino)methylene]-1H-1,2,3-triazolo[4,5-b]pyridinium 3-oxid hexafluorophosphate
HMPA hexamethylphosphoramide
HPLC high performance liquid chromatography
KHMDS potassium bis(trimethylsilyl)amide
NaHMDS sodium bis(trimethylsilyl)amide
LiHMDS lithium bis(trimethylsilyl)amide
LAH lithium aluminum anhydride
LCMS liquid chromatography mass spectrometry
Me methyl
MeOH methanol
MS mass spectroscopy
Ms mesyl
NBS N-bromosuccinimide
NMM N-methyl-morpholine
NMP N-methyl-pyrrolidin-2-one
NMR nuclear magnetic resonance
PCC pyridinium chlorochromate
Ph phenyl
PPTS pyridium p-toluenesulfonate
iPr/i-Pr iso-propyl
TB S tert-butyldimethylsilyl
RP-HPLC reverse phase-high pressure liquid chromatography
TFA trifluoroacetic acid
TEA triethylamine
THF tetrahydrofuran
TLC thin layer chromatography

### Intermediate 1

### Tert-Butyl (2-(bromomethyl)-3-fluoroallyl)carbamate

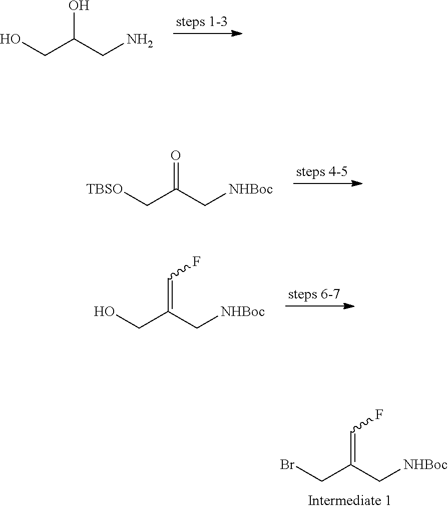 [SMILES: NCC(O)CO ; CC(C)(C)OC(=O)NCC(=O)CO[Si](C)(C)C(C)(C)C ; CC(C)(C)OC(=O)NCC(=CF)CO ; CC(C)(C)OC(=O)NCC(=CF)CBr]

### Step 1: Tert-Butyl (2,3-dihydroxypropyl)carbamate

Di-tert-butyl dicarbonate (264 g, 1.21 mol) was added to a stirred solution of 3-aminopropane-1,2-diol (100 g, 1.10 mol) and triethylamine (111 g, 1.10 mol) in MeOH (2000 mL) and the reaction stirred at room temperature overnight. The mixture was concentrated under reduced pressure and residual solvent removed by azeotrope with toluene to yield the crude material tert-butyl (2,3-dihydroxypropyl)carbamate (210 g) as a white solid. ¹H NMR (400 MHz, CDCl₃) δ 5.31 (s, 1H), 3.94-3.68 (m, 3H), 3.66-3.51 (m, 2H), 3.39-3.09 (m, 2H), 1.45 (s, 9H).

### Step 2: Tert-Butyl (3-((tert-butyldimethylsilyl)oxy)-2-hydroxypropyl)carbamate

A solution of tert-butyl (2,3-dihydroxypropyl)carbamate (210 g, 1.10 mol) and imidazole (82.2 g, 1.21 mol) in DCM (1500 mL) was cooled to 0° C. tert-Butyldimethylsilyl chloride (166 g, 1.10 mol) was added to the reaction at 0° C., the mixture allowed to slowly warm to room temperature and stirred overnight. The mixture was poured into water (1000 ml) and extracted with DCM (1000 mL). The combined organics were washed with water (2×1000 ml), brine (1000 ml), dried (Na₂SO₄) and concentrated under reduced pressure to yield the crude material tert-butyl (3-((tert-butyldimethylsilyl)oxy)-2-hydroxypropyl)carbamate (340 g) as a yellow oil. ¹H NMR (400 MHz, CDCl₃) δ 5.08 (br s, 1H), 3.75-3.65 (m, 1H), 3.63-3.56 (m, 1H), 3.55-3.46 (m, 1H), 3.36-3.26 (m, 1H), 3.12-3.05 (m, 1H), 3.03-2.98 (m, 1H), 1.41 (s, 9H), 0.86 (s, 9H), 0.04 (s, 6H).

### Step 3: Tert-Butyl (3-((tert-butyldimethylsilyl)oxy)-2-oxopropyl)carbamate

DMSO (25.6 g, 327 mmol) was added dropwise to a solution of oxalyl chloride (31.2 g, 246 mmol) in DCM (500 mL) at −78° C. and the reaction stirred at −78° C. for an additional 1 h. A solution of tert-butyl (3-((tert-butyldimethylsilyl)oxy)-2-hydroxypropyl)carbamate (50 g, 164 mmol) in DCM (100 mL) was then added dropwise at −78° C. and stirred at −78° C. for an additional 1 h. Et₃N (82.8 g, 818 mmol) was added dropwise at −78° C. and the reaction allowed to warm to room temperature. The mixture was poured into water (500 ml) and extracted with DCM (2×1000 mL). The combined organics were dried (Na₂SO₄), filtered and concentrated under reduced pressure. Purification by flash chromatography on silica gel (2% ethyl acetate in petroleum ether) gave tert-butyl (3-((tert-butyldimethylsilyl)oxy)-2-oxopropyl)carbamate (40 g, 81%) as a yellow oil. ¹H NMR (400 MHz, CDCl₃) δ 5.08 (br s, 1H), 4.21-4.02 (m, 4H), 1.41 (s, 9H), 0.86 (s, 9H), 0.04 (s, 6H).

### Step 4: Tert-Butyl (2-(((tert-butyldimethylsilyl)oxy)methyl)-3-fluoroallyl)carbamate

A solution of (fluoromethyl)triphenylphosphonium bromide (29.0 g, 77.3 mmol) in THF (300 mL) was cooled to −20° C. before the dropwise addition of a 1M NaHMDS solution in THF (206 mL, 206 mmol). The mixture was stirred at −20° C. for 30 min before the addition of a solution of tert-butyl (3-((tert-butyldimethylsilyl)oxy)-2-oxopropyl)carbamate (15.6 g, 51.5 mmol) in THF (100 mL). The reaction was warmed to room temperature and stirred for 2 h. The reaction mixture was poured into water (1000 mL) and extracted with EtOAc (3×500 mL). The combined organics were washed with brine (2×500 mL), dried (Na₂SO₄) and concentrated under reduced pressure. Purification by flash chromatography on silica gel (0-2% ethyl acetate in petroleum ether) gave a 4:1 mixture of E:Z regioisomers of tert-butyl (2-(((tert-butyldimethylsilyl)oxy)methyl)-3-fluoroallyl)carbamate (22.9 g, 69%) isolated as a yellow oil. ¹H NMR (400 MHz, CDCl₃) δ 6.61 (d, 0.8H), 6.57 (d, 0.2H), 5.05 (s, 0.2H), 4.90 (s, 0.8H), 4.34-4.09 (m, 2H), 3.90-3.68 (m, 2H), 1.45 (s, 9H), 0.91 (s, 9H), 0.08 (s, 6H).

### Step 5: Tert-Butyl (3-fluoro-2-(hydroxymethyl)allyl)carbamate

1M TBAF in THF (107 mL, 107 mmol) was added to a solution of a 4:1 mixture of E:Z isomers of tert-butyl (2-(((tert-butyldimethylsilyl)oxy)methyl)-3-fluoroallyl)carbamate (22.9 g, 71.7 mmol) in THF (200 mL) and the reaction stirred at room temperature for 1 h. The reaction mixture was poured into a saturated ammonium chloride solution (300 mL) and extracted with EtOAc (3×200 mL). The organic layers were combined, washed with brine (2×200 mL), dried (Na₂SO₄) and concentrated under reduced pressure. Purification by flash chromatography using silica gel (375:25:80 hexane:THF:ethyl acetate) gave a 4:1 mixture of E:Zregioisomers of tert-butyl (3-fluoro-2-(hydroxymethyl)allyl)carbamate (14 g, 80%) isolated as a yellow oil. ¹H NMR (400 MHz, CDCl₃): δ 6.61 (d, 0.8H), 6.51 (d, 0.2H), 4.93 (s, 1H), 4.29-4.27 (m, 0.4H), 4.05-3.87 (m, 3.6H), 3.74-3.72 (m, 1H), 1.46 (s, 9H).

### Step 6: 2-(((tert-Butoxycarbonyl)amino)methyl)-3-fluoroallyl Methanesulfonate

A solution of a 4:1 mixture of tert-butyl (3-fluoro-2-(hydroxymethyl)allyl)carbamate (3.0 g, 14.6 mmol) and Et₃N (4.0 mL, 29.2 mmol) in acetone (40 mL) was cooled to 0° C. Methanesulfonyl chloride (1.7 mL, 21.9 mmol) was added and the reaction stirred for 1 h. The mixture was filtered and the cake washed with acetone (5 ml) to give a solution of 2-(((tert-butoxycarbonyl)amino)methyl)-3-fluoroallyl methanesulfonate in acetone. Yield assumed quantitative (4.14 g).

### Step 7: Tert-Butyl (2-(bromomethyl)-3-fluoroallyl)carbamate

Lithium bromide (12.7 g, 146 mmol) was added to a solution of 2-(((tert-butoxycarbonyl)amino)methyl)-3-fluoroallyl methanesulfonate (4.14 g, 14.6 mmol) in acetone (50 mL) and the reaction stirred at room temperature for 1 h. The reaction mixture was poured into water (30 mL) and extracted with EtOAc (3×20 mL). The combined organics were washed with brine (2×20 mL), dried (Na₂SO₄) and concentrated under reduced pressure to give the crude product as a 4:1 mixture of E:Z regioisomers of tert-butyl (2-(bromomethyl)-3-fluoroallyl)carbamate (3 g) isolated as a yellow oil. ¹H NMR (400 MHz, CDCl₃) δ 6.77 (d, 0.8H), 6.63 (d, 0.2H), 4.76 (s, 1H), 4.08 (d, 0.4H), 4.01 (d, 1.6H), 3.98-3.93 (m, 1.6H), 3.80-3.75 (m, 0.4H), 1.46 (s, 9H); LCMS 212.1 [M+H-tBu]⁺.

The mixture of E:Z regioisomers of tert-butyl (2-(bromomethyl)-3-fluoroallyl)carbamate may be purified further. For example, a 5 g mixture of tert-butyl (2-(bromomethyl)-3-fluoroallyl)carbamate (E/Z=4:1) was loaded on ˜10 g of 100 mesh silica gel. This in turn was put on ˜400 g of 1000 mesh silica gel and eluted with n-hexane/THF/EtOAc=75/5/16 (3.8 L). The collected fractions were monitored by TLC (petroleum ether/EtOAc=1:1, KMnO₄ as color developing agent, R_{f}=0.5/0.55). After concentration, (E)-tert-butyl (3-fluoro-2-(hydroxymethyl)allyl)carbamate (2.5 g, >95% E by HNMR) was obtained as a yellow oil.

Intermediate 1 of different E:Z isomer ratios can be used to prepare the final compounds below. For example, a mixture of 4:1 E:Z regioisomers of Intermediate 1 can be used with isolation of final compounds to give final compounds of >95% (E) purity. Alternatively, >95% (E) Intermediate 1 can be used to prepare final compounds of >95% (E) purity.

### Compound 1

### (E)-1-(4-((2-(Aminomethyl)-3-fluoroallyl)oxy)phenyl)cyclohexanecarboxamide Hydrochloride

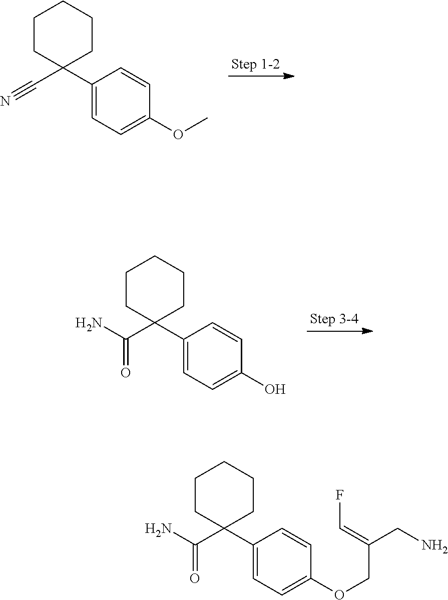 [SMILES: COc1ccc(C2(C#N)CCCCC2)cc1 ; NC(=O)C1(c2ccc(O)cc2)CCCCC1 ; NC/C(=C\F)COc1ccc(C2(C(N)=O)CCCCC2)cc1]

### Step 1: 1-(4-Methoxyphenyl)cyclohexanecarboxamide

To a solution of 1-(4-methoxyphenyl)cyclohexanecarbonitrile (3.0 g, 13.9 mmol) in 2-methylbutan-2-ol (60 mL) at room temperature, KOH (4.69 g, 83.6 mmol) was added. The mixture was stirred at 120° C. for 8 h under a nitrogen atmosphere. The reaction mixture was cooled to room temperature, poured into water (50 mL) and then extracted with EtOAc (2×50 mL). The combined organic layers were washed with an aqueous saturated NaHCO₃ solution (25 mL), brine (40 mL), dried (Na₂SO₄), filtered, concentrated and then purified by silica gel chromatography to give 1-(4-methoxyphenyl)cyclohexanecarboxamide (3.0 g, 92%) as a yellow solid. ¹H NMR (400 MHz, CDCl₃): δ 7.36 (d, 2H), 6.91 (d, 2H), 5.24-5.13 (m, 2H), 3.82 (s, 3H), 2.28-2.19 (m, 2H), 2.04-1.94 (m, 2H), 1.70-1.53 (m, 6H); MS:234.1 [M+H]⁺.

### Step 2: 1-(4-Hydroxyphenyl)cyclohexanecarboxamide

To a solution of 1-(4-methoxyphenyl)cyclohexanecarboxamide (1.0 g, 4.29 mmol) in DCM (20 mL) at −78° C., tribromoborane (1.61 g, 6.43 mmol) was added dropwise and the reaction stirred at −78° C. for an additional 3 h. The reaction mixture was poured carefully into a saturated aqueous NaHCO₃ solution (30 mL) and extracted with DCM (3×30 mL). The combined organic layers were washed with water (2×30 mL), brine (25 mL), dried (Na₂SO₄), filtered, concentrated and purified by silica gel chromatography to give 1-(4-hydroxyphenyl)cyclohexanecarboxamide (500 mg, 53%) as a white solid. ¹H NMR (400 MHz, DMSO): δ 9.22 (s, 1H), 7.16 (d, 2H), 6.85 (s, 1H), 6.75 (s, 1H), 6.67 (d, 2H), 2.35-2.22 (m, 2H), 1.51 (m, 8H).

### Step 3: (E)-tert-Butyl (2-((4-(1-carbamoylcyclohexyl)phenoxy)methyl)-3-fluoroallyl)carbamate

A mixture of 1-(4-hydroxyphenyl)cyclohexanecarboxamide (200 mg, 0.91 mmol), (E)-tert-butyl (2-(bromomethyl)-3-fluoroallyl)carbamate (269 mg, 1.00 mmol), Cs₂CO₃ (892 mg, 2.74 mmol) and acetonitrile (10 mL) was stirred at room temperature for 2.5 h. The reaction mixture was poured into water (25 mL), and then extracted with EtOAc (2×25 mL). The combined organic layers were washed with water (25 mL), brine (25 mL), dried (Na₂SO₄), filtered, concentrated and purified by silica gel chromatography to give (E)-tert-butyl (2-((4-(1-carbamoylcyclohexyl)phenoxy)methyl)-3-fluoroallyl)carbamate (250 mg, 57%) as a yellow solid. ¹H NMR (400 MHz, CDCl₃): δ 7.35 (d, 2H), 6.91 (d, 2H), 6.75 (d, 1H), 5.31-5.23 (s, 2H), 4.85-4.73 (m, 1H), 4.44 (d, 2H), 4.02-3.97 (m, 2H), 2.28-2.19 (m, 2H), 2.02-1.93 (m, 2H), 1.68-1.50 (m, 6H), 1.42 (s, 9H); MS:429.1 [M+Na]⁺.

### Step 4: (E)-1-(4-((2-(Aminomethyl)-3-fluoroallyl)oxy)phenyl)cyclohexanecarboxamide Hydrochloride

A solution of (E)-tert-butyl (2-((4-(1-carbamoylcyclohexyl)phenoxy)methyl)-3-fluoroallyl)carbamate (300 mg, 0.74 mmol) and TFA (2 mL) in DCM (5 mL) was stirred at room temperature for 2 h under a nitrogen atmosphere. The mixture was concentrated to dryness and then purified by reverse-phase HPLC to give (E)-1-(4-((2-(aminomethyl)-3-fluoroallyl)oxy)phenyl)cyclohexanecarboxamide hydrochloride (33 mg, 1%) as a white solid. ¹H NMR (400 MHz, DMSO): δ 8.26 (s, 3H), 7.31 (d, 2H), 7.28 (d, 1H), 6.99-6.90 (m, 3H), 6.79 (s, 1H), 4.60 (d, 2H), 3.58 (d, 2H), 2.36-2.27 (m, 2H), 1.65-1.38 (m, 7H), 1.32-1.13 (m, 1H); MS:307.1 [M+H]⁺.

### Compound 1.01

### (E)-1-(4-((2-(Aminomethyl)-3-fluoroallyl)oxy)phenyl)-N-methylcyclohexanecarboxamide

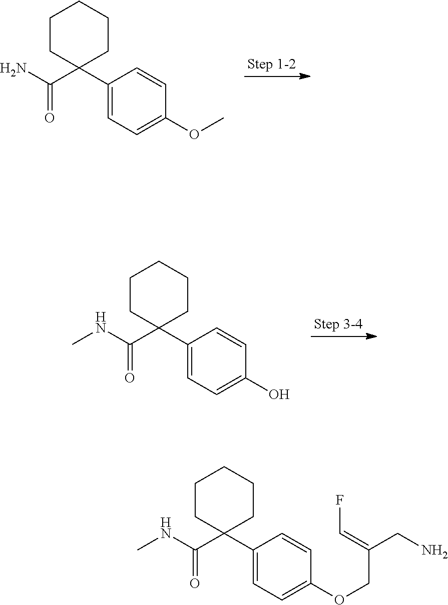 [SMILES: COc1ccc(C2(C(N)=O)CCCCC2)cc1 ; CNC(=O)C1(c2ccc(O)cc2)CCCCC1 ; CNC(=O)C1(c2ccc(OC/C(=C/F)CN)cc2)CCCCC1]

### Step 1: 1-(4-Methoxyphenyl)-N-methylcyclohexanecarboxamide

To a solution of 1-(4-methoxyphenyl)cyclohexanecarboxamide (1.0 g, 4.29 mmol) in THF (10 mL) at 0° C., 60% NaH (257 mg, 6.43 mmol) was added and the reaction stirred for 0.5 h. Iodomethane (608 mg, 4.29 mmol) was added at 0° C., the mixture warmed to room temperature and stirred at room temperature overnight. The reaction mixture was carefully poured into water (25 mL) and then extracted with EtOAc (2×25 mL). The combined organic layers were washed with water (25 mL), brine (25 ml), dried (Na₂SO₄), filtered, concentrated and purified by silica gel chromatography to give 1-(4-methoxyphenyl)-N-methylcyclohexanecarboxamide (740 mg, 70%) as a white solid. ¹H NMR (400 MHz, CDCl₃): δ 7.32 (d, 2H), 6.91 (d, 2H), 5.45-5.31 (m, 1H), 3.82 (s, 3H), 2.69 (d, 3H), 2.28-2.19 (m, 2H), 2.07-1.96 (m, 2H), 1.59 (m, 2H), 1.53-1.40 (m, 4H); MS:248.1 [M+H]+.

### Step 2: 1-(4-hydroxyphenyl)-N-methylcyclohexanecarboxamide

To a solution of 1-(4-methoxyphenyl)-N-methylcyclohexanecarboxamide (800 mg, 3.23 mmol) in DCM (15 mL) at −78° C., tribromoborane (9.3 g, 37.1 mmol) was added dropwise and the mixture stirred at −78° C. for 1 h. The reaction mixture was warmed to room temperature overnight. The reaction mixture was carefully poured into saturated aqueous NaHCO₃ solution (25 mL) and extracted with DCM (3×25 mL). The combined organic layers were washed with water (25 mL), brine (25 mL), and dried (Na₂SO₄), filtered, concentrated and purified by silica gel chromatography to give 1-(4-hydroxyphenyl)-N-methylcyclohexanecarboxamide (480 mg, 64%) as a white solid. ¹H NMR (400 MHz, DMSO-d₆): δ 9.19 (s, 1H), 7.31-7.25 (m, 1H), 7.11 (d, 2H), 6.67 (d, 2H), 2.52 (s, 3H), 2.27 (d, 2H), 1.65-1.44 (m, 5H), 1.43-1.30 (m, 2H), 1.28-1.15 (m, 1H); MS:232.0 [M−H]⁻.

### Step 3: (E)-tert-butyl (3-fluoro-2-((4-(1-(methylcarbamoyl)cyclohexyl)phenoxy)methyl)allyl)carbamate

A mixture of 1-(4-hydroxyphenyl)-N-methylcyclohexanecarboxamide (200 mg, 0.85 mmol), (E)-tert-butyl (2-(bromomethyl)-3-fluoroallyl)carbamate (276 mg, 1.03 mmol), Cs₂CO₃ (838 mg, 2.57 mmol) and acetonitrile (10 mL) was stirred at room temperature overnight. The reaction mixture was poured into water (15 mL) and then extracted with EtOAc (2×25 mL).The combined organic layers were washed with water (25 mL), brine (25 ml), dried (Na₂SO₄), filtered, concentrated and purified by silica gel chromatography to give (E)-tert-butyl (2-((4-(1-carbamoylcyclohexyl)phenoxy)methyl)-3-fluoroallyl)carbamate (320 mg, 89%) as yellow solid. ¹H NMR (400 MHz, CDCl₃): δ 7.31 (d, 2H), 6.89 (d, 2H), 6.74 (d, 1H), 5.29-5.20 (m, 1H) 4.90-4.74 (m, 1H), 4.43 (d, 2H), 4.02-3.96 (m, 2H), 2.26-2.17 (m, 2H), 2.04 (s, 3H), 2.01-1.92 (m, 2H), 1.62-1.53 (m, 2H), 1.51-1.43 (m, 4H), 1.41 (s, 9H); MS:421.2 [M+H]⁺.

### Step 4: (E)-1-(4-((2-(Aminomethyl)-3-fluoroallyl)oxy)phenyl)-N-methylcyclohexanecarboxamide

A mixture of (E)-tert-butyl (3-fluoro-2-((4-(1-(methylcarbamoyl)cyclohexyl)phenoxy)methyl)allyl)carbamate (320 mg, 0.76 mmol), TFA (2 mL) and DCM (5 mL) was stirred at room temperature for 2 h under a nitrogen atmosphere. The mixture was concentrated to dryness and then purified by reverse-phase HPLC to give (E)-1-(4-((2-(aminomethyl)-3-fluoroallyl)oxy)phenyl)-N-methylcyclohexanecarboxamide (143 mg, 59%) as a white solid. ¹H NMR (400 MHz, DMSO-d₆): δ 8.19-8.12 (m, 3H), 7.40 (d, 1H), 7.32 (d, 1H), 7.26 (d, 2H), 6.92 (d, 2H), 4.57 (d, 2H), 3.59 (d, 2H), 3.41 (s, 3H), 2.35-2.27 (m, 2H), 1.67-1.57 (m, 2H), 1.49 (m, 3H), 1.45-1.32 (m, 2H), 1.30-1.16 (m, 1H); MS:321.1[M+H]⁺.

### Compound 2

### (E)-4-(4-((2-(aminomethyl)-3-fluoroallyl)oxy)-3-methylphenyl)-N-methylbicyclo[2.2.2]octane-1-carboxamide

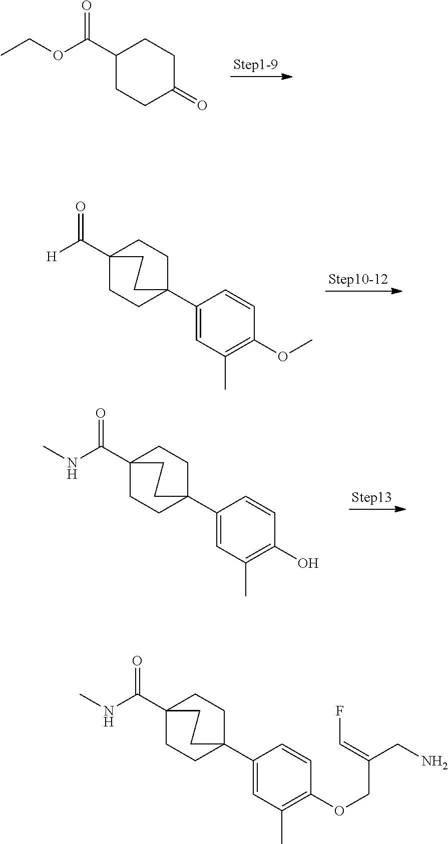

### Step 1: Ethyl 4-hydroxy-4-(4-methoxy-3-methylphenyl)cyclohexanecarboxylate

2.5 M n-Butyllithium in hexanes (60 mL, 150 mmol) was added dropwise to a solution of 4-bromo-1-methoxy-2-methylbenzene (27.8 g, 138 mmol) in THF (300 mL) at −78° C. The mixture was stirred at −78° C. for 1 h and then added dropwise to a solution of ethyl 4-oxocyclohexanecarboxylate (22.3 g, 131 mmol) and THF (300 mL) at −78° C. The reaction mixture was stirred at −78° C. for 2 h, added to saturated NH₄Cl (600 mL) and then extracted with EtOAc (2×600 mL). The combined organic extracts were washed with water (400 mL) and brine (400 mL), dried (Na₂SO₄), filtered and concentrated. The crude was purified by silica gel chromatography to give ethyl 4-hydroxy-4-(4-methoxy-3-methylphenyl)cyclohexanecarboxylate (18.9 g, 45%) as a yellow oil. ¹H NMR (400 MHz, DMSO-d₆): δ 7.26-7.11 (m, 2H), 6.84-6.75 (m, 1H), 4.64-4.59 (m, 1H), 4.11-3.98 (m, 2H), 3.72 (s, 3H), 2.39-2.25 (m, 1H), 2.13-2.07 (s, 3H), 1.93-1.77 (m, 3H), 1.75-1.42 (m, 5H), 1.23-1.11 (m, 3H); LCMS: 275.2 [M−OH]⁺.

### Step 2: Ethyl 4-allyl-4-(4-methoxy-3-methylphenyl)cyclohexanecarboxylate

Boron trifluoride diethyl etherate (24.9 g, 84.0 mmol) was added to a solution of ethyl 4-hydroxy-4-(4-methoxy-3-methylphenyl)cyclohexanecarboxylate (18.9 g, 64.6 mmol), allyltrimethylsilane (11.8 g, 103 mmol) and CH₂Cl₂ (400 mL) at −78° C. The mixture was stirred at −78° C. for 1 h, stirred at room temperature overnight, and then added to brine (200 mL) and CH₂Cl₂ (200 mL). The organic layer was separated, washed with saturated NaHCO₃ solution (2×200 mL), brine (200 mL), dried (Na₂SO₄), filtered and concentrated. The crude was purified by silica gel chromatography to give ethyl 4-allyl-4-(4-methoxy-3-methylphenyl)cyclohexanecarboxylate (15 g, 71%) as a yellow oil. ¹H NMR (400 MHz, CDCl₃): δ 7.10-7.00 (m, 2H), 6.76 (d, 1H), 5.50-5.26 (m, 1H), 4.98-4.81 (m, 2H), 4.15 (q, 0.5H), 4.03 (q, 1.5H), 3.81 (s, 3H), 2.42-2.26 (m, 3H), 2.21 (s, 3H), 2.15 (d, 1.5H), 1.98 (d, 0.5H), 1.88-1.75 (m, 2.5H), 1.72-1.60 (m, 0.5H), 1.55-1.33 (m, 3H), 1.27 (t, 0.8H), 1.18 (t, 2.2H); LCMS: 339.3 [M+Na]⁺.

### Step 3: Ethyl 4-(2,3-dihydroxypropyl)-4-(4-methoxy-3-methylphenyl)cyclohexane Carboxylate

0.1 M Osmium tetroxide in tert-butanol (7.6 mL, 0.76 mmol) was added to a solution of ethyl 4-allyl-4-(4-methoxy-3-methylphenyl)cyclohexanecarboxylate (4.81 g, 15.2 mmol), 4-methylmorpholine N-oxide (2.67 g, 22.8 mmol), CH₃CN (100 mL), and H₂O (25 mL) at 0° C. The reaction was stirred at room temperature overnight, and then saturated Na₂SO₃ (50 mL) was added. The mixture was stirred at room temperature for 30 min, concentrated, dissolved in water (80 mL) and extracted with EtOAc (2×100 mL). The organic layers were dried (Na₂SO₄), filtered and concentrated. The reside was purified by silica gel chromatography to give ethyl 4-(2,3-dihydroxypropyl)-4-(4-methoxy-3-methylphenyl)cyclohexanecarboxylate (5.23 g, 94%) as a yellow oil. ¹H NMR (400 MHz, CDCl₃): δ 7.16-7.05 (m, 2H), 6.78 (d, 1H), 4.17-4.06 (m, 0.5H), 4.05-3.95 (m, 1.5H), 3.80 (s, 3H), 3.66-3.48 (m, 1H), 3.32-3.18 (m, 2H), 2.53-2.40 (m, 2H), 2.37-2.27 (m, 1H), 2.19 (s, 3H), 1.80 (t, 3H), 1.68-1.32 (m, 7H), 1.24 (td, 0.8H), 1.17 (t, 2.2H); LCMS: 373.3 [M+Na]⁺.

### Step 4: Ethyl 4-(4-methoxy-3-methylphenyl)-4-(2-oxoethyl)cyclohexanecarboxylate

Sodium periodate (3.83 g, 17.9 mmol) was added to a solution of ethyl 4-(2,3-dihydroxypropyl)-4-(4-methoxy-3-methylphenyl)cyclohexanecarboxylate (5.23 g, 14.9 mmol), THF (70 mL), and H₂O (35 mL) at 0° C. The mixture was stirred at room temperature overnight, added to water (50 mL) and extracted with EtOAc (2×100 mL). The organic layers were combined, washed with water (80 mL), brine (80 mL), dried (Na₂SO₄), filtered and concentrated. The residue was purified by silica gel chromatography to give ethyl 4-(4-methoxy-3-methylphenyl)-4-(2-oxoethyl)cyclohexanecarboxylate (3.95 g, 82%) as a yellow oil. ¹H NMR (400 MHz, CDCl₃): δ 9.42-9.28 (m, 1H), 7.19-7.07 (m, 2H), 6.79 (d, 1H), 4.15 (q, 0.5H), 4.04 (q, 1.5H), 3.82 (s, 3H), 2.52-2.41 (m, 3H), 2.33 (s, 1H), 2.21 (s, 3H), 1.92-1.75 (m, 3H), 1.63-1.46 (m, 4H), 1.31-1.23 (t, 0.5H), 1.19 (t, 2.5H); LCMS: 341.3 [M+Na]⁺.

### Step 5: Ethyl 4-(2-hydroxyethyl)-4-(4-methoxy-3-methylphenyl)cyclohexanecarboxylate

Sodium borohydride (704 mg, 18.6 mmol) was added to a solution of ethyl 4-(4-methoxy-3-methylphenyl)-4-(2-oxoethyl)cyclohexanecarboxylate (3.95 g, 12.4 mmol) and THF (100 mL) at 0° C. The mixture was stirred at 0° C. for 1 h, stirred at room temperature overnight and then diluted with water (100 mL). The organic solvent was removed under reduced pressure and the aqueous layer was extracted with CH₂Cl₂ (2×300 mL). The organic extracts were dried (Na₂SO₄), filtered, and concentrated. The residue was purified by silica gel chromatography to give ethyl 4-(2-hydroxyethyl)-4-(4-methoxy-3-methylphenyl)cyclohexanecarboxylate (3.11 g, 67%) as a yellow oil. ¹H NMR (400 MHz, CDCl₃): δ 7.04-6.96 (m, 2H), 6.71 (d, 1H), 4.12-4.03 (q, 0.4H), 3.97 (q, 1.6H), 3.74 (s, 3H), 3.38-3.28 (m, 2H), 2.39-2.19 (m, 3H), 2.14 (s, 3H), 1.80-1.71 (m, 2H), 1.70-1.60 (m, 2H), 1.50-1.28 (m, 4H), 1.24-1.17 (t, 1H), 1.12 (t, 2H); LCMS: 343.2 [M+Na]⁺.

### Step 6: Ethyl 4-(2-bromoethyl)-4-(4-methoxy-3-methylphenyl)cyclohexanecarboxylate

A solution of triphenylphosphine (4.60 g, 17.5 mmol) and CH₂Cl₂ (20 mL) was added dropwise to a solution of ethyl 4-(2-hydroxyethyl)-4-(4-methoxy-3-methylphenyl)cyclohexanecarboxylate (2.81 g, 8.77 mmol), CBr₄ (4.36 g, 13.2 mmol) and CH₂Cl₂ (40 mL) at 0° C. The mixture was stirred at 0° C. for 1 h, stirred at room temperature overnight and then concentrated. The residue was purified by silica gel chromatography to give ethyl 4-(2-bromoethyl)-4-(4-methoxy-3-methylphenyl)cyclohexanecarboxylate (2.62 g, 77%) as a yellow oil. ¹H NMR (400 MHz, CDCl₃): δ 7.08-6.96 (m, 2H), 6.77 (d, 1H), 4.15 (q, 0.3H), 4.03 (q, 1.7H), 3.81 (s, 3H), 3.06-2.91 (m, 2H), 2.41-2.24 (m, 3H), 2.24-2.15 (s, 3H), 2.06-1.95 (m, 2H), 1.87-1.77 (m, 2H), 1.53-1.34 (m, 4H), 1.27 (t, 1H), 1.18 (t, 2H); LCMS: 405.1 [M+Na]⁺.

### Step 7: Ethyl 4-(4-methoxy-3-methylphenyl)bicyclo[2.2.2]octane-1-carboxylate

2 M Lithium diisopropylamide in THF (4.8 mL, 9.60 mmol) was added dropwise to a solution of ethyl 4-(2-bromoethyl)-4-(4-methoxy-3-methylphenyl)cyclohexanecarboxylate (1.81 g, 4.72 mmol), HMPA (4.23 g, 23.6 mmol) and THF (90 mL) at −78° C. The mixture was stirred at −78° C. for 3 h, added to saturated NH₄Cl (90 mL) and then extracted with EtOAc (2×150 mL). The combined organic layers were washed with H₂O (100 mL), brine (100 mL), dried (Na₂SO₄) filtered, and concentrated. The residue was purified by silica gel chromatography to give ethyl 4-(4-methoxy-3-methylphenyl)bicyclo[2.2.2]octane-1-carboxylate (1.17 g, 82%) as a yellow solid. ¹H NMR (400 MHz, CDCl₃): δ 7.05-6.98 (m, 2H), 6.69 (d, 1H), 4.05 (q, 2H), 3.73 (s, 3H), 2.14 (s, 3H), 1.87-1.70 (m, 12H), 1.18 (t, 3H); LCMS: 303.3 [M+H]⁺.

### Step 8: (4-(4-Methoxy-3-methylphenyl)bicyclo[2.2.2]octan-1-yl)methanol

1 M Diisobutylaluminum hydride in toluene (14 mL, 14.0 mmol) was added to a solution of ethyl 4-(4-methoxy-3-methylphenyl)bicyclo[2.2.2]octane-1-carboxylate (1.64 g, 5.42 mmol) and CH₂Cl₂ (100 mL) at −78° C. The mixture was stirred at −78° C. for 1 h, stirred at room temperature for 2 h and then added to ice H₂O (80 mL). The pH was adjusted (pH=6) with 1 N HCl and the mixture was filtered. The layers were separated and the aqueous layer was extracted with CH₂Cl₂ (2×200 mL). The combined organic layers were washed with water (100 mL) and brine (100 mL), dried (Na₂SO₄), filtered and concentrated. The residue was purified by silica gel chromatography to give (4-(4-methoxy-3-methylphenyl)bicyclo[2.2.2]octan-1-yl)methanol (1.22 g, 82%) as a yellow solid. ¹H NMR (400 MHz, CDCl₃): δ 7.07-6.99 (m, 2H), 6.72-6.64 (m, 1H), 3.73 (s, 3H), 3.25 (s, 2H), 2.14 (s, 3H), 1.81-1.69 (m, 6H), 1.50-1.40 (m, 6H); LCMS: 261.2 [M+H]+.

### Step 9: 4-(4-Methoxy-3-methylphenyl)bicyclo[2.2.2]octane-1-carbaldehyde

Pyridinium chlorochromate (1.03 g, 4.78 mmol) was added to a mixture of (4-(4-methoxy-3-methylphenyl)bicyclo[2.2.2]octan-1-yl)methanol (621 mg, 2.39 mmol), SiO₂ (1.93 g, 32.2 mmol) and CH₂Cl₂ (120 mL). The mixture was stirred at room temperature for 2 h, filtered through a neutral alumina plug and then concentrated to give 4-(4-methoxy-3-methylphenyl)bicyclo[2.2.2]octane-1-carbaldehyde (601 mg, 93%) as a white solid. ¹H NMR (400 MHz, CDCl₃): δ 9.56-9.48 (s, 1H), 7.11-7.06 (m, 2H), 6.78-6.72 (m, 1H), 3.81 (s, 3H), 2.22 (s, 3H), 1.91-1.83 (m, 6H), 1.80-1.71 (m, 6H); LCMS: 259.3 [M+H]⁺.

### Step 10: 4-(4-methoxy-3-methylphenyl)bicyclo[2.2.2]octane-1-carboxylic Acid

To a mixture of 4-(4-methoxy-3-methylphenyl)bicyclo[2.2.2]octane-1-carbaldehyde (258 mg, 1.0 mmol) in acetone was added Jones reagent (500 ul, 1.0 mmol) and the reaction stirred at room temperature for 2 h. EtOAc was added and reaction mixture was washed with sodium thiosulfate. Suspension was filtered, filter cake washed with EtOAc and dried to give the desired product (206 mg, 75%) as a white solid. ¹H NMR (400 MHz, DMSO): δ 12.02 (s, 1H), 7.06 (s, 1H), 7.04 (d, 1H); 6.82 (d, 1H), 3.75 (s, 3H); 2.09 (s, 3H), 1.69 (m, 12H). LCMS: 275.1 [M+H]⁺.

### Step 11: 4-(4-methoxy-3-methylphenyl)-N-methylbicyclo[2.2.2]octane-1-carboxamide

To a solution of 4-(4-methoxy-3-methylphenyl)bicyclo[2.2.2]octane-1-carboxylic acid (150 mg, 0.55 mmol) and HATU (312 mg, 0.82 mmol) in DMF (3 mL) was added 2M methylamine (820 ul, 1.64 mmol) followed by diisopropylethylamine (286 ul, 1.64 mmol). The reaction mixture was stirred at room temperature for 1 h, diluted with EtOAc, organics washed with 1N HCl, H₂O and brine, dried (MgSO₄) and concentrated. The residue was purified by silica gel chromatography to give the desired product (149 mg, 95%) as a white solid. ¹H NMR (400 MHz, DMSO): 7.08 (s, 1H), 7.06 (d, 1H); 6.82 (d, 1H), 3.75 (s, 3H); 2.55 (s, 3H); 2.12 (s, 3H), 1.72 (m, 12H).

### Step 12: 4-(4-hydroxy-3-methylphenyl)-N-methylbicyclo[2.2.2]octane-1-carboxamide

To a stirred solution of 4-(4-methoxy-3-methylphenyl)-N-methylbicyclo[2.2.2]octane-1-carboxamide (130 mg, 0.48 mmol) in DCM (5 mL) at −78° C. was added 1N boron tribromide in DCM (1.43 mL, 1.43 mmol) and the resulting mixture was stirred at room temperature for 1 h. The solvent was evaporated to give the crude product (130 mg) as a light brown solid. LCMS: 274.3 [M+H]⁺. The crude product was used without further purification.

### Step 13: (E)-4-(4-((2-(aminomethyl)-3-fluoroallyl)oxy)-3-methylphenyl)-N-methylbicyclo[2.2.2]octane-1-carboxamide Hydrochloride

A mixture of crude 4-(4-hydroxy-3-methylphenyl)-N-methylbicyclo[2.2.2]octane-1-carboxamide (130 mg), (E)-tert-butyl (2-(bromomethyl)-3-fluoroallyl)carbamate (153 mg, 0.57 mmol), Cs₂CO₃ (557 mg, 1.71 mmol) and DMF (5 mL) was stirred at 60° C. overnight. The reaction mixture was poured into water and then extracted with EtOAc. The combined organic layers were washed with water, brine, dried (Na₂SO₄), filtered and concentrated. The crude intermediate was taken up in TFA (2 mL) and DCM (5 mL) and stirred at room temperature for 2 h. The reaction was concentrated to dryness and then purified by reverse-phase HPLC. The material was taken up in 4M HCl in dioxane (2 mL), stirred at room temperature for 2 h and concentrated under reduced pressure to give (E)-4-(4-((2-(aminomethyl)-3-fluoroallyl)oxy)-3-methylphenyl)-N-methylbicyclo[2.2.2]octane-1-carboxamide HCl salt (105 mg, 51% over 2 steps) as a white solid. ¹H NMR (400 MHz, DMSO-d₆): δ 8.16 (s, 3H), 7.39-7.36 (m, 1H), 7.30 (d, 1H), 7.13-7.08 (m, 2H), 6.88-6.86 (m, 1H), 4.56 (d, 2H), 3.63-3.62 (m, 2H), 2.58 (d, 3H), 2.17 (s, 3H), 1.73 (s, 12H); MS:361.1 [M+H]⁺.

The compounds below were synthesized in a similar manner as described for compound 2.

| Compound | Structure | Chemical Name | m/z |
|---|---|---|---|
| 2.01 | 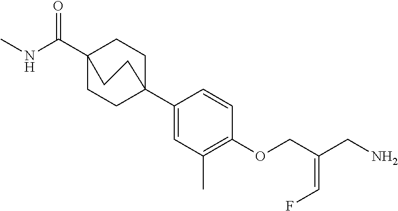 [SMILES: CNC(=O)C12CCC(c3ccc(OC/C(=C\F)CN)c(C)c3)(CC1)CC2] | (Z)-4-(4-((2-(aminomethyl)-3- fluoroallyl)oxy)-3- methylphenyl)-N- methylbicyclo[2.2.2]octane-1- carboxamide | 361.1 |
| 2.02 | 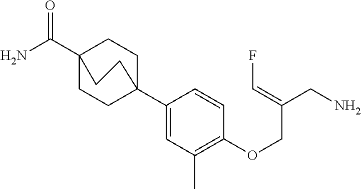 [SMILES: Cc1cc(C23CCC(C(N)=O)(CC2)CC3)ccc1OC/C(=C/F)CN] | (E)-4-(4-((2-(aminomethyl)-3- fluoroallyl)oxy)-3- methylphenyl)bicyclo[2.2.2] octane-1-carboxamide | 347.1 |

### Compound 2.03

### (E)-4-(4-((2-(aminomethyl)-3-fluoroallyl)oxy)phenyl)-N-methylbicyclo[2.2.2]octane-1-carboxamide Hydrochloride

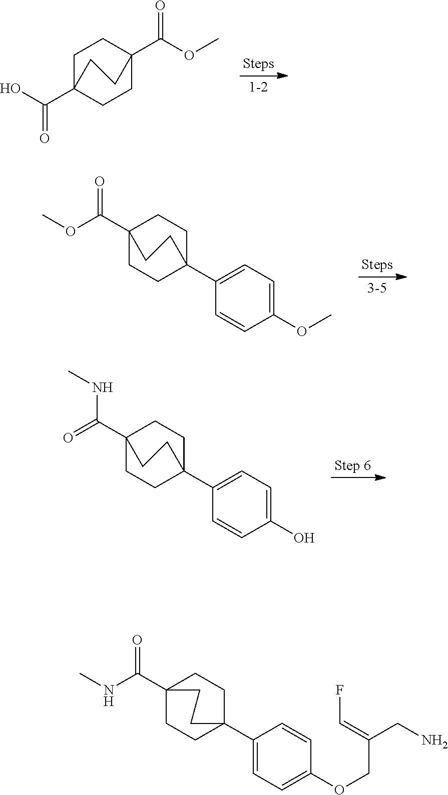

### Step 1: 1-(1,3-Dioxoisoindolin-2-yl) 4-methyl bicyclo[2.2.2]octane-1,4-dicarboxylate

N,N-Diisopropylcarbodiimide (18.0 g, 143 mmol) was added to a solution of 4-(methoxycarbonyl)bicyclo[2.2.2]octane-1-carboxylic acid (25.0 g, 118 mmol), 2-hydroxyisoindoline-1,3-dione (19.2 g, 118 mmol), DMAP (4.32 g, 35.3 mmol) and CH₂Cl₂ (500 mL) at room temperature under a nitrogen atmosphere. The mixture was stirred at room temperature overnight, washed with H₂O (2×300 ml), dried (Na₂SO₄), filtered, concentrated and then purified by silica gel chromatography to give 1-(1,3-dioxoisoindolin-2-yl) 4-methyl bicyclo[2.2.2]octane-1,4-dicarboxylate (23 g) as a white solid. ¹H NMR (400 MHz, CDCl₃): δ 7.88 (d, 2H), 7.78 (d, 2H), 3.68 (s, 3H), 2.10-2.04 (m, 6H), 1.93-1.87 (m, 6H); LCMS: 358.1 [M+H]⁺.

### Step 2a: (4-Methoxyphenyl)magnesium Lithium Bromide Chloride

Magnesium (4.08 g, 168 mmol) and LiCl (5.34 g, 126 mmol) were added to an oven-dried 500 mL 3-necked flask connected to a double manifold. The flask was evacuated and backfilled with nitrogen 3 times. Tetrahydrofuran (50 mL) was added, the mixture stirred for 15 min and then 1M DIBAL-H in PhMe (1.7 mL) added dropwise. The reaction was stirred for 15 min, cooled to 0° C. and a solution of 1-bromo-4-methoxybenzene (15.7 g, 83.9 mmol) in THF (50 mL) was added dropwise. The mixture was warmed to room temperature and stirred for 2 h to give (4-methoxyphenyl) magnesium lithium bromide chloride as a solution in THF.

### Step 2b: Bis(4-methoxyphenyl)zinc

1M Zinc (II) chloride in THF (50 mL) was added to the (4-methoxyphenyl)magnesium lithium bromide chloride THF solution (˜84 mmol) at room temperature. The mixture was stirred at room temperature for 1 h to give bis(4-methoxyphenyl)zinc as a solution in THF.

### Step 2c: Methyl 4-(4-methoxyphenyl)bicyclo[2.2.2]octane-1-carboxylate

The bis(4-methoxyphenyl)zinc THF solution (˜42 mmol) was added to a solution of 1-(1,3-dioxoisoindolin-2-yl) 4-methyl bicyclo[2.2.2]octane-1,4-dicarboxylate (6.0 g, 16.8 mmol), 2-methyl-6-(6-methyl-2-pyridyl)pyridine (1.86 g, 10.1 mmol), Ni(acac)₂ (2.16 g, 8.39 mmol) and CH₃CN (100 mL) at room temperature. The mixture was degassed with 3 vacuum-nitrogen cycles, stirred at 80° C. overnight, cooled to room temperature and concentrated to remove CH₃CN. The residue was diluted with water (50 mL) and extracted with EtOAc (3×50 mL). The combined organic layers were washed with brine (2×50 mL), dried (Na₂SO₄), filtered, concentrated and purified by silica gel chromatography to give methyl 1-(4-methoxyphenyl)bicyclo[2.2.2]octane-4-carboxylate (3.8 g, 41%) as a light yellow solid. ¹H NMR (400 MHz, CDCl₃): δ 7.26-7.21 (m, 2H), 6.88-6.82 (m, 2H), 3.80 (s, 3H), 3.68 (s, 3H), 1.96-1.88 (m, 6H), 1.88-1.81 (m, 6H); LCMS: 275.0 [M+H]+.

### Step 3: 4-(4-methoxyphenyl)bicyclo[2.2.2]octane-1-carboxylic Acid

To a solution of methyl 4-(4-methoxyphenyl)bicyclo[2.2.2]octane-1-carboxylate (100 mg, 0.37 mmol) in THF (2 ml) and H₂O (400 ul) was added lithium hydroxide (44 mg, 1.82 mmol) and the reaction stirred at 60° C. overnight. Solvent was evaporated and then 1N HCl was added, solid was filtered, washed with 1N HCl and dried to give the desired product as a white solid. (86 mg, 91%). ¹H NMR (400 MHz, DMSO): δ 12.03 (s, 1H), 7.23 (d, 2H), 6.84 (d, 2H); 3.71 (s, 3H), 1.80-1.74 (m, 12H). LCMS: 260.87 [M+H]+.

### Step 4: 4-(4-methoxyphenyl)-N-methylbicyclo[2.2.2]octane-1-carboxamide

To a solution of 4-(4-methoxyphenyl)bicyclo[2.2.2]octane-1-carboxylic acid (75 mg, 0.29 mmol) and HATU (164 mg, 0.43 mmol) in DMF (1.2 mL) was added 2M methylamine (432 ul, 0.87 mmol) followed by diisopropylethylamine (151 ul, 0.87 mmol). The reaction mixture was stirred at room temperature for 1 h. EtOAc was added and reaction mixture was washed with 1N HCl, H₂O and brine, dried (MgSO₄) and concentrated to give the crude product which will be used directly without purification. LCMS: 274.06 [M+H]⁺.

### Step 5: 4-(4-hydroxyphenyl)-N-methylbicyclo[2.2.2]octane-1-carboxamide

To a stirred solution of crude 4-(4-methoxyphenyl)-N-methylbicyclo[2.2.2]octane-1-carboxamide (79 mg) in DCM (1.5 mL) at −78° C. was added 1M boron tribromide in DCM (1.43 ml, 1.43 mmol), the resulting mixture warmed to room temperature and stirred at room temperature for 1 h. The solvent was evaporated to give the crude product (75 mg) as an off-white solid. LCMS: 260.11 [M+H]⁺. Crude product was used in subsequent reactions without further purification.

### Step 6: (E)-4-(4-((2-(aminomethyl)-3-fluoroallyl)oxy)phenyl)-N-methylbicyclo[2.2.2]octane-1-carboxamide Hydrochloride

A mixture of crude 4-(4-hydroxyphenyl)-N-methylbicyclo[2.2.2]octane-1-carboxamide (75 mg), (E)-tert-butyl (2-(bromomethyl)-3-fluoroallyl)carbamate (77 mg, 0.29 mmol), Cs₂CO₃ (279 mg, 0.86 mmol) and DMF (5 mL) was stirred at 60° C. overnight. The reaction mixture was poured into water and then extracted with EtOAc. The combined organic layers were washed with water, brine, dried (Na₂SO₄), filtered and concentrated. The crude intermediate was taken up in TFA (2 mL) and DCM (5 mL) and stirred at room temperature for 2 h. The reaction was concentrated to dryness and then purified by reverse-phase HPLC. The purified material was taken up in 4M HCl in dioxane (2 mL), stirred at room temperature for 2 h and concentrated under reduced pressure to give (E)-4-(4-((2-(aminomethyl)-3-fluoroallyl)oxy)phenyl)-N-methylbicyclo[2.2.2]octane-1-carboxamide HCl salt (78 mg, 70% over 2 steps) as a white solid. ¹H NMR (400 MHz, DMSO-d₆): δ 8.12 (s, 3H), 7.22 (d, 1H), 7.18 (d, 2H), 6.83 (d, 2H), 6.88-6.86 (m, 1H), 4.50 (d, 2H), 3.54-3.53 (m, 2H), 2.49 (d, 3H), 1.67 (s, 12H); MS:347.1 [M+H]+.

The compounds below were synthesized in a similar manner as described for compound 2.03.

| Com- | | | |
|---|---|---|---|
| pound | Structure | Chemical Name | m/z |
| 2.04 | 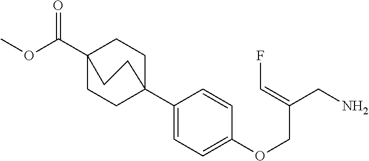 [SMILES: COC(=O)C12CCC(c3ccc(OC/C(=C/F)CN)cc3)(CC1)CC2] | (E)-methyl 4-(4-((2- (aminomethyl)-3- fluoroallyl)oxy)phenyl)bicyclo [2.2.2]octane-1-carboxylate | 348.0 |
| 2.05 | 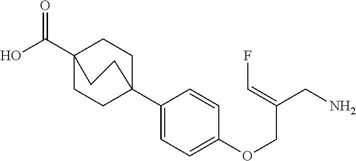 [SMILES: NC/C(=C\F)COc1ccc(C23CCC(C(=O)O)(CC2)CC3)cc1] | (E)-4-(4-((2-(amino- methyl)-3-fluoroallyl) oxy)phenyl) bicyclo[2.2.2]octane-1- carboxylic acid | 334.0 |
| 2.06b | 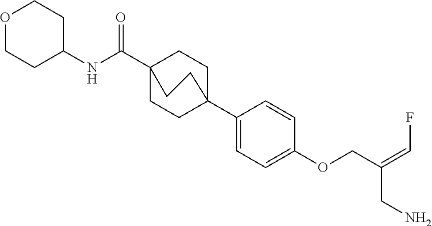 [SMILES: NC/C(=C/F)COc1ccc(C23CCC(C(=O)NC4CCOCC4)(CC2)CC3)cc1] | (Z)-4-(4-((2-(amino- methyl)-3- fluoroally)oxy)phenyl)-N- (tetrahydro-2H-pyran-4- yl)bicyclo[2.2.2]octane-1- carboxamide | 417.3 |
| 2.07 | 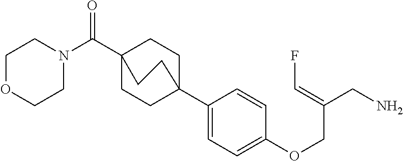 [SMILES: NC/C(=C\F)COc1ccc(C23CCC(C(=O)N4CCOCC4)(CC2)CC3)cc1] | (E)-(4-(4-((2-amino- methyl)-3-fluoroallyl) oxy)phenyl)bicyclo [2.2.2]octan-1- yl)(morpholino)methanone | 403.2 |
| 2.08 | 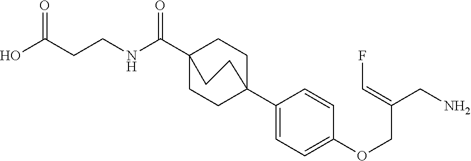 [SMILES: NC/C(=C\F)COc1ccc(C23CCC(C(=O)NCCC(=O)O)(CC2)CC3)cc1] | (E)-3-(4-(4-((2- (aminomethyl)- 3-fluoroallyl)oxy)phenyl) bicyclo[2.2.2]octane-1- carboxamido)propanoic acid | 405.2 |
| 2.09 | 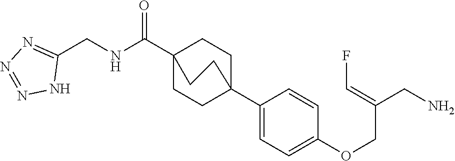 [SMILES: NC/C(=C\F)COc1ccc(C23CCC(C(=O)NCc4nnn[nH]4)(CC2)CC3)cc1] | (E)-N-((1H-tetrazol-5- yl)methyl)-4-(4-((2- (aminomethyl)-3- fluoroallyl)oxy)phenyl) bicyclo[2.2.2]octane- 1-carboxamide | 415.1 |
| 2.10 | 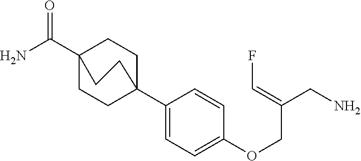 [SMILES: NC/C(=C\F)COc1ccc(C23CCC(C(N)=O)(CC2)CC3)cc1] | (E)-4-(4-((2- (aminomethyl)-3- fluoroallyl)oxy)phenyl) bicyclo[2.2.2]octane- 1-carboxamide | 333.1 |
| 2.11 | 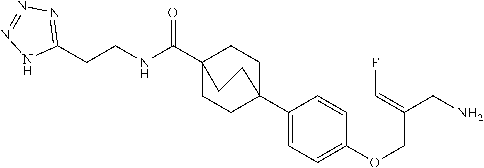 [SMILES: NC/C(=C\F)COc1ccc(C23CCC(C(=O)NCCc4nnn[nH]4)(CC2)CC3)cc1] | (E)-N-(2-(1H-tetrazol-5- yl)ethyl)-4-(4-((2- (aminomethyl)-3- fluoroallyl)oxy)phenyl) bicyclo[2.2.2]octane- 1-carboxamide | 429.1 |
| 2.12 | 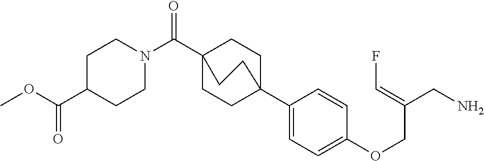 [SMILES: COC(=O)C1CCN(C(=O)C23CCC(c4ccc(OC/C(=C/F)CN)cc4)(CC2)CC3)CC1] | (E)-methyl 1-(4-(4-((2- (aminomethyl)-3- fluoroallyl)oxy)phenyl) bicyclo[2.2.2]octane-1- carbonyl)piperidine-4- carboxylate | 459.3 |
| 2.13 | 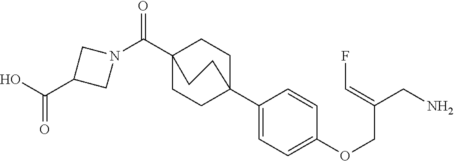 [SMILES: NC/C(=C\F)COc1ccc(C23CCC(C(=O)N4CC(C(=O)O)C4)(CC2)CC3)cc1] | (E)-1-(4-(4-((2- (aminomethyl)-3- fluoroallyl)oxy)phenyl) bicyclo[2.2.2]octane- 1-carbonyl)azetidine- 3-carboxylic acid | 417.1 |
| 2.14 | 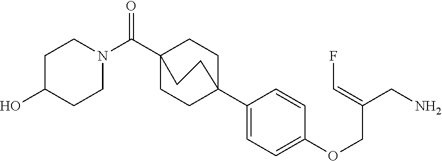 [SMILES: NC/C(=C\F)COc1ccc(C23CCC(C(=O)N4CCC(O)CC4)(CC2)CC3)cc1] | (E)-(4-(4-((2- (aminomethyl)-3- fluoroallyl)oxy)phenyl) bicyclo[2.2.2]octan-1-yl) (4-hydroxypiperidin-1- yl)methanone | 417.2 |
| 2.15 | 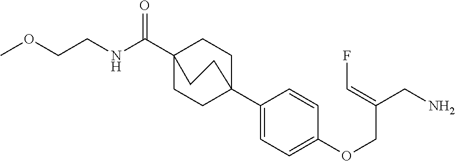 [SMILES: COCCNC(=O)C12CCC(c3ccc(OC/C(=C/F)CN)cc3)(CC1)CC2] | (E)-4-(4-((2- (aminomethyl)-3- fluoroallyl)oxy)phenyl)- N-(2-methoxyethyl) bicyclo[2.2.2] octane-1-carboxamide | 391.1 |
| 2.16 | 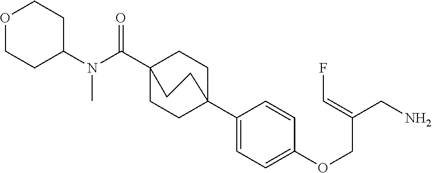 [SMILES: CN(C(=O)C12CCC(c3ccc(OC/C(=C/F)CN)cc3)(CC1)CC2)C1CCOCC1] | (E)-4-(4-((2-(amino- methyl)-3-fluoroallyl) oxy)phenyl)-N- methyl-N-(tetrahydro-2H- pyran-4-yl)bicyclo[2.2.2] octane-1-carboxamide | 431.3 |
| 2.17 | 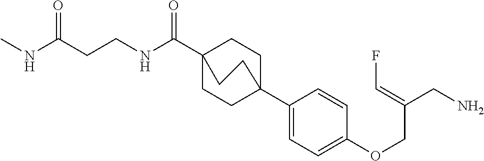 [SMILES: CNC(=O)CCNC(=O)C12CCC(c3ccc(OC/C(=C/F)CN)cc3)(CC1)CC2] | (E)-4-(4-((2-(amino- methyl)-3-fluoroallyl) oxy)phenyl)-N-(3- (methylamino)-3- oxopropyl)bicyclo[2.2.2] octane-1-carboxamide | 418.2 |
| 2.18 | 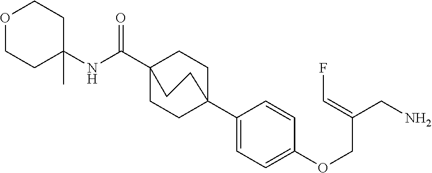 [SMILES: CC1(NC(=O)C23CCC(c4ccc(OC/C(=C/F)CN)cc4)(CC2)CC3)CCOCC1] | (E)-4-(4-((2-(amino- methyl)-3-fluoroallyl) oxy)phenyl)-N-(4- methyltetrahydro-2H- pyran-4-yl)bicyclo [2.2.2]octane-1- carboxamide | 431.3 |
| 2.19 | 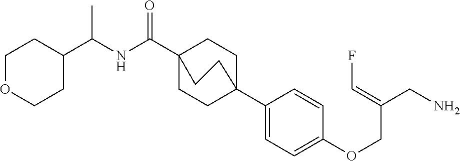 [SMILES: CC(NC(=O)C12CCC(c3ccc(OC/C(=C/F)CN)cc3)(CC1)CC2)C1CCOCC1] | (E)-4-(4-((2-(amino- methyl)-3-fluoroallyl) oxy)phenyl)-N-(1- (tetrahydro-2H-pyran-4- yl)ethyl)bicyclo[2.2.2] octane-1-carboxamide | 445.2 |
| 2.20 | 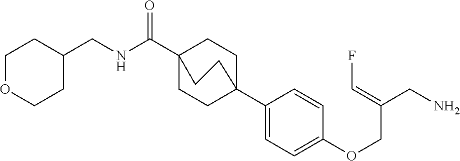 [SMILES: NC/C(=C\F)COc1ccc(C23CCC(C(=O)NCC4CCOCC4)(CC2)CC3)cc1] | (E)-4-(4-((2-amino- methyl)-3-fluoroallyl) oxy)phenyl)-N- ((tetrahydro-2H-pyran-4- yl)methyl)bicyclo[2.2.2] octane-1-carboxamide | 431.3 |
| 2.21 | 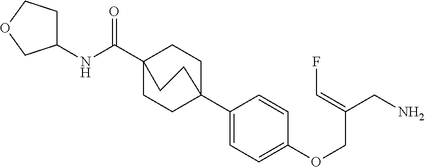 [SMILES: NC/C(=C\F)COc1ccc(C23CCC(C(=O)NC4CCOC4)(CC2)CC3)cc1] | (E)-4-(4-((2-(amino- methyl)-3-fluoroallyl) oxy)phenyl)-N- (tetrahydrofuran-3- yl)bicyclo[2.2.2]octane-1- carboxamide | 403.1 |
| 2.22 | 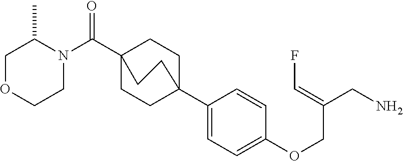 [SMILES: C[C@H]1COCCN1C(=O)C12CCC(c3ccc(OC/C(=C/F)CN)cc3)(CC1)CC2] | (S,E)-(4-(4-((2- (aminomethyl)-3- fluoroallyl)oxy)phenyl) bicyclo[2.2.2]octan- 1-yl)(3-methylmorpholino) methanone | 417.2 |
| 2.23 | 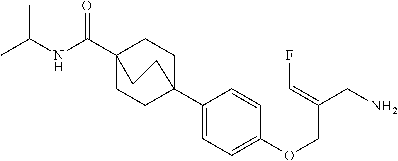 [SMILES: CC(C)NC(=O)C12CCC(c3ccc(OC/C(=C/F)CN)cc3)(CC1)CC2] | (E)-4-(4-((2-(aminomethyl)- 3-fluoroallyl)oxy) phenyl)-N-isopropylbicyclo [2.2.2]octane- 1-carboxamide | 375.1 |
| 2.24 | 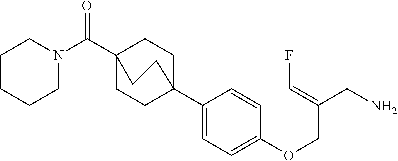 [SMILES: NC/C(=C\F)COc1ccc(C23CCC(C(=O)N4CCCCC4)(CC2)CC3)cc1] | (E)-(4-(4-((2-(amino- methyl)-3-fluoroallyl) oxy)phenyl)bicyclo [2.2.2]octan-1-yl)(piperidin- 1-yl)methanone | 401.2 |
| 2.25 | 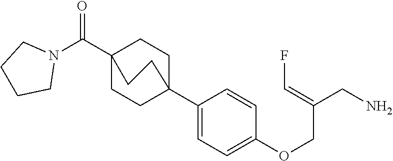 [SMILES: NC/C(=C\F)COc1ccc(C23CCC(C(=O)N4CCCC4)(CC2)CC3)cc1] | (E)-(4-(4-((2-(aminomethyl)-3- fluoroallyl)oxy)phenyl)bicyclo [2.2.2]octan-1-yl)(pyrrolidin-1- yl)methanone | 387.2 |
| 2.26 | 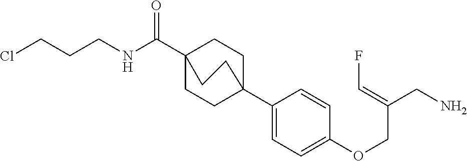 [SMILES: NC/C(=C\F)COc1ccc(C23CCC(C(=O)NCCCCl)(CC2)CC3)cc1] | (E)-4-(4-((2-(aminomethyl)-3- fluoroallyl)oxy)phenyl)-N-(3- chloropropyl)bicyclo[2.2.2] octane-1-carboxamide | 409.1 |
| 2.27 | 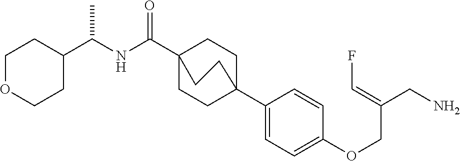 [SMILES: C[C@H](NC(=O)C12CCC(c3ccc(OC/C(=C/F)CN)cc3)(CC1)CC2)C1CCOCC1] | (S,E)-4-(4-((2-(aminomethyl)- 3-fluoroallyl)oxy)phenyl)-N-(1- (tetrahydro-2H-pyran-4- yl)ethyl)bicyclo[2.2.2]octane-1- carboxamide | 445.2 |
| 2.28 | 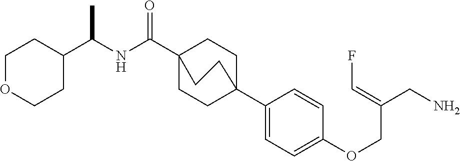 [SMILES: C[C@@H](NC(=O)C12CCC(c3ccc(OC/C(=C/F)CN)cc3)(CC1)CC2)C1CCOCC1] | (R,E)-4-(4-((2-(aminomethyl)- 3-fluoroallyl)oxy)phenyl)-N-(1- (tetrahydro-2H-pyran-4- yl)ethyl)bicyclo[2.2.2]octane-1- carboxamide | 445.2 |
| 2.29 | 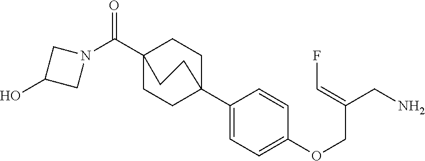 [SMILES: NC/C(=C\F)COc1ccc(C23CCC(C(=O)N4CC(O)C4)(CC2)CC3)cc1] | (E)-(4-(4-((2-(aminomethyl)-3- fluoroallyl)oxy)phenyl)bicyclo [2.2.2]octan-1-yl)(3- hydroxyazetidin-1- yl)methanone | 389.1 |
| 2.30 | 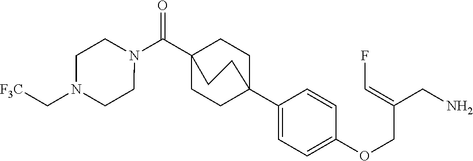 [SMILES: NC/C(=C\F)COc1ccc(C23CCC(C(=O)N4CCN(CC(F)(F)F)CC4)(CC2)CC3)cc1] | (E)-(4-(4-((2-(aminomethyl)-3- fluoroallyl)oxy)phenyl)bicyclo [2.2.2]octan-1-yl)(4-(2,2,2- trifluoroethyl)piperazin-1- yl)methanone | 484.1 |
| 2.31 | 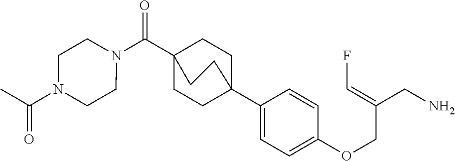 [SMILES: CC(=O)N1CCN(C(=O)C23CCC(c4ccc(OC/C(=C/F)CN)cc4)(CC2)CC3)CC1] | (E)-1-(4-(4-(4-((2- (aminomethyl)-3- fluoroallyl)oxy)phenyl)bicyclo [2.2.2]octane-1- carbonyl)piperazin-1- yl)ethanone | 444.2 |
| 2.32 | 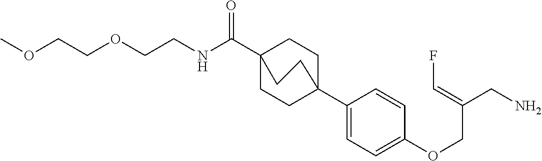 [SMILES: COCCOCCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- (2-methoxyethoxy)ethyl) bicyclo[2.2.2]octane-1- carboxamide | 435.2 |
| 2.33 | 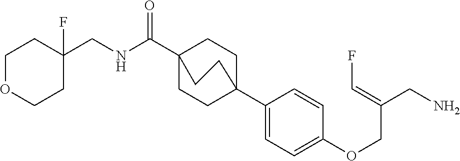 [SMILES: NC/C(=C\F)COc1ccc(C23CCC(C(=O)NCC4(F)CCOCC4)(CC2)CC3)cc1] | (E)-4-(4-((2-(aminomethyl)-3- fluoroallyl)oxy)phenyl)-N-((4- fluorotetrahydro-2H-pyran-4- yl)methyl)bicyclo[2.2.2]octane- 1-carboxamide | 449.2 |
| 2.34 | 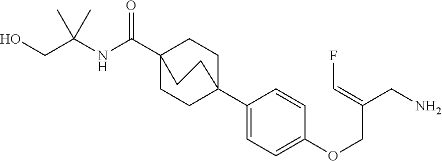 [SMILES: CC(C)(CO)NC(=O)C12CCC(c3ccc(OC/C(=C/F)CN)cc3)(CC1)CC2] | (E)-4-(4-((2-(aminomethyl)-3- fluoroallyl)oxy)phenyl)-N-(1- hydroxy-2-methylpropan-2- yl)bicyclo[2.2.2]octane-1- carboxamide | 405.2 |
| 2.35 | 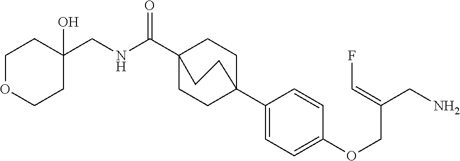 [SMILES: NC/C(=C\F)COc1ccc(C23CCC(C(=O)NCC4(O)CCOCC4)(CC2)CC3)cc1] | (E)-4-(4-((2-(aminomethyl)-3- fluoroallyl)oxy)phenyl)-N-((4- hydroxytetrahydro-2H-pyran-4- yl)methyl)bicyclo[2.2.2]octane- 1-carboxamide | 447.2 |
| 2.36 | 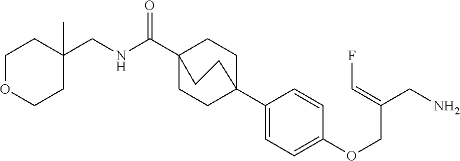 [SMILES: CC1(CNC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-((4- methyltetrahydro-2H-pyran-4- yl)methyl)bicyclo[2.2.2]octane- 1-carboxamide | 445.3 |
| 2.37 | 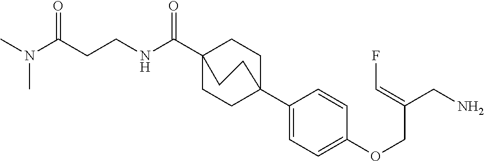 [SMILES: CN(C)C(=O)CCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- (dimethylamino)-3- oxopropyl)bicyclo[2.2.2]octane- 1-carboxamide | 432.2 |
| 2.38 | 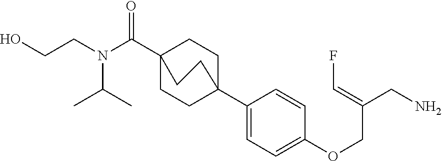 [SMILES: CC(C)N(CCO)C(=O)C12CCC(c3ccc(OC/C(=C/F)CN)cc3)(CC1)CC2] | (E)-4-(4-((2-(aminomethyl)-3- fluoroallyl)oxy)phenyl)-N-(2- hydroxyethyl)-N- isopropylbicyclo[2.2.2]octane- 1-carboxamide | 419.2 |
| 2.39 | 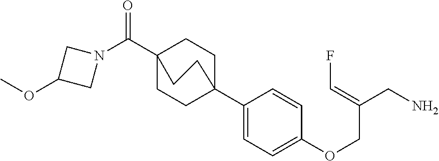 [SMILES: COC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo [2.2.2]octan-1-yl)(3- methoxyazetidin-1- yl)methanone | 403.2 |
| 2.40 | 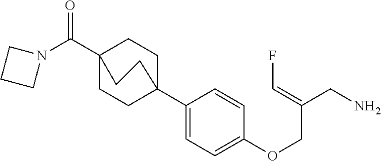 [SMILES: NC/C(=C\F)COc1ccc(C23CCC(C(=O)N4CCC4)(CC2)CC3)cc1] | (E)-(4-(4-((2-(aminomethyl)-3- fluoroallyl)oxy)phenyl)bicyclo [2.2.2]octan-1-yl)(azetidin-1- yl)methanone | 373.2 |
| 2.41 | 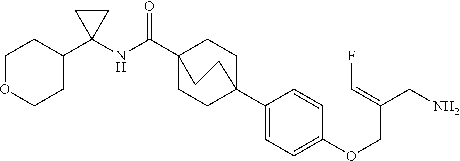 [SMILES: NC/C(=C\F)COc1ccc(C23CCC(C(=O)NC4(C5CCOCC5)CC4)(CC2)CC3)cc1] | (E)-4-(4-((2-(aminomethyl)-3- fluoroallyl)oxy)phenyl)-N-(1- (tetrahydro-2H-pyran-4- yl)cyclopropyl)bicyclo[2.2.2] octane-1-carboxamide | 457.3 |
| 2.42 | 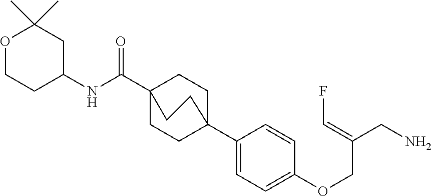 [SMILES: CC1(C)CC(NC(=O)C23CCC(c4ccc(OC/C(=C/F)CN)cc4)(CC2)CC3)CCO1] | (E)-4-(4-((2-(aminomethyl)-3- fluoroallyl)oxy)phenyl)-N-(2,2- dimethyltetrahydro-2H-pyran-4- yl)bicyclo[2.2.2]octane-1- carboxamide | 445.3 |
| 2.43 | 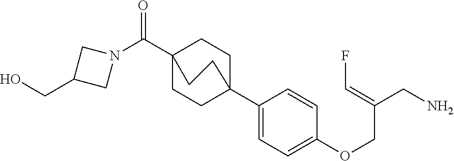 [SMILES: NC/C(=C\F)COc1ccc(C23CCC(C(=O)N4CC(CO)C4)(CC2)CC3)cc1] | (E)-(4-(4-((2-(aminomethyl)-3- fluoroallyl)oxy)phenyl)bicyclo [2.2.2]octan-1-yl)(3- (hydroxymethyl)azetidin-1- yl)methanone | 403.1 |
| 2.44 | 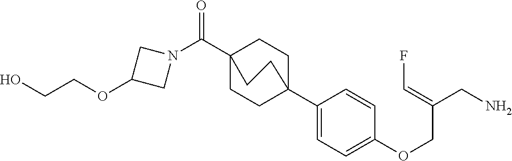 [SMILES: NC/C(=C\F)COc1ccc(C23CCC(C(=O)N4CC(OCCO)C4)(CC2)CC3)cc1] | (E)-(4-(4-((2-(aminomethyl)-3- fluoroallyl)oxy)phenyl)bicyclo [2.2.2]octan-1-yl)(3-(2- hydroxyethoxy)azetidin-1- yl)methanone | 433.2 |
| 2.45 | 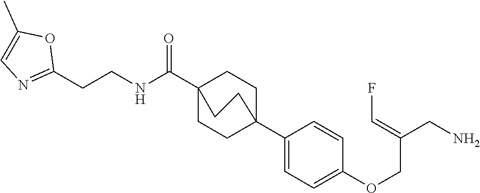 [SMILES: Cc1cnc(CCNC(=O)C23CCC(c4ccc(OC/C(=C/F)CN)cc4)(CC2)CC3)o1] | (E)-4-(4-((2-(aminomethyl)-3- fluoroallyl)oxy)phenyl)-N-(2- (5-methyloxazol-2- yl)ethyl)bicyclo[2.2.2]octane-1- carboxamide | 442.1 |
| 2.46 | 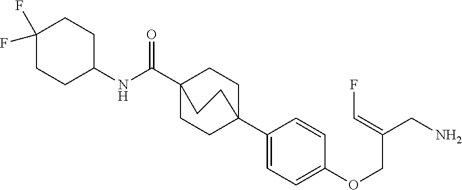 [SMILES: NC/C(=C\F)COc1ccc(C23CCC(C(=O)NC4CCC(F)(F)CC4)(CC2)CC3)cc1] | (E)-4-(4-((2-(aminomethyl)-3- fluoroallyl)oxy)phenyl)-N-(4,4- difluorocyclohexyl)bicyclo [2.2.2]octane-1-carboxamide | 451.1 |
| 2.47 | 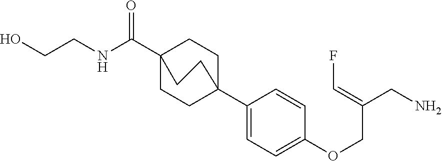 [SMILES: NC/C(=C\F)COc1ccc(C23CCC(C(=O)NCCO)(CC2)CC3)cc1] | (E)-4-(4-((2-(aminomethyl)-3- fluoroallyl)oxy)phenyl)-N-(2- hydroxyethyl)bicyclo[2.2.2] octane-1-carboxamide | 377.1 |
| 2.48 | 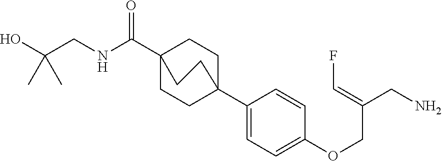 [SMILES: CC(C)(O)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- hydroxy-2- methylpropyl)bicyclo[2.2.2] octane-1-carboxamide | 405.2 |
| 2.49 | 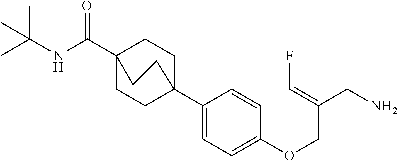 [SMILES: CC(C)(C)NC(=O)C12CCC(c3ccc(OC/C(=C/F)CN)cc3)(CC1)CC2] | (E)-4-(4-((2-(aminomethyl)-3- fluoroallyl)oxy)phenyl)-N-(tert- butyl)bicyclo[2.2.2]octane-1- carboxamide | 389.2 |
| 2.50 | 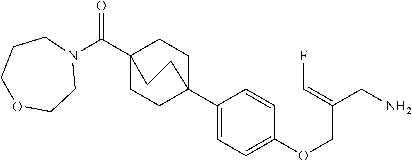 [SMILES: NC/C(=C\F)COc1ccc(C23CCC(C(=O)N4CCCOCC4)(CC2)CC3)cc1] | (E)-(4-(4-((2-(aminomethyl)-3- fluoroallyl)oxy)phenyl)bicyclo [2.2.2]octan-1-yl)(1,4-oxazepan- 4-yl)methanone | 417.1 |
| 2.51 | 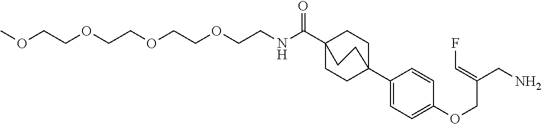 [SMILES: COCCOCCOCCOCCNC(=O)C12CCC(c3ccc(OC/C(=C/F)CN)cc3)(CC1)CC2] | (E)-4-(4-((2-(aminomethyl)-3- fluoroallyl)oxy)phenyl)-N- (2,5,8,11-tetraoxatridecan-13- yl)bicyclo[2.2.2]octane-1- carboxamide | 523.4 |
| 2.52 | 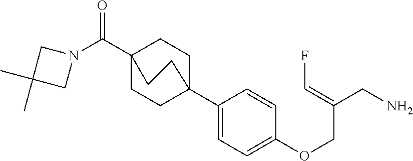 [SMILES: CC1(C)CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo [2.2.2]octan-1-yl)(3,3- dimethylazetidin-1- yl)methanone | 401.2 |
| 2.53 | 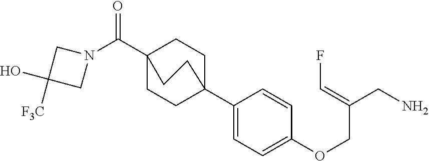 [SMILES: NC/C(=C\F)COc1ccc(C23CCC(C(=O)N4CC(O)(C(F)(F)F)C4)(CC2)CC3)cc1] | (E)-(4-(4-((2-(aminomethyl)-3- fluoroallyl)oxy)phenyl)bicyclo [2.2.2]octan-1-yl)(3-hydroxy-3- (trifluoromethyl)azetidin-1- yl)methanone | 457.2 |
| 2.54 | 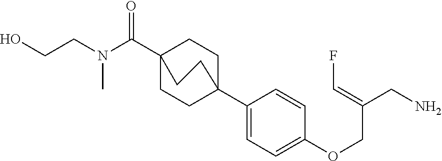 [SMILES: CN(CCO)C(=O)C12CCC(c3ccc(OC/C(=C/F)CN)cc3)(CC1)CC2] | (E)-4-(4-((2-(aminomethyl)-3- fluoroallyl)oxy)phenyl)-N-(2- hydroxyethyl)-N- methylbicyclo[2.2.2]octane-1- carboxamide | 391.1 |
| 2.55 | 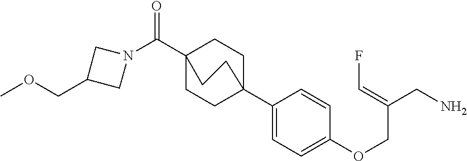 [SMILES: COCC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo [2.2.2]octan-1-yl)(3- (methoxymethyl)azetidin-1- yl)methanone | 417.2 |
| 2.56 | 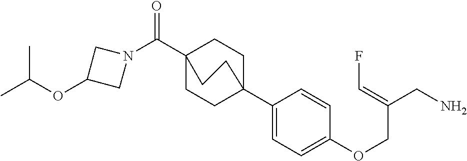 [SMILES: CC(C)OC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo [2.2.2]octan-1-yl)(3- isopropoxyazetidin-1- yl)methanone | 431.2 |
| 2.57 | 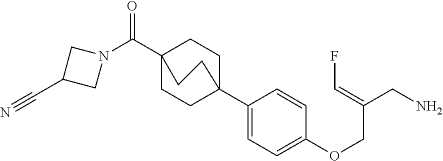 [SMILES: N#CC1CN(C(=O)C23CCC(c4ccc(OC/C(=C/F)CN)cc4)(CC2)CC3)C1] | (E)-1-(4-(4-((2-(aminomethyl)- 3-fluoroallyl)oxy)phenyl) bicyclo[2.2.2]octane-1- carbonyl)azetidine-3- carbonitrile | 398.1 |
| 2.58 | 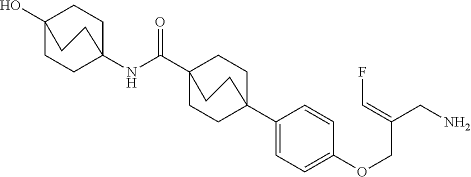 [SMILES: NC/C(=C\F)COc1ccc(C23CCC(C(=O)NC45CCC(O)(CC4)CC5)(CC2)CC3)cc1] | (E)-4-(4-((2-(aminomethyl)-3- fluoroallyl)oxy)phenyl)-N-(4- hydroxybicyclo[2.2.2]octan-1- yl)bicyclo[2.2.2]octane-1- carboxamide | 457.3 |
| 2.59 | 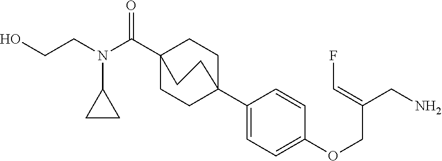 [SMILES: NC/C(=C\F)COc1ccc(C23CCC(C(=O)N(CCO)C4CC4)(CC2)CC3)cc1] | (E)-4-(4-((2-(aminomethyl)-3- fluoroallyl)oxy)phenyl)-N- cyclopropyl-N-(2- hydroxyethyl)bicyclo[2.2.2] octane-1-carboxamide | 417.1 |
| 2.60 | 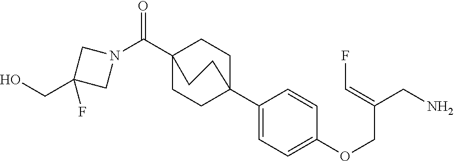 [SMILES: NC/C(=C\F)COc1ccc(C23CCC(C(=O)N4CC(F)(CO)C4)(CC2)CC3)cc1] | (E)-(4-(4-((2-(aminomethyl)-3- fluoroallyl)oxy)phenyl) bicyclo[2.2.2]octan-1-yl)(3- fluoro-3-(hydroxymethyl) azetidin-1-yl)methanone | 421.1 |
| 2.61 | 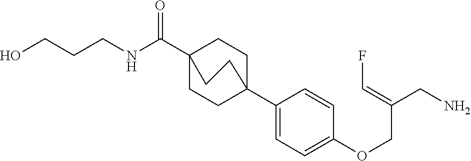 [SMILES: NC/C(=C\F)COc1ccc(C23CCC(C(=O)NCCCO)(CC2)CC3)cc1] | (E)-4-(4-((2-(aminomethyl)-3- fluoroallyl)oxy)phenyl)-N-(3- hydroxypropyl)bicyclo[2.2.2] octane-1-carboxamide | 391.2 |
| 2.62 | 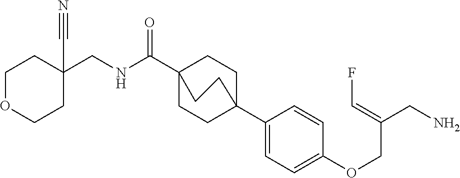 [SMILES: N#CC1(CNC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-((4- cyanotetrahydro-2H-pyran-4- yl)methyl)bicyclo[2.2.2]octane- 1-carboxamide | 456.2 |
| 2.63 | 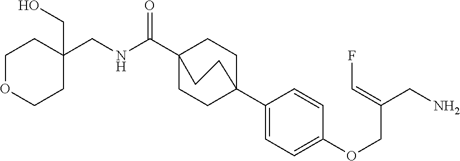 [SMILES: NC/C(=C\F)COc1ccc(C23CCC(C(=O)NCC4(CO)CCOCC4)(CC2)CC3)cc1] | (E)-4-(4-((2-(aminomethyl)-3- fluoroallyl)oxy)phenyl)-N-((4- (hydroxymethyl)tetrahydro-2H- pyran-4- yl)methyl)bicyclo[2.2.2]octane- 1-carboxamide | 461.2 |
| 2.64 | 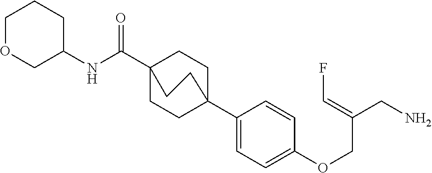 [SMILES: NC/C(=C\F)COc1ccc(C23CCC(C(=O)NC4CCCOC4)(CC2)CC3)cc1] | (E)-4-(4-((2-(aminomethyl)-3- fluoroallyl)oxy)phenyl)-N- (tetrahydro-2H-pyran-3- yl)bicyclo[2.2.2]octane-1- carboxamide | 417.2 |
| 2.65 | 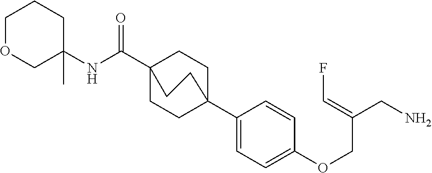 [SMILES: CC1(NC(=O)C23CCC(c4ccc(OC/C(=C/F)CN)cc4)(CC2)CC3)CCCOC1] | (E)-4-(4-((2-(aminomethyl)-3- fluoroallyl)oxy)phenyl)-N-(3- methyltetrahydro-2H-pyran-3- yl)bicyclo[2.2.2]octane-1- carboxamide | 431.2 |
| 2.66 | 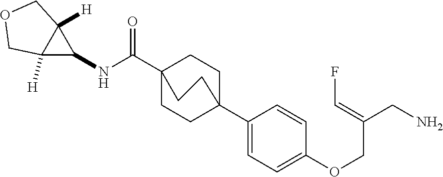 | 4-(4-(((E)-2-(aminomethyl)-3- fluoroallyl)oxy)phenyl)-N- ((1R,5S,6s)-3- oxabicyclo[3.1.0]hexan-6- yl)bicyclo[2.2.2]octane-1- carboxamide | 415.1 |
| 2.67 | 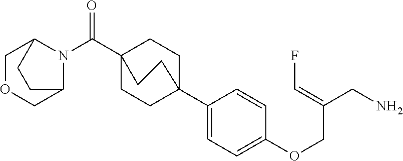 [SMILES: NC/C(=C\F)COc1ccc(C23CCC(C(=O)N4C5CCC4COC5)(CC2)CC3)cc1] | (E)-(4-(4-((2-(aminomethyl)-3- fluoroallyl)oxy)phenyl)bicyclo [2.2.2]octan-1-yl)(3-oxa-8- azabicyclo[3.2.1]octan-8- yl)methanone | 439.1 |
| 2.68 | 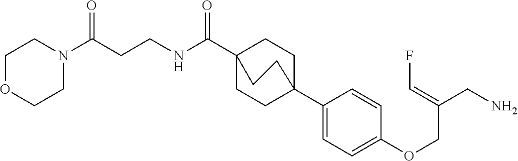 [SMILES: NC/C(=C\F)COc1ccc(C23CCC(C(=O)NCCC(=O)N4CCOCC4)(CC2)CC3)cc1] | (E)-4-(4-((2-(aminomethyl)-3- fluoroallyl)oxy)phenyl)-N-(3- morpholino-3- oxopropyl)bicyclo[2.2.2]octane- 1-carboxamide | 474.2 |
| 2.69 | 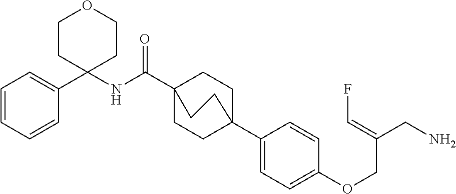 [SMILES: NC/C(=C\F)COc1ccc(C23CCC(C(=O)NC4(c5ccccc5)CCOCC4)(CC2)CC3)cc1] | (E)-4-(4-((2-(aminomethyl)-3- fluoroallyl)oxy)phenyl)-N-(4- phenyltetrahydro-2H-pyran-4- yl)bicyclo[2.2.2]octane-1- carboxamide | 493.3 |
| 2.70 | 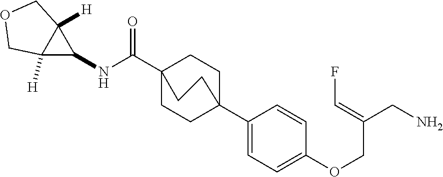 | 4-(4-(((E)-2-(aminomethyl)-3- fluoroallyl)oxy)phenyl)-N- ((1R,5S,6r)-3- oxabicyclo[3.1.0]hexan-6- yl)bicyclo[2.2.2]octane-1- carboxamide | 415.1 |
| 2.71 | 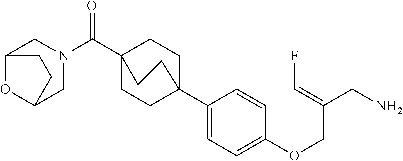 [SMILES: NC/C(=C\F)COc1ccc(C23CCC(C(=O)N4CC5CCC(C4)O5)(CC2)CC3)cc1] | (E)-(4-(4-((2-(aminomethyl)-3- fluoroallyl)oxy)phenyl)bicyclo [2.2.2]octan-1-yl)(8-oxa-3- azabicyclo[3.2.1]octan-3- yl)methanone | 429.2 |
| 2.72 | 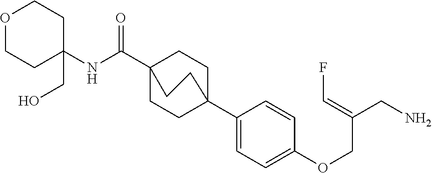 [SMILES: NC/C(=C\F)COc1ccc(C23CCC(C(=O)NC4(CO)CCOCC4)(CC2)CC3)cc1] | (E)-4-(4-((2-(aminomethyl)-3- fluoroallyl)oxy)phenyl)-N-(4- (hydroxymethyl)tetrahydro-2H- pyran-4- yl)bicyclo[2.2.2]octane-1- carboxamide | 447.2 |
| 2.73 | 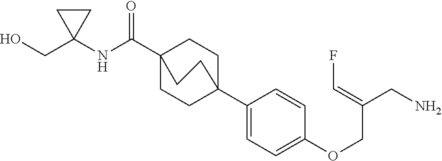 [SMILES: NC/C(=C\F)COc1ccc(C23CCC(C(=O)NC4(CO)CC4)(CC2)CC3)cc1] | (E)-4-(4-((2-(aminomethyl)-3- fluoroallyl)oxy)phenyl)-N-(1- (hydroxymethyl)cyclopropyl) bicyclo[2.2.2]octane-1- carboxamide | 403.1 |
| 2.74 | 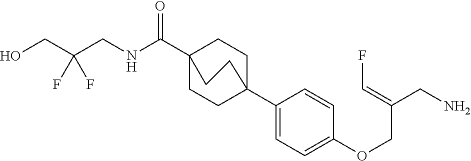 [SMILES: NC/C(=C\F)COc1ccc(C23CCC(C(=O)NCC(F)(F)CO)(CC2)CC3)cc1] | (E)-4-(4-((2-(aminomethyl)-3- fluoroallyl)oxy)phenyl)-N-(2,2- difluoro-3- hydroxypropyl)bicyclo[2.2.2] octane-1-carboxamide | 427.1 |
| 2.75 | 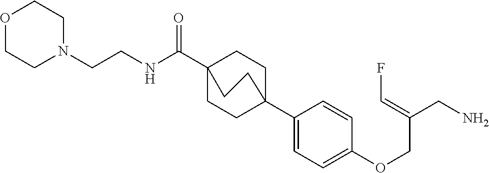 [SMILES: NC/C(=C\F)COc1ccc(C23CCC(C(=O)NCCN4CCOCC4)(CC2)CC3)cc1] | (E)-4-(4-((2-(aminomethyl)-3- fluoroallyl)oxy)phenyl)-N-(2- morpholinoethyl)bicyclo[2.2.2] octane-1-carboxamide | 446.2 |
| 2.76 | 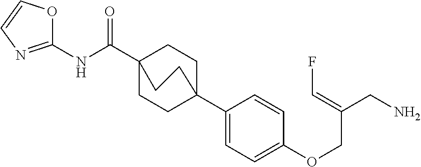 [SMILES: NC/C(=C\F)COc1ccc(C23CCC(C(=O)Nc4ncco4)(CC2)CC3)cc1] | (E)-4-(4-((2-(aminomethyl)-3- fluoroallyl)oxy)phenyl)-N- (oxazol-2- yl)bicyclo[2.2.2]octane-1- carboxamide | 400.1 |
| 2.77 | 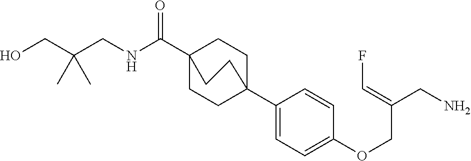 [SMILES: CC(C)(CO)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- hydroxy-2,2- dimethylpropyl)bicyclo[2.2.2] octane-1-carboxamide | 419.3 |
| 2.78 | 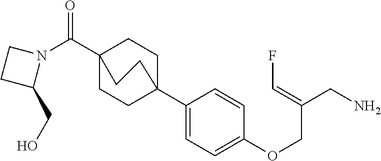 [SMILES: NC/C(=C\F)COc1ccc(C23CCC(C(=O)N4CC[C@@H]4CO)(CC2)CC3)cc1] | (R,E)-(4-(4-((2-(aminomethyl)-3- fluoroallyl)oxy)phenyl)bicyclo [2.2.2]octan-1-yl)(2- (hydroxymethyl)azetidin-1- yl)methanone | 403.2 |
| 2.79 | 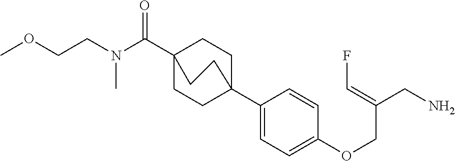 [SMILES: COCCN(C)C(=O)C12CCC(c3ccc(OC/C(=C/F)CN)cc3)(CC1)CC2] | (E)-4-(4-((2-(aminomethyl)-3- fluoroallyl)oxy)phenyl)-N-(2- methoxyethyl)-N- methylbicyclo[2.2.2]octane-1- carboxamide | 405.2 |
| 2.80 | 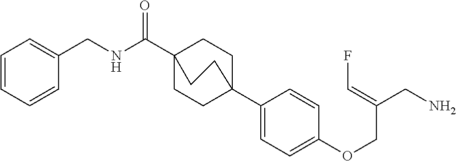 [SMILES: NC/C(=C\F)COc1ccc(C23CCC(C(=O)NCc4ccccc4)(CC2)CC3)cc1] | (E)-4-(4-((2-(aminomethyl)-3- fluoroallyl)oxy)phenyl)-N- benzylbicyclo[2.2.2]octane-1- carboxamide | 423.2 |
| 2.81 | 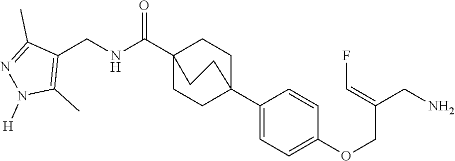 [SMILES: Cc1n[nH]c(C)c1CNC(=O)C12CCC(c3ccc(OC/C(=C/F)CN)cc3)(CC1)CC2] | (E)-4-(4-((2-(aminomethyl)-3- fluoroallyl)oxy)phenyl)-N- ((3,5-dimethyl-1H-pyrazol-4- yl)methyl)bicyclo[2.2.2]octane- 1-carboxamide | 441.2 |
| 2.83 | 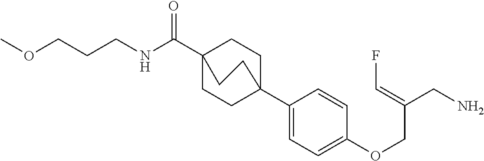 [SMILES: COCCCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- methoxypropyl)bicyclo[2.2.2] octane-1-carboxamide | 405.3 |
| 2.84* | 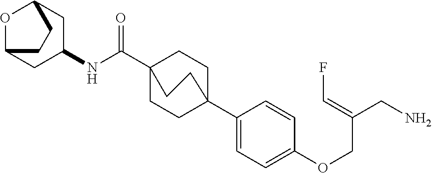 | (E)-4-(4-((2-(aminomethyl)-3- fluoroallyl)oxy)phenyl)-N-(8- oxabicyclo[3.2.1]octan-3- yl)bicyclo[2.2.2]octane-1- carboxamide | 443.3 |
| 2.84b* | 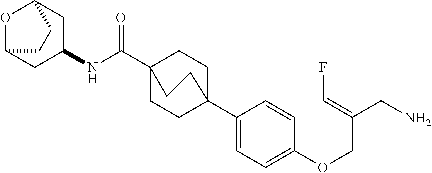 | (E)-4-(4-((2-(aminomethyl)-3- fluoroallyl)oxy)phenyl)-N-(8- oxabicyclo[3.2.1]octan-3-yl) bicyclo[2.2.2]octane-1- carboxamide | 443.3 |
| 2.85 | 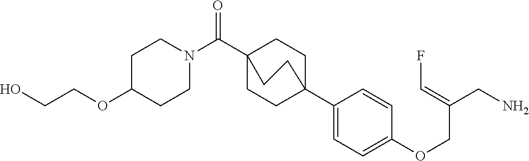 [SMILES: NC/C(=C\F)COc1ccc(C23CCC(C(=O)N4CCC(OCCO)CC4)(CC2)CC3)cc1] | (E)-(4-(4-((2-(aminomethyl)-3- fluoroallyl)oxy)phenyl)bicyclo [2.2.2]octan-1-yl)(4-(2- hydroxyethoxy)piperidin-1- yl)methanone | 461.2 |
| 2.86 | 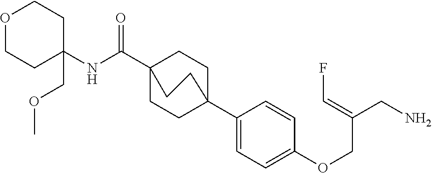 [SMILES: COCC1(NC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-(4- (methoxymethyl)tetrahydro-2H- pyran-4- yl)bicyclo[2.2.2]octane-1- carboxamide | 461.2 |
| 2.87 | 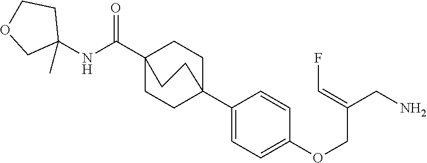 [SMILES: CC1(NC(=O)C23CCC(c4ccc(OC/C(=C/F)CN)cc4)(CC2)CC3)CCOC1] | (E)-4-(4-((2-(aminomethyl)-3- fluoroallyl)oxy)phenyl)-N-(3- methyltetrahydrofuran-3- yl)bicyclo[2.2.2]octane-1- carboxamide | 417.2 |
| 2.88 | 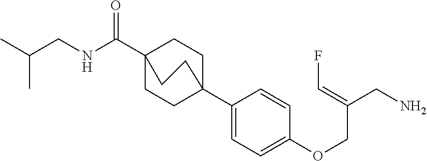 [SMILES: CC(C)CNC(=O)C12CCC(c3ccc(OC/C(=C/F)CN)cc3)(CC1)CC2] | (E)-4-(4-((2-(aminomethyl)-3- fluoroallyl)oxy)phenyl)-N- isobutylbicyclo[2.2.2]octane-1- carboxamide | 389.1 |
| 2.89 | 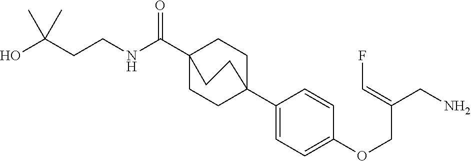 [SMILES: CC(C)(O)CCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- hydroxy-3- methylbutyl)bicyclo[2.2.2] octane-1-carboxamide | 419.2 |
| 2.90 | 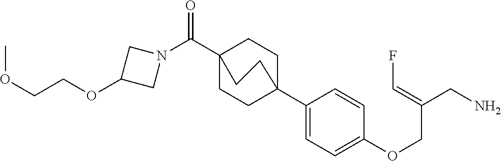 [SMILES: COCCOC1CN(C(=O)C23CCC(c4ccc(OC/C(=C/F)CN)cc4)(CC2)CC3)C1] | (E)-(4-(4-((2-(aminomethyl)-3- fluoroallyl)oxy)phenyl)bicyclo [2.2.2]octan-1-yl)(3-(2- methoxyethoxy)azetidin-1- yl)methanone | 447.2 |
| 2.91 | 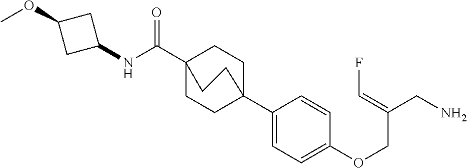 | 4-(4-(((E)-2-(aminomethyl)-3- fluoroallyl)oxy)phenyl)-N- ((1r,3r)-3- methoxycyclobutyl)bicyclo [2.2.2]octane-1-carboxamide | 417.2 |
| 2.92 | 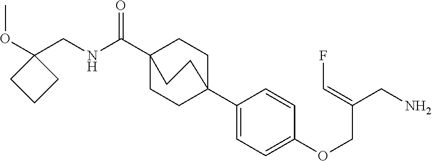 [SMILES: COC1(CNC(=O)C23CCC(c4ccc(OC/C(=C/F)CN)cc4)(CC2)CC3)CCC1] | (E)-4-(4-((2-(aminomethyl)-3- fluoroallyl)oxy)phenyl)-N-((1- methoxycyclobutyl)methyl) bicyclo[2.2.2]octane-1- carboxamide | 431.2 |
| 2.93 | 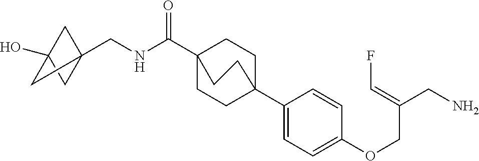 [SMILES: NC/C(=C\F)COc1ccc(C23CCC(C(=O)NCC45CC(O)(C4)C5)(CC2)CC3)cc1] | (E)-4-(4-((2-(aminomethyl)-3- fluoroallyl)oxy)phenyl)-N-(3- hydroxybicyclo[1.1.1]pentan-1- yl)bicyclo[2.2.2]octane-1- carboxamide | 415.2 |
| 2.94 | 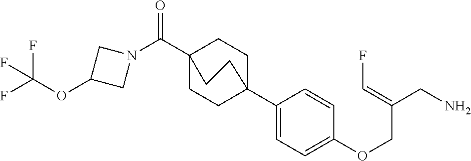 [SMILES: NC/C(=C\F)COc1ccc(C23CCC(C(=O)N4CC(OC(F)(F)F)C4)(CC2)CC3)cc1] | (E)-(4-(4-((2-(aminomethyl)-3- fluoroallyl)oxy)phenyl)bicyclo [2.2.2]octan-1-yl)(3- (trifluoromethoxy)azetidin-1- yl)methanone | 457.1 |
| 2.96 | 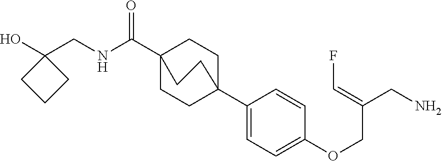 [SMILES: NC/C(=C\F)COc1ccc(C23CCC(C(=O)NCC4(O)CCC4)(CC2)CC3)cc1] | (E)-4-(4-((2-(aminomethyl)-3- fluoroallyl)oxy)phenyl)-N-((1- hydroxycyclobutyl)methyl) bicyclo[2.2.2]octane-1- carboxamide | 417.2 |
| 2.97 | 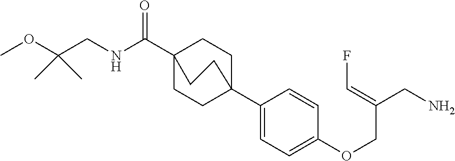 [SMILES: COC(C)(C)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- methoxy-2- methylpropyl)bicyclo[2.2.2] octane-1-carboxamide | 419.2 |
| 2.98 | 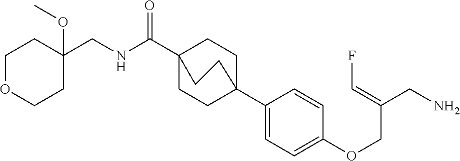 [SMILES: COC1(CNC(=O)C23CCC(c4ccc(OC/C(=C/F)CN)cc4)(CC2)CC3)CCOCC1] | (E)-4-(4-((2-(aminomethyl)-3- fluoroallyl)oxy)phenyl)-N-((4- methoxytetrahydro-2H-pyran-4- yl)methyl)bicyclo[2.2.2]octane- 1-carboxamide | 461.3 |
| 2.99 | 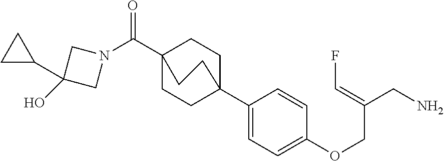 [SMILES: NC/C(=C\F)COc1ccc(C23CCC(C(=O)N4CC(O)(C5CC5)C4)(CC2)CC3)cc1] | (E)-(4-(4-((2-(aminomethyl)-3- fluoroallyl)oxy)phenyl)bicyclo [2.2.2]octan-1-yl)(3- cyclopropyl-3-hydroxyazetidin- 1-yl)methanone | 429.2 |
| 2.100 | 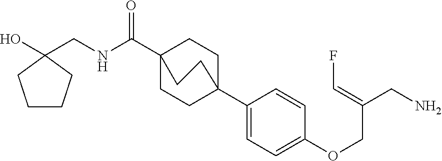 [SMILES: NC/C(=C\F)COc1ccc(C23CCC(C(=O)NCC4(O)CCCC4)(CC2)CC3)cc1] | (E)-4-(4-((2-(aminomethyl)-3- fluoroallyl)oxy)phenyl)-N-((1- hydroxycyclopentyl)methyl) bicyclo[2.2.2]octane-1- carboxamide | 431.2 |
| 2.101 | 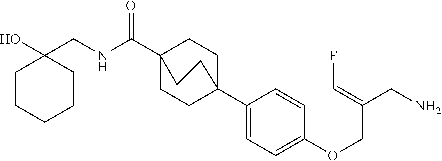 [SMILES: NC/C(=C\F)COc1ccc(C23CCC(C(=O)NCC4(O)CCCCC4)(CC2)CC3)cc1] | (E)-4-(4-((2-(aminomethyl)-3- fluoroallyl)oxy)phenyl)-N-((1- hydroxycyclohexyl)methyl) bicyclo[2.2.2]octane-1- carboxamide | 445.2 |
| 2.102 | 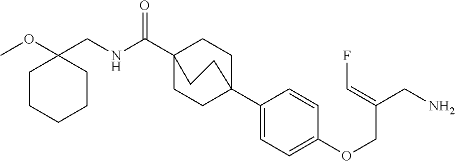 [SMILES: COC1(CNC(=O)C23CCC(c4ccc(OC/C(=C/F)CN)cc4)(CC2)CC3)CCCCC1] | (E)-4-(4-((2-(aminomethyl)-3- fluoroallyl)oxy)phenyl)-N-((1- methoxycyclohexyl)methyl) bicyclo[2.2.2]octane-1- carboxamide | 459.2 |
| 2.103 | 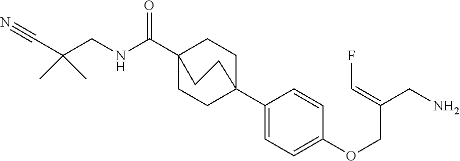 [SMILES: CC(C)(C#N)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- cyano-2- methylpropyl)bicyclo[2.2.2] octane-1-carboxamide | 414.2 |
| 2.104 | 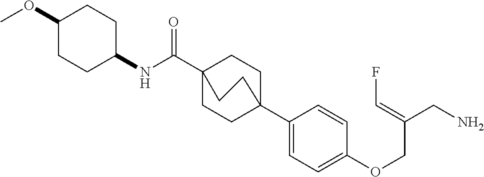 | 4-(4-((E)-2-(aminomethyl)-3- fluoroallyl)oxy)phenyl)-N- ((1r,4r)-4-methoxy- cyclohexyl)bicyclo[2.2.2] octane-1-carboxamide | 445.2 |
| 2.105 | 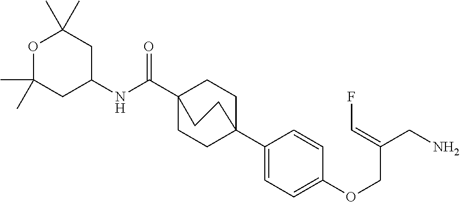 [SMILES: CC1(C)CC(NC(=O)C23CCC(c4ccc(OC/C(=C/F)CN)cc4)(CC2)CC3)CC(C)(C)O1] | (E)-4-(4-((2-(aminomethyl)-3- fluoroallyl)oxy)phenyl)-N- (2,2,6,6-tetramethyltetrahydro- 2H-pyran-4- yl)bicyclo[2.2.2]octane-1- carboxamide | 473.4 |
| 2.106 | 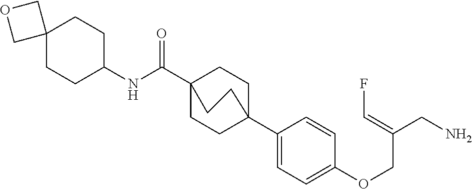 [SMILES: NC/C(=C\F)COc1ccc(C23CCC(C(=O)NC4CCC5(CC4)COC5)(CC2)CC3)cc1] | (E)-4-(4-((2-(aminomethyl)-3- fluoroallyl)oxy)phenyl)-N-(2- oxaspiro[3.5]nonan-7- yl)bicyclo[2.2.2]octane-1- carboxamide | 457.2 |
| 2.107 | 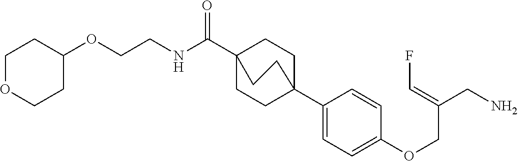 [SMILES: NC/C(=C\F)COc1ccc(C23CCC(C(=O)NCCOC4CCOCC4)(CC2)CC3)cc1] | (E)-4-(4-((2-(aminomethyl)-3- fluoroallyl)oxy)phenyl)-N-(2- yl)oxy)ethyl)bicyclo[2.2.2] octane-1-carboxamide | 461.3 |
| 2.108 | 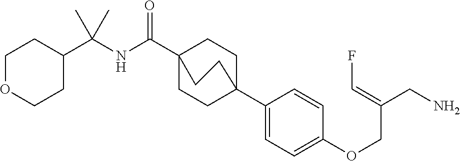 [SMILES: CC(C)(NC(=O)C12CCC(c3ccc(OC/C(=C/F)CN)cc3)(CC1)CC2)C1CCOCC1] | (E)-4-(4-((2-(aminomethyl)-3- fluoroallyl)oxy)phenyl)-N-(2- (tetrahydro-2H-pyran-4- yl)propan-2- yl)bicyclo[2.2.2]octane-1- carboxamide | 459.3 |
| 2.109 | 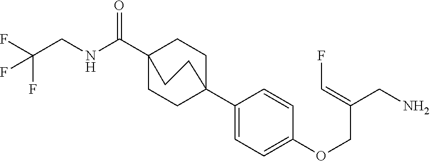 [SMILES: NC/C(=C\F)COc1ccc(C23CCC(C(=O)NCC(F)(F)F)(CC2)CC3)cc1] | (E)-4-(4-((2-(aminomethyl)-3- fluoroallyl)oxy)phenyl)-N- (2,2,2-trifluoroethyl)bicyclo [2.2.2]octane-1-carboxamide | 415.1 |
| 2.110 | 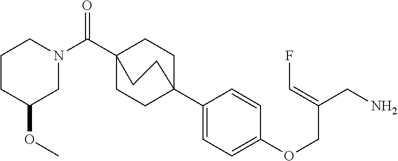 [SMILES: CO[C@H]1CCCN(C(=O)C23CCC(c4ccc(OC/C(=C/F)CN)cc4)(CC2)CC3)C1] | (S,E)-(4-(4-((2-(aminomethyl)-3- fluoroallyl)oxy)phenyl)bicyclo [2.2.2]octan-1-yl)(3- methoxypiperidin-1- yl)methanone | 431.2 |
| 2.111 | 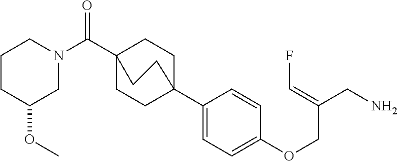 [SMILES: CO[C@@H]1CCCN(C(=O)C23CCC(c4ccc(OC/C(=C/F)CN)cc4)(CC2)CC3)C1] | (R,E)-(4-(4-((2-(aminomethyl)- 3-fluoroallyl)oxy)phenyl)bicyclo [2.2.2]octan-1-yl)(3- methoxypiperidin-1- yl)methanone | 431.2 |
| 2.112 | 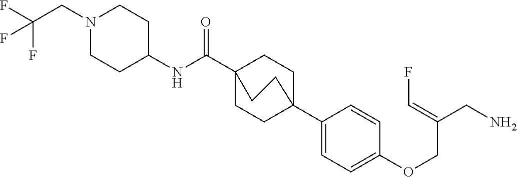 [SMILES: NC/C(=C\F)COc1ccc(C23CCC(C(=O)NC4CCN(CC(F)(F)F)CC4)(CC2)CC3)cc1] | (E)-4-(4-((2-(aminomethyl)-3- fluoroallyl)oxy)phenyl)-N-(1- (2,2,2-trifluoroethyl)piperidin- 4-yl)bicyclo[2.2.2]octane-1- carboxamide | 498.2 |
| 2.113 | 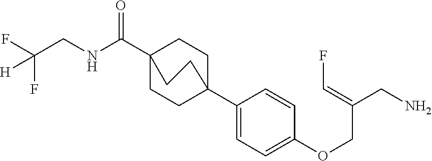 [SMILES: NC/C(=C\F)COc1ccc(C23CCC(C(=O)NCC(F)F)(CC2)CC3)cc1] | (E)-4-(4-((2-(aminomethyl)-3- fluoroallyl)oxy)phenyl)-N-(2,2- difluoroethyl)bicyclo[2.2.2] octane-1-carboxamide | 397.1 |
| 2.114 | 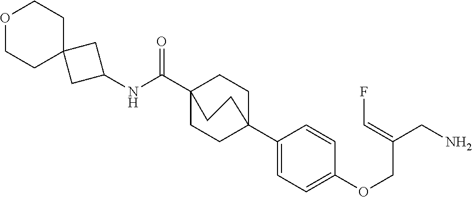 [SMILES: NC/C(=C\F)COc1ccc(C23CCC(C(=O)NC4CC5(CCOCC5)C4)(CC2)CC3)cc1] | (E)-4-(4-((2-(aminomethyl)-3- fluoroallyl)oxy)phenyl)-N-(7- oxaspiro[3.5]nonan-2- yl)bicyclo[2.2.2]octane-1- carboxamide | 457.4 |
| 2.115 | 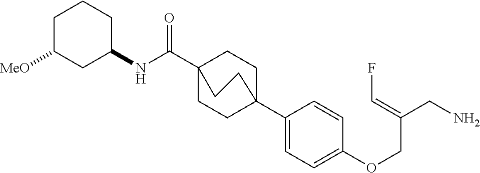 [SMILES: CO[C@@H]1CCC[C@@H](NC(=O)C23CCC(c4ccc(OC/C(=C/F)CN)cc4)(CC2)CC3)C1] | 4-(4-(((E)-2-(aminomethyl)-3- fluoroallyl)oxy)phenyl)-N- ((1S,3R)-3- methoxycyclohexyl)bicyclo [2.2.2]octane-1-carboxamide | 445.3 |
| 2.116 | 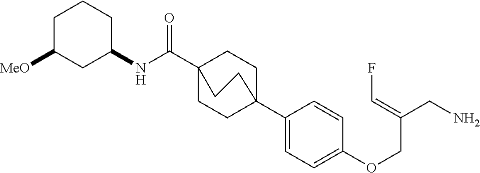 [SMILES: CO[C@H]1CCC[C@@H](NC(=O)C23CCC(c4ccc(OC/C(=C/F)CN)cc4)(CC2)CC3)C1] | 4-(4-(((E)-2-(aminomethyl)-3- fluoroallyl)oxy)phenyl)-N- ((1R,3S)-3- methoxycyclohexyl)bicyclo [2.2.2]octane-1-carboxamide | 445.3 |
| 2.117 | 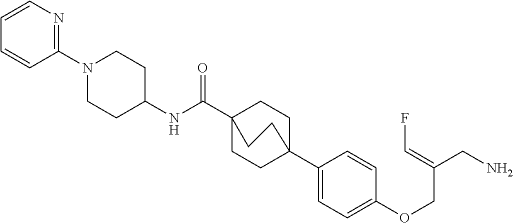 [SMILES: NC/C(=C\F)COc1ccc(C23CCC(C(=O)NC4CCN(c5ccccn5)CC4)(CC2)CC3)cc1] | (E)-4-(4-((2-(aminomethyl)-3- fluoroallyl)oxy)phenyl)-N-(1- (pyridin-2-yl)piperidin-4- yl)bicyclo[2.2.2]octane-1- carboxamide | 493.3 |
| 2.118 | 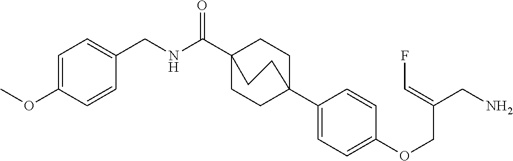 [SMILES: COc1ccc(CNC(=O)C23CCC(c4ccc(OC/C(=C/F)CN)cc4)(CC2)CC3)cc1] | (E)-4-(4-((2-(aminomethyl)-3- fluoroallyl)oxy)phenyl)-N-(4- methoxybenzyl)bicyclo[2.2.2] octane-1-carboxamide | 453.3 |
| 2.119 | 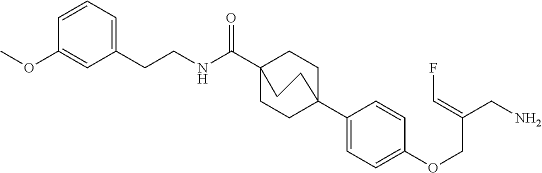 [SMILES: COc1cccc(CCNC(=O)C23CCC(c4ccc(OC/C(=C/F)CN)cc4)(CC2)CC3)c1] | (E)-4-(4-((2-(aminomethyl)-3- fluoroallyl)oxy)phenyl)-N-(3- methoxyphenethyl)bicyclo [2.2.2]octane-1-carboxamide | 467.3 |
| 2.120 | 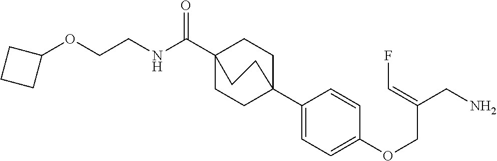 [SMILES: NC/C(=C\F)COc1ccc(C23CCC(C(=O)NCCOC4CCC4)(CC2)CC3)cc1] | (E)-4-(4-((2-(aminomethyl)-3- fluoroallyl)oxy)phenyl)-N-(2- cyclobutoxyethyl)bicyclo [2.2.2]octane-1-carboxamide | 431.3 |
| 2.121 | 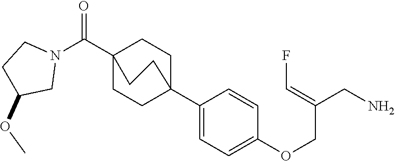 [SMILES: CO[C@H]1CCN(C(=O)C23CCC(c4ccc(OC/C(=C/F)CN)cc4)(CC2)CC3)C1] | (S,E)-(4-(4-((2-(aminomethyl)-3- fluoroallyl)oxy)phenyl)bicyclo [2.2.2]octan-1-yl)(3- methoxypyrrolidin-1- yl)methanone | 417.2 |
| 2.122 | 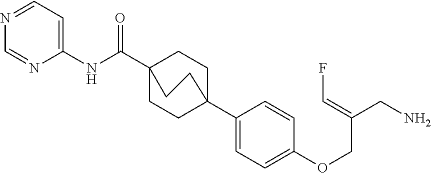 [SMILES: NC/C(=C\F)COc1ccc(C23CCC(C(=O)Nc4ccncn4)(CC2)CC3)cc1] | (E)-4-(4-((2-(aminomethyl)-3- fluoroallyl)oxy)phenyl)-N- (pyrimidin-4- yl)bicyclo[2.2.2]octane-1- carboxamide | 411.1 |
| 2.123 | 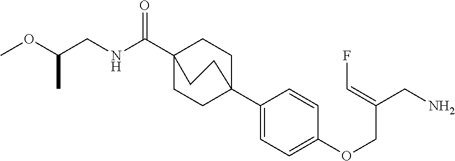 [SMILES: CO[C@H](C)CNC(=O)C12CCC(c3ccc(OC/C(=C/F)CN)cc3)(CC1)CC2] | (R,E)-4-(4-((2-(aminomethyl)-3- 3-fluoroallyl)oxy)phenyl)-N-(2- methoxypropyl)bicyclo[2.2.2] octane-1-carboxamide | 405.2 |
| 2.124 | 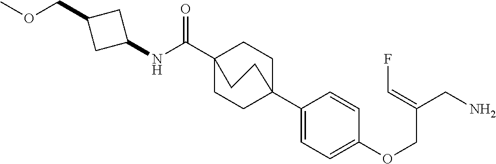 | 4-(4-(((E)-2-(aminomethyl)-3- fluoroallyl)oxy)phenyl)-N- ((1s,3s)-3- (methoxymethyl)cyclobutyl) bicyclo[2.2.2]octane-1- carboxamide | 431.3 |
| 2.125 | 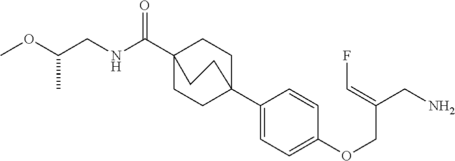 [SMILES: CO[C@@H](C)CNC(=O)C12CCC(c3ccc(OC/C(=C/F)CN)cc3)(CC1)CC2] | (S,E)-4-(4-((2-(aminomethyl)-3- 3-fluoroallyl)oxy)phenyl)-N-(2- methoxypropyl)bicyclo[2.2.2] octane-1-carboxamide | 405.2 |
| 2.126 | 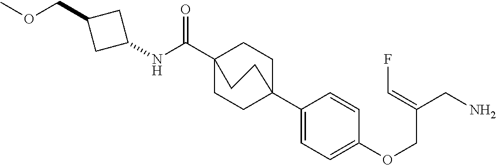 [SMILES: COC[C@H]1C[C@H](NC(=O)C23CCC(c4ccc(OC/C(=C/F)CN)cc4)(CC2)CC3)C1] | 4-(4-(((E)-2-(aminomethyl)-3- fluoroallyl)oxy)phenyl)-N- ((1r,3r)-3- (methoxymethyl)cyclobutyl) bicyclo[2.2.2]octane-1- carboxamide | 431.2 |
| 2.127 | 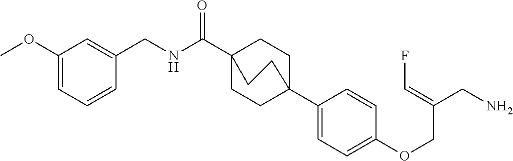 [SMILES: COc1cccc(CNC(=O)C23CCC(c4ccc(OC/C(=C/F)CN)cc4)(CC2)CC3)c1] | (E)-4-(4-((2-(aminomethyl)-3- fluoroallyl)oxy)phenyl)-N-(3- methoxybenzyl)bicyclo[2.2.2] octane-1-carboxamide | 453.2 |
| 2.128 | 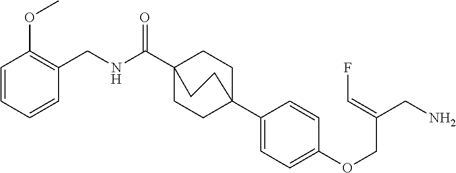 [SMILES: COc1ccccc1CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- methoxybenzyl)bicyclo[2.2.2] octane-1-carboxamide | 453.2 |
| 2.129 | 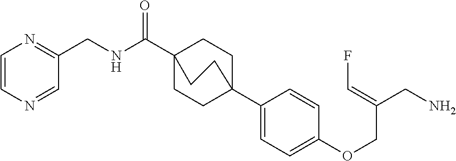 [SMILES: NC/C(=C\F)COc1ccc(C23CCC(C(=O)NCc4cnccn4)(CC2)CC3)cc1] | (E)-4-(4-((2-(aminomethyl)-3- fluoroallyl)oxy)phenyl)-N- (pyrazin-2- ylmethyl)bicyclo[2.2.2]octane- 1-carboxamide | 425.2 |
| 2.130 | 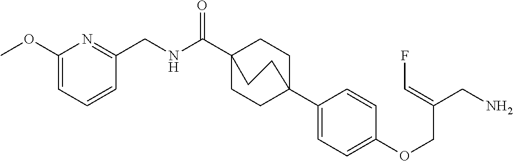 [SMILES: COc1cccc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((6- methoxypyridin-2- yl)methyl)bicyclo[2.2.2]octane- 1-carboxamide | 454.2 |
| 2.131 | 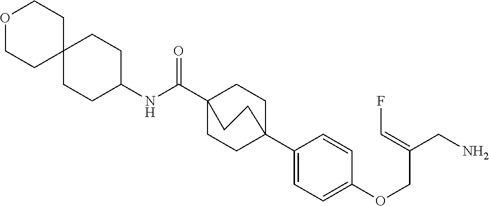 [SMILES: NC/C(=C\F)COc1ccc(C23CCC(C(=O)NC4CCC5(CCOCC5)CC4)(CC2)CC3)cc1] | (E)-4-(4-((2-(aminomethyl)-3- fluoroallyl)oxy)phenyl)-N-(3- oxaspiro[5.5]undecan-9- yl)bicyclo[2.2.2]octane-1- carboxamide | 485.4 |
| 2.132 | 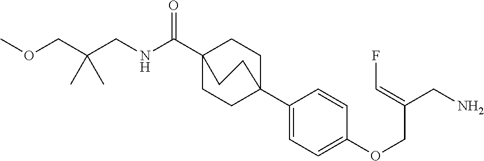 [SMILES: COCC(C)(C)CNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- methoxy-2,2- dimethylpropyl)bicyclo[2.2.2] octane-1-carboxamide | 433.2 |
| 2.133 | 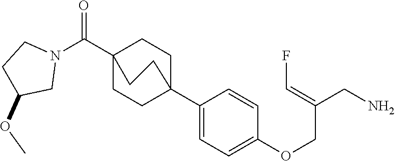 [SMILES: CO[C@H]1CCN(C(=O)C23CCC(c4ccc(OC/C(=C/F)CN)cc4)(CC2)CC3)C1] | (R,E)-(4-(4-((2-(aminomethyl)- 3-fluoroallyl)oxy)phenyl) bicyclo[2.2.2]octan-1-yl)(3- methoxypyrrolidin-1- yl)methanone | 417.2 |
| 2.134 | 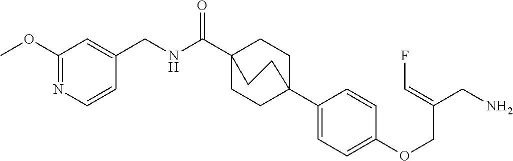 [SMILES: COc1cc(CNC(=O)C23CCC(c4ccc(OC/C(=C/F)CN)cc4)(CC2)CC3)ccn1] | (E)-4-(4-((2-(aminomethyl)-3- fluoroallyl)oxy)phenyl)-N-((2- methoxpyridin-4- yl)methyl)bicyclo[2.2.2]octane- 1-carboxamide | 454.2 |
| 2.135 | 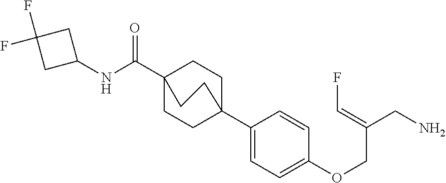 [SMILES: NC/C(=C\F)COc1ccc(C23CCC(C(=O)NC4CC(F)(F)C4)(CC2)CC3)cc1] | (E)-4-(4-((2-(aminomethyl)-3- fluoroallyl)oxy)phenyl)-N-(3,3- difluorocyclobutyl)bicyclo[2.2.2] octane-1-carboxamide | 423.1 |
| 2.136 | 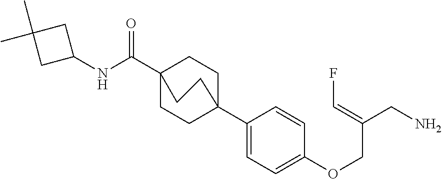 [SMILES: CC1(C)CC(NC(=O)C23CCC(c4ccc(OC/C(=C/F)CN)cc4)(CC2)CC3)C1] | (E)-4-(4-((2-(aminomethyl)-3- fluoroallyl)oxy)phenyl)-N-(3,3- dimethylcyclobutyl)bicyclo [2.2.2]octane-1-carboxamide | 415.3 |
| 2.137 | 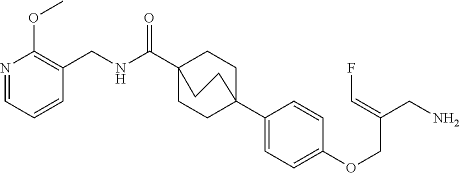 [SMILES: COc1ncccc1CNC(=O)C12CCC(c3ccc(OC/C(=C/F)CN)cc3)(CC1)CC2] | (E)-4-(4-((2-(aminomethyl)-3- fluoroallyl)oxy)phenyl)-N-((2- methoxypyridin-3- yl)methyl)bicyclo[2.2.2]octane- 1-carboxamide | 454.2 |
| 2.138 | 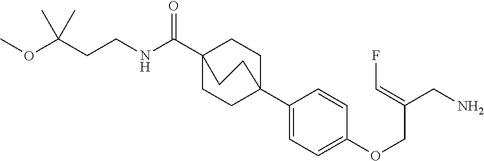 [SMILES: COC(C)(C)CCNC(=O)C12CCC(c3ccc(OC/C(=C/F)CN)cc3)(CC1)CC2] | (E)-4-(4-((2-(aminomethyl)-3- fluoroallyl)oxy)phenyl)-N-(3- methoxy-3- methylbutyl)bicyclo[2.2.2] octane-1-carboxamide | 433.2 |
| 2.139 | 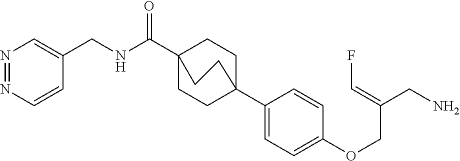 [SMILES: NC/C(=C\F)COc1ccc(C23CCC(C(=O)NCc4ccnnc4)(CC2)CC3)cc1] | (E)-4-(4-((2-(aminomethyl)-3- fluoroallyl)oxy)phenyl)-N- (pyridazin-4- ylmethyl)bicyclo[2.2.2]octane- 1-carboxamide | 425.2 |
| 2.140 | 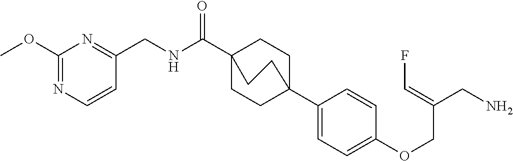 [SMILES: COc1nccc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((2- methoxypyrimidin-4- yl)methyl)bicyclo[2.2.2]octane- 1-carboxamide | 455.2 |
| 2.141 | 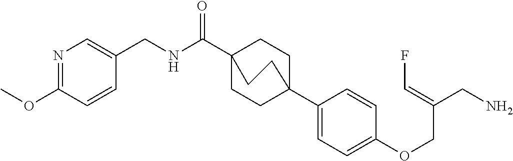 [SMILES: COc1ccc(CNC(=O)C23CCC(c4ccc(OC/C(=C/F)CN)cc4)(CC2)CC3)cn1] | (E)-4-(4-((2-(aminomethyl)-3- fluoroallyl)oxy)phenyl)-N-((6- methoxpyridin-3- yl)methyl)bicyclo[2.2.2]octane- 1-carboxamide | 454.2 |
| 2.142 | 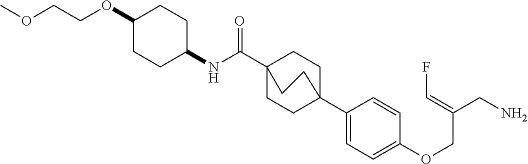 | 4-(4-(((E)-2-(aminomethyl)-3- fluoroallyl)oxy)phenyl)-N- ((1r,4r)-4-(2- methoxyethoxy)cyclohexyl) bicyclo[2.2.2]octane-1- carboxamide | 489.3 |
| 2.143 | 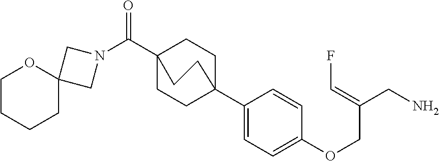 [SMILES: NC/C(=C\F)COc1ccc(C23CCC(C(=O)N4CC5(CCCCO5)C4)(CC2)CC3)cc1] | (E)-(4-(4-((2-(aminomethyl)-3- fluoroallyl)oxy)phenyl)bicyclo [2.2.2]octan-1-yl)(5-oxa-2- azaspiro[3.5]nonan-2- yl)methanone | 443.2 |
| 2.144 | 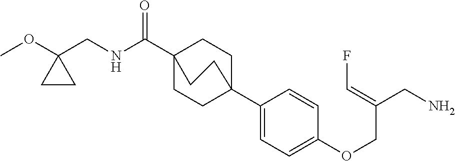 [SMILES: COC1(CNC(=O)C23CCC(c4ccc(OC/C(=C/F)CN)cc4)(CC2)CC3)CC1] | (E)-4-(4-((2-(aminomethyl)-3- fluoroallyl)oxy)phenyl)-N-((1- methoxycyclopropyl)methyl) bicyclo[2.2.2]octane-1- carboxamide | 417.2 |
| 2.145 | 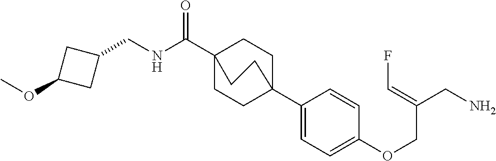 | 4-(4-(((E)-2-(aminomethyl)-3- fluoroallyl)oxy)phenyl)-N- (((1r,3r)-3- methoxycyclobutyl)methyl) bicyclo[2.2.2]octane-1- carboxamide | 431.2 |
| 2.146 | 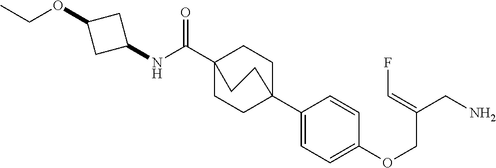 | 4-(4-(((E)-2-(aminomethyl)-3- fluoroallyl)oxy)phenyl)-N- ((1r,3r)-3- ethoxycyclobutyl)bicyclo[2.2.2] octane-1-carboxamide | 431.2 |
| 2.147 | 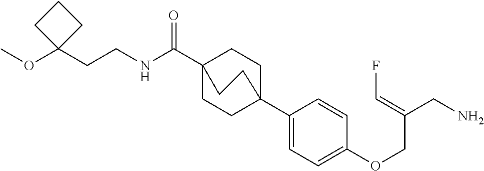 [SMILES: COC1(CCNC(=O)C23CCC(c4ccc(OC/C(=C/F)CN)cc4)(CC2)CC3)CCC1] | (E)-4-(4-((2-(aminomethyl)-3- fluoroallyl)oxy)phenyl)-N-(2- (1-methoxycyclobutyl)ethyl) bicyclo[2.2.2]octane-1- carboxamide | 445.2 |
| 2.148 | 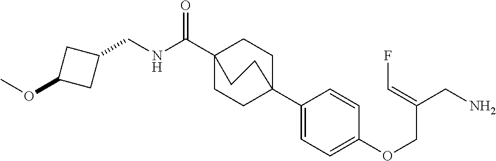 | 4-(4-(((E)-2-(aminomethyl)-3- fluoroallyl)oxy)phenyl)-N- (((1s,3s)-3- methoxycyclobutyl)methyl) bicyclo[2.2.2]octane-1- carboxamide | 431.3 |
| 2.149 | 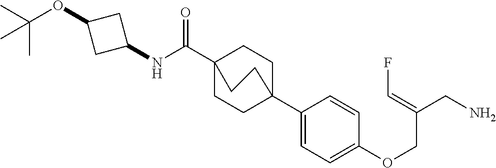 | 4-(4-(((E)-2-(aminomethyl)-3- fluoroallyl)oxy)phenyl)-N- ((1s,3s)-3-(tert- butoxy)cyclobutyl)bicyclo [2.2.2]octane-1-carboxamide | 459.2 |
| 2.150 | 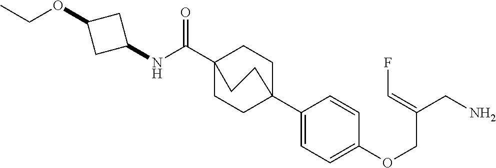 | 4-(4-(((E)-2-(aminomethyl)-3- fluoroallyl)oxy)phenyl)-N- ((1s,3s)-3- ethoxycyclobutyl)bicyclo[2.2.2] octane-1-carboxamide | 431.3 |
| 2.151 | 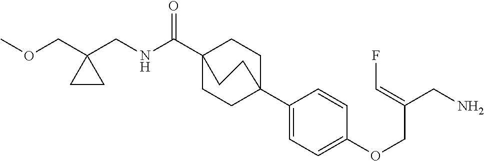 [SMILES: COCC1(CNC(=O)C23CCC(c4ccc(OC/C(=C/F)CN)cc4)(CC2)CC3)CC1] | (E)-4-(4-((2-(aminomethyl)-3- fluoroallyl)oxy)phenyl)-N-((1- (methoxymethyl)cyclopropyl) methyl)bicyclo[2.2.2]octane-1- carboxamide | 431.3 |
| 2.152 | 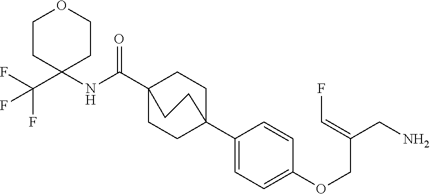 [SMILES: NC/C(=C\F)COc1ccc(C23CCC(C(=O)NC4(C(F)(F)F)CCOCC4)(CC2)CC3)cc1] | (E)-4-(4-((2-(aminomethyl)-3- fluoroallyl)oxy)phenyl)-N-(4- (trifluoromethyl)tetrahydro-2H- pyran-4- yl)bicyclo[2.2.2]octane-1- carboxamide | 485.8 |
| 2.153 | 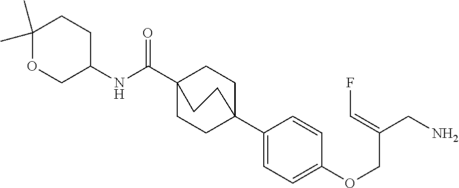 [SMILES: CC1(C)CCC(NC(=O)C23CCC(c4ccc(OC/C(=C/F)CN)cc4)(CC2)CC3)CO1] | (E)-4-(4-((2-(aminomethyl)-3- fluoroallyl)oxy)phenyl)-N-(6,6- dimethyltetrahydro-2H-pyran-3- yl)bicyclo[2.2.2]octane-1- carboxamide | 445.4 |
| 2.154 | 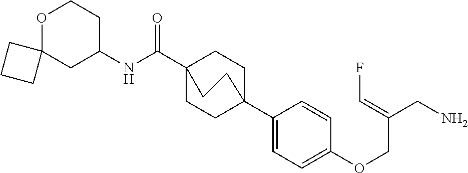 [SMILES: NC/C(=C\F)COc1ccc(C23CCC(C(=O)NC4CCOC5(CCC5)C4)(CC2)CC3)cc1] | (E)-4-(4-((2-(aminomethyl)-3- fluoroallyl)oxy)phenyl)-N-(5- oxaspiro[3.5]nonan-8- yl)bicyclo[2.2.2]octane-1- carboxamide | 457.4 |
| 2.155 | 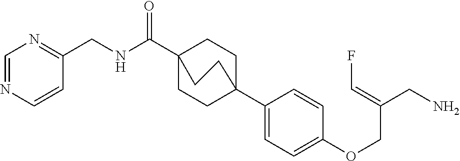 [SMILES: NC/C(=C\F)COc1ccc(C23CCC(C(=O)NCc4ccncn4)(CC2)CC3)cc1] | (E)-4-(4-((2-(aminomethyl)-3- fluoroallyl)oxy)phenyl)-N- (pyrimidin-4- ylmethyl)bicyclo[2.2.2]octane- 1-carboxamide | 425.1 |
| 2.156 | 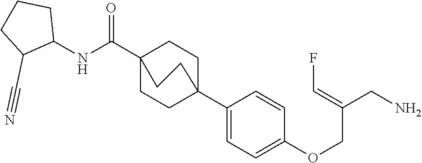 [SMILES: N#CC1CCCC1NC(=O)C12CCC(c3ccc(OC/C(=C/F)CN)cc3)(CC1)CC2] | (E)-4-(4-((2-(aminomethyl)-3- fluoroallyl)oxy)phenyl)-N-(2- cyanocyclopentyl)bicyclo[2.2.2] octane-1-carboxamide | 426.2 |
| 2.157 | 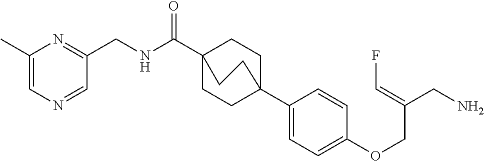 [SMILES: Cc1cncc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((6- methylpyrazin-2- yl)methyl)bicyclo[2.2.2]octane- 1-carboxamide | 439.2 |
| 2.158 | 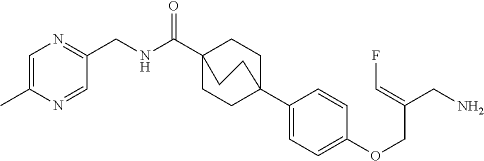 [SMILES: Cc1cnc(CNC(=O)C23CCC(c4ccc(OC/C(=C/F)CN)cc4)(CC2)CC3)cn1] | (E)-4-(4-((2-(aminomethyl)-3- fluoroallyl)oxy)phenyl)-N-((5- methylpyrazin-2- yl)methyl)bicyclo[2.2.2]octane- 1-carboxamide | 439.2 |
| 2.159 | 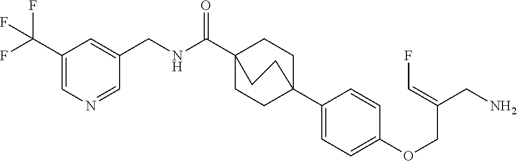 [SMILES: NC/C(=C\F)COc1ccc(C23CCC(C(=O)NCc4cncc(C(F)(F)F)c4)(CC2)CC3)cc1] | (E)-4-(4-((2-(aminomethyl)-3- fluoroallyl)oxy)phenyl)-N-((5- (trifluoromethyl)pyridin-3- yl)methyl)bicyclo[2.2.2]octane- 1-carboxamide | 492.1 |
| 2.160 | 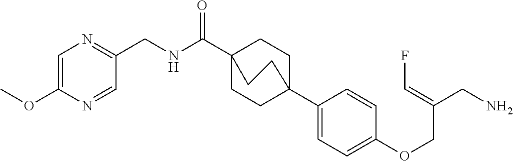 [SMILES: COc1cnc(CNC(=O)C23CCC(c4ccc(OC/C(=C/F)CN)cc4)(CC2)CC3)cn1] | (E)-4-(4-((2-(aminomethyl)-3- fluoroallyl)oxy)phenyl)-N-((5- methoxypyrazin-2- yl)methyl)bicyclo[2.2.2]octane- 1-carboxamide | 455.2 |
| 2.161 | 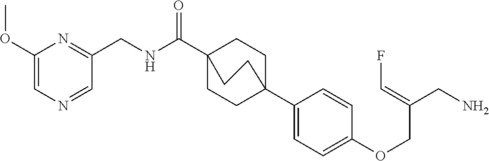 [SMILES: COc1cncc(CNC(=O)C23CCC(c4ccc(OC/C(=C/F)CN)cc4)(CC2)CC3)n1] | (E)-4-(4-((2-(aminomethyl)-3- fluoroallyl)oxy)phenyl)-N-((6- methoxypyrazin-2- yl)methyl)bicyclo[2.2.2]octane- 1-carboxamide | 455.1 |
| 3 | 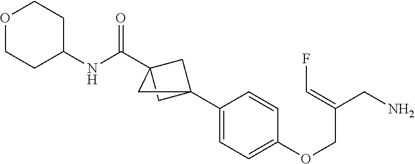 [SMILES: NC/C(=C\F)COc1ccc(C23CC(C(=O)NC4CCOCC4)(C2)C3)cc1] | (E)-3-(4-((2-(aminomethyl)-3- fluoroallyl)oxy)phenyl)-N- (tetrahydro-2H-pyran-4- yl)bicyclo[1.1.1]pentane-1- carboxamide | 375.0 |
| 3.01 | 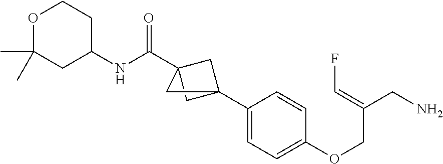 [SMILES: CC1(C)CC(NC(=O)C23CC(c4ccc(OC/C(=C/F)CN)cc4)(C2)C3)CCO1] | (E)-3-(4-((2-(aminomethyl)-3- fluoroallyl)oxy)phenyl)-N-(2,2- dimethyltetrahydro-2H-pyran-4- yl)bicyclo[1.1.1]pentane-1- carboxamide | 403.1 |
| 3.02 | 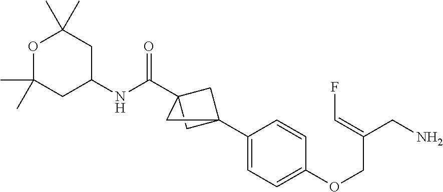 [SMILES: CC1(C)CC(NC(=O)C23CC(c4ccc(OC/C(=C/F)CN)cc4)(C2)C3)CC(C)(C)O1] | (E)-3-(4-((2-(aminomethyl)-3- fluoroallyl)oxy)phenyl)-N- (2,2,6,6-tetramethyltetrahydro- 2H-pyran-4- yl)bicyclo[1.1.1]pentane-1- carboxamide | 431.3 |
| 3.03 | 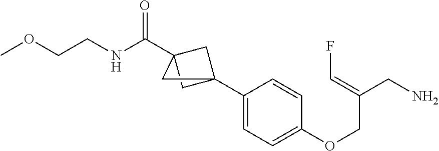 [SMILES: COCCNC(=O)C12CC(c3ccc(OC/C(=C/F)CN)cc3)(C1)C2] | (E)-3-(4-((2-(aminomethyl)-3- fluoroallyl)oxy)phenyl)-N-(2- methoxyethyl)bicyclo[1.1.1] pentane-1-carboxamide | 349.0 |
| 3.04 | 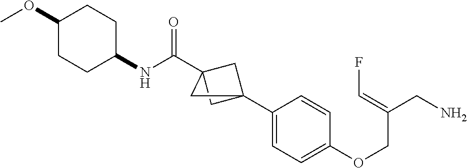 [SMILES: CO[C@H]1CC[C@@H](NC(=O)C23CC(c4ccc(OC/C(=C/F)CN)cc4)(C2)C3)CC1] | 3-(4-(((E)-2-(aminomethyl)-3- fluoroallyl)oxy)phenyl)-N- ((1r,4r)-4- methoxycyclohexyl)bicyclo [1.1.1]pentane-1-carboxamide | 403.2 |
| 3.05 | 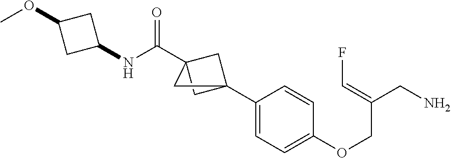 [SMILES: CO[C@H]1C[C@@H](NC(=O)C23CC(c4ccc(OC/C(=C/F)CN)cc4)(C2)C3)C1] | 3-(4-(((E)-2-(aminomethyl)-3- fluoroallyl)oxy)phenyl)-N- ((1r,3r)-3- methoxycyclobutyl)bicyclo [1.1.1]pentane-1-carboxamide | 375.1 |

*denotes arbitary assignment of bridgehead isomer

### Compound 2.06

### (E)-4-(4-((2-(Aminomethyl)-3-fluoroallyl)oxy)phenyl)-N-(tetrahydro-2H-pyran-4-yl)bicyclo[2.2.2]octane-1-carboxamide Hydrochloride

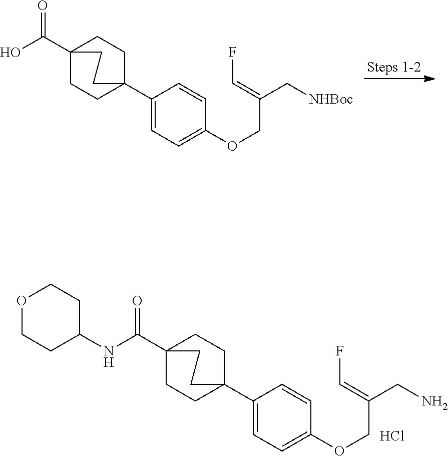 [SMILES: CC(C)(C)OC(=O)NC/C(=C\F)COc1ccc(C23CCC(C(=O)O)(CC2)CC3)cc1 ; NC/C(=C\F)COc1ccc(C23CCC(C(=O)NC4CCOCC4)(CC2)CC3)cc1 ; Cl]

### Step 1: (E)-tert-Butyl (3-fluoro-2-((4-(4-((tetrahydro-2H-pyran-4-yl)carbamoyl)bicyclo[2.2.2]octan-1-yl)phenoxy)methyl)allyl)carbamate

Diisopropylethylamine (2.0 mL, 11.5 mmol) was added to a solution of (E)-4-(4-((2-(((tert-butoxycarbonyl)amino)methyl)-3-fluoroallyl)oxy)phenyl)bicyclo[2.2.2]octane-1-carboxylic acid (1.51 g, 3.48 mmol), HATU (1.53 g, 4.19 mmol) and DMF (40 mL). After stirring the reaction mixture at room temperature for 15 min, tetrahydro-2H-pyran-4-amine (2.5 mL, 24.2 mmol) was added. The mixture was stirred for 45 min, diluted with EtOAc (100 ml), washed with water (2×100 mL) and brine (100 mL). The organic phase was diluted with DCM (100 mL) to dissolve all solids, washed with brine (100 mL), dried (Na₂SO₄), filtered and then concentrated. The residue was triturated with EtOAc (25 mL) to give (E)-tert-butyl (3-fluoro-2-((4-(4-((tetrahydro-2H-pyran-4-yl)carbamoyl)bicyclo[2.2.2]octan-1-yl)phenoxy)methyl)allyl)carbamate (1.63 g, 91%) as a white solid. ¹H NMR (400 MHz, DMSO-d₆): δ 7.22 (d, 2H), 7.19 (d, 1H), 7.06 (t, 0.8H), 7.01 (d, J=83 Hz, 1H), 6.85 (d, 2H), 6.80-6.70 (m, 0.2H), 4.39 (d, 2H), 3.87-3.69 (m, 5H), 3.33-3.26 (m, 2H), 1.80-1.69 (m, 12H), 1.64-1.55 (m, 2H), 1.54-1.41 (m, 2H), 1.34 (s, 9H); LCMS: 517.3 [M+H]⁺.

### Step 2: (E)-4-(4-((2-(Aminomethyl)-3-fluoroallyl)oxy)phenyl)-N-(tetrahydro-2H-pyran-4-yl)bicyclo[2.2.2]octane-1-carboxamide Hydrochloride

Trifluoroacetic acid (6 mL) was added to a suspension of (E)-tert-butyl (3-fluoro-2-((4-(4-((tetrahydro-2H-pyran-4-yl)carbamoyl)bicyclo[2.2.2]octan-1-yl)phenoxy)methyl)allyl)carbamate (1.59 g, 3.08 mmol) in DCM (18 mL). The reaction was stirred at room temperature for 20 min, concentrated, and dissolved in methanol (10 mL). 2 M Hydrogen chloride in Et₂O (3.0 mL, 6.0 mmol) was added to the solution and the mixture concentrated to give (E)-4-(4-((2-(aminomethyl)-3-fluoroallyl)oxy)phenyl)-N-(tetrahydro-2H-pyran-4-yl)bicyclo[2.2.2]octane-1-carboxamide hydrochloride (1.37 g, 98%) as a white solid. ¹H NMR (400 MHz, DMSO-d₆): δ 8.21 (s, 3H), 7.28 (d, J=83 Hz, 1H), 7.26 (d, 2H), 7.19 (d, 1H), 6.91 (d, 2H), 4.58 (d, 2H), 3.87-3.71 (m, 3H), 3.65-3.55 (m, 2H), 3.35-3.26 (m, 2H), 1.80-1.70 (m, 12H), 1.63-1.55 (m, 2H), 1.54-1.41 (m, 2H); LCMS: 417.2 [M+H]⁺.

### Compound 2.82

### (E)-(4-(4-((2-(Aminomethyl)-3-fluoroallyl)oxy)phenyl)bicyclo[2.2.2]octan-1-yl)(7-oxa-2-azaspiro[3.5]nonan-2-yl)methanone Hydrochloride

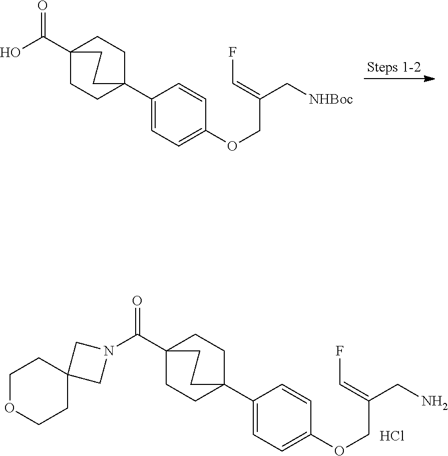 [SMILES: CC(C)(C)OC(=O)NC/C(=C\F)COc1ccc(C23CCC(C(=O)O)(CC2)CC3)cc1 ; NC/C(=C\F)COc1ccc(C23CCC(C(=O)N4CC5(CCOCC5)C4)(CC2)CC3)cc1 ; Cl]

### Step 1: (E)-tert-Butyl (2-((4-(4-(7-oxa-2-azaspiro[3.5]nonane-2-carbonyl)bicyclo[2.2.2]octan-1-yl)phenoxy)methyl)-3-fluoroallyl)carbamate

Diisopropylethylamine (1.8 mL, 10.3 mmol) was added to a solution of (E)-4-(4-((2-(((tert-butoxycarbonyl)amino)methyl)-3-fluoroallyl)oxy)phenyl)bicyclo[2.2.2]octane-1-carboxylic acid (1.50 g, 3.46 mmol), HATU (1.59 g, 4.18 mmol) and DMF (23 mL). After stirring the reaction mixture at room temperature for 15 min, 7-oxa-2-azaspiro[3.5]nonane hydrochloride (0.85 g, 5.21 mmol) was added. The mixture was stirred for 2 h, diluted with EtOAc (100 ml), washed with water (2×100 mL) and brine (100 mL), dried (Na₂SO₄), filtered and concentrated. The residue was triturated with MTBE (30 mL) to give (E)-tert-butyl (2-((4-(4-(7-oxa-2-azaspiro[3.5]nonane-2-carbonyl)bicyclo[2.2.2]octan-1-yl)phenoxy)methyl)-3-fluoroallyl)carbamate (1.66 g, 89%) as a white solid. ¹H NMR (400 MHz, DMSO-d₆): δ 7.21 (d, 2H), 7.06 (t, 0.8H), 7.01 (d, J=83 Hz, 1H), 6.85 (d, 2H), 6.80-6.70 (m, 0.2H). 4.39 (d, 2H), 4.10 (s, 2H), 3.74 (d, 2H), 3.60-3.43 (m, 6H), 1.86-1.70 (m, 12H), 1.69-1.60 (m, 4H), 1.34 (s, 9H); LCMS: 543.4 [M+H]⁺.

### Step 2: (E)-(4-(4-((2-(Aminomethyl)-3-fluoroallyl)oxy)phenyl)bicyclo[2.2.2]octan-1-yl)(7-oxa-2-azaspiro[3.5]nonan-2-yl)methanone Hydrochloride

Trifluoroacetic acid (5 mL) was added to a mixture of (E)-tert-butyl (2-((4-(4-(7-oxa-2-azaspiro[3.5]nonane-2-carbonyl)bicyclo[2.2.2]octan-1-yl)phenoxy)methyl)-3-fluoroallyl)carbamate (1.63 g, 3.00 mmol) in DCM (15 mL). The reaction was stirred at room temperature for 30 min, concentrated and dissolved in IPA (15 mL). 2 M Hydrogen chloride in Et₂O (3.0 mL, 6.0 mmol) was added to the solution. Additional IPA (20 mL) was added to the mixture to allow for better stirring. The resulting precipitate was collected by filtration and dried under reduced pressure to (E)-(4-(4-((2-(aminomethyl)-3-fluoroallyl)oxy)phenyl)bicyclo[2.2.2]octan-1-yl)(7-oxa-2-azaspiro[3.5]nonan-2-yl)methanone hydrochloride (1.08 g, 78%) as a white solid. ¹H NMR (400 MHz, DMSO-d₆): δ 8.16 (s, 3H), 7.28 (d, J=83 Hz, 1H), 7.24 (d, 2H), 6.90 (d, 2H), 4.57 (d, 2H), 4.10 (s, 2H), 3.65-3.43 (m, 8H), 1.86-1.70 (m, 12H), 1.69-1.59 (m, 4H); LCMS: 443.3 [M+H]⁺.

### Compound 2.95

### 4-(4-(((E)-2-(Aminomethyl)-3-fluoroallyl)oxy)phenyl)-N-(cis-3-methoxycyclobutyl)bicyclo[2.2.2]octane-1-carboxamide Hydrochloride

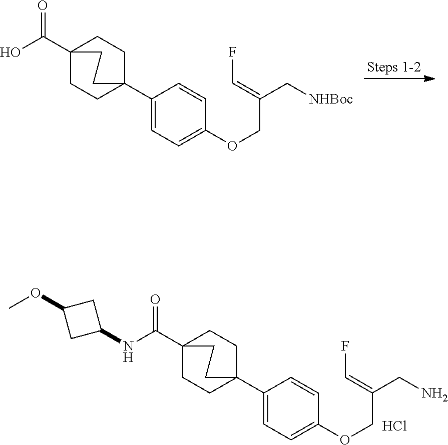

### Step 1: Tert-Butyl ((E)-3-fluoro-2-((4-(4-((cis-3-methoxycyclobutyl)carbamoyl)bicyclo[2.2.2]octan-1-yl)phenoxy)methyl)allyl)carbamate

Diisopropylethylamine (1.8 mL, 10.3 mmol) was added to a solution of (E)-4-(4-((2-(((tert-butoxycarbonyl)amino)methyl)-3-fluoroallyl)oxy)phenyl)bicyclo[2.2.2]octane-1-carboxylic acid (1.50 g, 3.46 mmol), HATU (1.59 g, 4.18 mmol) and DMF (25 mL). After stirring the reaction mixture at room temperature for 15 min, cis-3-methoxycyclobutanamine hydrochloride (0.72 g, 5.25 mmol) was added. The mixture was stirred for 1 h, diluted with EtOAc (100 mL), washed with water (2×100 mL) and brine (100 mL), dried (Na₂SO₄), filtered and concentrated. The residue was triturated with 1:1 MTBE:EtOAc (50 mL) to give tert-butyl ((E)-3-fluoro-2-((4-(4-((cis-3-methoxycyclobutyl)carbamoyl)bicyclo[2.2.2]octan-1-yl)phenoxy)methyl)allyl)carbamate (1.59 g, 89%) as a white solid. ¹H NMR (400 MHz, DMSO-d₆): δ 7.50 (d, 1H), 7.22 (d, 2H), 7.06 (t, 0.8H), 7.01 (d, J=83 Hz, 1H), 6.85 (d, 2H), 6.80-6.70 (m, 0.2H). 4.39 (d, 2H), 3.86-3.76 (m, 1H), 3.70-3.67 (m, 2H), 3.58-3.47 (m, 1H), 3.12 (s, 3H), 2.50-2.41 (m, 2H), 1.87-1.77 (m, 2H), 1.76-1.67 (m, 12H), 1.34 (s, 9H); LCMS: 517.3 [M+H]+.

### Step 2: 4-(4-(((E)-2-(Aminomethyl)-3-fluoroallyl)oxy)phenyl)-N-(cis-3-methoxycyclobutyl)bicyclo[2.2.2]octane-1-carboxamide Hydrochloride

Trifluoroacetic acid (5 mL) was added to a mixture of tert-butyl ((E)-3-fluoro-2-((4-(4-((cis-3-methoxycyclobutyl)carbamoyl)bicyclo[2.2.2]octan-1-yl)phenoxy)methyl)allyl)carbamate (1.57 g, 3.04 mmol) and DCM (15 mL). The reaction mixture was stirred at room temperature for 35 min, concentrated and dissolved in IPA (30 mL). 2 M Hydrogen chloride in Et₂O (3.0 mL, 6.0 mmol) was added to the solution and the resulting precipitate was collected by filtration to give 4-(4-(((E)-2-(aminomethyl)-3-fluoroallyl)oxy)phenyl)-N-(cis-3-methoxycyclobutyl)bicyclo[2.2.2]octane-1-carboxamide hydrochloride (1.19 g, 86%) as a white solid. ¹H NMR (400 MHz, DMSO-d₆): δ 8.21 (s, 3H), 7.52 (d, 1H), 7.29 (d, J=83 Hz, 1H), 7.25 (d, 2H), 6.90 (d, 2H), 4.58 (d, 2H), 3.86-3.74 (m, 1H), 3.63-3.49 (m, 3H), 3.12 (s, 3H), 2.50-2.42 (m, 2H), 1.88-1.78 (m, 2H), 1.77-1.66 (m, 12H); LCMS: 417.2 [M+H]⁺.

### Compound 4

### (E)-4-(4-((2-(Aminomethyl)-3-fluoroallyl)oxy)phenyl)-N-methylbicyclo[2.2.1]heptane-1-carboxamide Hydrochloride

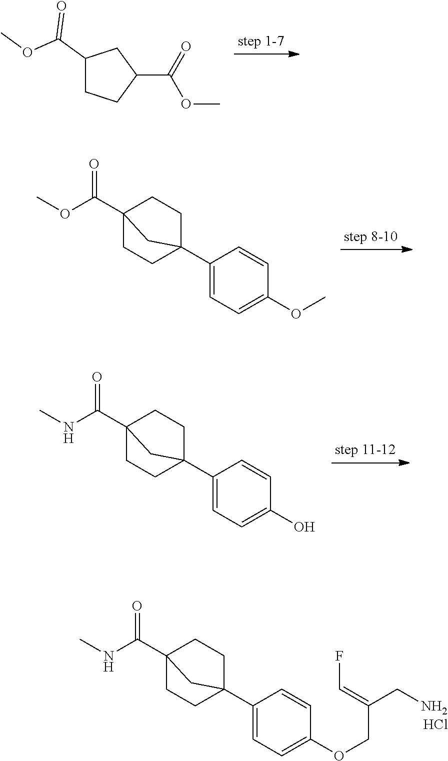

### Step 1: Dimethyl bicyclo[2.2.1]heptane-1,4-dicarboxylate

2.5 M n-BuLi in n-hexane (107 mL) was added slowly to a solution of diisopropylamine (37.2 g, 279 mmol) in anhydrous THF (60 mL) at −78° C. under a nitrogen atmosphere. The reaction was heated to 0° C. and stirred for 0.5 h at 0° C. DMPU (110 g, 859 mmol) was added via an addition funnel at 0° C., cooled to −78° C. and a solution of dimethyl cyclopentane-1,3-dicarboxylate (20.0 g, 107 mmol) in anhydrous THF (60 mL) added slowly via an addition funnel. The reaction was warmed to 0° C. and stirred for 0.5 h, cooled to −78° C. and treated with a solution of 1-bromo-2-chloroethane (15.4 g, 107 mmol) in anhydrous THF (60 mL). The reaction was warmed to room temperature and stirred at room temperature overnight. The mixture was poured into sat. aq. NH₄Cl (250 mL) and extracted with EtOAc (3×250 mL). The organic layers were combined, washed with brine (2×250 mL), dried (Na₂SO₄), filtered, concentrated and then purified by chromatography on silica gel (petroleum ether/ethyl acetate=2/1) to give dimethyl bicyclo[2.2.1]heptane-1,4-dicarboxylate (11.5 g, 50%) as a white solid. ¹H NMR (400 MHz, CDCl₃): δ 3.67 (s, 6H), 2.02 (d, 4H), 1.90 (s, 2H), 1.67 (d, 4H); LCMS:213.1 [M+H]⁺.

### Step 2: 4-(Methoxycarbonyl)bicyclo[2.2.1]heptane-1-carboxylic Acid

To a solution of dimethyl bicyclo[2.2.1]heptane-1,4-dicarboxylate (6.5 g, 30.6 mmol) in THF (80 mL) a solution of NaOH (1.10 g, 27.6 mmol) in MeOH (8 mL) was added at room temperature. The mixture was warmed to 40° C. and stirred at 40° C. overnight. The mixture was concentrated to give the crude product and then diluted with water (30 ml) and extracted with DCM (2×30 mL). The aqueous phase was adjusted to pH=3 with 1 M HCl and then extracted with DCM (2×30 mL). The organic layers were combined, washed with brine (25 ml), dried (Na₂SO₄), filtered and concentrated to give 4-(methoxycarbonyl)bicyclo[2.2.1]heptane-1-carboxylic acid (3.0 g, crude) as a white solid. ¹H NMR (400 MHz, CDCl₃): δ 12.09-10.65 (m, 1H), 3.70-3.68 (m, 3H), 2.10-2.03 (m, 4H), 1.94 (s, 2H), 1.76-1.66 (m, 4H); LCMS: 197.1 [M−H]⁻.

### Step 3: Methyl 4-(chlorocarbonyl)bicyclo[2.2.1]heptane-1-carboxylate

To a solution of 4-(methoxycarbonyl)bicyclo[2.2.1]heptane-1-carboxylic acid (3.0 g, 15.1 mmol), DMF (55.3 mg, 0.76 mmol) in DCM (60 mL), (COCl)₂ (2.88 g, 22.7 mmol) was added. The mixture was stirred at room temperature for 3 h. The mixture concentrated to give methyl 4-(chlorocarbonyl)bicyclo[2.2.1]heptane-1-carboxylate (3.28 g, crude) as a yellow oil.

### Step 4: 1-(1,3-Dioxoisoindolin-2-yl) 4-methyl bicyclo[2.2.1]heptane-1,4-dicarboxylate

To a solution of 2-hydroxyisoindoline-1,3-dione (2.47 g, 15.1 mmol), pyridine (7.18 g, 90.8 mmol) in DCM (50 mL), methyl 4-(chlorocarbonyl)bicyclo[2.2.1]heptane-1-carboxylate (3.28 g, 15.1 mmol) in DCM (20 mL) was added. The mixture was stirred at room temperature overnight. The mixture was diluted with DCM (35 mL), washed with 1M HCl (3×35 mL), washed with NaHCO₃ (2×35 mL), brine (35 mL), dried (Na₂SO₄), filtered, concentrated and then purified by chromatography on silica gel (petroleum ether/ethyl acetate=2/1) to give 1-(1,3-dioxoisoindolin-2-yl) 4-methyl bicyclo[2.2.1]heptane-1,4-dicarboxylate (4.2 g, 80%) as a white solid. ¹H NMR (400 MHz, CDCl₃): δ 7.89 (d, 2H), 7.61 (d, 2H), 3.72 (s, 3H), 2.34-2.24 (m, 2H), 2.20-2.10 (m, 4H), 1.98-1.89 (m, 2H), 1.84-1.75 (m, 2H).

### Step 5: (4-Methoxyphenyl)magnesium Lithium Bromide Chloride

In an oven-dried 250 mL 3-necked flask equipped with a double manifold, Mg (1.09 g, 44.9 mmol) and LiCl (1.90 g, 44.9 mmol) were weighed. The flask was sealed, evacuated and purged with nitrogen 3 times. THF (60 mL) was added at room temperature and stirred for 15 minutes. 1 M DIBAL-H in toluene (0.6 mL) was added dropwise at room temperature and stirred for 15 minutes. The mixture was cooled to 0° C. and 1-bromo-4-methoxybenzene (5.6 g, 29.9 mmol) in THF (20 mL) added dropwise. The mixture was warmed to room temperature and stirred at room temperature for 1.5 h to give (4-methoxyphenyl)magnesium lithium bromide chloride (7.6 g, crude) as a solution in THF.

### Step 6: Bis(4-methoxyphenyl)zinc

To a solution of (4-methoxyphenyl)magnesium lithium bromide chloride (29.9 mmol) in THF at room temperature, 1M ZnCl₂ in THF (18 mL) was added dropwise. The mixture was stirred at room temperature for 1 h to give bis(4-methoxyphenyl)zinc (4.19 g, crude) as a solution in THF.

### Step 7: Methyl 4-(4-methoxyphenyl)bicyclo[2.2.1]heptane-1-carboxylate

To a mixture of Ni(acac)₂ (748 mg, 2.91 mmol), 2,2′-bipyridine (573 mg, 3.67 mmol), 1-(1,3-dioxoisoindolin-2-yl) 4-methyl bicyclo[2.2.1]heptane-1,4-dicarboxylate (2.0 g, 5.83 mmol) in DMF (20 mL) at 0° C., bis(4-methoxyphenyl)zinc (4.19 g, 15.0 mmol) was added. The mixture was warmed to room temperature and stirred at room temperature overnight. Water (20 ml) was added, the reaction stirred at room temperature for 5 minutes. 1M HCl (40 mL) was added and the reaction stirred at room temperature 0.5 h. The mixture was extracted with EtOAc (3×60 mL). The organic layers were combined, washed with NaHCO₃ (60 mL), brine (60 mL), dried (Na₂SO₄), filtered, concentrated and purified by chromatography on silica gel (petroleum ether/ethyl acetate=10/1) to give methyl 4-(4-methoxyphenyl)bicyclo[2.2.1]heptane-1-carboxylate (2 g, crude) as a white solid. ¹H NMR (400 MHz, CDCl₃): δ 7.15-7.04 (m, 2H), 6.79-6.75 (m, 2H), 3.76 (s, 6H), 2.11-1.98 (m, 2H), 1.93-1.87 (m, 2H), 1.79-1.71 (m, 6H); LCMS: 261.2 [M+H]⁺.

### Step 8: 4-(4-Methoxyphenyl)bicyclo[2.2.1]heptane-1-carboxylic Acid

A mixture of methyl 4-(4-methoxyphenyl)bicyclo[2.2.1]heptane-1-carboxylate (2.0 g, 3.84 mmol), LiOH.H₂O (483 mg, 11.5 mmol), THF (100 mL), MeOH (20 mL) and H₂O (20 mL) was stirred at room temperature overnight. The mixture was concentrated to remove organic solvents. Water (30 ml) was added and the mixture extracted with EtOAc (3×30 mL). The aqueous phase was adjusted to pH=3 with 1M HCl and extracted with EtOAc (3×30 mL). The organic layers were combined, washed with brine (25 ml), dried (Na₂SO₄), filtered and concentrated to give 4-(4-methoxyphenyl)bicyclo[2.2.1]heptane-1-carboxylic acid (400 mg, 34%) as white solid. ¹H NMR (400 MHz, CDCl₃): δ 11.37-10.40 (m, 1H), 7.22 (d, 2H), 6.87 (d, 2H), 3.80 (s, 3H), 2.17-2.13 (m, 2H), 2.05-2.01 (m, 2H), 1.91-1.78 (m, 6H); LCMS: 245.1 [M−H]⁻.

### Step 9: 4-(4-Methoxyphenyl)-N-methylbicyclo[2.2.1]heptane-1-carboxamide

A mixture of 4-(4-methoxyphenyl)bicyclo[2.2.1]heptane-1-carboxylic acid (150 mg, 0.61 mmol), DIPEA (157 mg, 1.22 mmol), HATU (347 mg, 0.91 mmol), methanamine hydrochloride (82 mg, 1.22 mmol) and DCM (10 mL) was stirred at 30° C. overnight. The reaction mixture was poured into water (10 mL) and then extracted with DCM (3×10 mL). The combined organic layers were washed with H₂O (10 mL), brine (10 mL), dried (Na₂SO₄), filtered, concentrated and then purified by column chromatography (SiO₂, petroleum ether/ethyl acetate=0/1) to give 4-(4-methoxyphenyl)-N-methylbicyclo[2.2.1]heptane-1-carboxamide (110 mg, 70%) as a white solid. ¹H NMR (400 MHz, CDCl₃): δ 7.21 (d, 2H), 6.86 (d, 2H), 5.63-5.61 (m, 1H), 3.80 (s, 3H), 2.86 (d, 3H), 2.11-2.00 (m, 2H), 1.97 (s, 2H), 1.92-1.83 (m, 4H), 1.81-1.73 (m, 2H) MS: 260. [M+H]⁺.

### Step 10: 4-(4-Hydroxyphenyl)-N-methylbicyclo[2.2.1]heptane-1-carboxamide

To a solution of 4-(4-methoxyphenyl)-N-methylbicyclo[2.2.1]heptane-1-carboxamide (150 mg, 0.58 mmol) in DCM (15 mL), BBr₃ (430 mg, 1.72 mmol) was slowly added at −78° C. and stirred at −78° C. for 1 h. The mixture was warmed to room temperature and stirred at room temperature for 2 h. The reaction mixture was slowly poured into MeOH (30 mL) and then concentrated to give 4-(4-hydroxyphenyl)-N-methylbicyclo[2.2.1]heptane-1-carboxamide (150 mg, crude) as yellow solid. MS: 244.1 [M−H]⁻.

### Step 11: (E)-tert-Butyl (3-fluoro-2-((4-(4-(methylcarbamoyl)bicyclo[2.2.1]heptan-1-yl)phenoxy)methyl)allyl)carbamate

A solution of 4-(4-hydroxyphenyl)-N-methylbicyclo[2.2.1]heptane-1-carboxamide (150 mg, 0.61 mmol), (E)-tert-butyl (2-(bromomethyl)-3-fluoroallyl)carbamate (197 mg, 0.73 mmol), Cs₂CO₃ (598 mg, 1.83 mmol) and MeCN (10 mL) was stirred at room temperature overnight. The reaction mixture was poured into water (15 mL) and then extracted with EtOAc (2×20 mL). The combined organic layers were washed with H₂O (20 mL), brine (20 mL), dried (Na₂SO₄), filtered, concentrated and then purified by column chromatography (SiO₂, petroleum ether/ethyl acetate=0/1) to give (E)-tert-butyl (3-fluoro-2-((4-(4-(methylcarbamoyl)bicyclo[2.2.1]heptan-1-yl)phenoxy)methyl)allyl)carbamate (160 mg, 61%) as a yellow oil. ¹H NMR (400 MHz, CDCl₃): δ 7.22 (d, 2H), 6.87 (d, 2H), 6.76 (d, 1H), 5.68-5.54 (m, 1H), 4.84-4.68 (m, 1H), 4.49-4.41 (m, 2H), 4.08-3.98 (m, 2H), 2.87 (d, 3H), 2.11-2.03 (m, 2H), 2.00-1.94 (m, 2H), 1.91-1.72 (m, 6H), 1.47-1.40 (s, 9H); LCMS:455.2 [M+Na]⁺.

### Step 12: (E)-4-(4-((2-(Aminomethyl)-3-fluoroallyl)oxy)phenyl)-N-methylbicyclo[2.2.1]heptane-1-carboxamide Hydrochloride

To a solution of (E)-tert-butyl (3-fluoro-2-((4-(4-(methylcarbamoyl)bicyclo[2.2.1]heptan-1-yl)phenoxy)methyl)allyl)carbamate (140 mg, 0.32 mmol) in DCM (7 mL) at room temperature TFA (2 mL) was added and the reaction stirred at room temperature for 0.5 h. The mixture was concentrated to dryness and purified by reverse-phase HPLC (water(0.04% HCl)-MeCN) to give (E)-4-(4-((2-(aminomethyl)-3-fluoroallyl)oxy)phenyl)-N-methylbicyclo[2.2.1]heptane-1-carboxamide hydrochloride (61 mg, 54%) as a white solid. ¹H NMR (400 MHz, DMSO-d₆): δ 8.28-8.07 (m, 3H), 7.52-7.46 (m, 1H), 7.28 (d, 1H), 7.25-7.15 (d, 2H), 6.98-6.86 (d, 2H), 4.57 (d, 2H), 3.58 (d, 2H), 2.58 (d, 3H), 1.95-1.61 (m, 10H); LCMS: 333.1 [M+H]⁺.

### Compound 4.01

### (E)-4-(4-((2-(Aminomethyl)-3-fluoroallyl)oxy)phenyl)-N-(tetrahydro-2H-pyran-4-yl)bicyclo[2.2.1]heptane-1-carboxamide Hydrochloride

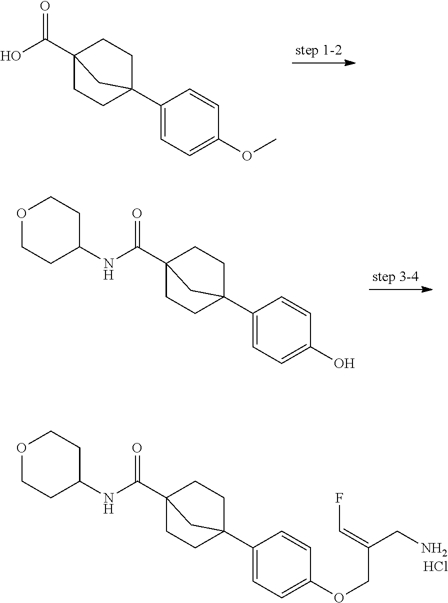 [SMILES: COc1ccc(C23CCC(C(=O)O)(CC2)C3)cc1 ; O=C(NC1CCOCC1)C12CCC(c3ccc(O)cc3)(CC1)C2 ; NC/C(=C\F)COc1ccc(C23CCC(C(=O)NC4CCOCC4)(CC2)C3)cc1 ; Cl]

### Step 1: 4-(4-Methoxyphenyl)-N-(tetrahydro-2H-pyran-4-yl)bicyclo[2.2.1]heptane-1-carboxamide

A mixture of 4-(4-methoxyphenyl)bicyclo[2.2.1]heptane-1-carboxylic acid (200 mg, 0.81 mmol), tetrahydro-2H-pyran-4-amine (164 mg, 1.62 mmol), HATU (463 mg, 1.22 mmol), DIEA (210 mg, 1.62 mmol) and DCM (10 mL) was stirred at room temperature overnight. The reaction mixture was poured into water (20 mL) and extracted with DCM (2×20 mL). The combined organic layers were washed with H₂O (20 mL), brine (20 mL), dried (Na₂SO₄), filtered, concentrated and then purified by column chromatography (SiO₂, petroleum ether/ethyl acetate=0/1) to give 4-(4-methoxyphenyl)-N-(tetrahydro-2H-pyran-4-yl)bicyclo[2.2.1]heptane-1-carboxamide (220 mg, 82%) as a white solid. ¹H NMR (400 MHz, CDCl₃) δ 7.21 (d, 2H), 6.86 (d, 2H), 5.48-5.40 (m, 1H), 4.00-3.92 (m, 2H), 3.54-3.44 (m, 2H), 2.82-2.80 (m, 4H), 2.05-1.99 (m, 2H), 1.96 (s, 2H), 1.94-1.83 (m, 6H), 1.82-1.73 (m, 2H), 1.53-1.41 (m, 2H); LCMS: 330.1 [M+H]⁺.

### Step 2: 4-(4-Hydroxyphenyl)-N-(tetrahydro-2H-pyran-4-yl)bicyclo[2.2.1]heptane-1-carboxamide

To a solution of 4-(4-methoxyphenyl)-N-(tetrahydro-2H-pyran-4-yl)bicyclo[2.2.1]heptane-1-carboxamide (200 mg, 0.61 mmol) in DCM (10 mL), BBr₃ (910 mg, 3.63 mmol) in DCM (1 mL) was slowly added at −78° C. under a nitrogen atmosphere. The mixture was stirred at −78° C. for 1 h. The mixture was warmed to room temperature and stirred at room temperature for 3 h. The mixture was slowly poured into MeOH (10 ml), stirred at room temperature for 0.5 h and concentrated to give 4-(4-hydroxyphenyl)-N-(tetrahydro-2H-pyran-4-yl)bicyclo[2.2.1]heptane-1-carboxamide (190 mg, crude) as a yellow oil. LCMS: 314.1 [M−H]⁻.

### Step 3: (E)-tert-Butyl (3-fluoro-2-((4-(4-((tetrahydro-2H-pyran-4-yl)carbamoyl)bicyclo[2.2.1]heptan-1-yl)phenoxy)methyl)allyl)carbamate

A mixture of 4-(4-hydroxyphenyl)-N-(tetrahydro-2H-pyran-4-yl)bicyclo[2.2.1]heptane-1-carboxamide (190 mg, 0.60 mmol), (E)-tert-butyl (2-(bromomethyl)-3-fluoroallyl)carbamate (162 mg, 0.60 mmol), Cs₂CO₃ (1.96 g, 6.02 mmol) and MeCN (10 mL) was stirred at room temperature for 3 h, poured into water (15 mL) and extracted with EtOAc (2×20 mL). The combined organic layers were washed with H₂O (20 mL), brine (20 mL), dried (Na₂SO₄), filtered, concentrated and then purified by prep-TLC (SiO₂, petroleum ether/ethyl acetate=0/1) to give (E)-tert-butyl (3-fluoro-2-((4-(4-((tetrahydro-2H-pyran-4-yl)carbamoyl)bicyclo[2.2.1]heptan-1-yl)phenoxy)methyl)allyl)carbamate (170 mg, 56%) as a yellow oil. ¹H NMR (400 MHz, CDCl₃) δ 7.21 (d, 2H), 6.86 (d, 2H), 6.75 (d, 1H), 5.47-5.38 (m, 1H), 4.84-4.72 (m, 1H), 4.43 (d, 2H), 4.05-3.89 (m, 5H), 3.55-3.46 (m, 2H), 2.05-2.01 (m, 2H), 1.97-1.83 (m, 8H), 1.82-1.73 (m, 2H), 1.45-1.39 (m, 11H); LCMS: 503.2 [M+H]⁺.

### Step 4: (E)-4-(4-((2-(Aminomethyl)-3-fluoroallyl)oxy)phenyl)-N-(tetrahydro-2H-pyran-4-yl)bicyclo[2.2.1]heptane-1-carboxamide Hydrochloride

To a solution of (E)-tert-butyl (3-fluoro-2-((4-(4-((tetrahydro-2H-pyran-4-yl)carbamoyl)bicyclo[2.2.1]heptan-1-yl)phenoxy)methyl)allyl)carbamate (160 mg, 0.32 mmol) in DCM (5 mL) at room temperature TFA (2 mL) was added and the reaction stirred at room temperature for 1 h. The mixture was concentrated to dryness and then purified by reverse-phase HPLC (water(0.04% HCl)-MeCN) to give (E)-4-(4-((2-(aminomethyl)-3-fluoroallyl)oxy)phenyl)-N-(tetrahydro-2H-pyran-4-yl)bicyclo[2.2.1]heptane-1-carboxamide hydrochloride (64 mg, 46%) as a white solid ¹H NMR (400 MHz, DMSO-d₆): δ 8.15-7.95 (m, 3H), 7.32 (d, 1H), 7.29 (d, 1H), 7.26-7.18 (m, 2H), 6.92 (d, 2H), 4.56 (d, 2H), 3.86-3.77 (m, 3H), 3.61 (d, 2H), 3.32 (d, 2H), 1.95-1.83 (m, 4H), 1.78-1.60 (m, 8H), 1.52-1.43 (m, 2H); LCMS: 403.3 [M+H]⁺.

### Compound 5

### 5-(4-(((E)-2-(Aminomethyl)-3-fluoroallyl)oxy)phenyl)-N-methylbicyclo[3.2.1]octane-1-carboxamide Hydrochloride

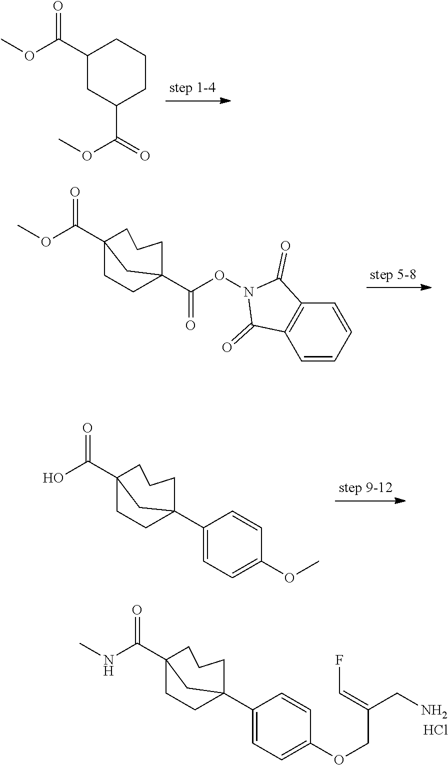

### Step 1: Dimethyl bicyclo[3.2.1]octane-1,5-dicarboxylate

2.5 M n-BuLi in n-hexane (37 mL) was added slowly into a solution of diisopropylamine (9.85 g, 97.4 mmol) in anhydrous THF (37 mL) at −78° C. under a nitrogen atmosphere. The reaction mixture was warmed to 0° C. and stirred at 0° C. for 0.5 h. DMPU (38.4 g, 300 mmol) was added via an addition funnel at 0° C. The reaction was cooled to −78° C. and a solution of dimethyl cyclohexane-1,3-dicarboxylate (7.5 g, 37.5 mmol) in anhydrous THF (37 mL) was added slowly via an addition funnel. The reaction was warmed to 0° C. and stirred for 0.5 h, cooled to −78° C. and treated with a solution of 1-bromo-2-chloroethane (9.13 g, 63.7 mmol) in anhydrous THF (37 mL). The reaction was warmed slowly to room temperature and stirred at room temperature overnight. The reaction mixture was poured into saturated aqueous NH₄Cl (200 mL) at 0° C. and extracted with EtOAc (3×200 mL). The combined organic layers were washed with brine (300 mL), dried (Na₂SO₄), filtered, concentrated and purified by chromatography on silica gel (petroleum ether/EtOAc=2/1) to give dimethyl bicyclo[3.2.1]octane-1,5-dicarboxylate (5.6 g, 66%) as a colorless oil. ¹H NMR (400 MHz, CDCl₃): δ 3.68 (s, 6H), 2.31-2.28 (m, 1H), 2.11-2.09 (m, 2H), 2.00-1.98 (m, 1H), 1.81-1.72 (m, 6H), 1.65-1.61 (m, 2H); LCMS: 227.1 [M+H]⁺.

### Step 2: 5-(Methoxycarbonyl)bicyclo[3.2.1]octane-1-carboxylic Acid

To a solution of dimethyl bicyclo[3.2.1]octane-1,5-dicarboxylate (5.0 g, 22.1 mmol) in THF (50 mL) at room temperature a solution of NaOH (795 mg, 19.9 mmol) in MeOH (5 mL) was added. The resulting mixture was stirred at 30° C. overnight. The reaction mixture was concentrated under reduced pressure, the residue diluted with H₂O (30 mL) and extracted with DCM (3×35 mL). The aqueous layer was acidified with HCl (1 M) to pH=3 and extracted with DCM (3×30 mL). The combined organic layers were washed with brine (35 mL), dried (Na₂SO₄), filtered and concentrated under reduced pressure to give 5-(methoxycarbonyl)bicyclo[3.2.1]octane-1-carboxylic acid (2.2 g, crude) as a white solid. ¹H NMR (400 MHz, CDCl₃): δ 11.12 (s, 1H), 3.69 (s, 3H), 2.35-2.31 (m, 1H), 2.18-2.12 (m, 2H), 2.07-1.97 (m, 1H), 1.84-1.74 (m, 6H), 1.69-1.64 (m, 2H); LCMS: 211.1 [M−H]⁻

### Step 3: Methyl 5-(chlorocarbonyl)bicyclo[3.2.1]octane-1-carboxylate

To a solution of 5-(methoxycarbonyl)bicyclo[3.2.1]octane-1-carboxylic acid (3.2 g, crude) and DMF (55 mg, 0.75 mmol) in DCM (32 mL) at room temperature, (COCl)₂ (2.3 g, 18.1 mmol) was added slowly. The mixture was stirred at room temperature for 1 h. The reaction mixture was concentrated under reduced pressure to give methyl 5-(chlorocarbonyl)bicyclo[3.2.1]octane-1-carboxylate (3.48 g, crude) as yellow oil.

### Step 4: 1-(1,3-dioxoisoindolin-2-yl) 5-methyl bicyclo[3.2.1]octane-1,5-dicarboxylate

To a solution of 2-hydroxyisoindoline-1,3-dione (2.46 g, 15.1 mmol) in DCM (34 mL) at room temperature pyridine (7.16 g, 90.5 mmol) was added. A solution of methyl 5-(chlorocarbonyl)bicyclo[3.2.1]octane-1-carboxylate (3.48 g, crude) in DCM (34 mL) was added dropwise at 0° C., the mixture warmed to room temperature and stirred at room temperature overnight. The reaction mixture was diluted with H₂O (70 mL) and extracted with DCM (3×70 mL). The combined organic layers were washed with brine (3×70 mL), dried (Na₂SO₄), filtered, concentrated and then purified by reverse-phase HPLC (water (0.04% HCl)/MeOH) to give 1-(1,3-dioxoisoindolin-2-yl) 5-methyl bicyclo[3.2.1]octane-1,5-dicarboxylate (1.25 g, 23%) as a white solid. ¹H NMR (400 MHz, CDCl₃): δ 7.94-7.84 (m, 2H), 7.84-7.75 (m, 2H), 3.71 (s, 3H), 2.52 (d, 1H), 2.42-2.27 (m, 1H), 2.22-2.20 (m, 1H), 2.12-1.83 (m, 7H), 1.82-1.64 (m, 2H).

### Step 5: (4-Methoxyphenyl)magnesium Lithium Bromide Chloride

To a mixture of Mg (287 mg, 11.8 mmol), LiCl (751 mg, 17.7 mmol) in THF (22 mL) at room temperature, 1 M DIBAL-H in toluene (0.24 mL) was added. The reaction mixture was stirred at room temperature for 0.25 h. 1-Bromo-4-methoxybenzene (2.21 g, 11.8 mmol) was added at 0° C., the mixture warmed to room temperature and stirred at room temperature for 1 h. The resulting bromo-(4-methoxyphenyl)magnesium solution in THF was used directly in subsequent reactions.

### Step 6: Bis(4-methoxyphenyl)zinc

To a solution of bromo-(4-methoxyphenyl)magnesium in THF at room temperature, 1M ZnCl₂ in THF (11.8 mL) was added. The mixture was stirred at room temperature for 1 h. The resulting bis(4-methoxyphenyl)zinc (11.8 mmol) solution in THF was used directly in subsequent reactions.

### Step 7: Methyl 5-(4-methoxyphenyl)bicyclo[3.2.1]octane-1-carboxylate

To a solution of 1-(1,3-dioxoisoindolin-2-yl) 5-methyl-bicyclo[3.2.1]octane-1,5-dicarboxylate (0.85 g, 2.38 mmol) 2-(2-pyridyl)pyridine (186 mg, 1.19 mmol) and bis[(Z)-1-methyl-3-oxo-but-1-enoxy]nickel (385 mg, 1.50 mmol) in DMF (10 mL) at room temperature was added a solution of bis(4-methoxyphenyl)zinc in THF. The resulting mixture was stirred at room temperature overnight. The mixture was poured into water (50 mL), adjusted to pH=2 with 1 M HCl and extracted with EtOAc (4×50 mL). The organic layers were combined, washed with brine (100 mL), dried (Na₂SO₄), filtered, concentrated and purified by chromatography on silica gel (petroleum ether/EtOAc=10/1) to give methyl 5-(4-methoxyphenyl)bicyclo[3.2.1]octane-1-carboxylate (544 mg, crude) as a white solid. LCMS: 275.2 [M+H]⁺.

### Step 8: 5-(4-Methoxyphenyl)bicyclo[3.2.1]octane-1-carboxylic Acid

To a solution of methyl 5-(4-methoxyphenyl)bicyclo[3.2.1]octane-1-carboxylate (0.70 g, 2.55 mmol) in THF (35 mL) at room temperature, LiOH.H₂O (321 mg, 7.65 mmol), MeOH (3.5 mL) and H₂O (3.5 mL) were added. The mixture was stirred at room temperature overnight. The reaction mixture was concentrated to dryness, diluted with H₂O (20 mL) and extracted with DCM (2×20 mL). The aqueous layer was acidified with 1M HCl to pH=3 and extracted with DCM (3×20 mL). The combined organic layers were washed with brine (20 mL), dried (Na₂SO₄), filtered and concentrated under reduced pressure to give 5-(4-methoxyphenyl)bicyclo[3.2.1]octane-1-carboxylic acid (280 mg, crude) as a white solid. ¹H NMR (400 MHz, CDCl₃): δ 11.24 (s, 1H), 7.18-7.15 (m, 2H), 6.89-6.83 (m, 2H), 3.80 (s, 3H), 2.33-2.15 (m, 2H), 2.09-2.02 (m, 1H), 1.99-1.94 (m, 1H), 1.91-1.69 (m, 7H), 1.54-1.47 (m, 1H); LCMS: 259.1 [M−H]⁻.

### Step 9: 5-(4-Methoxyphenyl)-N-methylbicyclo[3.2.1]octane-1-carboxamide

To a solution of 5-(4-methoxyphenyl)bicyclo[3.2.1]octane-1-carboxylic acid (150 mg, 0.58 mmol) and HATU (329 mg, 0.86 mmol) in DCM (7 mL) at 0° C., DIPEA (745 mg, 5.76 mmol) was added followed by methanamine hydrochloride (85 mg, 1.27 mmol) at 0° C. The mixture was stirred at 0° C. for 0.5 h and then warmed to 30° C. overnight. The reaction mixture was diluted with H₂O (10 mL), acidified to pH=4 with 1 M HCl and extracted with DCM (3×10 mL). The combined organic layers were washed with brine (10 mL), dried (Na₂SO₄), filtered, concentrated and purified by prep-TLC (petroleum ether/EtOAc=0/1) to give 5-(4-methoxyphenyl)-N-methylbicyclo[3.2.1]octane-1-carboxamide (130 mg, 83%) as a colorless oil. ¹H NMR (400 MHz, CDCl₃): δ 7.17 (d, 2H), 6.84 (d, 2H), 5.79 (d, 1H), 3.80 (s, 3H), 2.85 (d, 3H), 2.20 (br d, 1H), 2.11-2.03 (m, 2H), 1.97-1.79 (m, 6H), 1.71-1.62 (m, 2H), 1.54-1.46 (m, 1H); LCMS: 274.2 [M+H]⁺.

### Step 10: 5-(4-Hydroxyphenyl)-N-methylbicyclo[3.2.1]octane-1-carboxamide

To a solution of 5-(4-methoxyphenyl)-N-methylbicyclo[3.2.1]octane-1-carboxamide (141 mg, 0.52 mmol) in DCM (15 mL) at −78° C., BBr₃ (388 mg, 1.55 mmol) was added dropwise. The mixture was warmed slowly to room temperature and stirred at room temperature overnight. The reaction mixture was poured into MeOH (20 mL) at 0° C. and concentrated to give 5-(4-hydroxyphenyl)-N-methylbicyclo[3.2.1]octane-1-carboxamide (150 mg, crude) as a red solid. LCMS: 258.1 [M−H]⁻.

### Step 11: tert-Butyl ((E)-3-fluoro-2-((4-(5-(methylcarbamoyl)bicyclo[3.2.1]octan-1-yl)phenoxy)methyl)allyl)carbamate

To a mixture of 5-(4-hydroxyphenyl)-N-methylbicyclo[3.2.1]octane-1-carboxamide (150 mg, crude) in MeCN (15 mL) at room temperature, tert-butyl (E)-(2-(bromomethyl)-3-fluoroallyl)carbamate (186.1 mg, 0.69 mmol) and Cs₂CO₃ (565 mg, 1.74 mmol) were added. The resulting mixture was stirred at room temperature overnight, diluted with H₂O (30 mL) and extracted with EtOAc (3×30 mL). The combined organic layers were washed with brine (45 mL), dried (Na₂SO₄), filtered, concentrated and purified by prep-TLC (petroleum ether/EtOAc=0/1) to give tert-butyl ((E)-3-fluoro-2-((4-(5-(methylcarbamoyl)bicyclo[3.2.1]octan-1-yl)phenoxy)methyl)allyl)carbamate (210 mg, 75%) as a white solid. ¹H NMR (400 MHz, CDCl₃): δ 7.16 (d, 2H), 6.84 (d, 2H), 6.74 (d, 1H), 5.60 (d, 1H), 4.78 (s, 1H), 4.42 (d, 2H), 4.01 (d, 2H), 2.84 (d, 3H), 2.19 (d, 1H), 2.13-2.00 (m, 2H), 1.97-1.76 (m, 6H), 1.69-1.61 (m, 2H), 1.53-1.45 (m, 1H), 1.42 (s, 9H); LCMS: 447.3 [M+H]⁺.

### Step 12: 5-(4-(((E)-2-(Aminomethyl)-3-fluoroallyl)oxy)phenyl)-N-methylbicyclo[3.2.1]octane-1-carboxamide Hydrochloride

To a solution of tert-butyl ((E)-3-fluoro-2-((4-(5-(methylcarbamoyl)bicyclo[3.2.1]octan-1-yl)phenoxy)methyl)allyl)carbamate (175 mg, 0.39 mmol) in DCM (20 mL) at room temperature, TFA (10.8 g, 94.5 mmol) was added. The resulting mixture was stirred at room temperature for 0.5 h. The reaction mixture was concentrated under reduced pressure to dryness and then purified by reverse-phase HPLC (water (0.04% HCl)/MeCN) to give 5-(4-(((E)-2-(aminomethyl)-3-fluoroallyl)oxy)phenyl)-N-methylbicyclo[3.2.1]octane-1-carboxamide hydrochloride (83 mg, 55%) as a white solid. ¹H NMR (400 MHz, DMSO-d₆): δ 8.31-8.21 (m, 3H), 7.47 (d, 1H), 7.27 (d, 1H), 7.17 (d, 2H), 6.91 (d, 2H), 4.59 (d, 2H), 3.59 (d, 2H), 2.57 (d, 3H), 2.03-1.84 (m, 3H), 1.78-1.64 (m, 6H), 1.58-1.44 (m, 2H), 1.40-1.34 (m, 1H); LCMS: 347.2 [M+H]⁺.

### Compound 5.01

### 5-(4-(((E)-2-(Aminomethyl)-3-fluoroallyl)oxy)phenyl)-N-(tetrahydro-2H-pyran-4-yl)bicyclo[3.2.1]octane-1-carboxamide Hydrochloride

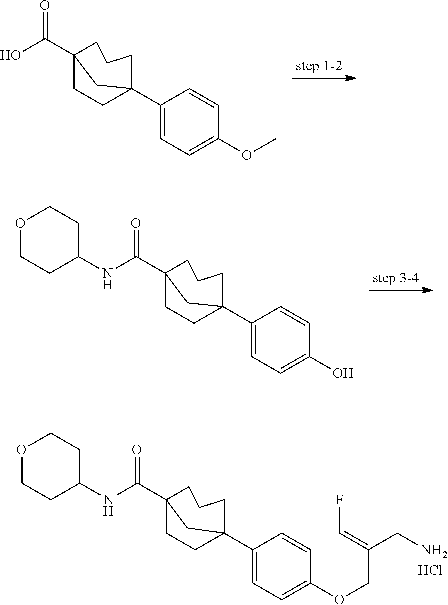 [SMILES: COc1ccc(C23CCCC(C(=O)O)(CC2)C3)cc1 ; O=C(NC1CCOCC1)C12CCCC(c3ccc(O)cc3)(CC1)C2 ; NC/C(=C\F)COc1ccc(C23CCCC(C(=O)NC4CCOCC4)(CC2)C3)cc1 ; Cl]

### Step 1: 5-(4-Methoxyphenyl)-N-(tetrahydro-2H-pyran-4-yl)bicyclo[3.2.1]octane-1-carboxamide

To a solution of 5-(4-methoxyphenyl)bicyclo[3.2.1]octane-1-carboxylic acid (80 mg, 0.31 mmol) in DCM (6 mL) at 0° C., HATU (175 mg, 0.46 mmol) and DIPEA (397 mg, 3.07 mmol) were added. Tetrahydro-2H-pyran-4-amine (62 mg, 0.61 mmol) was added at 0° C., the mixture was stirred at 0° C. for 0.5 h, heated to 30° C. and stirred at 30° C. overnight. The reaction mixture was diluted with H₂O (8 mL), acidified to pH=4 with 1M HCl and extracted with DCM (3×8 mL). The combined organic layers were washed with brine (10 mL), dried (Na₂SO₄), filtered, concentrated and purified by prep-TLC (petroleum ether/EtOAc=0/1) to give 5-(4-methoxyphenyl)-N-(tetrahydro-2H-pyran-4-yl)bicyclo[3.2.1]octane-1-carboxamide (90 mg, crude) as a colorless oil. ¹H NMR (400 MHz, CDCl₃): δ 7.17 (d, 2H), 6.85 (d, 2H), 5.42 (d, 1H), 4.07-3.91 (m, 3H), 3.80 (s, 3H), 3.49 (d, 2H), 2.16 (d, 1H), 2.10-2.01 (m, 2H), 1.98-1.77 (m, 8H), 1.74-1.57 (m, 2H), 1.54-1.40 (m, 3H); MS: 344.3 [M+H]⁺.

### Step 2: 5-(4-Hydroxyphenyl)-N-(tetrahydro-2H-pyran-4-yl)bicyclo[3.2.1]octane-1-carboxamide

To a solution of 5-(4-methoxyphenyl)-N-(tetrahydro-2H-pyran-4-yl)bicyclo[3.2.1]octane-1-carboxamide (130 mg, 0.38 mmol) in DCM (9 mL) at −78° C., BBr₃ (285 mg, 1.14 mmol) was added dropwise. The mixture was warmed to room temperature and stirred at room temperature for 2 h. The reaction mixture was poured into MeOH (5 mL) at 0° C. and concentrated to give 5-(4-hydroxyphenyl)-N-(tetrahydro-2H-pyran-4-yl)bicyclo[3.2.1]octane-1-carboxamide (130 mg, crude) as a red solid. MS: 328.1 [M−H]⁻.

### Step 3: tert-Butyl ((E)-3-fluoro-2-((4-(5-((tetrahydro-2H-pyran-4-yl)carbamoyl)bicyclo[3.2.1]octan-1-yl)phenoxy)methyl)allyl)carbamate

To a solution of 5-(4-hydroxyphenyl)-N-(tetrahydro-2H-pyran-4-yl)bicyclo[3.2.1]octane-1-carboxamide (130 mg, crude) in MeCN (7 mL) at room temperature, tert-butyl (E)-(2-(bromomethyl)-3-fluoroallyl)carbamate (127 mg, 0.47 mmol) and Cs₂CO₃ (386 mg, 1.18 mmol) were added and the resulting mixture stirred at room temperature overnight. The reaction was diluted with H₂O (20 mL) and extracted with EtOAc (3×20 mL). The combined organic layers were washed with brine (30 mL), dried (Na₂SO₄), filtered and concentrated to give tert-butyl ((E)-3-fluoro-2-((4-(5-((tetrahydro-2H-pyran-4-yl)carbamoyl)bicyclo[3.2.1]octan-1-yl)phenoxy)methyl)allyl)carbamate (156 mg, 77%) as a white solid. ¹H NMR (400 MHz, CDCl₃): δ 7.17 (d, 2H), 6.85 (d, 2H), 6.76 (d, 1H), 5.41 (d, 1H), 4.76-4.74 (m, 1H), 4.43 (d, 2H), 4.06-3.95 (m, 5H), 3.49 (t, 2H), 2.16 (d, 1H), 2.10-2.01 (m, 2H), 1.98-1.76 (m, 9H), 1.73-1.60 (m, 2H), 1.51-1.46 (m, 2H), 1.42 (s, 9H); MS: 517.4 [M+H]⁺.

### Step 4: 5-(4-(((E)-2-(Aminomethyl)-3-fluoroallyl)oxy)phenyl)-N-(tetrahydro-2H-pyran-4-yl)bicyclo[3.2.1]octane-1-carboxamide Hydrochloride

To a solution of tert-butyl ((E)-3-fluoro-2-((4-(5-((tetrahydro-2H-pyran-4-yl)carbamoyl)bicyclo[3.2.1]octan-1-yl)phenoxy)methyl)allyl)carbamate (160 mg, 0.31 mmol) in DCM (7 mL) at room temperature, TFA (6.16 g, 54.0 mmol) was added. The resulting mixture was stirred at room temperature for 0.5 h. The reaction mixture was concentrated to dryness and then purified by reverse-phase HPLC (water (0.04% HCl)/MeCN) to give 5-(4-(((E)-2-(aminomethyl)-3-fluoroallyl)oxy)phenyl)-N-(tetrahydro-2H-pyran-4-yl)bicyclo[3.2.1]octane-1-carboxamide hydrochloride (73 mg, 56%) as a white solid. ¹H NMR (400 MHz, DMSO-d₆): δ 8.24 (s, 3H), 7.29 (d, 1H), 7.27 (d, 1H), 7.18 (d, 2H), 6.91 (d, 2H), 4.59 (d, 2H), 3.84-3.74 (m, 3H), 3.58 (d, 2H), 3.31 (t, 2H), 2.01-1.85 (m, 3H), 1.81-1.66 (m, 6H), 1.64-1.44 (m, 6H), 1.43-1.32 (m, 1H); MS: 417.3 [M+H]⁺.

### Compound 6

### anti-5-(4-(((E)-2-(Aminomethyl)-3-fluoroallyl)oxy)phenyl)-N-methyladamantane-2-carboxamide Hydrochloride

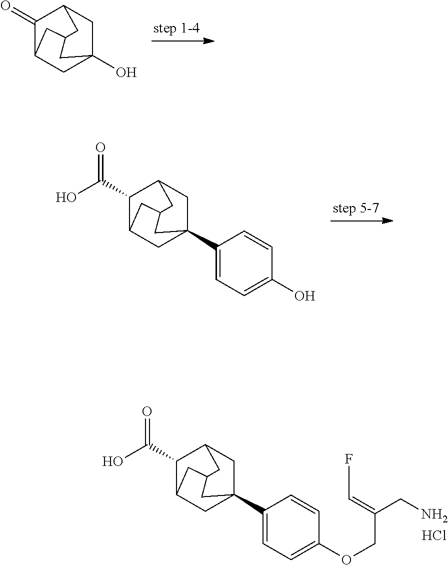 [SMILES: O=C1C2CC3CC1CC(O)(C3)C2 ; O=C(O)[C@H]1C2CC3CC1C[C@@](c1ccc(O)cc1)(C3)C2 ; NC/C(=C\F)COc1ccc([C@]23CC4CC(C2)[C@H](C(=O)O)C(C4)C3)cc1 ; Cl]

### Step 1: 5-(4-Hydroxyphenyl)adamantan-2-one

A stirred solution of 5-hydroxyadamantan-2-one (5.00 g, 30.1 mmol) in methanesulfonic acid (30 mL) was heated to 40° C. under a nitrogen atmosphere. Phenol (4.25 g, 45.1 mmol) was added, the mixture was stirred at 50° C. overnight, cooled to room temperature, diluted with DCM (30 mL) and poured into ice-water (30 mL). The aqueous phase extracted with DCM (3×30 mL). The combined organics were washed with brine (100 mL), dried (Na₂SO₄), filtered, concentrated and then purified by silica gel chromatography (petroleum ether/EtOAc 30:1-10:1) to give 5-(4-hydroxyphenyl)adamantan-2-one (747 mg, 10%) as a white solid. ¹H NMR (400 MHz, CDCl₃): δ 7.20 (d, 2H), 6.80 (d, 2H), 2.67-2.57 (m, 2H), 2.28-2.17 (m, 2H), 2.14-2.09 (m, 3H), 2.08-2.07 (m, 4H), 2.07-2.06 (m, 2H); LCMS: 241.1 [M−H]⁻.

### Step 2: 5-(4-Methoxyphenyl)adamantan-2-one

A mixture of 5-(4-hydroxyphenyl)adamantan-2-one (747 mg, 3.08 mmol), K₂CO₃ (1.70 g, 12.3 mmol), MeI (1.31 g, 9.25 mmol) and acetone (50 mL) was heated to 65° C. and stirred overnight. The solvent was removed under reduce pressure, the mixture diluted with water (20 mL) and extracted with ethyl acetate (3×20 mL). The organic phase was washed with brine (40 mL), dried (Na₂SO₄), filtered, concentrated and then purified by silica gel chromatography (petroleum ether/EtOAc 50:1-20:1) to give 5-(4-methoxyphenyl)adamantan-2-one (325 mg, 41%) as a white solid. ¹H NMR (400 MHz, CDCl₃): δ 7.29-7.26 (m, 2H), 6.90-6.87 (m, 2H), 3.82 (s, 3H), 2.70-2.55 (m, 2H), 2.29-2.24 (m, 3H), 2.19-2.16 (m, 4H), 2.09-2.07 (m, 4H); LCMS: 257.2 [M+H]⁺.

### Step 3: 5-(4-Methoxyphenyl)adamantane-2-carbonitrile

To a solution of 5-(4-methoxyphenyl)adamantan-2-one (1.20 g, 4.68 mmol) and 2-(p-tolylsulfonyl)acetonitrile (1.37 g, 7.02 mmol) in 1,2-dimethoxyethane (30 mL) at 0° C. under a nitrogen atmosphere t-BuOK (1.58 g, 14.0 mmol) was added. The reaction was slowly warmed to room temperature and stirred overnight. Most of the solvent was evaporated in vacuo, water (30 mL) added and the mixture extracted with ethyl acetate (3×30 mL). The organic phase was collected and washed with brine (60 mL), dried (Na₂SO₄), filtered, concentrated and then purified by silica gel chromatography (petroleum ether/EtOAc 50:1-30:1) to give 5-(4-methoxyphenyl)adamantane-2-carbonitrile (1.02 g, 82%) as a white solid. ¹H NMR (400 MHz, CDCl₃): δ 7.29-7.24 (m, 2H), 6.88-6.85 (m, 2H), 3.79 (s, 3H), 2.91-2.80 (m, 1H), 2.37-2.28 (m, 3H), 2.20-2.17 (m, 2H), 2.02 (d, 1H), 1.92-1.84 (m, 5H), 1.77-1.73 (m, 2H); LCMS: 268.2 [M+H]⁺.

### Step 4: anti-5-(4-Hydroxyphenyl)adamantane-2-carboxylic Acid

A solution of 5-(4-methoxyphenyl)adamantane-2-carbonitrile (600 mg, 2.24 mmol) in a mixed solvent of AcOH (20 mL) and 40% aqueous HBr solution (80 mL, 589 mmol) was stirred at 120° C. overnight. The resulted mixture was cooled to room temperature and dried under vacuum. The residue was purified by reverse-phase HPLC (water(0.1% TFA)-MeCN) to give anti-5-(4-hydroxyphenyl)adamantane-2-carboxylic acid* (200 mg, 33%) as a yellow solid. ¹H NMR (400 MHz, DMSO-d₆): δ 12.13 (s, 1H), 9.11 (s, 1H), 7.06 (d, 2H), 6.66 (d, 2H), 2.53-2.50 (m, 1H), 2.37-2.20 (m, 2H), 2.00-1.90 (m, 1H), 1.84 (d, 2H), 1.78-1.65 (m, 8H); LCMS: 271.1 [M−H]⁻; and

syn-5-(4-hydroxyphenyl)adamantane-2-carboxylic acid* (173 mg, 28%) as a yellow solid. ¹H NMR (400 MHz, DMSO-d₆): δ 12.12 (s, 1H), 9.10 (s, 1H), 7.11 (d, 2H), 6.66 (d, 2H), 2.59-2.55 (m, 1H), 2.33-2.25 (m, 2H), 1.98-1.92 (m, 1H), 1.86-1.71 (m, 8H), 1.53 (d, 2H).
* arbitrary assignment of syn and anti isomers

### Step 5: anti-5-(4-Hydroxyphenyl)-N-methyladamantane-2-carboxamide

To a solution of anti-5-(4-hydroxyphenyl)adamantane-2-carboxylic acid (133 mg, 0.49 mmol) in DCM (10 mL) was added DIPEA (631 mg, 4.88 mmol) and HATU (279 mg, 0.73 mmol) at 0° C. Methanamine HCl salt (72.5 mg, 1.07 mmol) was added, the mixture stirred at 0° C. for 30 min, heated to 30° C. stirred overnight, dried under vacuum and purified by reverse-phase HPLC (water(0.1% TFA)-MeOH) to give anti-5-(4-hydroxyphenyl)-N-methyladamantane-2-carboxamide (118 mg, 85%) as a white solid. ¹H NMR (400 MHz, DMSO-d₆): δ 9.09 (s, 1H), 7.53 (d, 1H), 7.13 (d, 2H), 6.67 (d, 2H), 2.59 (d, 3H), 2.38-2.32 (m, 3H), 1.92-1.76 (m, 9H), 1.47 (d, 2H); LCMS: 286.2 [M+H]⁺.

### Step 6: Tert-Butyl ((E)-3-fluoro-2-((4-(anti-4-(methylcarbamoyl)adamantan-1-yl)phenoxy)methyl)allyl)carbamate

To a solution of anti-5-(4-hydroxyphenyl)-N-methyladamantane-2-carboxamide (118 mg, 0.41 mmol), tert-butyl N—[(E)-2-(bromomethyl)-3-fluoro-allyl]carbamate (133 mg, 0.50 mmol) in MeCN (20 mL) was added Cs₂CO₃ (404 mg, 1.24 mmol). The mixture was stirred at room temperature overnight. The reaction mixture was poured into water (20 mL) and extracted with ethyl acetate (3×20 mL). The organic phase was collected and washed with brine (40 mL), dried (Na₂SO₄), filtered, concentrated and then purified by silica gel chromatography (petroleum ether/EtOAc 10:1-1:1) to give tert-butyl ((E)-3-fluoro-2-((4-(anti-4-(methylcarbamoyl)adamantan-1-yl)phenoxy)methyl)allyl)carbamate (116 mg, 59%) as a white solid. ¹H NMR (400 MHz, DMSO-d₆): δ 7.28 (d, 2H), 6.87 (d, 2H), 6.74 (d, 1H), 5.55 (d, 1H), 4.77 (s, 1H), 4.43 (d, 2H), 4.00 (d, 2H), 2.87 (d, 3H), 2.52-2.41 (m, 3H), 2.05-2.00 (m, 5H), 1.91-1.85 (m, 4H), 1.61-1.57 (m, 2H), 1.42 (s, 9H); LCMS: 417.1 [M+H-56]+.

### Step 7: anti-5-(4-(((E)-2-(Aminomethyl)-3-fluoroallyl)oxy)phenyl)-N-methyladamantane-2-carboxamide Hydrochloride

To a solution of tert-butyl ((E)-3-fluoro-2-((4-(anti-4-(methylcarbamoyl)adamantan-1-yl)phenoxy)methyl)allyl)carbamate (132 mg, 0.28 mmol) in DCM (2 mL) was added TFA (6.5 mL, 89.1 mmol) via syringe and the mixture stirred at room temperature for 2 h. The reaction was concentrated and purified by reverse-phase HPLC (water(0.04% HCl)-MeCN) to give anti-5-(4-(((E)-2-(aminomethyl)-3-fluoroallyl)oxy)phenyl)-N-methyladamantane-2-carboxamide hydrochloride (33 mg, 32%) as a white solid. ¹H NMR (400 MHz, DMSO-d₆): δ 8.25-8.10 (m, 3H), 7.56 (d, 1H), 7.29-7.27 (m, 2H), 7.29 (d, 1H), 6.93-6.91 (m, 2H), 4.58 (d, 2H), 3.59 (d, 2H), 2.60 (d, 3H), 2.40-2.35 (m, 1H), 2.34-2.01 (m, 2H), 1.90-1.78 (m, 9H), 1.49 (d, 2H); LCMS: 373.3 [M+H]⁺.

### Compound 6.01

### syn-5-(4-(((E)-2-(Aminomethyl)-3-fluoroallyl)oxy)phenyl)-N-methyladamantane-2-carboxamide Hydrochloride

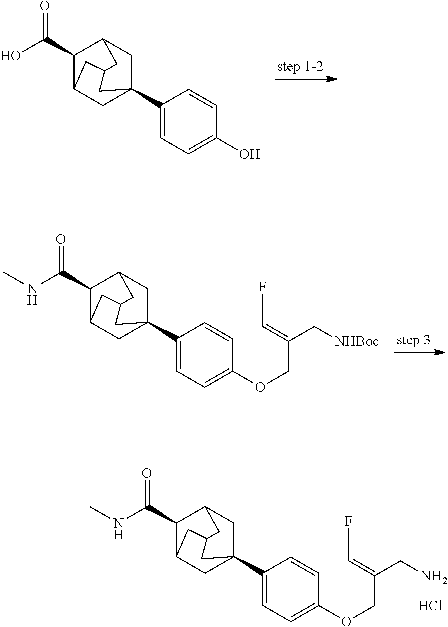 [SMILES: O=C(O)[C@H]1C2CC3CC1C[C@](c1ccc(O)cc1)(C3)C2 ; CNC(=O)[C@H]1C2CC3CC1C[C@](c1ccc(OC/C(=C/F)CNC(=O)OC(C)(C)C)cc1)(C3)C2 ; CNC(=O)[C@H]1C2CC3CC1C[C@](c1ccc(OC/C(=C/F)CN)cc1)(C3)C2 ; Cl]

### Step 1: syn-5-(4-Hydroxyphenyl)-N-methyladamantane-2-carboxamide

To a solution of syn-5-(4-hydroxyphenyl)adamantane-2-carboxylic acid (106 mg, 0.39 mmol) in DCM (10 mL) was added HATU (222 mg, 0.58 mmol) and DIPEA (0.7 mL, 3.89 mmol) at 0° C. Methanamine HCl salt (64 mg, 0.86 mmol) was added at 0° C., the mixture stirred at 0° C. for 30 min and then heated to 30° C. and stirred overnight. The mixture was poured into H₂O (20 mL), aqueous layer extracted with EtOAc (3×20 mL), the combined organic layers were washed with brine (20 mL), dried (Na₂SO₄), filtered and concentrated to give syn-5-(4-hydroxyphenyl)-N-methyladamantane-2-carboxamide (110 mg, crude) as a yellow solid. LCMS: 286.1 [M+H]⁺.

### Step 2: Tert-Butyl ((E)-3-fluoro-2-((4-(syn-4-(methylcarbamoyl)adamantan-1-yl)phenoxy)methyl)allyl)carbamate

A mixture of syn-5-(4-hydroxyphenyl)-N-methyladamantane-2-carboxamide (110 mg, 0.39 mmol), (E)-tert-butyl (2-(bromomethyl)-3-fluoroallyl)carbamate (124 mg, 0.46 mmol), Cs₂CO₃ (377 mg, 1.16 mmol) and MeCN (4 mL) was stirred at room temperature overnight. The mixture was poured into H₂O (10 mL), extracted with EtOAc (3×10 mL), the combined organic layers were washed with brine (10 mL), dried (Na₂SO₄), filtered, concentrated and then purified by column chromatography (SiO₂, petroleum ether/ethyl acetate=10/1-1/1) to give tert-butyl ((E)-3-fluoro-2-((4-(syn-4-(methylcarbamoyl)adamantan-1-yl)phenoxy)methyl)allyl)carbamate (145 mg, crude) as a yellow oil. LCMS: 417.1 [M+H-56]⁺.

### Step 3: syn-5-(4-(((E)-2-(Aminomethyl)-3-fluoroallyl)oxy)phenyl)-N-methyladamantane-2-carboxamide Hydrochloride

To a solution of tert-butyl ((E)-3-fluoro-2-((4-(syn-4-(methylcarbamoyl)adamantan-1-yl)phenoxy)methyl)allyl)carbamate (145 mg, 0.31 mmol) in DCM (3 mL) was added TFA (1.2 mL, 16.2 mmol) and the mixture was stirred at room temperature for 1 h. The mixture was concentrated and purified by reverse-phase HPLC (water(0.04% HCl)-MeCN) to give syn-5-(4-(((E)-2-(aminomethyl)-3-fluoroallyl)oxy)phenyl)-N-methyladamantane-2-carboxamide hydrochloride (40 mg, 35%) as a white solid. ¹H NMR (400 MHz, DMSO-d₆): δ 8.32-8.15 (m, 3H), 7.60 (d, 1H), 7.28 (d, 1H), 7.21 (d, 2H), 6.91 (d, 2H), 4.58 (d, 2H), 3.62-3.50 (m, 2H), 2.57 (d, 3H), 2.40-2.28 (m, 3H), 2.09-1.99 (m, 3H), 1.82-1.67 (m, 6H), 1.65-1.55 (m, 2H); LCMS: 373.2 [M+H]⁺.

### Compound 6.02

### syn-5-(4-(((E)-2-(Aminomethyl)-3-fluoroallyl)oxy)phenyl)-N-(tetrahydro-2H-pyran-4-yl)adamantane-2-carboxamide Hydrochloride

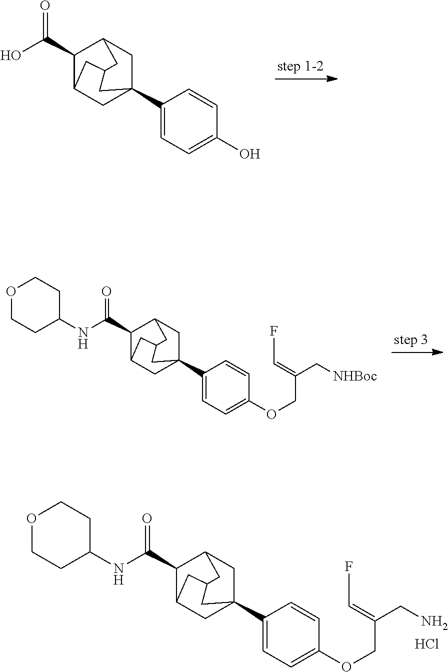 [SMILES: O=C(O)[C@H]1C2CC3CC1C[C@](c1ccc(O)cc1)(C3)C2 ; CC(C)(C)OC(=O)NC/C(=C\F)COc1ccc([C@]23CC4CC(C2)[C@@H](C(=O)NC2CCOCC2)C(C4)C3)cc1 ; NC/C(=C\F)COc1ccc([C@]23CC4CC(C2)[C@@H](C(=O)NC2CCOCC2)C(C4)C3)cc1 ; Cl]

### Step 1: syn-5-(4-Hydroxyphenyl)-N-(tetrahydro-2H-pyran-4-yl)adamantane-2-carboxamide

To a solution of syn-5-(4-hydroxyphenyl)adamantane-2-carboxylic acid (106 mg, 0.39 mmol) in DCM (10 mL) was added HATU (222 mg, 0.58 mmol) and DIPEA (0.70 mL, 3.89 mmol) at 0° C. Tetrahydro-2H-pyran-4-amine (87 mg, 0.86 mmol) was added at 0° C., the mixture was stirred at 0° C. for 30 min, heated to 30° C. and stirred overnight. The mixture was poured into H₂O (20 mL), extracted with EtOAc (3×20 mL), the combined organic layers were washed with brine (20 mL), dried (Na₂SO₄), filtered and concentrated to give syn-5-(4-hydroxyphenyl)-N-(tetrahydro-2H-pyran-4-yl)adamantane-2-carboxamide (100 mg, crude) as a yellow oil. LCMS: 356. [M+H]⁺.

### Step 2: Tert-Butyl ((E)-3-fluoro-2-((4-(syn-4-((tetrahydro-2H-pyran-4-yl)carbamoyl)adamantan-1-yl)phenoxy)methyl)allyl)carbamate

To a mixture of syn-5-(4-hydroxyphenyl)-N-(tetrahydro-2H-pyran-4-yl)adamantane-2-carboxamide (100 mg, 0.28 mmol), (E)-tert-butyl (2-(bromomethyl)-3-fluoroallyl)carbamate (91 mg, 0.34 mmol) in MeCN (4 mL) was added Cs₂CO₃ (275 mg, 0.84 mmol) at room temperature and the mixture stirred at room temperature overnight. The mixture was poured into H₂O (10 mL), extracted with EtOAc (3×10 mL), the combined organic layers were washed with brine (10 mL), dried (Na₂SO₄), filtered, concentrated and then purified by column chromatography (SiO₂, petroleum ether/ethyl acetate=10/1-1/1) to give tert-butyl ((E)-3-fluoro-2-((4-(syn-4-((tetrahydro-2H-pyran-4-yl)carbamoyl)adamantan-1-yl)phenoxy)methyl)allyl)carbamate (102 mg, crude) as a yellow solid. LCMS: 487.2 [M+H-56]⁺.

### Step 3: syn-5-(4-(((E)-2-(Aminomethyl)-3-fluoroallyl)oxy)phenyl)-N-(tetrahydro-2H-pyran-4-yl)adamantane-2-carboxamide Hydrochloride

To a solution of tert-butyl ((E)-3-fluoro-2-((4-(syn-4-((tetrahydro-2H-pyran-4-yl)carbamoyl)adamantan-1-yl)phenoxy)methyl)allyl)carbamate (102 mg, 0.188 mmol) in DCM (2 mL) was added TFA (0.8 mL, 10.8 mmol) at room temperature and the mixture stirred at room temperature for 1 h. The mixture was concentrated and purified by reverse-phase HPLC (water(0.04% HCl)-MeCN) to give syn-5-(4-(((E)-2-(aminomethyl)-3-fluoroallyl)oxy)phenyl)-N-(tetrahydro-2H-pyran-4-yl)adamantane-2-carboxamide hydrochloride (35 mg, 39%) as a pink solid. ¹H NMR (400 MHz, DMSO-d₆): δ 8.21-8.01 (m, 3H), 7.57 (d, 1H), 7.29 (d, 1H), 7.21 (d, 2H), 6.91 (d, 2H), 4.56 (d, 2H), 3.87-3.71 (m, 3H), 3.65-3.55 (m, 2H), 3.34-3.28 (m, 2H), 2.41-2.26 (m, 3H), 2.12 (d, 2H), 2.05-2.03 (m, 1H), 1.83-1.69 (m, 6H), 1.67-1.54 (m, 4H), 1.46-1.32 (m, 2H); LCMS: 373.2 [M+H]⁺.

### Compound 6.03

### anti-5-(4-(((E)-2-(Aminomethyl)-3-fluoroallyl)oxy)phenyl)-N-(tetrahydro-2H-pyran-4-yl)adamantane-2-carboxamide Hydrochloride

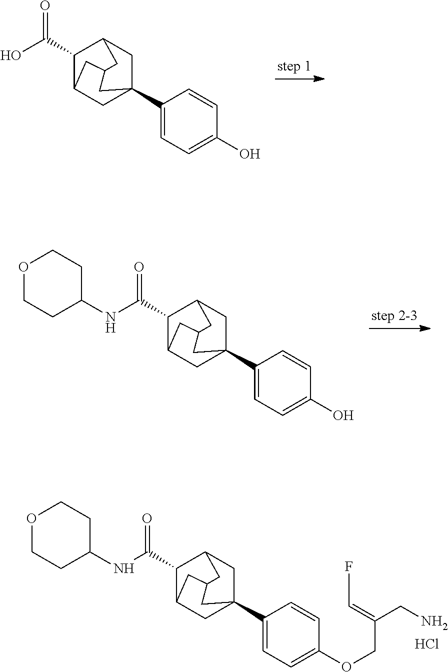 [SMILES: O=C(O)[C@H]1C2CC3CC1C[C@@](c1ccc(O)cc1)(C3)C2 ; O=C(NC1CCOCC1)[C@H]1C2CC3CC1C[C@@](c1ccc(O)cc1)(C3)C2 ; NC/C(=C\F)COc1ccc([C@]23CC4CC(C2)[C@H](C(=O)NC2CCOCC2)C(C4)C3)cc1 ; Cl]

### Step 1: anti-5-(4-Hydroxyphenyl)-N-(tetrahydro-2H-pyran-4-yl)adamantane-2-carboxamide

To a solution of anti-5-(4-hydroxyphenyl)adamantane-2-carboxylic acid (134 mg, 0.49 mmol) and DIPEA (636 mg, 4.92 mmol) in DCM (5 mL) was added HATU (281 mg, 0.74 mmol) at 0° C. followed by tetrahydropyran-4-amine (109 mg, 1.08 mmol). The mixture was stirred at 0° C. for 0.5 h then 30° C. overnight. The mixture was poured into water (10 mL), extracted with DCM (3×10 mL), the combined organics washed with brine (40 mL), dried (Na₂SO₄), filtered and concentrated. The residue was re-dissolved in DCM (18 mL), DIPEA (2.0 g, 10.6 mmol) and methanamine (54.9 mg, 0.813 mmol) added and the mixture stirred at room temperature for 1 h. The mixture was poured into water (25 mL) and extracted with DCM (3×30 mL). The organic phase was washed with brine (40 mL), dried (Na₂SO₄), filtered and concentrated to give anti-5-(4-hydroxyphenyl)-N-(tetrahydro-2H-pyran-4-yl)adamantane-2-carboxamide (190 mg, crude) as a yellow solid. LCMS: 356.3 [M+H]⁺.

### Step 2: Tert-Butyl ((E)-3-fluoro-2-((4-(anti-4-((tetrahydro-2H-pyran-4-yl)carbamoyl)adamantan-1-yl)phenoxy)methyl)allyl)carbamate

To a mixture of anti-5-(4-hydroxyphenyl)-N-(tetrahydro-2H-pyran-4-yl)adamantane-2-carboxamide (180 mg, 0.51 mmol) and Cs₂CO₃ (495 mg, 1.52 mmol) in MeCN (9 mL) was added tert-butyl N—[(E)-2-(bromomethyl)-3-fluoro-allyl]carbamate (163 mg, 0.61 mmol) and the mixture stirred at room temperature overnight. The reaction mixture was poured into water (10 mL) and extracted with ethyl acetate (2×10 mL). The organic phase was washed with brine (30 mL), dried (Na₂SO₄), filtered, concentrated and purified by chromatography on silica gel (dichloromethane/methanol=10/1) to give tert-butyl ((E)-3-fluoro-2-((4-(anti-4-((tetrahydro-2H-pyran-4-yl)carbamoyl)adamantan-1-yl)phenoxy)methyl)allyl)carbamate (72 mg, 26%) as a white solid. LCMS: 487.3 [M+H-56]+.

### Step 3: anti-5-(4-(((E)-2-(aminomethyl)-3-fluoroallyl)oxy)phenyl)-N-(tetrahydro-2H-pyran-4-yl)adamantane-2-carboxamide Hydrochloride

To a solution of tert-butyl ((E)-3-fluoro-2-((4-(anti-4-((tetrahydro-2H-pyran-4-yl)carbamoyl)adamantan-1-yl)phenoxy)methyl)allyl)carbamate (76 mg, 0.14 mmol) in DCM (3 mL) was added TFA (1.5 mL, 20.3 mmol) via syringe and the mixture stirred at room temperature for 2 h. The reaction mixture was concentrated and purified by reverse-phase HPLC (water(0.04% HCl)-MeCN) to give anti-5-(4-(((E)-2-(aminomethyl)-3-fluoroallyl)oxy)phenyl)-N-(tetrahydro-2H-pyran-4-yl)adamantane-2-carboxamide hydrochloride (9 mg, 15%) as white solid. ¹H NMR (400 MHz, DMSO-d₆): δ 8.18-8.10 (m, 3H), 7.51 (d, 1H), 7.29 (d, 1H), 7.28 (d, 2H), 6.93 (d, 2H), 4.57 (d, 2H), 3.83-3.80 (m, 3H), 3.59-3.55 (m, 2H), 3.36-3.30 (m, 2H), 2.45-2.40 (m, 1H), 2.36-2.33 (m, 2H), 1.99-1.78 (m, 9H), 1.63 (d, 2H), 1.50-1.39 (m, 4H); LCMS: 443.2 [M+H]⁺.

The compounds below were synthesized in a similar manner as described for previous compounds.

| Compound | Structure | Chemical Name | m/z |
|---|---|---|---|
| 6.04 | 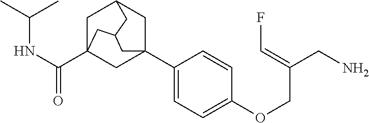 [SMILES: CC(C)NC(=O)C12CC3CC(C1)CC(c1ccc(OC/C(=C/F)CN)cc1)(C3)C2] | (E)-3-(4-((2-(aminomethyl)-3- fluoroallyl)oxy)phenyl)-N- isopropyladamantane-1- carboxamide | 401.3 |

### Compound 7

### (E)-N-(4-(4-((2-(Aminomethyl)-3-fluoroallyl)oxy)phenyl)bicyclo[2.2.2]octan-1-yl)tetrahydro-2H-pyran-4-carboxamide Hydrochloride

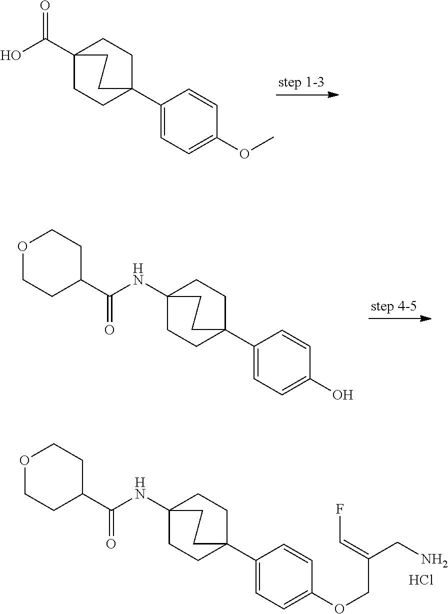 [SMILES: COc1ccc(C23CCC(C(=O)O)(CC2)CC3)cc1 ; O=C(NC12CCC(c3ccc(O)cc3)(CC1)CC2)C1CCOCC1 ; NC/C(=C\F)COc1ccc(C23CCC(NC(=O)C4CCOCC4)(CC2)CC3)cc1 ; Cl]

### Step 1: 4-(4-Methoxyphenyl)bicyclo[2.2.2]octan-1-amine hydrochloride

To a solution of 4-(4-methoxyphenyl)bicyclo[2.2.2]octane-1-carboxylic acid (1.0 g, 3.84 mmol) in toluene (20 mL) at room temperature, DPPA (1.59 g, 5.76 mmol) and TEA (1.75 g, 17.3 mmol) were added. The mixture was stirred at 100° C. overnight. Then 6 M HCl (˜10 mL) was added at 40° C., the mixture stirred at 40° C. for 3 h and concentrated to dryness. The residue was triturated with EtOAc at 20° C. for 2 h and filtered to give 4-(4-methoxyphenyl)bicyclo[2.2.2]octan-1-amine hydrochloride (300 mg, 29%) as a yellow solid. ¹H NMR (400 MHz, DMSO-d₆): δ 8.19 (s, 3H), 7.22 (d, 2H), 6.84 (d, 2H), 3.70 (s, 3H), 1.83-1.80 (m, 12H); MS: 232.1 [M+H]⁺.

### Step 2: N-(4-(4-Methoxyphenyl)bicyclo[2.2.2]octan-1-yl)tetrahydro-2H-pyran-4-carboxamide

A mixture of 4-(4-methoxyphenyl)bicyclo[2.2.2]octan-1-amine hydrochloride (300 mg, 1.12 mmol), tetrahydro-2H-pyran-4-carboxylic acid (219 mg, 1.68 mmol), DIPEA (579 mg, 4.48 mmol), HATU (639 mg, 1.68 mmol) and DMF (10 mL) was stirred at room temperature for 24 h. The mixture was diluted with H₂O (20 mL), extracted with EtOAc (2×20 mL), the organic layers were washed by brine (3×20 mL), dried (Na₂SO₄), concentrated and then purified by silica column (petroleum ether/EtOAc=10/1 to 1/1) to give N-(4-(4-Methoxyphenyl)bicyclo[2.2.2]octan-1-yl)tetrahydro-2H-pyran-4-carboxamide (351 mg, 82%) as a yellow solid. ¹H NMR (400 MHz, CDCl₃): δ 7.23 (d, 2H), 6.84 (d, 2H), 5.12 (s, 1H), 4.09-3.95 (m, 2H), 3.79 (s, 3H), 3.48-3.36 (m, 2H), 2.28-2.17 (m, 1H), 2.07-1.97 (m, 6H), 1.97-1.88 (m, 6H), 1.82-1.55 (m, 4H); MS: 342.2 [M−H]⁻.

### Step 3: N-(4-(4-Hydroxyphenyl)bicyclo[2.2.2]octan-1-yl)tetrahydro-2H-pyran-4-carboxamide

To a solution of N-(4-(4-methoxyphenyl)bicyclo[2.2.2]octan-1-yl)tetrahydro-2H-pyran-4-carboxamide (0.18 g, 0.52 mmol) in DCM (9 mL) at −78° C., BBr₃ (394 mg, 1.57 mmol) was added dropwise. After addition the mixture was warmed slowly to room temperature and stirred at room temperature for 2 h. The reaction mixture was poured into MeOH (10 mL) at 0° C. and concentrated to dryness to give N-(4-(4-hydroxyphenyl)bicyclo[2.2.2]octan-1-yl)tetrahydro-2H-pyran-4-carboxamide (180 mg, crude) as a red solid. MS: 330.2 [M+H]⁺.

### Step 4: Tert-Butyl (E)-(3-fluoro-2-((4-(4-(tetrahydro-2H-pyran-4-carboxamido)bicyclo[2.2.2]octan-1-yl)phenoxy)methyl)allyl)carbamate

To a solution of N-(4-(4-hydroxyphenyl)bicyclo[2.2.2]octan-1-yl)tetrahydro-2H-pyran-4-carboxamide (180 mg, 0.55 mmol) in MeCN (9 mL) at room temperature, tert-butyl (E)-(2-(bromomethyl)-3-fluoroallyl)carbamate (176 mg, 0.66 mmol) and Cs₂CO₃ (534 mg, 1.64 mmol) were added. The resulting mixture was stirred at room temperature overnight. The reaction mixture was diluted with H₂O (10 mL) and extracted with EtOAc (3×10 mL). The combined organic layers were washed with brine (15 mL), dried (Na₂SO₄), filtered, concentrated and purified by prep-TLC (petroleum ether/EtOAc=0/1) to give tert-butyl (E)-(3-fluoro-2-((4-(4-(tetrahydro-2H-pyran-4-carboxamido)bicyclo[2.2.2]octan-1-yl)phenoxy)methyl)allyl)carbamate (30 mg 11%) as a white solid. ¹H NMR (400 MHz, CDCl₃): δ 7.22 (d, 2H) 6.84 (d, 2H), 6.75 (d, 1H), 5.12 (s, 1H), 4.76-4.73 (m, 1H), 4.42 (d, 2H), 4.03-3.95 (m, 4H), 3.46-3.41 (m, 2H), 2.28-2.15 (m, 1H), 2.06-1.96 (m, 6H), 1.95-1.87 (m, 6H), 1.83-1.70 (m, 4H), 1.42 (s, 9H); MS: 517.3 [M+H]⁺.

### Step 5: (E)-N-(4-(4-((2-(Aminomethyl)-3-fluoroallyl)oxy)phenyl)bicyclo[2.2.2]octan-1-yl)tetrahydro-2H-pyran-4-carboxamide Hydrochloride

To a solution of tert-butyl (E)-(3-fluoro-2-((4-(4-(tetrahydro-2H-pyran-4-carboxamido)bicyclo[2.2.2]octan-1-yl)phenoxy)methyl)allyl)carbamate (27 mg, 0.052 mmol) in DCM (0.5 mL) at room temperature TFA (1.39 g, 12.2 mmol) was added. The resulting mixture was stirred at room temperature for 1 h. The reaction mixture was concentrated to dryness and then purified by reverse-phase HPLC (water (0.04% HCl)-MeCN) to give (E)-N-(4-(4-((2-(aminomethyl)-3-fluoroallyl)oxy)phenyl)bicyclo[2.2.2]octan-1-yl)tetrahydro-2H-pyran-4-carboxamide hydrochloride (10 mg, 43%) as a white solid. ¹H NMR (400 MHz, DMSO-d₆): δ 8.18 (br s, 3H), 7.28 (d, 1H), 7.26-7.21 (m, 3H), 6.89 (d, 2H), 4.57 (d, 2H), 3.90-3.76 (m, 2H), 3.59-3.55 (m, 2H), 3.29-3.22 (m, 2H), 2.40-2.23 (m, 1H), 1.93-1.85 (m, 6H), 1.85-1.75 (m, 6H), 1.61-1.43 (m, 4H); MS: 417.2 [M+H]⁺.

The compounds below were synthesized in a similar manner as previously described.

| Compound | Structure | Chemical Name | m/z |
|---|---|---|---|
| 7.01 | 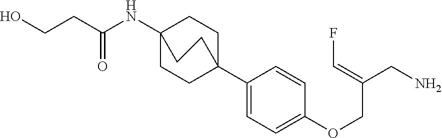 [SMILES: NC/C(=C\F)COc1ccc(C23CCC(NC(=O)CCO)(CC2)CC3)cc1] | (E)-N-(4-(4-((2-(aminomethyl)-3- fluoroallyl)oxy)phenyl)bicyclo [2.2.2]octan-1-yl)-3- hydroxypropanamide | 377.2 |
| 7.02 | 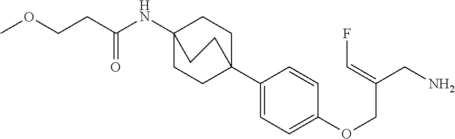 [SMILES: COCCC(=O)NC12CCC(c3ccc(OC/C(=C/F)CN)cc3)(CC1)CC2] | (E)-N-(4-(4-((2-(aminomethyl)-3- fluoroallyl)oxy)phenyl)bicyclo [2.2.2]octan-1-yl)-3- methoxypropanamide | 391.1 |
| 7.03 | 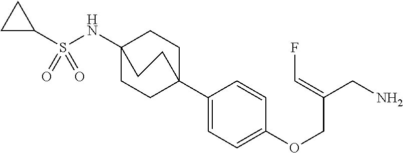 [SMILES: NC/C(=C\F)COc1ccc(C23CCC(NS(=O)(=O)C4CC4)(CC2)CC3)cc1] | (E)-N-(4-(4-((2-(aminomethyl)-3- fluoroallyl)oxy)phenyl) bicyclo[2.2.2]octan-1- yl)cyclopropanesulfonamide | 409.1 |

### Compound 8

### (E)-2-((4-(4-((2-(aminomethyl)-3-fluoroallyl)oxy)phenyl)bicyclo[2.2.2]octan-1-yl)oxy)-N-methylacetamide

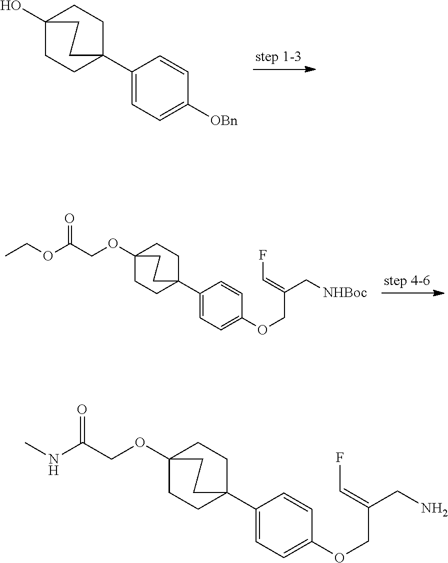 [SMILES: OC12CCC(c3ccc(OCc4ccccc4)cc3)(CC1)CC2 ; CCOC(=O)COC12CCC(c3ccc(OC/C(=C/F)CNC(=O)OC(C)(C)C)cc3)(CC1)CC2 ; CNC(=O)COC12CCC(c3ccc(OC/C(=C/F)CN)cc3)(CC1)CC2]

### Step 1: Ethyl 2-((4-(4-(benzyloxy)phenyl)bicyclo[2.2.2]octan-1-yl)oxy)acetate

To a mixture of 4-(4-(benzyloxy)phenyl)bicyclo[2.2.2]octan-1-ol (1.0 g, 3.24 mmol) in DCM (10 mL) at 0° C., diacetoxyrhodium (7.2 mg, 0.032 mmol) was added. The mixture was stirred at 0° C. for 5 min. Ethyl 2-diazoacetate (370 mg, 3.24 mmol) was added at 0° C. and the mixture warmed to room temperature overnight. The mixture was poured into water (10 mL) and extracted with DCM (5×10 mL). The organic layers were washed with brine (20 mL), dried (Na₂SO₄), filtered and then purified by chromatography on silica gel (petroleum ether/EtOAc=5/1) to give ethyl 2-((4-(4-(benzyloxy)phenyl)bicyclo[2.2.2]octan-1-yl)oxy)acetate (700 mg, 55%) as a white solid. ¹H NMR (400 MHz, CDCl₃): δ 7.50-7.28 (m, 5H), 7.22 (d, 2H), 6.91 (d, 2H), 5.04 (s, 2H), 4.24 (q, 2H), 4.06 (s, 2H), 2.02-1.91 (m, 6H), 1.87-1.77 (m, 6H), 1.31 (t, 3H); MS: 393.2 [M−H]⁻.

### Step 2: Ethyl 2-((4-(4-hydroxyphenyl)bicyclo[2.2.2]octan-1-yl)oxy)acetate

To a mixture of Pd/C (200 mg, 10% purity) in EtOH (40 mL), ethyl 2-((4-(4-(benzyloxy)phenyl)bicyclo[2.2.2]octan-1-yl)oxy)acetate (700 mg, 1.77 mmol) was added under an argon atmosphere. The suspension was degassed under vacuum, purged with hydrogen several times and stirred under a hydrogen atmosphere (45 psi) at room temperature overnight. The mixture was filtered through a Celite pad. The filtrate was concentrated to give ethyl 2-((4-(4-hydroxyphenyl)bicyclo[2.2.2]octan-1-yl)oxy)acetate (520 mg) as a yellow oil. ¹H NMR (400 MHz, CDCl₃): δ 7.17 (d, 2H), 6.76 (d, 2H), 4.72 (s, 1H), 4.23 (q, 2H), 4.06 (s, 2H), 2.01-1.90 (m, 6H), 1.86-1.77 (m, 6H), 1.29 (t, 3H).

### Step 3: (E)-Ethyl 2-((4-(4-((2-(((tert-butoxycarbonyl)amino)methyl)-3-fluoroallyl)oxy)phenyl)bicyclo[2.2.2]octan-1-yl)oxy)acetate

To a solution of ethyl 2-((4-(4-(benzyloxy)phenyl)bicyclo[2.2.2]octan-1-yl)oxy)acetate (520 mg), (E)-tert-butyl (2-(bromomethyl)-3-fluoroallyl)carbamate (550 mg, 2.05 mmol) in MeCN (5 mL), Cs₂CO₃ (1.67 g, 5.13 mmol) was added. The mixture was stirred at room temperature overnight. The mixture was poured into water (30 mL) and extracted with DCM (3×10 mL). The organic layers were combined, washed with brine (20 mL), dried (Na₂SO₄), filtered, concentrated and purified by chromatography on silica gel (petroleum ether/EtOAc=5/1) to give (E)-ethyl 2-((4-(4-((2-(((tert-butoxycarbonyl)amino)methyl)-3-fluoroallyl)oxy)phenyl)bicyclo[2.2.2]octan-1-yl)oxy)acetate (700 mg, 56% for 2 steps) as a yellow solid. ¹H NMR (400 MHz, CDCl₃): δ 7.22 (d, 2H), 6.89-6.81 (m, 2H), 6.73 (d, 1H), 4.88-4.64 (m, 1H), 4.42 (d, 2H), 4.22 (q, 2H), 4.06 (s, 2H), 4.03-3.92 (m, 2H), 1.99-1.90 (m, 6H), 1.87-1.78 (m, 6H), 1.42 (s, 9H), 1.29 (t, 3H); MS: 392.1 [M+H-100]⁺.

### Step 4: (E)-2-((4-(4-((2-(((tert-Butoxycarbonyl)amino)methyl)-3-fluoroallyl)oxy)phenyl)bicyclo[2.2.2]octan-1-yl)oxy)acetic Acid

To a solution of (E)-ethyl 2-((4-(4-((2-(((tert-butoxycarbonyl)amino)methyl)-3-fluoroallyl)oxy)phenyl)bicyclo[2.2.2]octan-1-yl)oxy)acetate (700 mg, 1.42 mmol) in THF (3 mL), MeOH (1.5 mL), water (1.5 mL) and LiOH—H₂O (299 mg, 7.12 mmol) were added. The mixture was stirred at 50° C. for 1 h. The reaction mixture was concentrated under reduced pressure to remove solvent. The residue was diluted with water (10 mL), adjusted to pH=3 with 1N HCl and extracted with EtOAc (3×5 mL). The organic layer was dried (Na₂SO₄), filtered and concentrated under reduced pressure to give (E)-2-((4-(4-((2-(((tert-butoxycarbonyl)amino)methyl)-3-fluoroallyl)oxy)phenyl)bicyclo[2.2.2]octan-1-yl)oxy)acetic acid (630 mg) as a yellow solid. ¹H NMR (400 MHz, DMSO-d₆): δ 12.37 (s, 1H), 7.20 (d, 2H), 7.02 (s, 1H), 6.96 (d, 1H), 6.94-6.79 (m, 2H), 4.38 (d, 2H), 3.92 (s, 2H), 3.81-3.64 (m, 2H), 1.92-1.79 (m, 6H), 1.77-1.64 (m, 6H), 1.33 (s, 9H); MS: 364.1 [M+H-100]⁺.

### Step 5: (E)-tert-Butyl (3-fluoro-2-((4-(4-(2-(methylamino)-2-oxoethoxy)bicyclo[2.2.2]octan-1-yl)phenoxy)methyl)allyl)carbamate

To a solution of (E)-2-((4-(4-((2-(((tert-butoxycarbonyl)amino)methyl)-3-fluoroallyl)oxy)phenyl)bicyclo[2.2.2]octan-1-yl)oxy)acetic acid (150 mg, 0.32 mmol), methanamine hydrochloride (33 mg, 0.49 mmol), DIPEA (126 mg, 0.97 mmol) in DCM (3 mL), HATU (148 mg, 0.39 mmol) was added. The mixture was stirred at room temperature for 4.5 h. The mixture was poured into water (10 mL) and extracted with DCM (3×10 mL). The organic layers were combined, washed with brine (20 mL), dried (Na₂SO₄), filtered, concentrated and then purified by chromatography on silica gel (petroleum ether/EtOAc=5/1) to give (E)-tert-butyl (3-fluoro-2-((4-(4-(2-(methylamino)-2-oxoethoxy)bicyclo[2.2.2]octan-1-yl)phenoxy)methyl)allyl)carbamate (200 mg) as a yellow oil. ¹H NMR (400 MHz, CDCl₃): δ 7.21 (d, 2H), 6.90-6.77 (m, 2H), 6.73 (d, 1H), 6.70-6.60 (m, 1H), 4.78-4.73 (m, 1H), 4.42 (d, 2H), 3.99 (d, 2H), 3.92 (s, 2H), 2.86 (d, 3H), 2.02-1.89 (m, 6H), 1.84-1.74 (m, 6H), 1.49-1.37 (m, 9H); MS:477.2 [M+H]⁺.

### Step 6: (E)-2-((4-(4-((2-(Aminomethyl)-3-fluoroallyl)oxy)phenyl)bicyclo[2.2.2]octan-1-yl)oxy)-N-methylacetamide Trifluoroacetate

A mixture of (E)-tert-butyl (3-fluoro-2-((4-(4-(2-(methylamino)-2-oxoethoxy)bicyclo[2.2.2]octan-1-yl)phenoxy)methyl)allyl)carbamate (200 mg), TFA (3.08 g, 27.0 mmol) and DCM (2 mL) was stirred at room temperature for 1 h. The mixture was concentrated to dryness and then purified by reverse-phase HPLC (water(0.1% TFA)-MeCN) to give (E)-2-((4-(4-((2-(aminomethyl)-3-fluoroallyl)oxy)phenyl)bicyclo[2.2.2]octan-1-yl)oxy)-N-methylacetamide trifluoroacetate (70 mg, 46% for 3 steps) as a white solid. ¹H NMR (400 MHz, DMSO-d₆) δ 8.09-8.01 (m, 3H), 7.37 (d, 1H), 7.30 (d, 1H), 7.25 (d, 2H), 6.89 (d, 2H), 4.54 (d, 2H), 3.77 (s, 2H), 3.61 (d, 2H), 2.62 (d, 3H), 1.92-1.81 (m, 6H), 1.77-1.66 (m, 6H); MS: 377.3 [M+H]⁺.

The compounds below were synthesized in a similar manner as described for compound 8.

| Compound | Structure | Chemical Name | m/z |
|---|---|---|---|
| 8.01 | 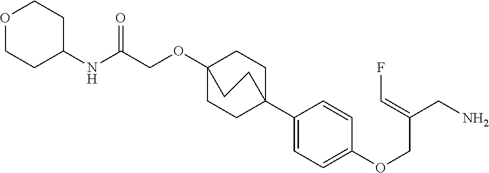 [SMILES: NC/C(=C\F)COc1ccc(C23CCC(OCC(=O)NC4CCOCC4)(CC2)CC3)cc1] | (E)-2-((4-(4-((2-(aminomethyl)-3- fluoroallyl)oxy)phenyl)bicyclo [2.2.2]octan-1-yl)oxy)-N- (tetrahydro-2H-pyran-4- yl)acetamide | 447.2 |
| 8.02 | 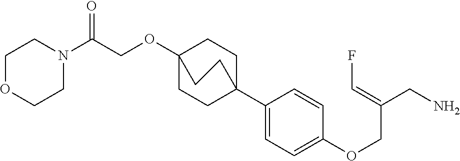 [SMILES: NC/C(=C\F)COc1ccc(C23CCC(OCC(=O)N4CCOCC4)(CC2)CC3)cc1] | (E)-2-((4-(4-((2-(aminomethyl)-3- fluoroallyl)oxy)phenyl)bicyclo [2.2.2]octan-1-yl)oxy)-1- morpholinoethanone | 433.1 |

### Compound 8.03

### (E)-3-Fluoro-2-((4-(4-(2-methoxyethoxy)bicyclo[2.2.2]octan-1-yl)phenoxy)methyl)prop-2-en-1-amine Hydrochloride

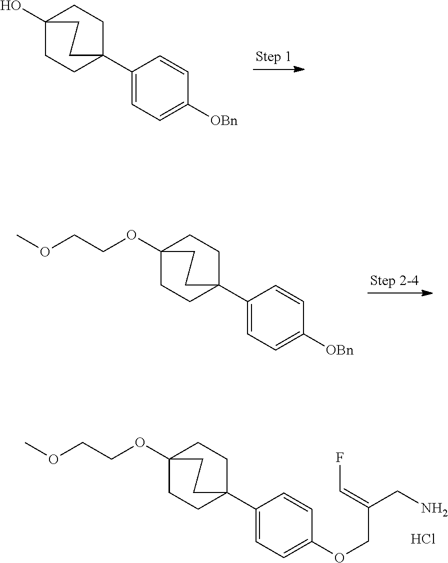 [SMILES: OC12CCC(c3ccc(OCc4ccccc4)cc3)(CC1)CC2 ; COCCOC12CCC(c3ccc(OCc4ccccc4)cc3)(CC1)CC2 ; COCCOC12CCC(c3ccc(OC/C(=C/F)CN)cc3)(CC1)CC2 ; Cl]

### Step 1: 1-(4-(Benzyloxy)phenyl)-4-(2-methoxyethoxy)bicyclo[2.2.2]octane

To a solution of 4-(4-(benzyloxy)phenyl)bicyclo[2.2.2]octan-1-ol (300 mg, 0.97 mmol) in DMF (4 mL), NaH (249 mg, 6.23 mmol, 60% purity) was added at 0° C. under a nitrogen atmosphere, the mixture warmed to room temperature, stirred for 30 min and then 1-bromo-2-methoxyethane (0.85 mL, 8.75 mmol) added. The mixture was stirred at room temperature overnight, the mixture carefully poured into H₂O (10 mL), extracted with EtOAc (3×10 mL), the combined organic layers washed with brine (10 mL), dried (Na₂SO₄), filtered, concentrated and purified by column chromatography (SiO₂, petroleum ether/EtOAc=10/1-5/1) to give 1-(4-(benzyloxy)phenyl)-4-(2-methoxyethoxy)bicyclo[2.2.2]octane (230 mg, 65%) as a yellow solid. ¹H NMR (400 MHz, CDCl₃): δ 7.47-7.30 (m, 5H), 7.26-7.20 (m, 2H), 6.95-6.86 (m, 2H), 5.04 (s, 2H), 3.60-3.47 (m, 4H), 3.40 (s, 3H), 2.01-1.89 (m, 6H), 1.86-1.74 (m, 6H).

### Step 2: 4-(4-(2-Methoxyethoxy)bicyclo[2.2.2]octan-1-yl)phenol

Pd/C (100 mg, 10% purity) was carefully added to 1-(4-(benzyloxy)phenyl)-4-(2-methoxyethoxy)bicyclo[2.2.2]octane (230 mg, 0.63 mmol) in EtOH (10 mL). The suspension was degassed under vacuum, purged with hydrogen several times then stirred under a hydrogen atmosphere (45 psi) at 30° C. overnight. The reaction mixture was filtered and concentrated to give 4-(4-(2-methoxyethoxy)bicyclo[2.2.2]octan-1-yl)phenol (150 mg, crude) as a white solid. ¹H NMR (400 MHz, CDCl₃): δ 7.14-7.06 (m, 2H), 6.72-6.63 (m, 2H), 4.64 (s, 1H), 3.49-3.42 (m, 4H), 3.31 (s, 3H), 1.89-1.82 (m, 6H), 1.77-1.67 (m, 6H); LCMS: 277.2 [M+H]⁺.

### Step 3: (E)-tert-Butyl (3-fluoro-2-((4-(4-(2-methoxyethoxy)bicyclo[2.2.2]octan-1-yl)phenoxy)methyl)allyl)carbamate

Cs₂CO₃ (530 mg, 1.63 mmol) was added to a mixture of 4-(4-(2-methoxyethoxy)bicyclo[2.2.2]octan-1-yl)phenol (150 mg, 0.54 mmol) and (E)-tert-butyl (2-(bromomethyl)-3-fluoroallyl)carbamate (175 mg, 0.65 mmol) in MeCN (4 mL). The mixture was stirred at room temperature overnight, poured into H₂O (10 mL), extracted with EtOAc (3×10 mL), the combined organic layer was washed with brine (10 mL), dried (Na₂SO₄), filtered and concentrated to give (E)-tert-butyl (3-fluoro-2-((4-(4-(2-methoxyethoxy)bicyclo[2.2.2]octan-1-yl)phenoxy)methyl)allyl)carbamate (200 mg, 80%) as a yellow oil. ¹H NMR (400 MHz, CDCl₃): δ 7.25-7.20 (m, 2H), 6.87-6.80 (m, 2H), 6.72 (d, 1H), 4.42 (d, 2H), 4.03-3.96 (m, 2H), 3.57-3.50 (m, 4H), 3.39 (s, 3H), 1.98-1.90 (m, 6H), 1.85-1.77 (m, 6H), 1.42 (s, 9H); LCMS: 408.5 [M+H-56]⁺.

### Step 4: (E)-3-Fluoro-2-((4-(4-(2-methoxyethoxy)bicyclo[2.2.2]octan-1-yl)phenoxy)methyl)prop-2-en-1-amine Hydrochloride

TFA (1.60 mL, 21.6 mmol) was added to a solution of (E)-tert-butyl (3-fluoro-2-((4-(4-(2-methoxyethoxy)bicyclo[2.2.2]octan-1-yl)phenoxy)methyl)allyl)carbamate (200 mg, 0.43 mmol) in DCM (3 mL). The mixture was stirred at room temperature for 1 h. The mixture was concentrated and purified by reverse-phase HPLC (water(0.04% HCl)-MeCN) to give (E)-3-fluoro-2-((4-(4-(2-methoxyethoxy)bicyclo[2.2.2]octan-1-yl)phenoxy)methyl)prop-2-en-1-amine hydrochloride (98 mg, 63%) as a white solid. ¹H NMR (400 MHz, DMSO-d₆): δ 8.25-8.00 (m, 3H), 7.28 (d, 1H), 7.24 (d, 2H), 6.89 (d, 2H), 4.56 (d, 2H), 3.58 (d, 2H), 3.46-3.41 (m, 2H), 3.39-3.35 (m, 2H), 3.23 (s, 3H), 1.89-1.81 (m, 6H), 1.73-1.64 (m, 6H); LCMS: 364.2 [M+H]⁺.

### Compound 9

### (E)-4-(4-((2-(Aminomethyl)-3-fluoroallyl)amino)phenyl)-N-(tetrahydro-2H-pyran-4-yl)bicyclo[2.2.2]octane-1-carboxamide Hydrochloride

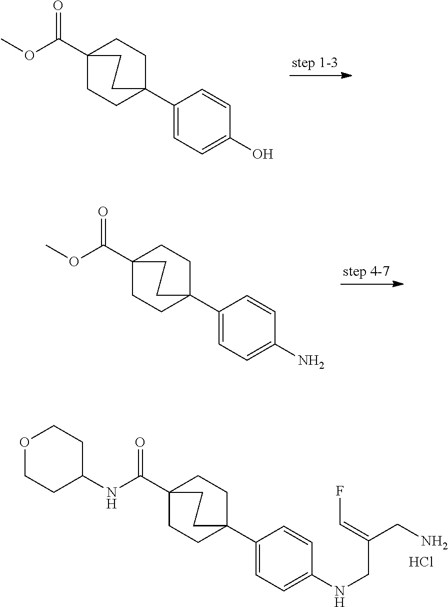 [SMILES: COC(=O)C12CCC(c3ccc(O)cc3)(CC1)CC2 ; COC(=O)C12CCC(c3ccc(N)cc3)(CC1)CC2 ; NC/C(=C\F)CNc1ccc(C23CCC(C(=O)NC4CCOCC4)(CC2)CC3)cc1 ; Cl]

### Step 1: Methyl 4-(4-(((trifluoromethyl)sulfonyl)oxy)phenyl)bicyclo[2.2.2]octane-1-carboxylate

To a solution of methyl 4-(4-hydroxyphenyl)bicyclo[2.2.2]octane-1-carboxylate (2.0 g, 7.68 mmol), triethylamine (3.89 g, 38.4 mmol) in DCM (30 mL) at 0° C., trifluoromethanesulfonic anhydride (3.25 g, 11.5 mmol) was added. The mixture was warmed to room temperature and stirred overnight. The reaction mixture was poured into water (20 mL) and extracted with EtOAc (2×20 mL). The combined organic layers were washed with H₂O (20 mL), brine (20 mL), dried (Na₂SO₄), filtered, concentrated and then purified by column chromatography (SiO₂, petroleum ether/ethyl acetate=10/1) to give methyl 4-(4-(((trifluoromethyl)sulfonyl)oxy)phenyl)bicyclo[2.2.2]octane-1-carboxylate (2.3 g, 76%) as a yellow solid. ¹H NMR (400 MHz, CDCl₃): δ 7.30 (d, 2H), 7.11 (d, 2H), 3.61 (s, 3H), 1.92-1.71 (m, 12H); LCMS: 393.2 [M+H]⁺.

### Step 2: Methyl 4-(4-((tert-butoxycarbonyl)amino)phenyl)bicyclo[2.2.2]octane-1-carboxylate

A mixture of methyl 4-(4-(((trifluoromethyl)sulfonyl)oxy)phenyl)bicyclo[2.2.2]octane-1-carboxylate (2.3 g, 5.9 mmol), tert-butyl carbamate (1.37 g, 11.7 mmol), Cs₂CO₃ (3.82 g, 11.7 mmol), Xantphos (1.36 g, 2.34 mmol), Pd₂(dba)₃ (1.07 g, 1.17 mmol) and dioxane (30 mL) was stirred at 100° C. overnight under a nitrogen atmosphere. The reaction mixture was cooled to room temperature, poured into water (40 mL) and extracted with EtOAc (3×40 mL). The combined organic layers were washed with H₂O (50 mL), brine (50 mL), dried (Na₂SO₄), filtered, concentrated and purified by column chromatography (SiO₂, petroleum ether/ethyl acetate=5/1) to give methyl 4-(4-((tert-butoxycarbonyl)amino)phenyl)bicyclo[2.2.2]octane-1-carboxylate (1.2 g, 57%) as a yellow solid. ¹H NMR (400 MHz, CDCl₃): δ 7.30 (d, 2H), 7.23 (d, 2H), 6.41 (s, 1H), 3.69 (s, 3H), 1.96-1.82 (m, 12H), 1.53 (s, 9H); LCMS: 304.1[M+H-56]⁺.

### Step 3: Methyl 4-(4-aminophenyl)bicyclo[2.2.2]octane-1-carboxylate

To a solution of methyl 4-(4-((tert-butoxycarbonyl)amino)phenyl)bicyclo[2.2.2]octane-1-carboxylate (1.2 g, 3.34 mmol) in DCM (20 mL) at room temperature, TFA (8 mL) was added and stirred at room temperature for 1.5 h. The reaction mixture was concentrated, diluted with water (40 mL) and then extracted with DCM (3×30 mL). The combined organic layers were washed with NaHCO₃ (50 mL), brine (50 mL), dried (Na₂SO₄), filtered, concentrated and then purified by column chromatography (SiO₂, petroleum ether/ethyl acetate=5/1) to give methyl 4-(4-aminophenyl)bicyclo[2.2.2]octane-1-carboxylate (750 mg, 87%) as a yellow solid. ¹H NMR (400 MHz, CDCl₃): δ 7.11 (d, 2H), 6.64 (d, 2H), 3.67 (s, 3H), 3.56 (s, 2H), 1.96-1.76 (m, 12H); LCMS: 260.1 [M+H]⁺.

### Step 4: (Z)-Methyl 4-(4-((2-(((tert-butoxycarbonyl)amino)methyl)-3-fluoroallyl)amino)phenyl)bicyclo[2.2.2]octane-1-carboxylate

A mixture of methyl 4-(4-aminophenyl)bicyclo[2.2.2]octane-1-carboxylate (200 mg, 0.77 mmol), (E)-tert-butyl (2-(bromomethyl)-3-fluoroallyl)carbamate (248 mg, 0.93 mmol), Cs₂CO₃ (754 mg, 2.31 mmol) and MeCN (10 mL) was stirred at 40° C. for 3.5 h. The reaction mixture was poured into water (40 mL) and extracted with EtOAc (3×40 mL). The combined organic layers were washed with H₂O (50 mL), brine (50 mL), dried (Na₂SO₄), filtered, concentrated and then purified by column chromatography (SiO₂, petroleum ether/ethyl acetate=5/1) to give (Z)-methyl 4-(4-((2-(((tert-butoxycarbonyl)amino)methyl)-3-fluoroallyl)amino)phenyl)bicyclo[2.2.2]octane-1-carboxylate (189 mg, 55%) as a yellow solid. ¹H NMR (400 MHz, CDCl₃): δ 7.05 (d, 2H), 6.71 (s, 1H), 6.45 (d, 1H), 6.58-6.41 (m, 2H), 4.78-4.63 (m, 1H), 3.83-3.57 (m, 7H), 1.86-1.70 (m, 12H), 1.38 (s, 9H); LCMS: 447.2 [M+H]⁺.

### Step 5: (Z)-4-(4-((2-(((tert-Butoxycarbonyl)amino)methyl)-3-fluoroallyl)amino)phenyl)bicyclo[2.2.2]octane-1-carboxylic Acid

A mixture of (Z)-methyl 4-(4-((2-(((tert-butoxycarbonyl)amino)methyl)-3-fluoroallyl)amino)phenyl)bicyclo[2.2.2]octane-1-carboxylate (189 mg, 0.42 mmol), LiOH.H₂O (62 mg, 1.48 mmol), THF (5 mL), MeOH (5 mL) and H₂O (5 mL) was stirred at room temperature overnight. The reaction mixture was concentrated to remove the organic solvent, adjusted to pH=3 with 1M HCl then filtered. The cake was collected and dried in vacuum to give (Z)-4-(4-((2-(((tert-butoxycarbonyl)amino)methyl)-3-fluoroallyl)amino)phenyl)bicyclo[2.2.2]octane-1-carboxylic acid (139 mg, 76%) as a yellow solid. ¹H NMR (400 MHz, CDCl₃): δ 7.12 (d, 2H), 6.64 (d, 1H), 6.59 (d, 2H), 4.80-4.68 (m, 1H), 3.92-3.83 (m, 2H), 3.68 (d, 2H), 1.98-1.90 (m, 6H), 1.87-1.80 (m, 6H), 1.46 (s, 9H); LCMS: 431.2 [M−H]⁻.

### Step 6: (Z)-tert-Butyl (3-fluoro-2-(((4-(4-((tetrahydro-2H-pyran-4-yl)carbamoyl)bicyclo[2.2.2]octan-1-yl)phenyl)amino)methyl)allyl)carbamate

A mixture of (Z)-4-(4-((2-(((tert-butoxycarbonyl)amino)methyl)-3-fluoroallyl)amino)phenyl)bicyclo[2.2.2]octane-1-carboxylic acid (139 mg, 0.32 mmol), tetrahydro-2H-pyran-4-amine (49 mg, 0.48 mmol), HATU (184 mg, 0.48 mmol,), DIEA (83 mg, 0.64 mmol) and DCM (10 mL) was stirred at 30° C. for 3 h. The reaction mixture was poured into water (20 mL) and extracted with DCM (3×20 mL). The combined organic layers were washed with H₂O (50 mL), brine (50 mL), dried (Na₂SO₄), filtered, concentrated and then purified by reverse-phase HPLC (water(0.04% HCl)-MeCN) to give (Z)-tert-butyl (3-fluoro-2-(((4-(4-((tetrahydro-2H-pyran-4-yl)carbamoyl)bicyclo[2.2.2]octan-1-yl)phenyl)amino)methyl)allyl)carbamate (120 mg, 69%) as a yellow oil. ¹H NMR (400 MHz, CDCl₃): δ 7.17-7.09 (m, 2H), 6.63 (d, 1H), 6.65-6.57 (m, 2H), 5.41 (d, 1H), 4.83-4.68 (m, 1H), 4.05-3.80 (m, 5H), 3.68 (d, 2H), 3.54-3.41 (m, 2H), 1.75 (m, 14H), 1.48-1.37 (m, 11H); LCMS: 516.2 [M+H]⁺.

### Step 7: (E)-4-(4-((2-(Aminomethyl)-3-fluoroallyl)amino)phenyl)-N-(tetrahydro-2H-pyran-4-yl)bicyclo[2.2.2]octane-1-carboxamide Hydrochloride

To a solution of (Z)-tert-butyl (3-fluoro-2-(((4-(4-((tetrahydro-2H-pyran-4-yl)carbamoyl)bicyclo[2.2.2]octan-1-yl)phenyl)amino)methyl)allyl)carbamate (120 mg, 0.23 mmol) in DCM (5 mL) at room temperature, TFA (2 mL) was added and stirred at room temperature for 1 h. The mixture was concentrated to dryness and purified by reverse-phase HPLC (water(0.04% HCl)-MeCN) to give (E)-4-(4-((2-(aminomethyl)-3-fluoroallyl)amino)phenyl)-N-(tetrahydro-2H-pyran-4-yl)bicyclo[2.2.2]octane-1-carboxamide hydrochloride (20 mg, 18%) as a white solid. ¹H NMR (400 MHz, DMSO-d₆): δ 8.24-7.98 (m, 3H), 7.22-7.13 (m, 1H), 7.09 (d, 1H), 7.08 (d, 2H), 6.64 (d, 2H), 3.86-3.68 (m, 7H), 3.37-3.24 (m, 3H), 1.77-1.68 (m, 12H), 1.62-1.53 (m, 2H), 1.51-1.38 (m, 2H); LCMS: 416.3 [M+H]⁺.

### Compound 10

### (E)-5-(4-((2-(Aminomethyl)-3-fluoroallyl)oxy)phenyl)-N-methylbicyclo[3.2.2]nonane-1-carboxamide Hydrochloride

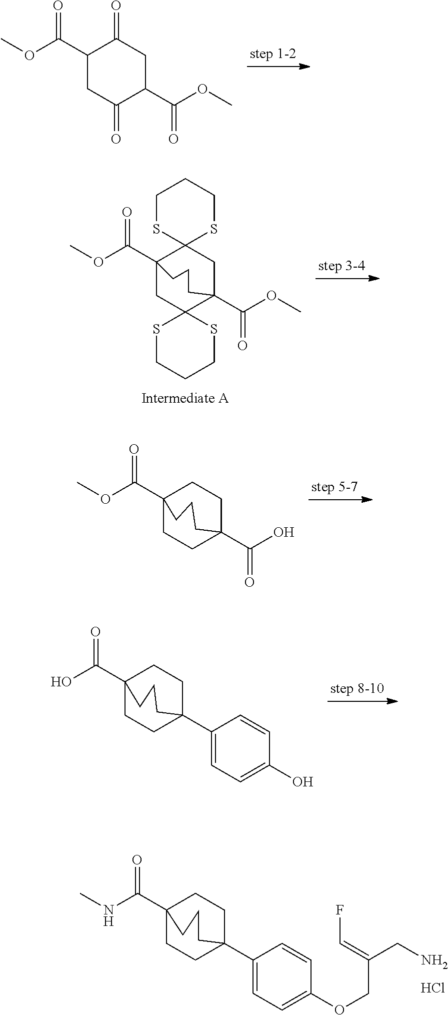

### Step 1: Dimethyl 6,8-dioxobicyclo[3.2.2]nonane-1,5-dicarboxylate

NaH (19.3 g, 482 mmol, 60% purity) was added to a mixture of dimethyl 2,5-dioxocyclohexane-1,4-dicarboxylate (50 g, 219 mmol) in DME (500 mL) at 0° C. under a nitrogen atmosphere. The reaction was heated to 80° C. and stirred for 3 h. The mixture was concentrated, 1,3-dibromopropane (250 mL, 2.45 mol) was added to the residue, the reaction stirred at 145° C. overnight, cooled to room temperature, filtered, concentrated and purified by column chromatography (SiO₂, petroleum ether/ethyl acetate=50/1-20/1) to give the crude product (23 g). The crude product was dissolved in EtOH (100 mL) at 80° C., the solution slowly cooled to room temperature and stirred overnight. The reaction was filtered, the filter cake collected and dried to give dimethyl 6,8-dioxobicyclo[3.2.2]nonane-1,5-dicarboxylate (8.0 g, 35%) as a white solid. H NMR (400 MHz, CDCl₃): δ 3.80 (s, 6H), 3.42 (s, 1H), 3.37 (s, 1H), 2.81 (s, 1H), 2.76 (s, 1H), 2.59-2.44 (m, 2H), 2.07-1.91 (m, 2H), 1.87-1.77 (m, 2H).

### Step 2: Bis-1,3-dithiane Intermediate A

BF₃.Et₂O (21 mL, 168 mmol) was added to a mixture of dimethyl 6,8-dioxobicyclo[3.2.2]nonane-1,5-dicarboxylate (9.0 g, 33.6 mmol), propane-1,3-dithiol (13.5 mL, 134 mmol) and CHCl₃ (150 mL) at 0° C. under a nitrogen atmosphere. Na₂SO₄ (19.1 g, 134 mmol) was added and the reaction stirred at 30° C. overnight. The reaction was poured carefully into saturated NaHCO₃ solution (50 mL), extracted with EtOAc (3×50 mL), the combined organic layers were washed with brine (50 mL), dried (Na₂SO₄), filtered, concentrated and purified by column chromatography (SiO₂, petroleum ether/ethyl acetate=20/1-10/1) to give bis-1,3-dithiane intermediate A (8.0 g, crude) as a yellow oil. LCMS: 449.1 [M+H]⁺.

### Step 3: Dimethyl bicyclo[3.2.2]nonane-1,5-dicarboxylate

To a solution of Bis-1,3-dithiane intermediate A (2.0 g, 4.46 mmol) in EtOH (150 mL) was added Raney Ni (20.0 g, 341 mmol). The suspension was degassed under vacuum and purged with hydrogen 3 times. The mixture was stirred under hydrogen (45 psi) at 80° C. for 72 h, cooled to room temperature, filtered and concentrated to give dimethyl bicyclo[3.2.2]nonane-1,5-dicarboxylate (1.5 g, crude) as a yellow oil. LCMS: 241.1 [M+H]⁺.

### Step 4: 5-(Methoxycarbonyl)bicyclo[3.2.2]nonane-1-carboxylic Acid

NaOH (499 mg, 12.5 mmol) was added to a mixture of dimethyl bicyclo[3.2.2]nonane-1,5-dicarboxylate (3.0 g, 12.5 mmol) in MeOH (4 mL) and THF (40 mL). The mixture was stirred at 30° C. for 18 h, concentrated to remove organic solvents, 1M HCl was added until pH=3, extracted with EtOAc (3×20 mL), the organic layers were washed with brine (20 mL), dried (Na₂SO₄), filtered and concentrated to give 5-(methoxycarbonyl)bicyclo[3.2.2]nonane-1-carboxylic acid (2.7 g, crude) as a white solid. ¹H NMR (400 MHz, CDCl₃): δ 11.59 (s, 1H), 3.65 (s, 3H), 1.98-1.83 (m, 12H), 1.81-1.64 (m, 2H); LCMS: 225.1 [M−H]⁻.

### Step 5: Methyl 5-bromobicyclo[3.2.2]nonane-1-carboxylate

To a stirred suspension 5-(methoxycarbonyl)bicyclo[3.2.2]nonane-1-carboxylic acid (250 mg, 1.10 mmol) in acetone (5 mL) was added 1 M NaOH (1.2 mL) and the resulting clear pale yellow solution was allowed to stir at room temperature for 10 min. A solution of AgNO₃ (199 mg, 1.17 mmol) in H₂O (0.5 mL) was added dropwise and an immediate thick brown suspension formed. The reaction was stirred at room temperature for a further 1 h. The suspension was filtered, washed with water (10 mL), acetone (10 mL) and dried under vacuum to give a brown solid.

To a suspension of the silver salt in petroleum ether (10 mL) was added dropwise bromine (0.06 mL, 0.93 mmol) under a nitrogen atmosphere. The resulting orange suspension was stirred at room temperature for 0.5 h then at 60° C. for 1 h. The reaction mixture was cooled to room temperature, the suspension was filtered, the solid was washed with petroleum ether (3×20 mL) and 1M sodium carbonate (2×20 mL). The organic phase was separated, washed with brine (20 mL), dried (Na₂SO₄), filtered and concentrated to give methyl 5-bromobicyclo[3.2.2]nonane-1-carboxylate (123 mg) as a yellow oil. ¹H NMR (400 MHz, CDCl₃): δ 3.65 (s, 3H), 2.50-2.43 (m, 6H), 2.08-1.81 (m, 6H), 1.79-1.65 (m, 2H).

### Step 6: Methyl 5-(4-hydroxyphenyl)bicyclo[3.2.2]nonane-1-carboxylate

FeCl₃ (850 mg, 5.24 mmol) was added to a mixture of methyl 5-bromobicyclo[3.2.2]nonane-1-carboxylate (370 mg, 1.42 mmol) in phenol (3.96 g, 42.1 mmol) under a nitrogen atmosphere. The resulting reaction mixture was stirred at 50° C. overnight, concentrated and purified by column chromatography (SiO₂, petroleum ether/ethyl acetate=50/1-5/1) to give methyl 5-(4-hydroxyphenyl)bicyclo[3.2.2]nonane-1-carboxylate (100 mg, 26%) as a yellow solid. LCMS: 273.1 [M−H]⁻.

### Step 7: 5-(4-Hydroxyphenyl)bicyclo[3.2.2]nonane-1-carboxylic Acid

LiOH.H₂O (128 mg, 3.06 mmol) was added to a mixture of methyl 5-(4-hydroxyphenyl)bicyclo[3.2.2]nonane-1-carboxylate (140 mg, 0.51 mmol), THF (4 mL), H₂O (0.8 mL) and MeOH (0.8 mL). The mixture was stirred at 35° C. for 20 h, 1M HCl carefully added until pH=3, extracted with EtOAc (3×10 mL), the combined organic layers were washed with brine (10 mL), dried (Na₂SO₄), filtered and concentrated to give 5-(4-hydroxyphenyl)bicyclo[3.2.2]nonane-1-carboxylic acid (120 mg, crude) as a yellow solid. ¹H NMR (400 MHz, CDCl₃): δ 7.18 (d, 2H), 6.76 (d, 2H), 2.22-2.13 (m, 2H), 2.08-1.93 (m, 6H), 1.91-1.72 (m, 6H); LCMS: 259.1 [M−H]⁻.

### Step 8: 5-(4-Hydroxyphenyl)-N-methylbicyclo[3.2.2]nonane-1-carboxamide

To a solution of 5-(4-hydroxyphenyl)bicyclo[3.2.2]nonane-1-carboxylic acid (50 mg, 0.19 mmol) in DCM (6 mL), HATU (110 mg, 0.29 mmol) and DIPEA (0.30 mL, 1.92 mmol) then methanamine HCl salt (38.9 mg, 0.58 mmol) was added and the mixture stirred at 30° C. overnight. The reaction was poured into H₂O (20 mL), extracted with EtOAc (3×20 mL), the combined organic layers washed with brine (20 mL), dried (Na₂SO₄), filtered and concentrated to give 5-(4-hydroxyphenyl)-N-methylbicyclo[3.2.2]nonane-1-carboxamide (50 mg, crude) as a yellow oil. LCMS: 274.1 [M+H]⁺.

### Step 9: (E)-tert-Butyl (3-fluoro-2-((4-(5-(methylcarbamoyl)bicyclo[3.2.2]nonan-1-yl)phenoxy)methyl)allyl)carbamate

Cs₂CO₃ (179 mg, 0.55 mmol) was added to a mixture of 5-(4-hydroxyphenyl)-N-methylbicyclo[3.2.2]nonane-1-carboxamide (50 mg, 0.18 mmol) and (E)-tert-butyl (2-(bromomethyl)-3-fluoroallyl)carbamate (59 mg, 0.22 mmol) in MeCN (4 mL) and the reaction stirred at room temperature overnight. The reaction was poured into H₂O (10 mL), extracted with EtOAc (3×10 mL), the combined organic layers were washed with brine (10 mL), dried (Na₂SO₄), filtered, concentrated and purified by column chromatography (SiO₂, petroleum ether/ethyl acetate=10/1-1/1) to give (E)-tert-butyl (3-fluoro-2-((4-(5-(methylcarbamoyl)bicyclo[3.2.2]nonan-1-yl)phenoxy)methyl)allyl)carbamate (70 mg, crude) as a yellow oil. LCMS: 461.4 [M+H]⁺.

### Step 10: (E)-5-(4-((2-(Aminomethyl)-3-fluoroallyl)oxy)phenyl)-N-methylbicyclo[3.2.2]nonane-1-carboxamide Hydrochloride

TFA (0.40 mL, 5.45 mmol) was added to a solution of (E)-tert-butyl (3-fluoro-2-((4-(5-(methylcarbamoyl)bicyclo[3.2.2]nonan-1-yl)phenoxy)methyl)allyl)carbamate (70 mg, 0.15 mmol) in DCM (2 mL) and stirred at room temperature for 1 h. The mixture was concentrated and purified by reverse-phase HPLC (water(0.04% HCl)-MeCN) to give (E)-5-(4-((2-(aminomethyl)-3-fluoroallyl)oxy)phenyl)-N-methylbicyclo[3.2.2]nonane-1-carboxamide hydrochloride (17 mg, 28%) as a white solid. ¹H NMR (400 MHz, DMSO-d₆): δ 8.31-8.01 (m, 3H), 7.28 (d, 1H), 7.33 (d, 1H), 7.23 (d, 2H), 6.86 (d, 2H), 4.56 (d, 2H), 3.65-3.55 (m, 2H), 2.54 (d, 3H), 2.16-2.01 (m, 2H), 1.94-1.83 (m, 2H), 1.79-1.66 (m, 10H); LCMS: 361.2 [M+H]+.

### Compound 10.01

### (E)-5-(4-((2-(Aminomethyl)-3-fluoroallyl)oxy)phenyl)-N-(tetrahydro-2H-pyran-4-yl)bicyclo[3.2.2]nonane-1-carboxamide Hydrochloride

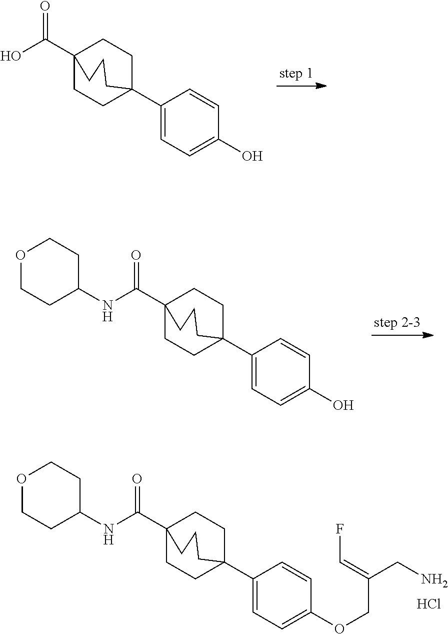 [SMILES: O=C(O)C12CCCC(c3ccc(O)cc3)(CC1)CC2 ; O=C(NC1CCOCC1)C12CCCC(c3ccc(O)cc3)(CC1)CC2 ; NC/C(=C\F)COc1ccc(C23CCCC(C(=O)NC4CCOCC4)(CC2)CC3)cc1 ; Cl]

### Step 1: 5-(4-Hydroxyphenyl)-N-(tetrahydro-2H-pyran-4-yl)bicyclo[3.2.2]nonane-1-carboxamide

To a solution of 5-(4-hydroxyphenyl)bicyclo[3.2.2]nonane-1-carboxylic acid (50 mg, 0.19 mmol) in DCM (5 mL), HATU (110 mg, 0.29 mmol) and DIPEA (0.30 mL, 1.92 mmol) then tetrahydropyran-4-amine (58 mg, 0.58 mmol) were added and the mixture stirred at 30° C. overnight. The reaction was poured into H₂O (10 mL), extracted with EtOAc (3×10 mL), the combined organic layers were washed with brine (10 mL), dried (Na₂SO₄), filtered and concentrated to give 5-(4-hydroxyphenyl)-N-(tetrahydro-2H-pyran-4-yl)bicyclo[3.2.2]nonane-1-carboxamide (65 mg, crude) as a yellow oil. LCMS: 344.3 [M+H]⁺.

### Step 2: (E)-tert-Butyl (3-fluoro-2-((4-(5-((tetrahydro-2H-pyran-4-yl)carbamoyl)bicyclo[3.2.2]nonan-1-yl)phenoxy)methyl)allyl)carbamate

Cs₂CO₃ (185 mg, 0.57 mmol) was added to a mixture of 5-(4-hydroxyphenyl)-N-(tetrahydro-2H-pyran-4-yl)bicyclo[3.2.2]nonane-1-carboxamide (65 mg, 0.19 mmol), (E)-tert-butyl (2-(bromomethyl)-3-fluoroallyl)carbamate (61 mg, 0.23 mmol) in MeCN (4 mL) and stirred at room temperature overnight. The reaction was poured into H₂O (10 mL), extracted with EtOAc (3×10 mL), the combined organic layers were washed with brine (10 mL), dried (Na₂SO₄), filtered, concentrated and purified by column chromatography (SiO₂, petroleum ether/EtOAc=10/1-1/1) to give (E)-tert-butyl (3-fluoro-2-((4-(5-((tetrahydro-2H-pyran-4-yl)carbamoyl)bicyclo[3.2.2]nonan-1-yl)phenoxy)methyl)allyl)carbamate (100 mg, crude) as a yellow oil. LCMS: 531.4 [M+H]⁺.

### Step 3: (E)-5-(4-((2-(Aminomethyl)-3-fluoroallyl)oxy)phenyl)-N-(tetrahydro-2H-pyran-4-yl)bicyclo[3.2.2]nonane-1-carboxamide Hydrochloride

TFA (0.45 mL, 6.08 mmol) was added to a solution of (E)-tert-butyl (3-fluoro-2-((4-(5-((tetrahydro-2H-pyran-4-yl)carbamoyl)bicyclo[3.2.2]nonan-1-yl)phenoxy)methyl)allyl)carbamate (100 mg, 0.17 mmol) in DCM (2 mL) and stirred at room temperature for 1 h. The mixture was concentrated and purified by reverse-phase HPLC (water(0.04% HCl)-MeCN) to give (E)-5-(4-((2-(aminomethyl)-3-fluoroallyl)oxy)phenyl)-N-(tetrahydro-2H-pyran-4-yl)bicyclo[3.2.2]nonane-1-carboxamide hydrochloride (25 mg, 34%) as a pink solid. ¹H NMR (400 MHz, DMSO-d₆): δ 8.30-8.05 (m, 3H), 7.28 (d, 1H), 7.23 (d, 2H), 7.13 (d, 1H), 6.88 (d, 2H), 4.57 (d, 2H), 3.85-3.83 (m, 2H), 3.74-3.69 (m, 1H), 3.58 (d, 2H), 3.35-3.23 (m, 2H), 2.16-2.05 (m, 2H), 1.97-1.86 (m, 2H), 1.80-1.65 (m, 10H), 1.62-1.53 (m, 2H), 1.51-1.40 (m, 2H); LCMS: 431.3 [M+H]⁺.

### Compound 11

### (2E)-3-fluoro-2-({4-[4-(5-methyl(1,3,4-oxadiazol-2-yl))bicyclo[2.2.2]octyl]phenoxy}methyl)prop-2-enylamine

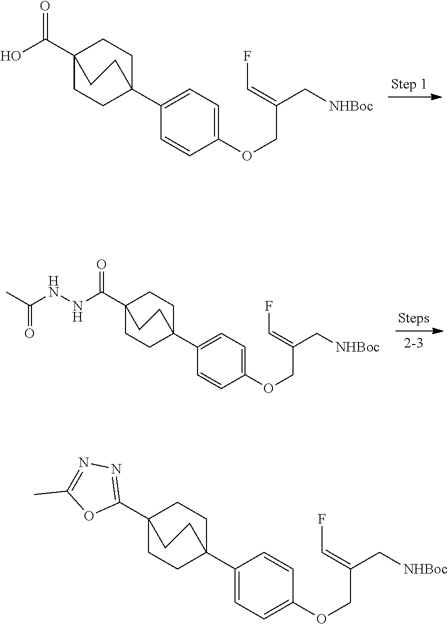 [SMILES: CC(C)(C)OC(=O)NC/C(=C\F)COc1ccc(C23CCC(C(=O)O)(CC2)CC3)cc1 ; CC(=O)NNC(=O)C12CCC(c3ccc(OC/C(=C/F)CNC(=O)OC(C)(C)C)cc3)(CC1)CC2 ; Cc1nnc(C23CCC(c4ccc(OC/C(=C/F)CNC(=O)OC(C)(C)C)cc4)(CC2)CC3)o1]

### Step 1: N-({4-[4-((2E)-2-{[(tert-butoxy)carbonylamino]methyl}-3-fluoroprop-2-enyloxy)phenyl]bicyclo[2.2.2]octyl}carbonylamino)acetamide

4-[4-((2E)-2-{[(tert-butoxy)carbonylamino]methyl}-3-fluoroprop-2-enyloxy)phenyl]bicyclo[2.2.2]octanecarboxylic acid (0.15 g, 0.34 mmol) was dissolved in DMF (3 mL), acethydrazide (0.028 g, 0.38 mmol), DIEA (0.18 mL, 1.04 mmol) and HATU (0.17 g, 0.45 mmol) were added and the reaction stirred at room temperature for 1.5 h. The reaction mixture was diluted with ethyl acetate, washed with water and brine, organics dried (MgSO₄) and concentrated. The oily residue was triturated with ethyl acetate and heptane and the white precipitate filtered, washed with heptane and dried under vacuum to give N-({4-[4-((2E)-2-{[(tert-butoxy)carbonyl amino]methyl}-3-fluoroprop-2-enyloxy)phenyl]bicyclo[2.2.2]octyl}carbonylamino)acetamide (0.16 g, 94%) as a white solid. LCMS: 390.1 [M+H-100]⁺.

### Step 2: (E)-tert-butyl (3-fluoro-2-((4-(4-(5-methyl-1,3,4-oxadiazol-2-yl)bicyclo[2.2.2]octan-1-yl)phenoxy)methyl)allyl)carbamate

p-Toluenesulfonyl chloride (0.093 g, 0.49 mmol) was added to a solution of N-({4-[4-((2E)-2-{[(tert-butoxy)carbonylamino]methyl}-3-fluoroprop-2-enyloxy)phenyl]bicyclo[2.2.2]octyl}carbonylamino)acetamide (0.16 g, 0.32 mmol) and TEA (0.09 mL, 0.65 mmol) in DCM (8 mL). The reaction mixture was stirred at room temperature for 45 min, diluted with DCM and washed with water. The aqueous layer was extracted with DCM, combined organics washed with brine, dried (MgSO₄) and concentrated to give (E)-tert-butyl (3-fluoro-2-((4-(4-(5-methyl-1,3,4-oxadiazol-2-yl)bicyclo[2.2.2]octan-1-yl)phenoxy)methyl)allyl)carbamate (0.15 g) which was used without further purification. LCMS: 472.4 [M+H]⁺.

### Step 3: (E)-3-fluoro-2-((4-(4-(5-methyl-1,3,4-oxadiazol-2-yl)bicyclo[2.2.2]octan-1-yl)phenoxy)methyl)prop-2-en-1-amine

A solution of N-[(2E)-3-fluoro-2-({4-[4-(5-methyl(1,3,4-oxadiazol-2-yl))bicyclo[2.2.2]octyl]phenoxy}methyl)prop-2-enyl](tert-butoxy)carboxamide (0.15 g, 0.32 mmol) in trifluoroacetic acid (1 mL) and DCM (2 mL) was stirred at room temperature overnight. The reaction was concentrated and purified by prep HPLC eluting with 10-50 ACN/water, 0.1% TFA. The combined product fractions were diluted with ethyl acetate, washed with saturated NaHCO₃ and brine, dried (MgSO₄) and concentrated to give (E)-3-fluoro-2-((4-(4-(5-methyl-1,3,4-oxadiazol-2-yl)bicyclo[2.2.2]octan-1-yl)phenoxy)methyl)prop-2-en-1-amine (0.03 g, 22%) as a white solid. ¹H NMR (400 MHz, CDCl₃): δ 7.19-7.17 (d, 2H), 6.81-6.79 (d, 2H), 6.73-6.52 (d, 1H), 4.40 (s, 2H), 3.46 (s, 2H), 2.43 (s, 3H), 2.01-1.83 (m, 12H); LCMS: 372.2 [M+H]⁺.

The compounds below were synthesized in a similar manner as described for compound 11.

| Compound | Structure | Chemical Name | m/z |
|---|---|---|---|
| 11.01 | 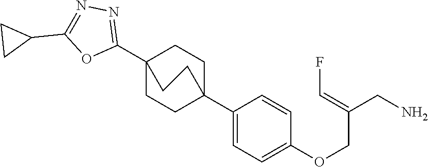 [SMILES: NC/C(=C\F)COc1ccc(C23CCC(c4nnc(C5CC5)o4)(CC2)CC3)cc1] | (E)-2-((4-(4-(5-cyclopropyl- 1,3,4-oxadiazol-2- yl)bicyclo[2.2.2]octan-1- yl)phenoxy)methyl)-3- fluoroprop-2-en-1-amine | 398.2 |
| 11.02 | 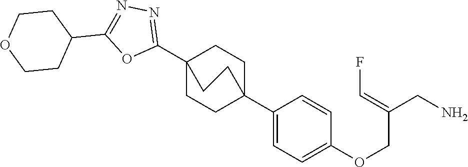 [SMILES: NC/C(=C\F)COc1ccc(C23CCC(c4nnc(C5CCOCC5)o4)(CC2)CC3)cc1] | (E)-3-fluoro-2-((4-(4-(5- (tetrahydro-2H-pyran-4-yl)- 1,3,4-oxadiazol-2- yl)bicyclo[2.2.2]octan-1- yl)phenoxy)methyl)prop-2- en-1-amine | 442.2 |

### Example A-1: Parenteral Pharmaceutical Composition

To prepare a parenteral pharmaceutical composition suitable for administration by injection (subcutaneous, intravenous), 1-1000 mg of a compound described herein, or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection

### Example A-2: Oral Solution

To prepare a pharmaceutical composition for oral delivery, a sufficient amount of a compound described herein, or a pharmaceutically acceptable salt thereof, is added to water (with optional solubilizer(s), optional buffer(s) and taste masking excipients) to provide a 20 mg/mL solution.

### Example A-3: Oral Tablet

A tablet is prepared by mixing 20-50% by weight of a compound described herein, or a pharmaceutically acceptable salt thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100-1000 mg.

### Example A-4: Oral Capsule

To prepare a pharmaceutical composition for oral delivery, 1-1000 mg of a compound described herein, or a pharmaceutically acceptable salt thereof, is mixed with starch or other suitable powder blend. The mixture is incorporated into an oral dosage unit such as a hard gelatin capsule, which is suitable for oral administration.

In another embodiment, 1-1000 mg of a compound described herein, or a pharmaceutically acceptable salt thereof, is placed into a size 4 capsule, or a size 1 capsule (hypromellose or hard gelatin) and the capsule is closed.

### Example A-5: Topical Gel Composition

To prepare a pharmaceutical topical gel composition, a compound described herein, or a pharmaceutically acceptable salt thereof, is mixed with hydroxypropyl celluose, propylene glycol, isopropyl myristate and purified alcohol USP. The resulting gel mixture is then incorporated into containers, such as tubes, which are suitable for topical administration.

### Example B-1: Human Semicarbazide-Sensitive Amine Oxidase (SSAO) Assay

### Test Compounds

Compounds were dissolved in DMSO to provide a Concentration Response Curve (CRC; at 500 times the final concentration) over a range of 6-10 dilutions.

### Assay

Recombinant Human SSAO/VAP-1/AOC3 (R&D systems; Catalog #3957-AO) was used to screen compound potency in vitro, according to previously described methods (J Pharmacol Exp Ther. 2013 November; 347(2):365-74). SSAO enzyme was suspended in 50 mM HEPES buffer to a working concentration of 2.5 μg/ml and 40 μL of this enzyme mixture was then added to each well of a F16 Black Maxisorp 96 well Plate (Nunc, Catalog #475515). Ten microliters of each test compound (at 5 times the final concentration) were added to each well, resulting in a final well volume of 50 μL of the enzyme and inhibitor mixture. The compounds were preincubated with the enzyme for 30 minutes at 37° C., prior to the addition of a 40 μL volume of Amplex Ultra Red (125 uM AUR; Molecular Probes, Catalog # A36006)/Horseradish peroxidase (2.5 U/ml HRP; Sigma-Aldrich Catalog # P8375) oxidase detection reagent containing cytochrome C (7.5 M; Sigma-Aldrich Catalog # C7752). Cytochrome C was included in the AUR/HRP detection mixture to reduce the background fluorescence that can occur via the spontaneous redox reaction between AUR and HRP. The SSAO enzyme reaction was then initiated by adding 10 μL of the SSAO substrate, benzylamine (Sigma-Aldrich Catalog # B5136), and SSAO activity was measured in kinetic mode over a 30-120 minute sampling period (excit. 544 nm; emit 590 nm; cut off 570 nm; medium gain) to obtain IC₅₀ values for enzyme activity in each treatment well.

Representative data for exemplary compounds disclosed herein is presented in Table 3.

TABLE 3

| | Human SSAO |
|---|---|
| Compound | IC₅₀ (uM) |
| 1 | ++++ |
| 1.01 | +++ |
| 2 | ++++ |
| 2.01 | ++++ |
| 2.02 | ++++ |
| 2.03 | ++++ |
| 2.04 | ++++ |
| 2.05 | +++ |
| 2.06 | ++++ |
| 2.06b | ++++ |
| 2.07 | ++++ |
| 2.08 | ++++ |
| 2.09 | ++++ |
| 2.10 | ++++ |
| 2.11 | ++++ |
| 2.12 | ++++ |
| 2.13 | ++++ |
| 2.14 | ++++ |
| 2.15 | ++++ |
| 2.16 | ++++ |
| 2.17 | ++++ |
| 2.18 | ++++ |
| 2.19 | ++++ |
| 2.20 | ++++ |
| 2.21 | ++++ |
| 2.22 | ++++ |
| 2.23 | ++++ |
| 2.24 | ++++ |
| 2.25 | ++++ |
| 2.26 | ++++ |
| 2.27 | ++++ |
| 2.28 | ++++ |
| 2.29 | ++++ |
| 2.30 | ++++ |
| 2.31 | ++++ |
| 2.32 | ++++ |
| 2.33 | ++++ |
| 2.34 | ++++ |
| 2.35 | ++++ |
| 2.36 | ++++ |
| 2.37 | ++++ |
| 2.38 | ++++ |
| 2.39 | ++++ |
| 2.40 | ++++ |
| 2.41 | ++++ |
| 2.42 | ++++ |
| 2.43 | ++++ |
| 2.44 | ++++ |
| 2.45 | ++++ |
| 2.46 | ++++ |
| 2.47 | ++++ |
| 2.48 | ++++ |
| 2.49 | ++++ |
| 2.50 | ++++ |
| 2.51 | ++++ |
| 2.52 | ++++ |
| 2.53 | ++++ |
| 2.54 | ++++ |
| 2.55 | ++++ |
| 2.56 | ++++ |
| 2.57 | ++++ |
| 2.58 | ++++ |
| 2.59 | ++++ |
| 2.60 | ++++ |
| 2.61 | ++++ |
| 2.62 | ++++ |
| 2.63 | ++++ |
| 2.64 | ++++ |
| 2.65 | ++++ |
| 2.66 | ++++ |
| 2.67 | ++++ |
| 2.68 | ++++ |
| 2.69 | ++++ |
| 2.70 | ++++ |
| 2.71 | ++++ |
| 2.72 | ++++ |
| 2.73 | ++++ |
| 2.74 | ++++ |
| 2.75 | ++++ |
| 2.76 | ++++ |
| 2.77 | ++++ |
| 2.78 | ++++ |
| 2.79 | ++++ |
| 2.80 | ++++ |
| 2.81 | ++++ |
| 2.82 | ++++ |
| 2.83 | ++++ |
| 2.84 | ++++ |
| 2.85 | ++++ |
| 2.86 | ++++ |
| 2.87 | ++++ |
| 2.88 | ++++ |
| 2.89 | ++++ |
| 2.90 | ++++ |
| 2.91 | ++++ |
| 2.92 | ++++ |
| 2.93 | ++++ |
| 2.94 | ++++ |
| 2.95 | ++++ |
| 2.96 | ++++ |
| 2.97 | ++++ |
| 2.98 | ++++ |
| 2.99 | ++++ |
| 2.100 | ++++ |
| 2.101 | ++++ |
| 2.102 | +++ |
| 2.103 | ++++ |
| 2.104 | ++++ |
| 2.105 | ++++ |
| 2.106 | ++++ |
| 2.107 | ++++ |
| 2.108 | ++++ |
| 2.109 | ++++ |
| 2.110 | ++++ |
| 2.111 | ++++ |
| 2.112 | ++++ |
| 2.113 | ++++ |
| 2.114 | ++++ |
| 2.115 | ++++ |
| 2.116 | ++++ |
| 2.117 | ++++ |
| 2.118 | ++++ |
| 2.119 | ++++ |
| 2.120 | ++++ |
| 2.121 | ++++ |
| 2.122 | ++++ |
| 2.123 | ++++ |
| 2.124 | ++++ |
| 2.125 | ++++ |
| 2.126 | ++++ |
| 2.127 | ++++ |
| 2.128 | ++++ |
| 2.129 | ++++ |
| 2.130 | ++++ |
| 2.131 | ++++ |
| 2.132 | ++++ |
| 2.133 | ++++ |
| 2.134 | ++++ |
| 2.135 | ++++ |
| 2.136 | ++++ |
| 2.137 | ++++ |
| 2.138 | ++++ |
| 2.139 | ++++ |
| 2.140 | ++++ |
| 2.141 | ++++ |
| 2.142 | ++++ |
| 2.143 | ++++ |
| 2.144 | ++++ |
| 2.145 | ++++ |
| 2.146 | ++++ |
| 2.147 | ++++ |
| 2.148 | ++++ |
| 2.149 | ++++ |
| 2.150 | ++++ |
| 2.151 | ++++ |
| 2.152 | +++ |
| 2.153 | ++++ |
| 2.154 | ++++ |
| 2.155 | ++++ |
| 2.156 | ++++ |
| 2.157 | ++++ |
| 2.158 | ++++ |
| 2.159 | ++++ |
| 2.160 | ++++ |
| 2.161 | ++++ |
| 3 | ++++ |
| 3.01 | ++++ |
| 3.02 | ++++ |
| 3.03 | ++++ |
| 3.04 | ++++ |
| 3.05 | ++++ |
| 4 | ++++ |
| 4.01 | ++++ |
| 5 | ++++ |
| 5.01 | ++++ |
| 6 | ++++ |
| 6.01 | ++++ |
| 6.02 | ++++ |
| 6.03 | ++++ |
| 6.04 | ++++ |
| 7 | ++++ |
| 7.01 | ++++ |
| 7.02 | ++++ |
| 7.03 | ++++ |
| 8 | ++++ |
| 8.01 | ++++ |
| 8.02 | ++++ |
| 8.03 | ++++ |
| 9 | +++ |
| 10 | ++++ |
| 10.01 | ++++ |
| 11 | ++++ |
| 11.01 | ++++ |
| 11.02 | ++++ |

where
‘++++’ means IC₅₀ ≤ 0.01 uM;
where ‘+++’ means 0.01 uM < IC₅₀ ≤ 0.1 uM.

### Example B-2: Mouse Semicarbazide-Sensitive Amine Oxidase (SSAO) Assay

Recombinant Mouse SSAO/VAP-1/AOC3 (R&D systems; Catalog #6107-AO) can be used to screen compound selectivity in vitro. The Mouse SSAO assay can be run in the same manner as described for the human SSAO assay.

### Example B-3: Human Diamine Oxidase (DAO) Enzyme Assay

Inhibition of recombinant human DAO (R& D systems; Catalog #8298-AO) activity can be used to screen compound selectivity in vitro. The human DAO assay can be run in the same manner as described for the human SSAO assay, with the exception that putrescine (Sigma-Aldrich Catalog # P5780) and aminoguanidine bicarbonate (Sigma-Aldrich Catalog #109266-100G) can be used as the substrate and positive control, respectively.

### Example B-4: Human Monoamine Oxidase A and B Assays

Inhibition of recombinant human MAO-A (Sigma-Aldrich Catalog # M7316) and MAO-B (Sigma-Aldrich Catalog # M7441) can be used to screen compound selectivity in vitro. The MAO-A and MAO-B assays can be run in the same manner as described above for the human SSAO assay, with tyramine (Sigma-Aldrich Catalog # T2879) and benzylamine (Sigma-Aldrich Catalog # B5136) being used as the substrates for MAO-A and MAO-B, respectively. The positive controls for MAO-A and MAO-B can be clorgyline (Sigma-Aldrich Catalog # M3778) and mofegiline (MedChem Express Catalog # HY-16677A), respectively.

### Example B-5: Human Lysyl Oxidase (LOX) Assay

Recombinant human lysyl oxidase (LOX) can be isolated from concentrated conditioned media (CCM) of cells that transiently or stably overexpress the human LOX enzyme. Once isolated, the CCM can be concentrated using a centrifugation column with 10 kDa molecular weight cut-off (MWCO). Inhibition of LOX activity can then be tested suing the same fluorescence readout as for SSAO with the exception that the 1,5-diaminopentane can be used as the LOX substrate and β-aminopropionitrile (Sigma-Aldrich Catalog # A3134) used as positive control.

### Example B-6: Peroxide Scavenging/Amplex Ultra Red Interference Assay

A counter assay can be run to assess compound interference with the AUR enzyme and to identify compounds that might scavenge H₂O₂ directly, leading to a false positive readout regarding SSAO enzyme inhibition. To do this, H₂O₂ solution can be added to compound and the AUR mixture, in the absence of SSAO enzyme, and the effects on H₂O2-induced fluorescence can then be measured. The peroxide scavenger compound N-Acetyl-L-cysteine (NAC: Sigma-Aldrich Catalog # A7250) and the enzyme catalase (Sigma-Aldrich Catalog # C1345), which catalyzes the degradation of H₂O₂ into H₂O and 02, can be used as positive controls in this interference assay.

### Example B-7: Compound Oxidation Assay

Because the SSAO compounds are mechanism-based inhibitors, the potential for compound turnover by SSAO/VAP-1 can be assessed to determine the substrate propensity of the compounds relative to background (dimethyl sulfoxide only). The assay can be run in a similar manner as that described for the SSAO enzyme assay. Briefly, compounds can be incubated with recombinant human SSAO enzyme, in the absence of benzylamine substrate, and oxidase activity can be measured for 30-120 min after addition of the AUR/HRP mixture.

### Example B-8: Mouse Pharmacodynamic Model

SSAO activity can be measured using a modification of previously described methods (J Pharmacol Exp Ther. 2013 November; 347(2):365-74). To measure compound activity in vivo, mice can be orally administered with compounds at predetermined concentrations. Animals can then be killed after 2-48 hours for collection of plasma, abdominal fat and other tissues of interest. Tissue samples can be homogenized in HES buffer (20 mM HEPES, 1 mM EDTA, sucrose 250 mM, 1X protease and phosphatases inhibitor, pH 7.4). Homogenates can then be centrifuged at 2000×g for 5-10 min at 4° C. and the supernatants collected and diluted 1:5 in assay buffer (0.1 M sodium phosphate buffer, pH7.2) for the fluorometric measurement of SSAO activity. When measuring SSAO activity from tissue, pargyline can be included in the assay buffer to inhibit any potential endogenous monoamine oxidase A and B which could interfere with the assay. SSAO activity in the plasma and tissue homogenates can then be analyzed as described in the in vitro methods for human SSAO.

### Example B-9: Carbon Tetrachloride (CCl₄)-Induced Liver Fibrosis Model

Analysis of the use of SSAO inhibitors to treat liver fibrosis can be performed using the CCl₄-induced liver fibrosis model. To do this, C57BL/6 mice can be dosed with vehicle (olive oil) or CCl₄ (0.5 L/g; 2 times per week) by oral gavage (PO) for a period of 3 week to induce liver injury and fibrosis. Following the 3 week induction period, mice can then be dosed, therapeutically, with SSAO inhibitors for an additional 3 weeks (therapeutic dosing). At the end of the 6 week study period, plasma and tissue can be harvested to determine drug concentrations, SSAO activity, liver enzymes and liver fibrosis, inflammation and pro-fibrotic gene or protein expression in both the vehicle and drug-treated groups.

### Example B-10: Rat Pharmacodynamic Model

SSAO activity can be measured using a modification of previously described methods (J Pharmacol Exp Ther. 2013 November; 347(2):365-74). To measure compound activity in vivo, rats can be orally administered with compounds at predetermined concentrations. Animals can then be killed after 2-48 hours for collection of plasma, abdominal fat, liver and other tissues of interest. Tissue samples can be homogenized in HES buffer (20 mM HEPES, 150 mM NaCl, 1 mM EDTA, 1 mM EGTA with 1% triton X100, 1X protease and phosphatases inhibitor, pH 7.4). For tissues, homogenates can then be centrifuged at 10,000×g for 30 min at 4° C. and the supernatants collected for the fluorometric measurement of SSAO activity. Plasma can be assayed directly. When measuring SSAO activity, pargyline can be included in the assay buffer to inhibit any potential endogenous monoamine oxidase A and B which could interfere with the assay. SSAO activity in the plasma and tissue homogenates can then be analyzed as described in the in vitro methods for human SSAO.

### Example B-11: Carbon Tetrachloride (CCl₄)-Induced Liver Fibrosis Model

Analysis of the use of SSAO inhibitors to treat liver fibrosis can be performed using the CCl₄-induced liver fibrosis model. To do this, Sprague-Dawley rats can be dosed with vehicle (olive oil) or CCl₄ (1-2 L/g (1:1 in olive oil); 2 times per week) by oral gavage (PO) for a period of 4-8 weeks to induce liver injury and fibrosis. Rats can then be dosed with inhibitors either: 1) in a preventative manner from day 0 onward or 2) in a therapeutic manner, starting 2 or 4 weeks after the initiation of CCl₄ dosing. At the end of the study period, plasma and tissue can be harvested to determine drug concentrations, SSAO activity, liver enzymes and liver fibrosis, inflammation and pro-fibrotic gene or protein expression in both the vehicle and drug-treated groups

### Example B-12: NASH Liver Fibrosis Model

Analysis of the use of SSAO inhibitors to treat liver steatosis/inflammation/fibrosis can be performed using rodent high fat diet-induced models of non-alcoholic steatohepatitis (NASH). To test the use of SSAO inhibitors in NASH, mouse NASH models can be run as previously described (World J Hepatol 2016 Jun. 8; 8(16): 673-684).

### Example B-13: Lipopolysaccharide (LPS) Airway Inflammation Model

To assess the use of SSAO inhibitors on inflammation, mice can undergo pulmonary challenge with LPS to induce inflammatory cell infiltration and cytokine production. To do this, mice can be administered with vehicle or SSAO inhibitor by oral gavage, 1-2 hr prior to LPS challenge. Inflammation can then be induced by oropharyngeal instillation of vehicle (phosphate-buffered saline) or LPS. Six hours later, mice can be killed and bronchoalveolar lavage (BAL) fluid collected for recovery of airway luminal cells and cytokine analysis. To isolate BAL, the trachea can be cannulated and lavaged with 1.0 mL heparinized (10 U/ml) saline. An aliquot of the lavage can then be reserved for total and differential white cell counts and the remaining fluid can be centrifuged and the supernatants used to measure cytokines.

### Example B-14: Mouse Bleomycin Lung Fibrosis Model

Briefly, lung fibrosis can be induced by oropharyngeal instillation of bleomycin (Blenoxane, Henry Schein Catalog #1045785). To do this, mice can be anesthetized with isoflurane (5% in 100% O2) and then be hung on a board by their teeth in a reclined position. Bleomycin (BLM; 1-5.0 U/kg) can be delivered by oropharyngeal instillation whereby BLM is dripped onto the vocal chords (2.5 L/g volume) facilitating aspiration. SSAO compounds can be administered prior to BLM challenge (preventative dosing) or at different timepoints after BLM challenge (therapeutic dosing). The route and frequency of dosing can be based on previously determined pharmacokinetic properties for each compound in mice. At various timepoints after BLM challenge (i.e. 7-28 days), mice can killed for analysis of lung inflammation and cytokine release, pulmonary vascular leakage and lung fibrosis.

The examples and embodiments described herein are for illustrative purposes only and various modifications or changes suggested to persons skilled in the art are to be included within the spirit and purview of this application and scope of the appended claims.

What is claimed is:

1. A compound having the structure of Formula (I): 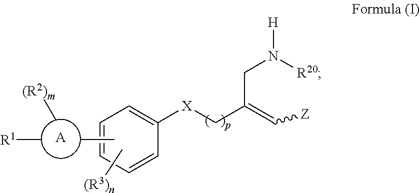
wherein: 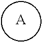
is a C₃₋₁₀cycloalkyl ring;
X is —O—, —S—, —S(O)₂—, —N(R¹³)—, or —C(R¹³)₂—;
Z is H, F, or Cl;
R¹ is halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁴, —SR⁴, —N(R⁴)(R⁵), —C(O)OR⁴, —OC(O)N(R⁴)(R⁵), —N(R⁶)C(O)N(R⁴)(R⁵), —N(R⁶)C(O)OR⁷, —N(R⁶)S(O)₂R⁷, —C(O)R⁷, —S(O)R⁷, —OC(O)R⁷, —C(O)N(R⁴)(R⁵), —C(O)C(O)N(R⁴)(R⁵), —N(R⁶)C(O)R⁷, —S(O)₂R⁷, —S(O)₂N(R⁴)(R⁵)—, S(═O)(═NH)N(R⁴)(R⁵), —CH₂C(O)N(R⁴)(R⁵), —CH₂N(R⁶)C(O)R⁷, —CH₂S(O)₂R⁷, or —CH₂S(O)₂N(R⁴)(R⁵), wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14a};
each R² and each R³ are each independently selected from halogen, —CN, C₁₋₆alkyl, C₂₋₆alkenyl, C₂₋₆alkynyl, C₃₋₆cycloalkyl, C₂₋₉heterocycloalkyl, C₆₋₁₀aryl, C₁₋₉heteroaryl, —OR⁸, —SR⁸, —N(R⁹)(R¹⁰), —C(O)OR⁹, —C(O)N(R⁹)(R¹⁰), —OC(O)N(R⁹)(R¹⁰), —N(R¹¹)C(O)N(R⁹)(R¹⁰), —N(R¹¹)C(O)OR¹², —N(R¹¹)C(O)R¹², —N(R^{11)S(O)}₂R¹², —C(O)R¹², —S(O)R¹², —S(O)₂R¹², —S(O)₂N(R⁹)(R¹⁰), and —OC(O)R¹², wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14b};
R⁴ is selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14c};
R⁵ is selected from H, C₁₋₆alkyl, and C₁₋₆haloalkyl; or R⁴ and R⁵, together with the nitrogen to which they are attached, form a C₂₋₉heterocycloalkyl ring optionally substituted with one, two, or three R^{14d};
R⁶ is selected from H, C₁₋₆alkyl, and C₁₋₆haloalkyl;
R⁷ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14e};
each R⁸ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14f};
each R⁹ is independently selected from H, C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R^{14g};
each R¹⁰ is independently selected from H and C₁₋₆alkyl; or R⁹ and R¹⁰, together with the nitrogen to which they are attached, form a C₂₋₉heterocycloalkyl ring optionally substituted with one, two, or three R^{14h};
each R¹¹ is independently selected from H, C₁₋₆alkyl, and C₁₋₆haloalkyl;
each R¹² is independently selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl, wherein C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl are optionally substituted with one, two, or three R¹⁴ⁱ;
each R¹³ is independently selected from H, C₁₋₆alkyl, and C₁₋₆haloalkyl;
each R^{14a}, R^{14b}, R^{14c}, R^{14d}, R^{14e}, R^{14f}, R^{14g}, R^{14h}, and R¹⁴ⁱ are each independently selected from halogen, —CN, C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, C₁₋₉heteroaryl, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), —OCH₂C(O)OR¹⁶, and —OC(O)R¹⁹, wherein C₁₋₆alkyl, C₂₋₆alkenyl, C₂₋₆alkynyl, C₃₋₆cycloalkyl, —CH₂—C₃₋₆cycloalkyl, C₂₋₉heterocycloalkyl, —CH₂—C₂₋₉heterocycloalkyl, C₆₋₁₀aryl, —CH₂—C₆₋₁₀aryl, and C₁₋₉heteroaryl are optionally substituted with one, two, or three groups independently selected from halogen, oxo, —CN, C₁₋₆alkyl, C₁₋₆haloalkyl, C₁₋₆alkoxy, C₁₋₆haloalkoxy, —OR¹⁵, —SR¹⁵, —N(R¹⁶)(R¹⁷), —C(O)OR¹⁶, —C(O)N(R¹⁶)(R¹⁷), —C(O)C(O)N(R¹⁶)(R¹⁷), —OC(O)N(R¹⁶)(R¹⁷), —N(R¹⁸)C(O)N(R¹⁶)(R¹⁷), —N(R¹⁸)C(O)OR¹⁹, —N(R¹⁸)C(O)R¹⁹, —N(R¹⁸)S(O)₂R¹⁹, —C(O)R¹⁹, —S(O)₂R¹⁹, —S(O)₂N(R¹⁶)(R¹⁷), and —OC(O)R¹⁹;
each R¹⁵ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁶ is independently selected from H, C₁₋₆alkyl, C₁₋₆haloalkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
each R¹⁷ is independently selected from H and C₁₋₆alkyl; or R¹⁶ and R¹⁷, together with the nitrogen to which they are attached, form a C₂₋₉heterocycloalkyl ring;
each R¹⁸ is independently selected from H and C₁₋₆alkyl;
each R¹⁹ is selected from C₁₋₆alkyl, C₂₋₆alkenyl, C₂₋₆alkynyl, C₃₋₆cycloalkyl, C₂₋₉heterocycloalkyl, C₆₋₁₀aryl, and C₁₋₉heteroaryl;
R²⁰ is selected from H and C₁₋₆alkyl;
m is 0, 1, 2, 3, or 4;
n is 0, 1, 2, 3, or 4; and
p is 0 or 1;
or a pharmaceutically acceptable salt or solvate thereof.

2. The compound of claim 1, or a pharmaceutically acceptable salt or solvate thereof, wherein m is 0.

3. The compound of claim 2, or a pharmaceutically acceptable salt or solvate thereof, having the structure of Formula (Ibb):

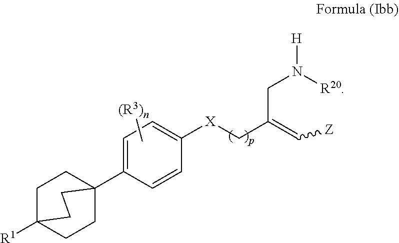

4. The compound of claim 3, or a pharmaceutically acceptable salt or solvate thereof, having the structure of Formula (Ibb′):

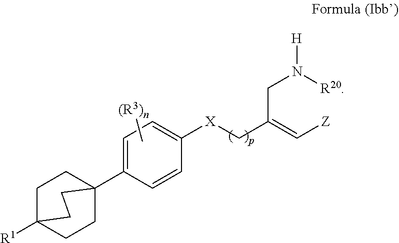

5. The compound of claim 4, or a pharmaceutically acceptable salt or solvate thereof, wherein n is 0.

6. The compound of claim 5, or a pharmaceutically acceptable salt or solvate thereof, wherein X is —O—.

7. The compound of claim 6, or a pharmaceutically acceptable salt or solvate thereof, wherein R²⁰ is H.

8. The compound of claim 7, or a pharmaceutically acceptable salt or solvate thereof, wherein Z is F.

9. The compound of claim 8, or a pharmaceutically acceptable salt or solvate thereof, wherein p is 1.

10. The compound of claim 9, or a pharmaceutically acceptable salt or solvate thereof, wherein R¹ is —OR⁴, —N(R⁶)C(O)R⁷, —N(R⁶)C(O)N(R⁴)(R⁵), —N(R⁶)S(O)₂R⁷, —C(O)R⁷, —C(O)N(R⁴)(R⁵), or —S(O)₂N(R⁴)(R⁵).

11. The compound of claim 10, or a pharmaceutically acceptable salt or solvate thereof, wherein R¹ is —C(O)N(R⁴)(R⁵).

12. The compound of claim 11, or a pharmaceutically acceptable salt or solvate thereof, wherein R⁴ is selected from H, C₁₋₆alkyl, C₃₋₆cycloalkyl, and C₂₋₉heterocycloalkyl, wherein C₁₋₆alkyl, C₃₋₆cycloalkyl and C₂₋₉heterocycloalkyl are optionally substituted with one, two, or three R^{14c}.

13. The compound of claim 12, or a pharmaceutically acceptable salt or solvate thereof, wherein R⁴ is selected from H, C₁₋₆alkyl, and C₂₋₉heterocycloalkyl, wherein C₁₋₆alkyl and C₂₋₉heterocycloalkyl are optionally substituted with one, two, or three R^{14c}.

14. The compound of claim 13, or a pharmaceutically acceptable salt or solvate thereof, wherein R⁴ is unsubstituted C₂₋₉heterocycloalkyl.

15. The compound of claim 11, or a pharmaceutically acceptable salt or solvate thereof, wherein R⁵ is H.

16. The compound of claim 11, or a pharmaceutically acceptable salt or solvate thereof, wherein R⁴ and R⁵, together with the nitrogen to which they are attached, form a C₂₋₉heterocycloalkyl ring optionally substituted with one, two, or three R^{14d}.

17. A compound selected from: 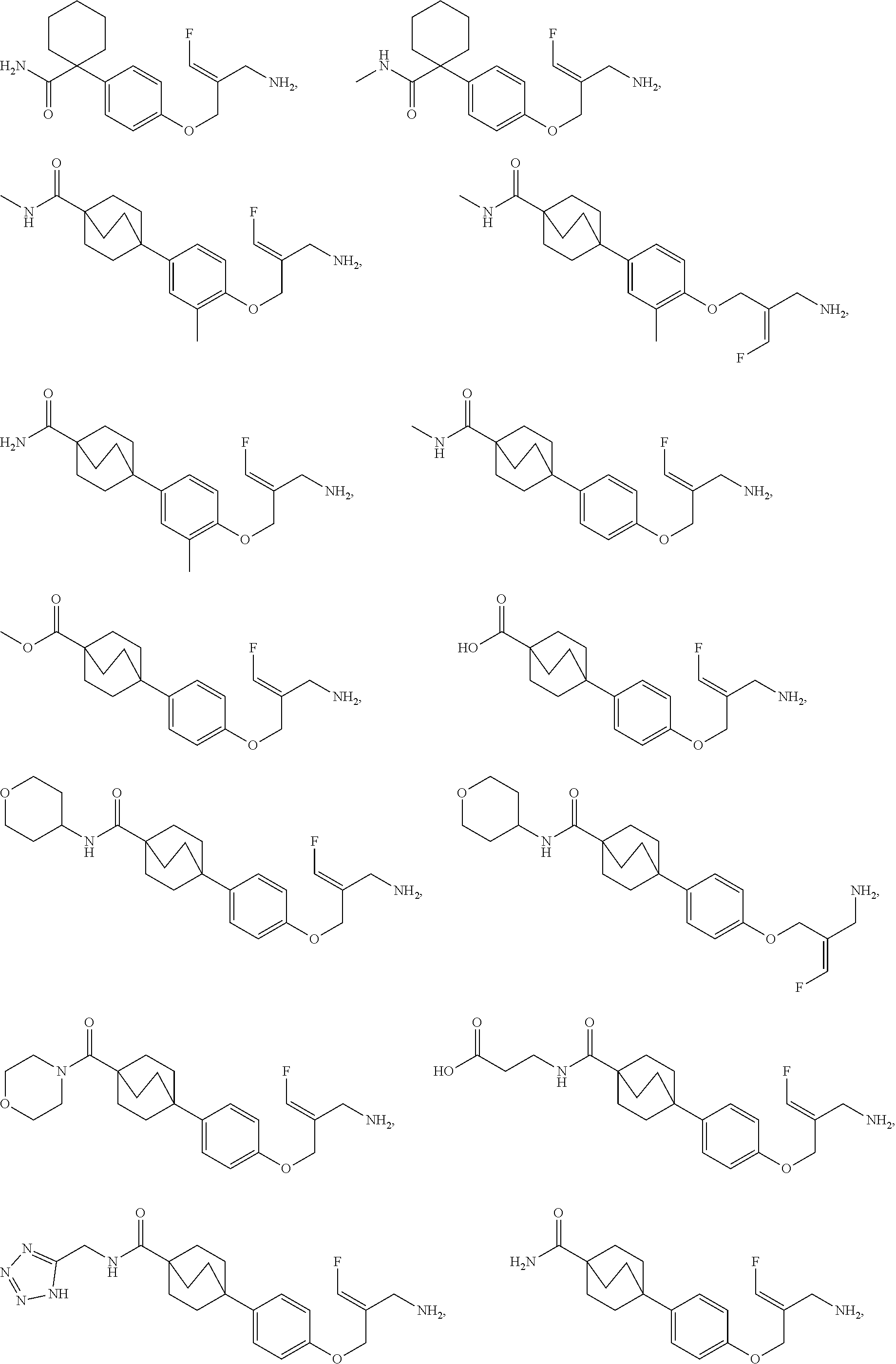 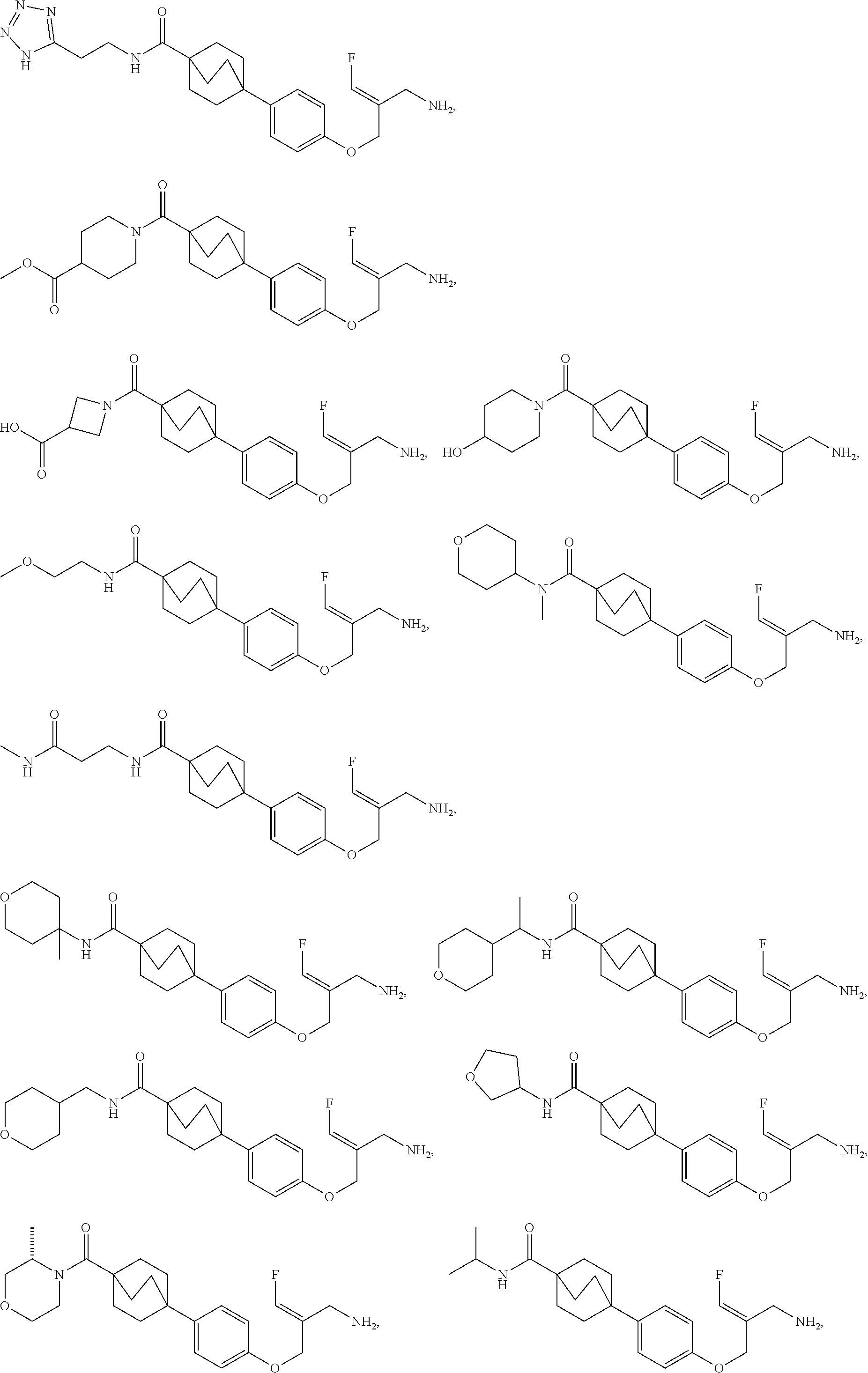 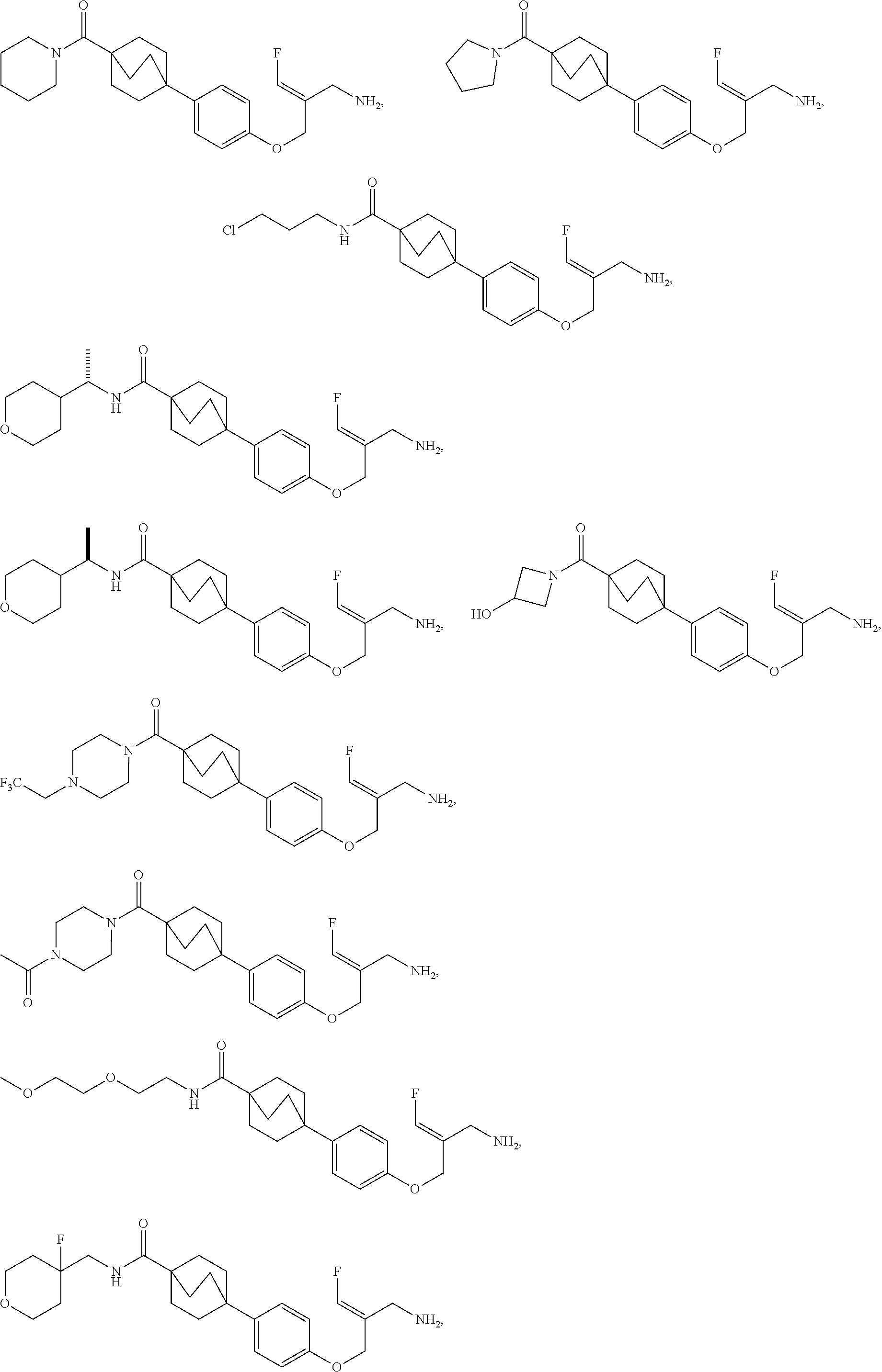 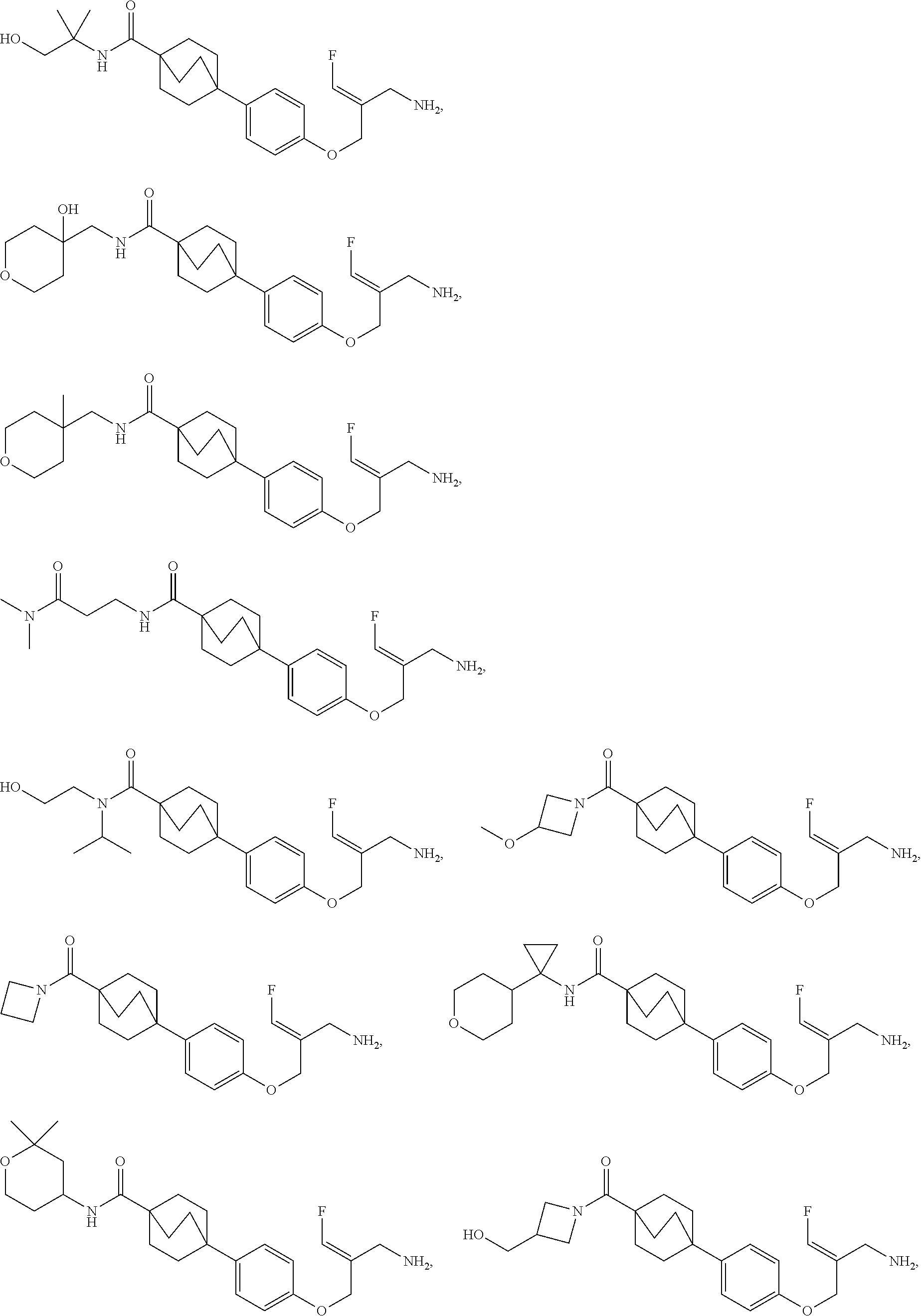 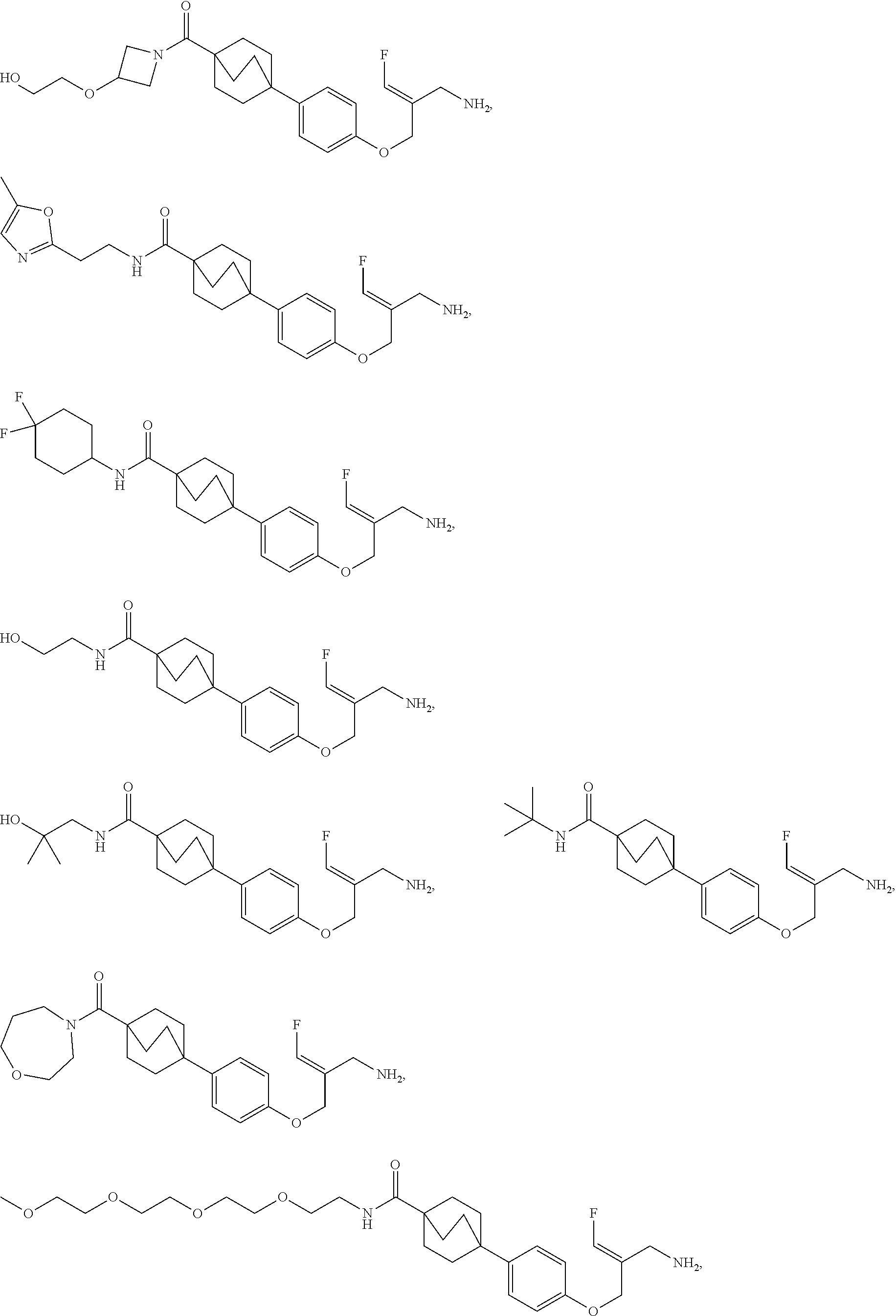 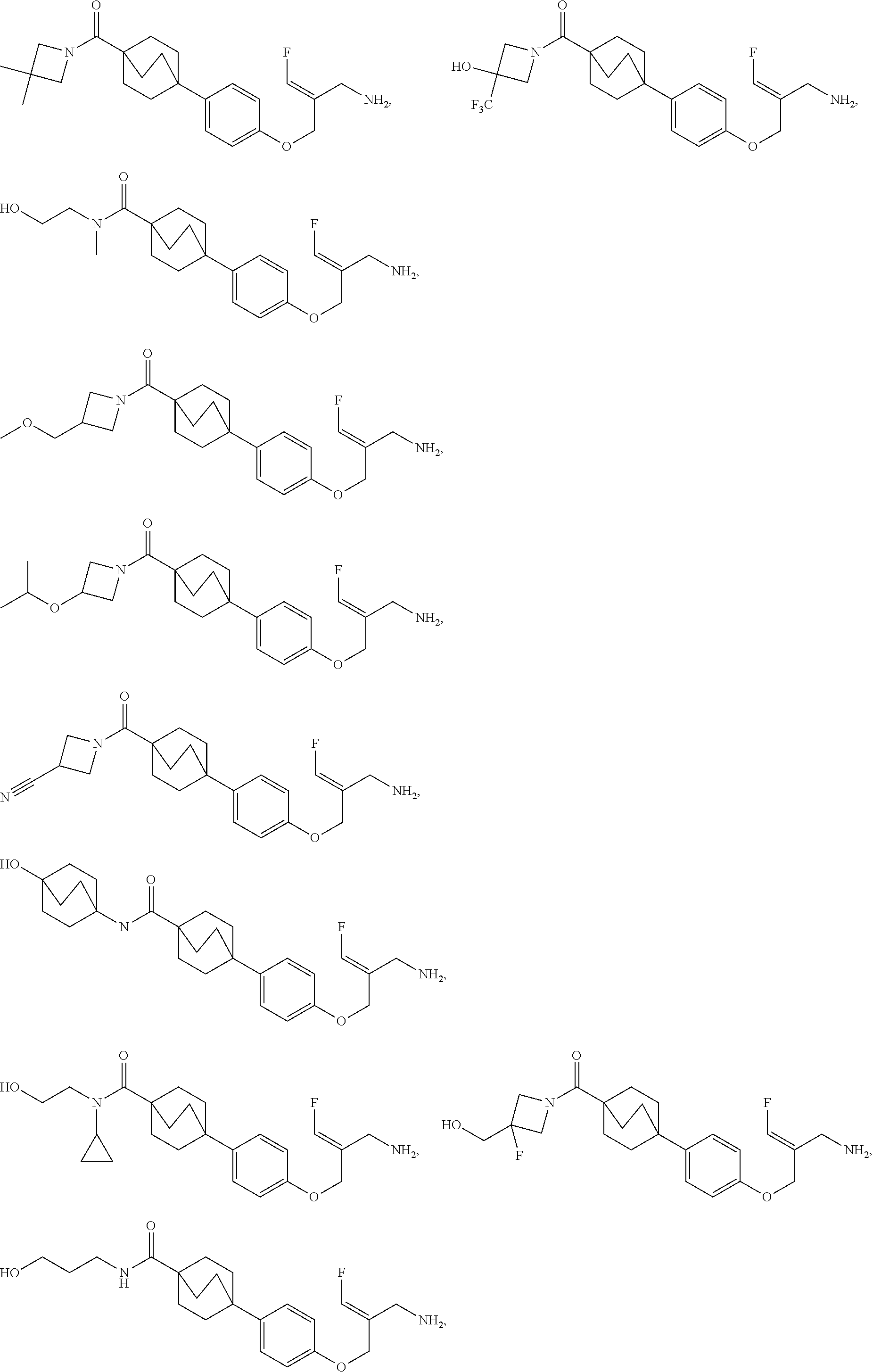 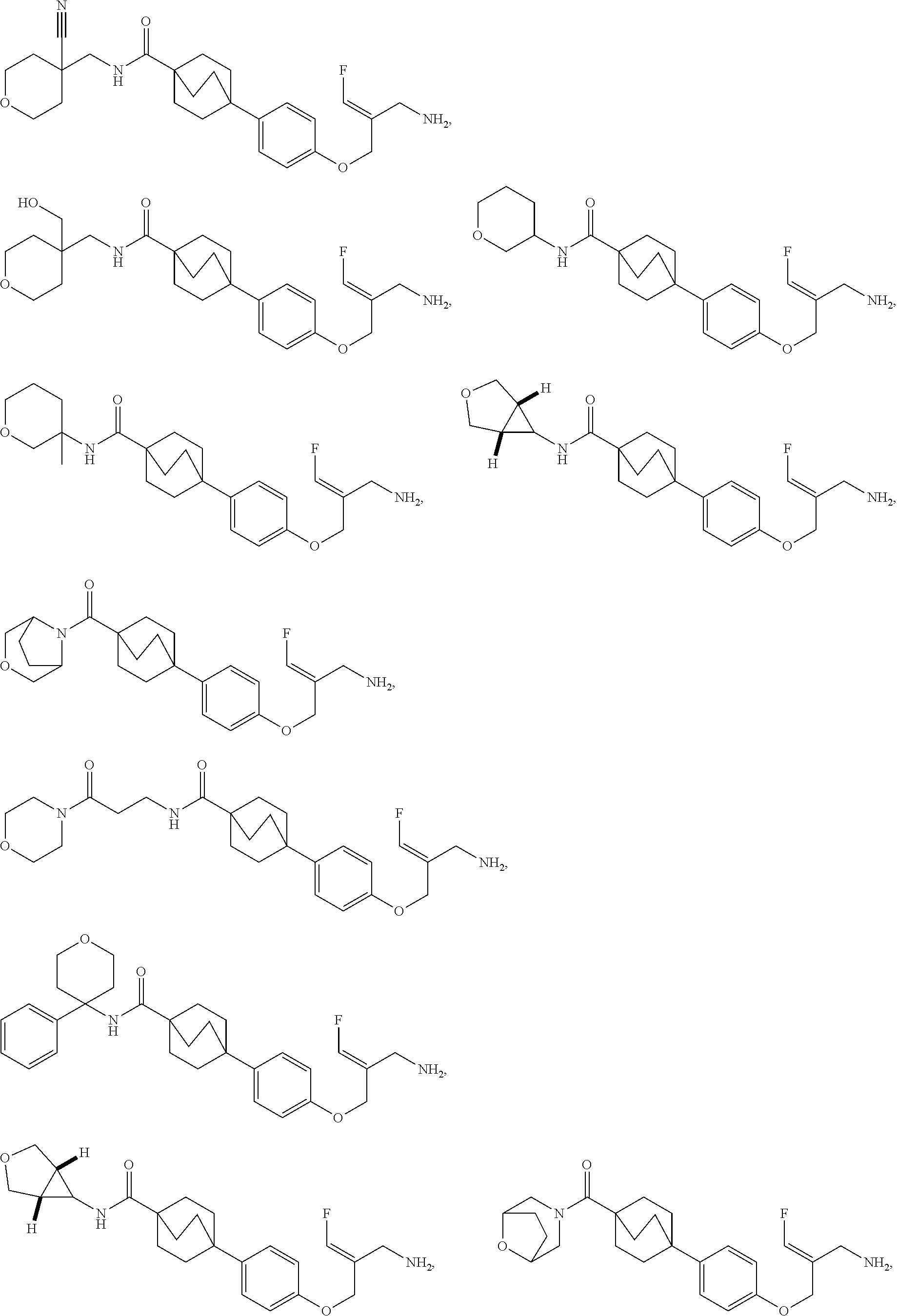 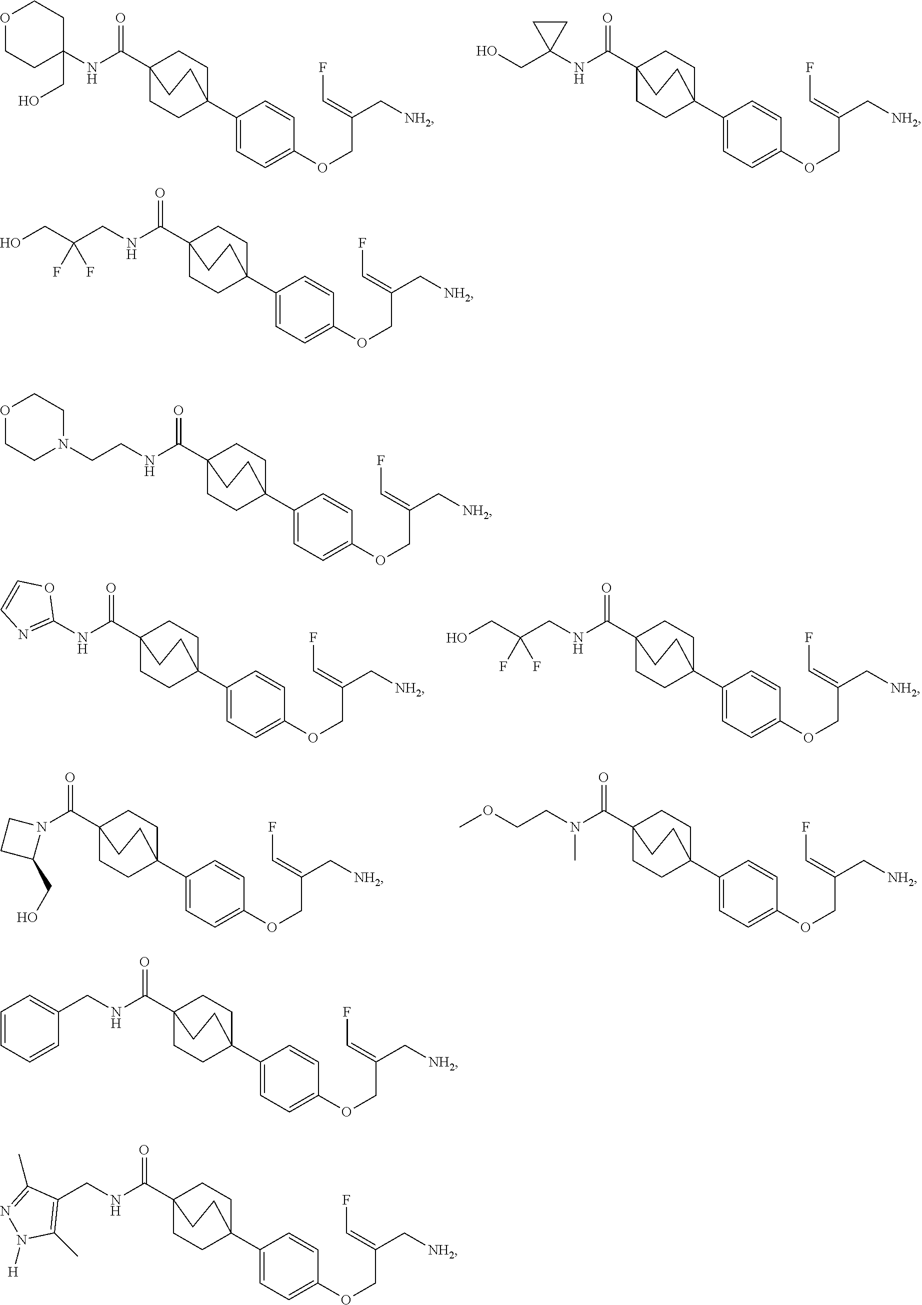 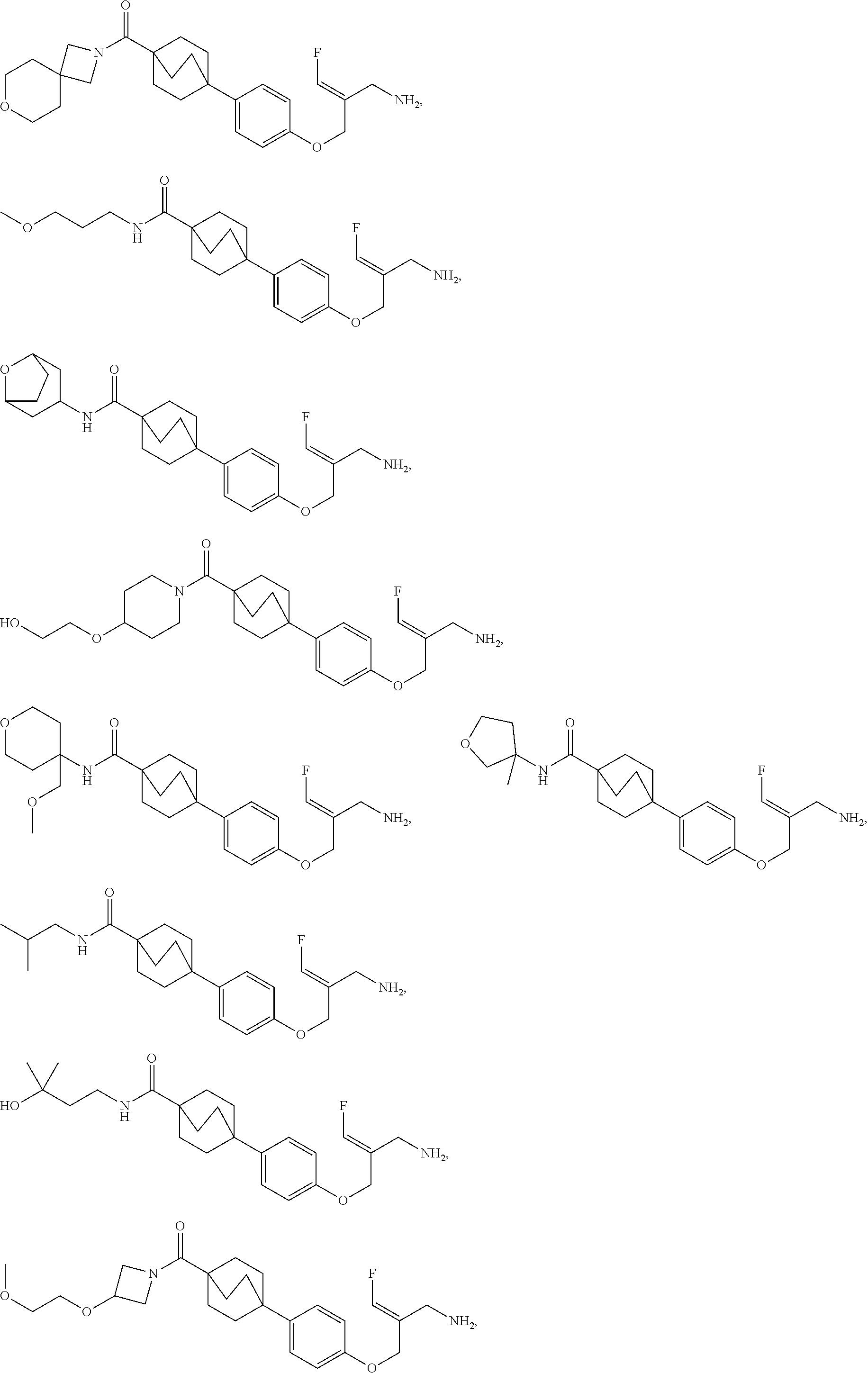 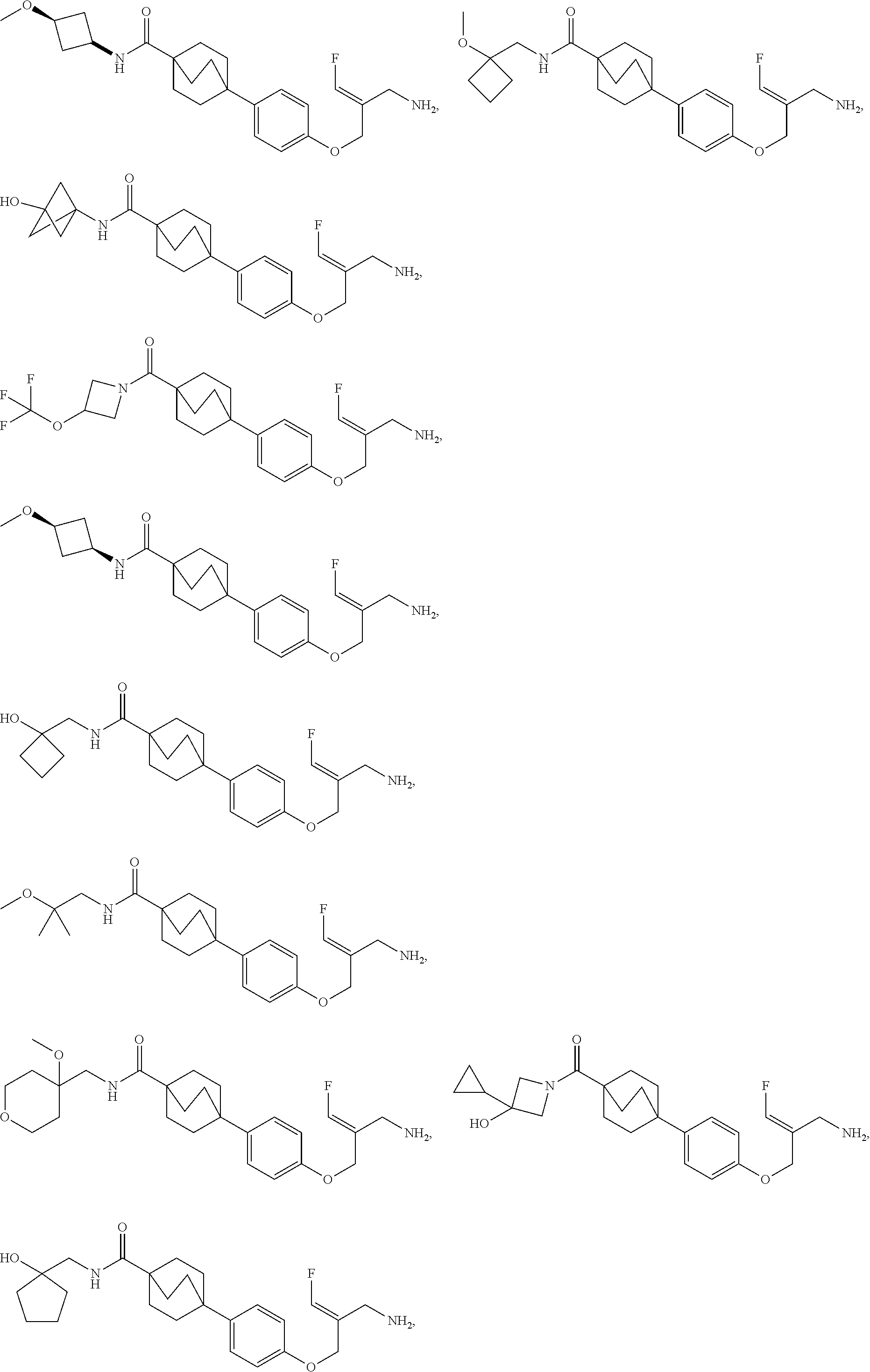 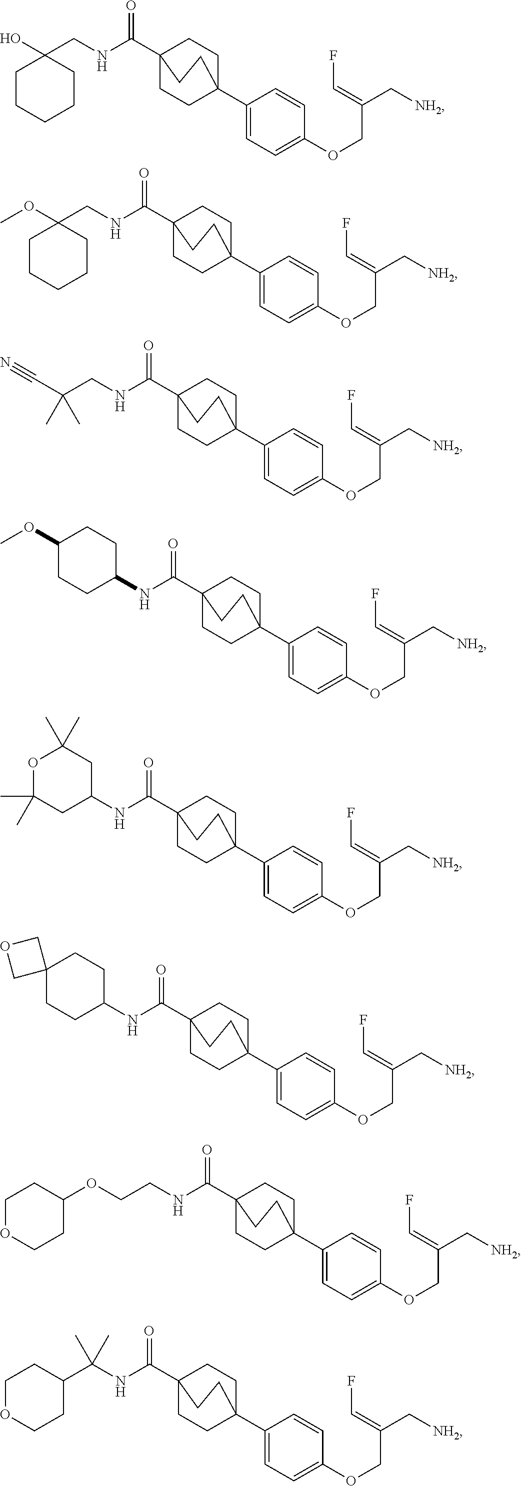 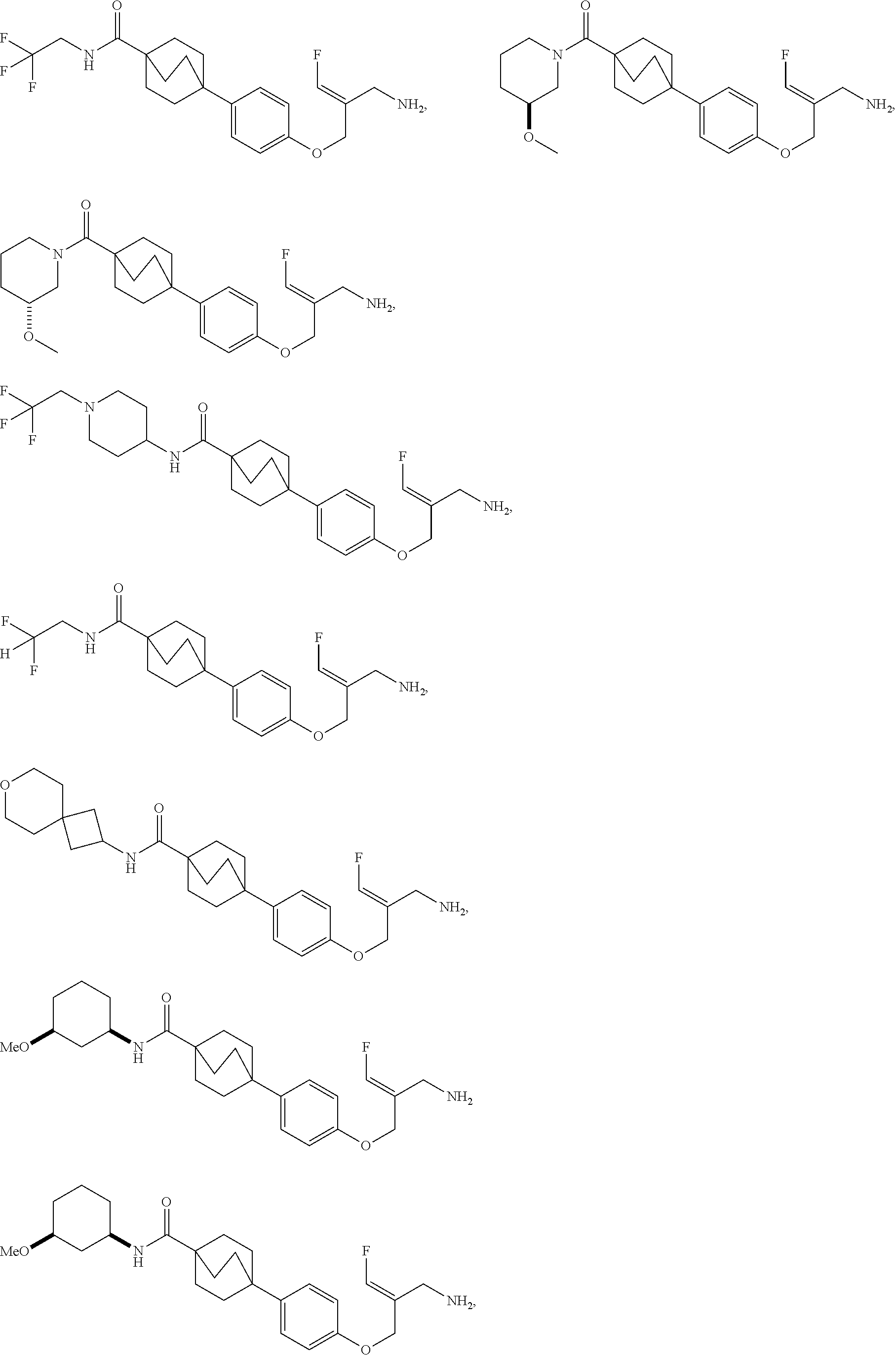 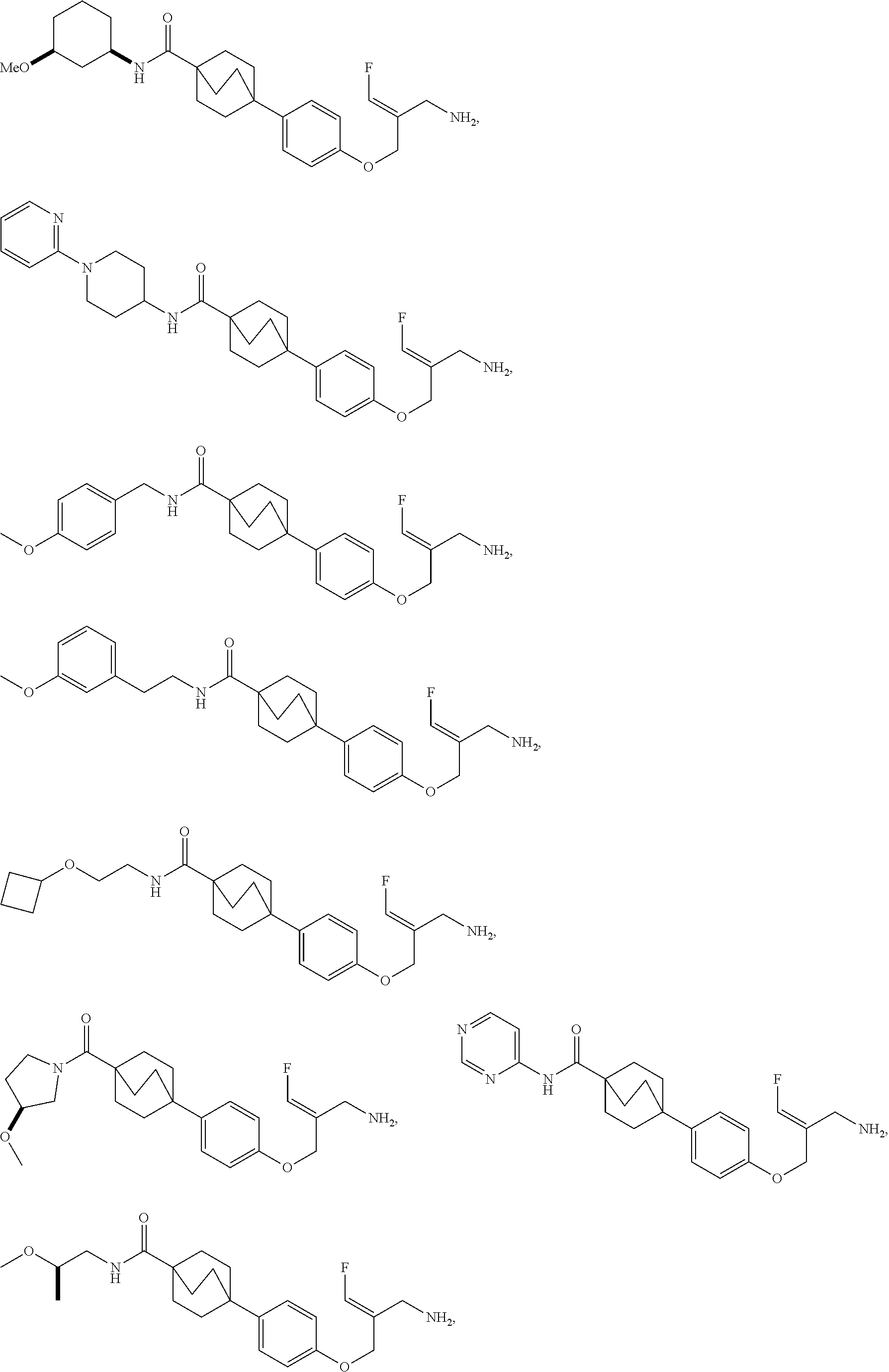 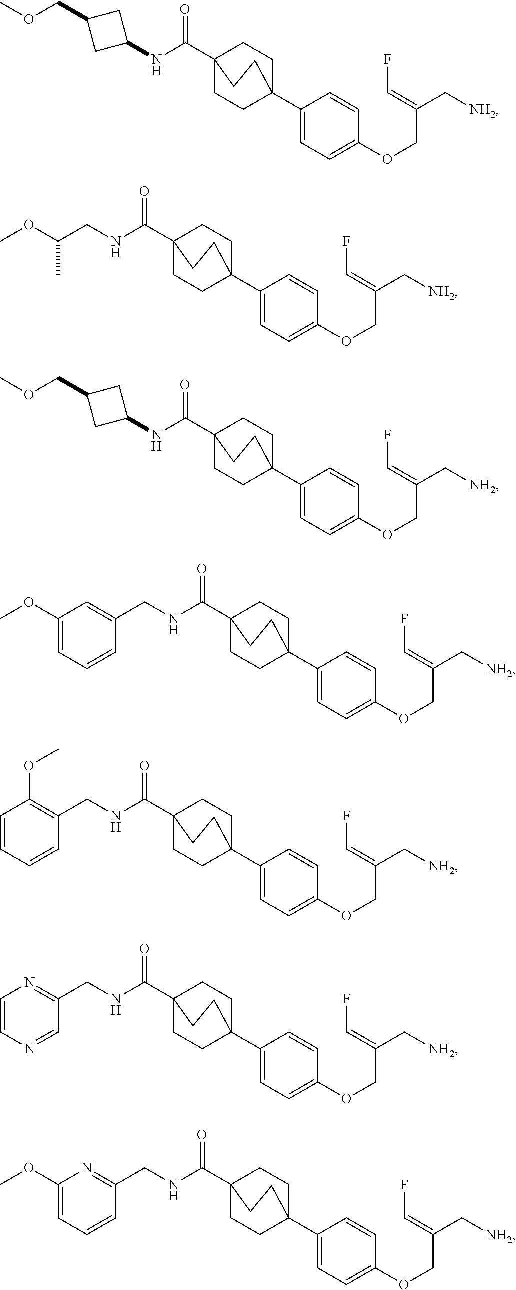 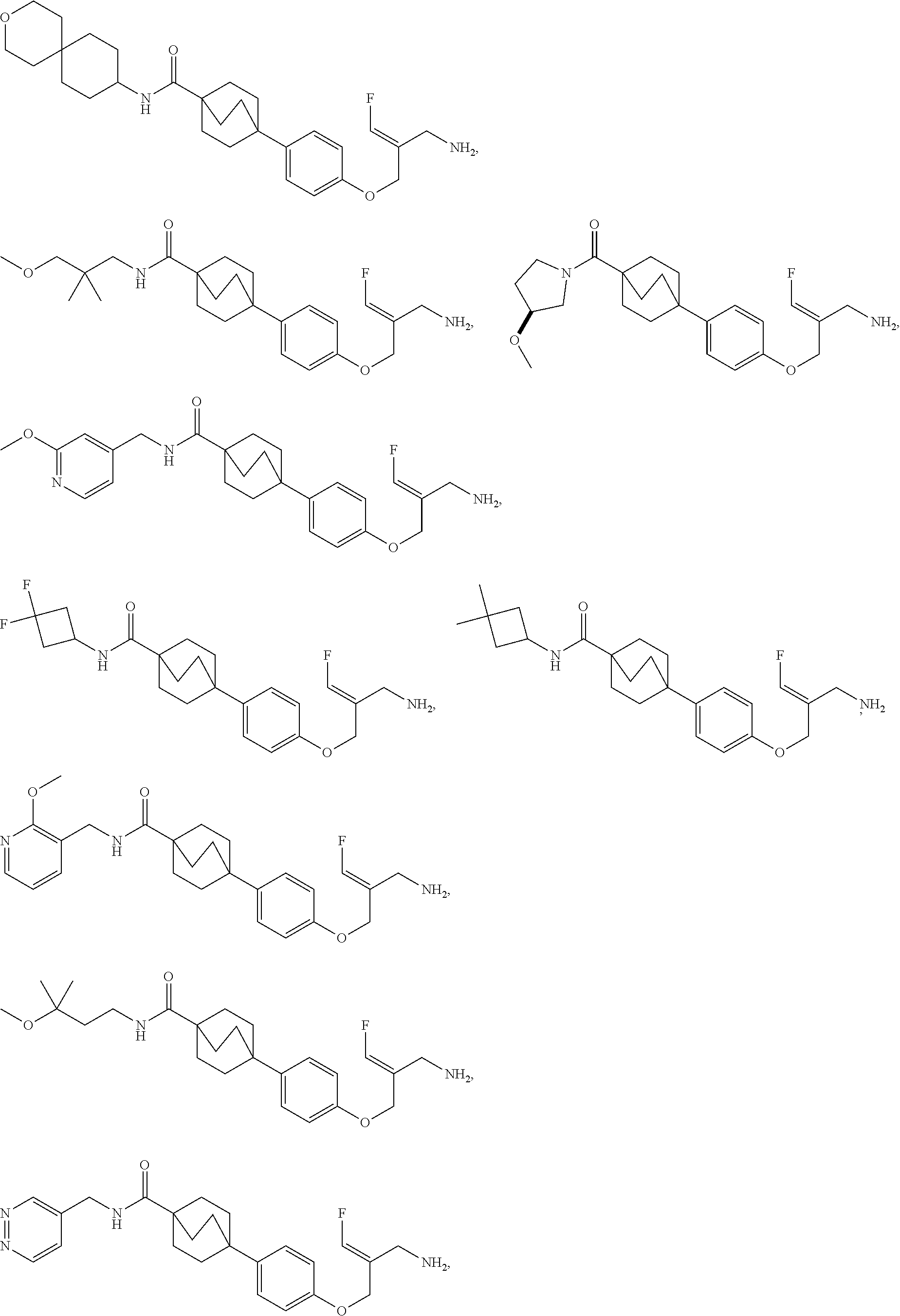 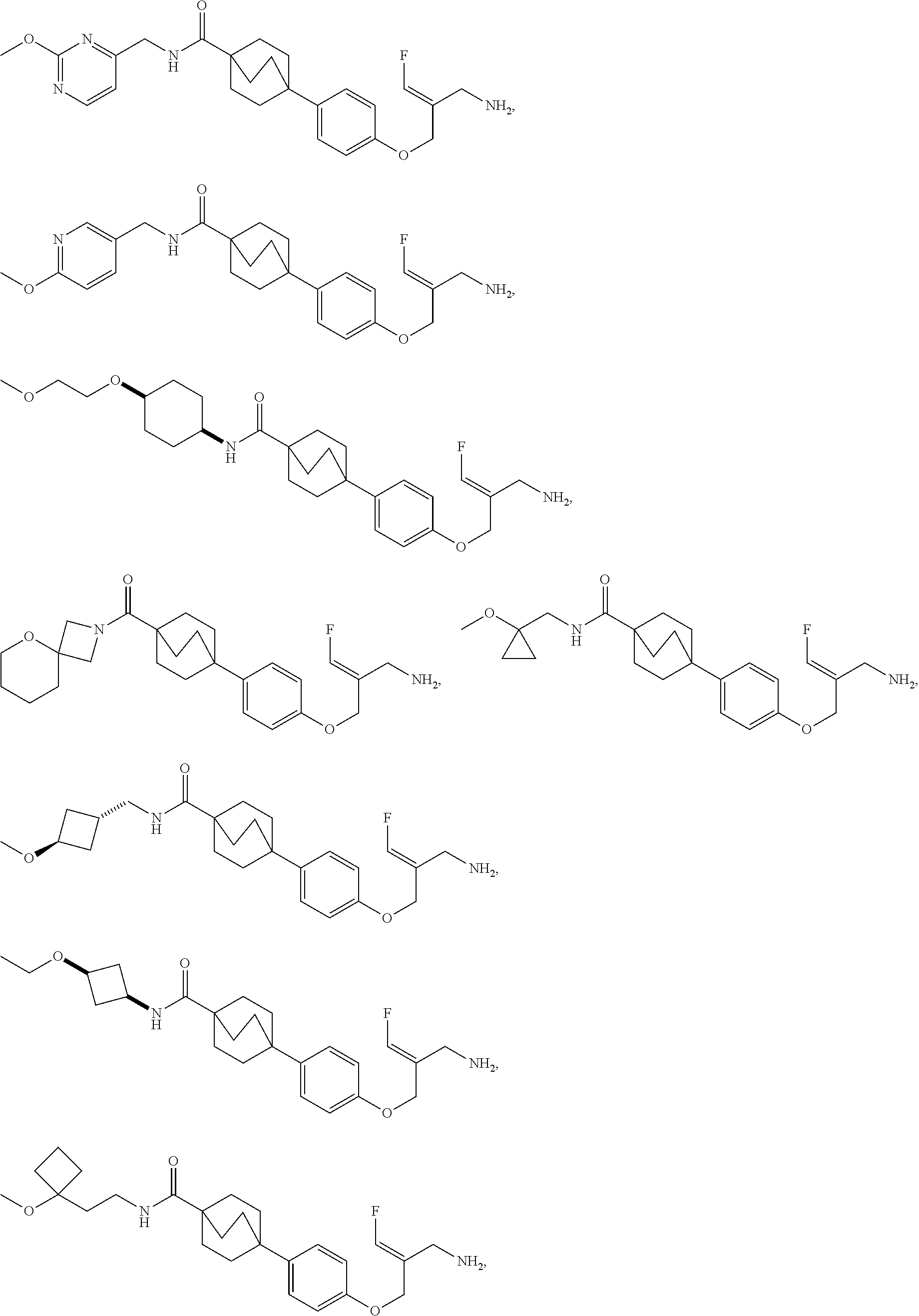 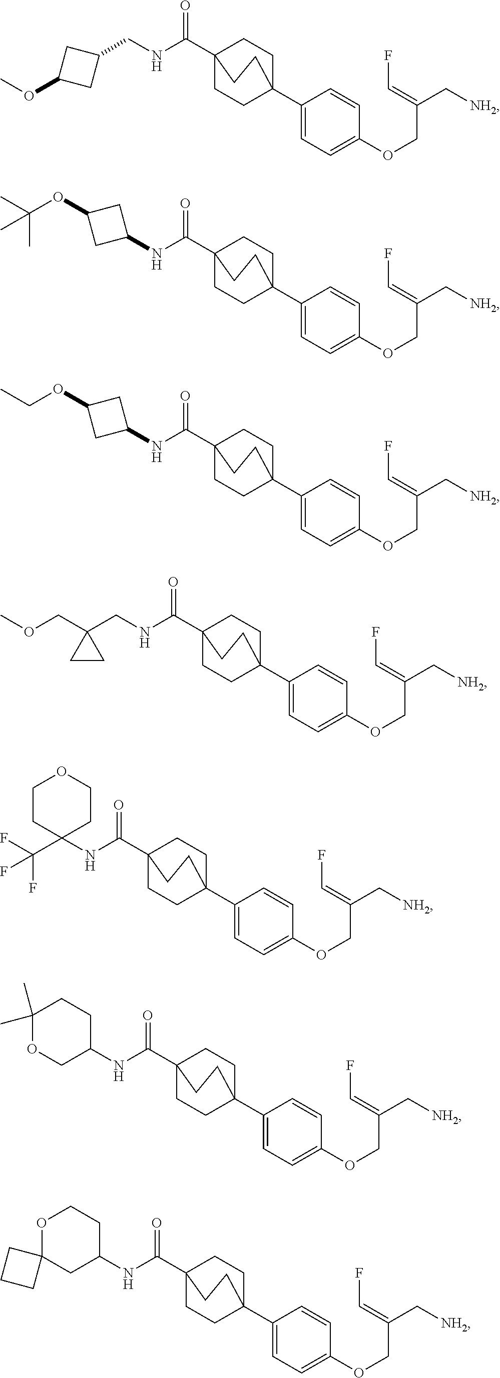 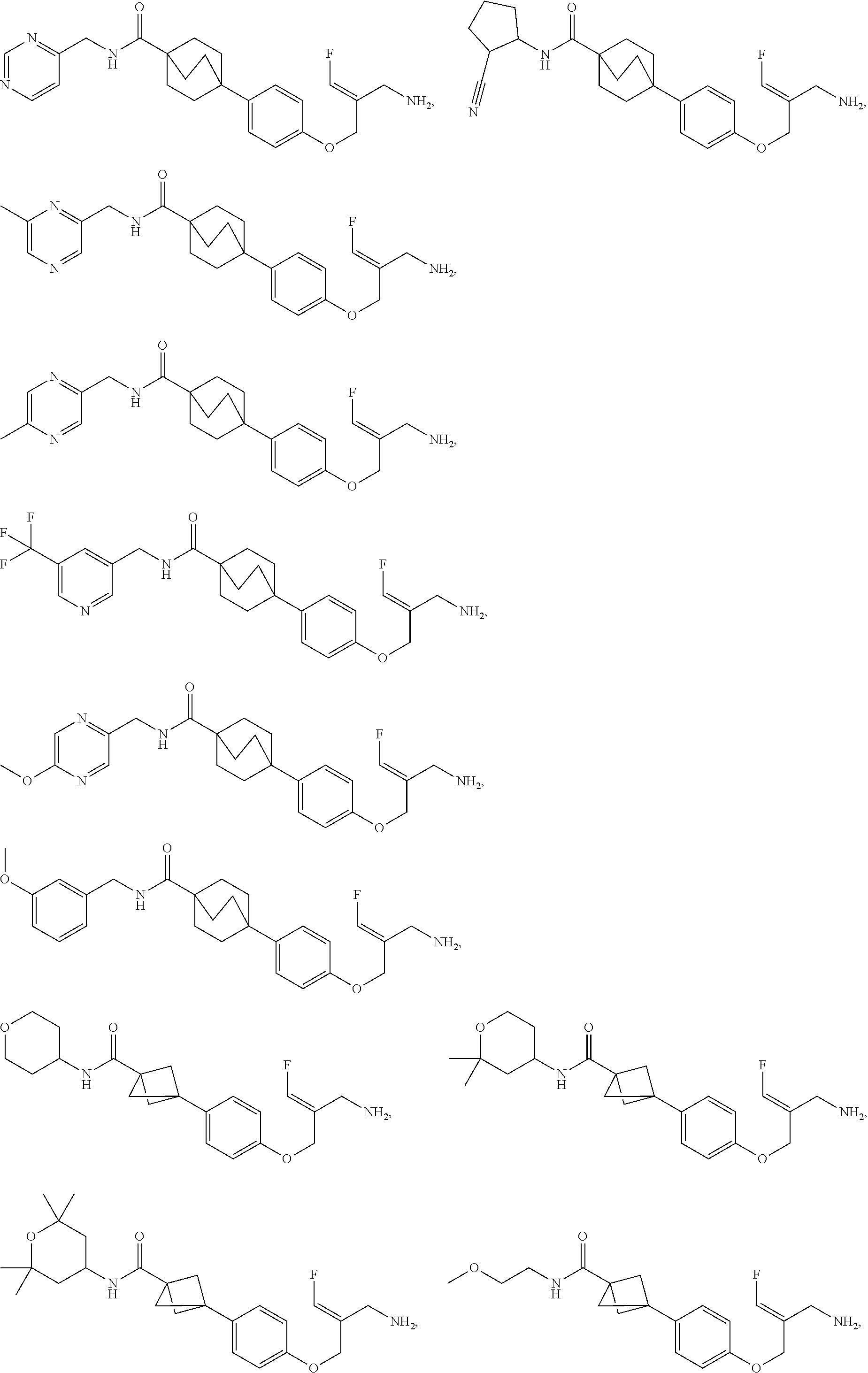 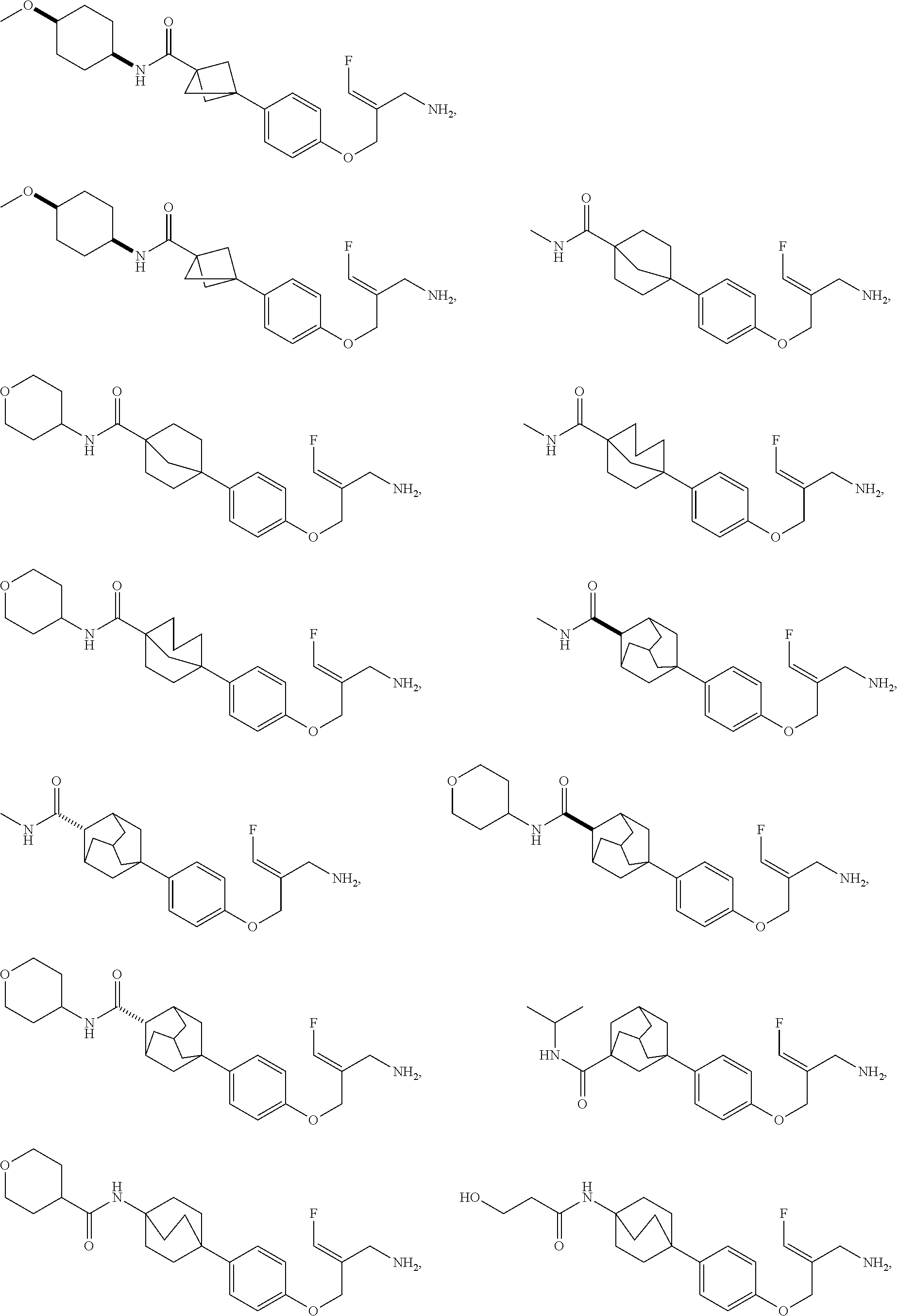 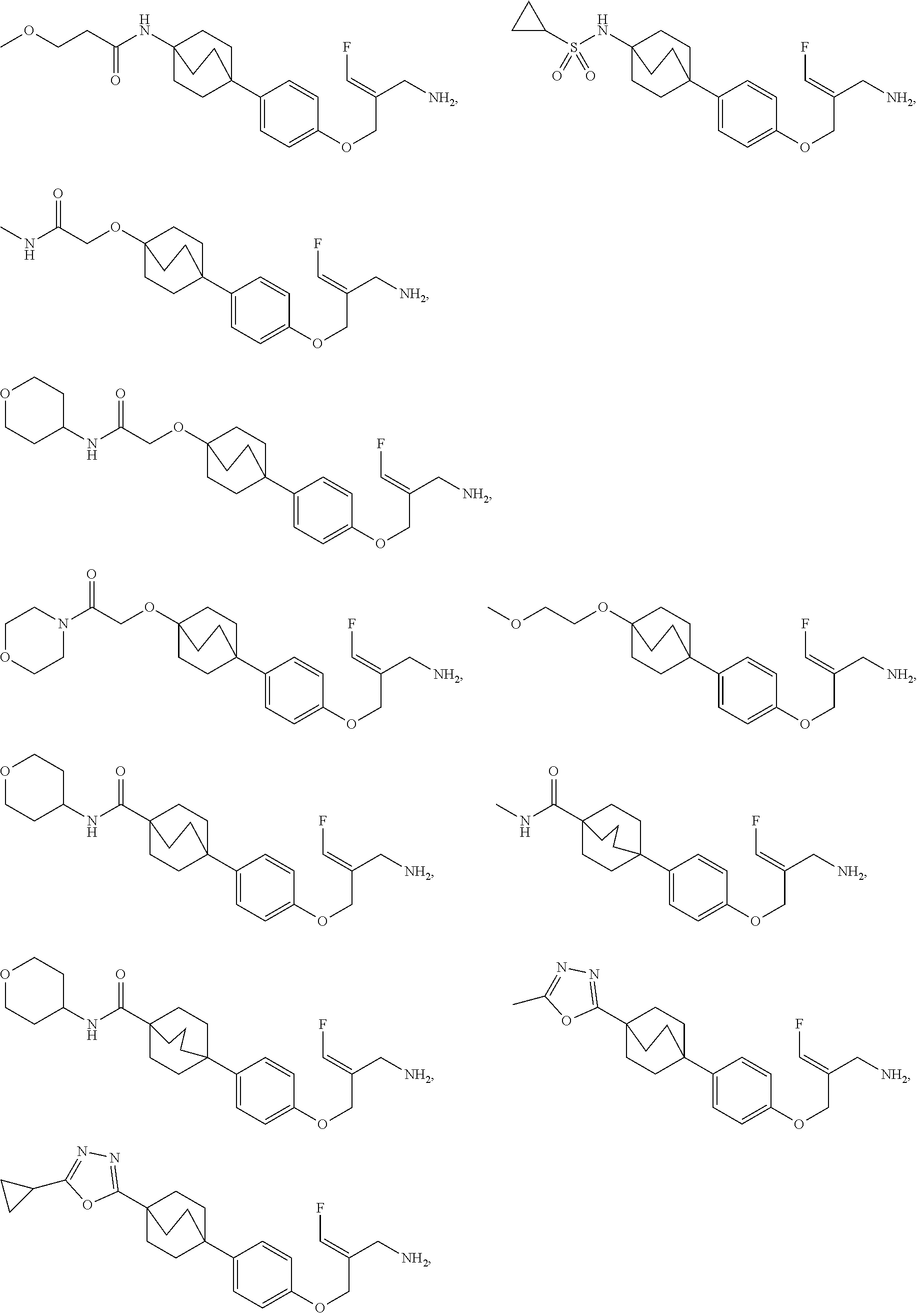 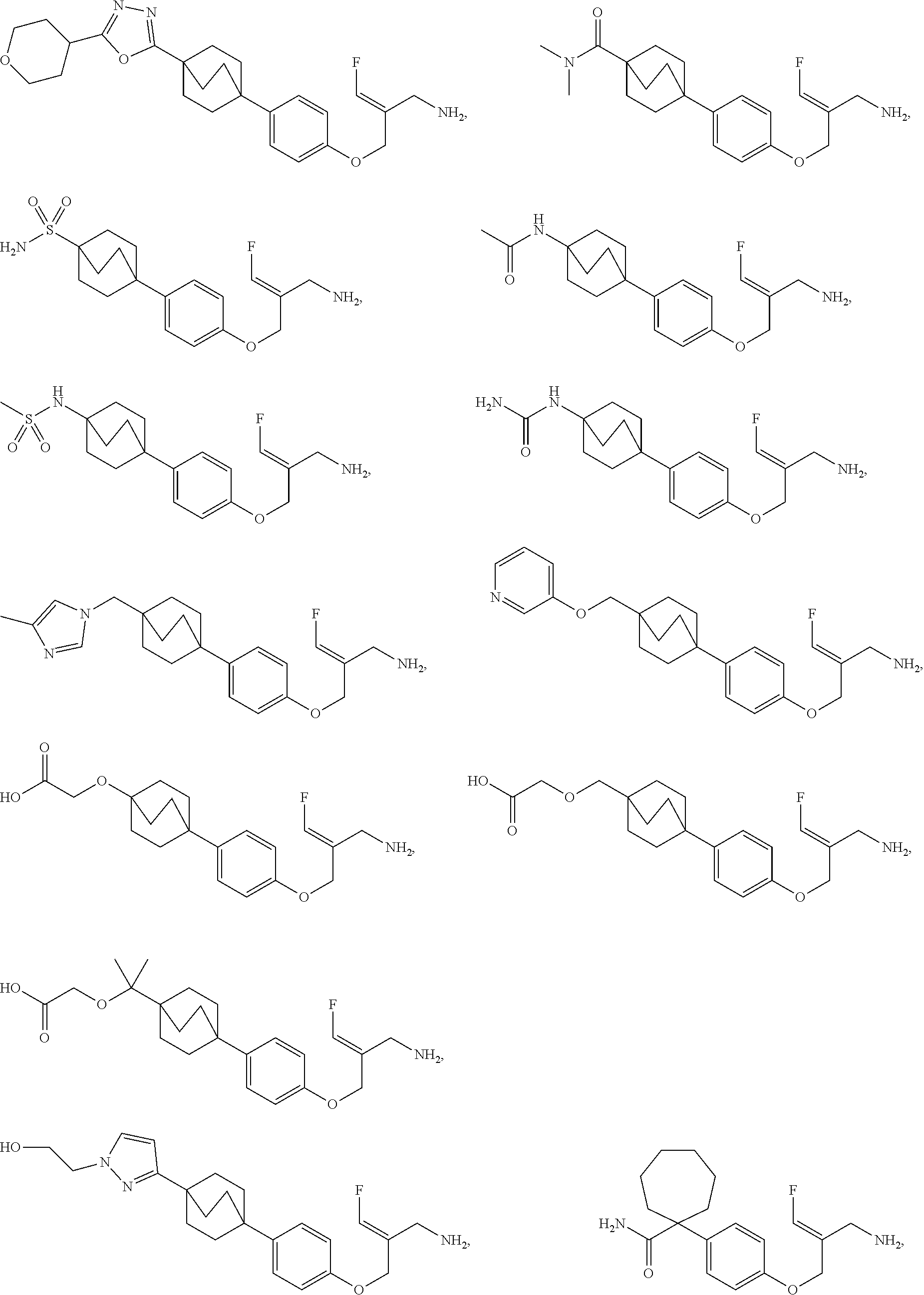 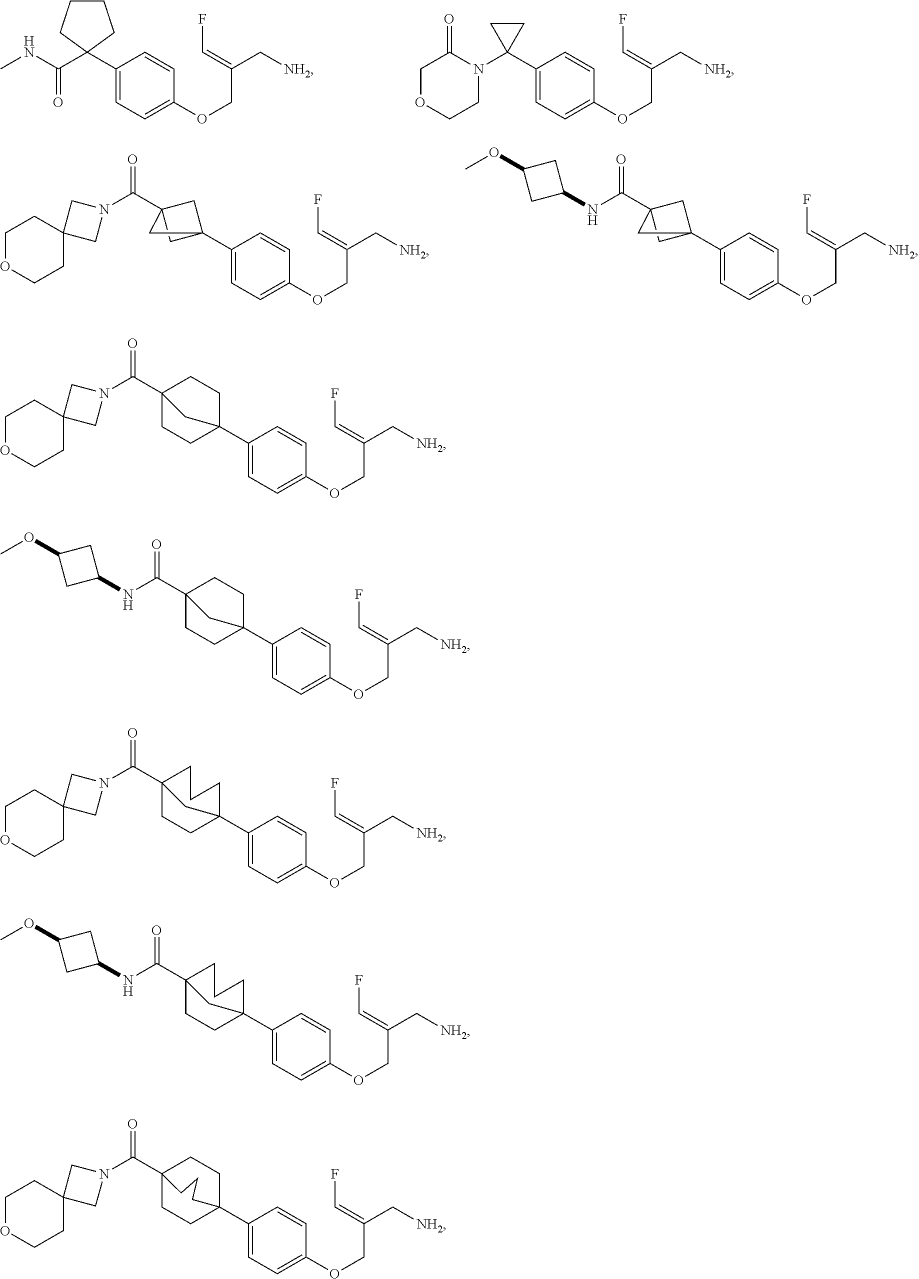 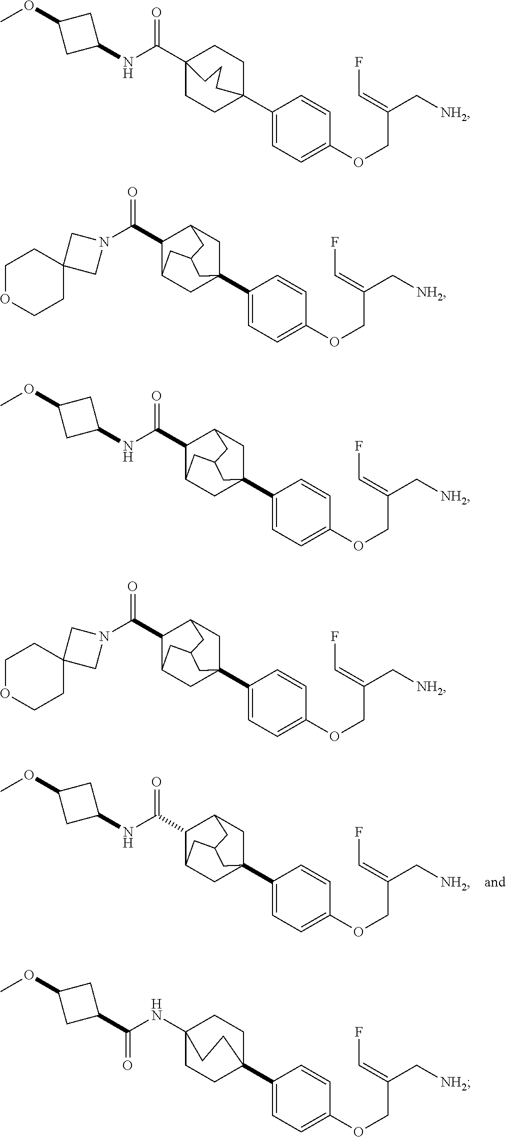
or a pharmaceutically acceptable salt or solvate thereof.

18. A pharmaceutical composition comprising a compound of claim 1, or a pharmaceutically acceptable salt or solvate thereof, and at least one pharmaceutically acceptable excipient.

19. A method of treating or preventing a liver disease or condition in a mammal, comprising administering to the mammal a compound of claim 1, or a pharmaceutically acceptable salt or solvate thereof.

20. The method of claim 19, wherein the liver disease or condition is nonalcoholic steatohepatitis (NASH) or nonalcoholic fatty liver disease (NAFLD).
